(19) United States
(12) Patent Application Publication
(10) Pub. No.: US 2025/0120952 A1
(43) Pub. Date: Apr. 17, 2025

(54) 17-Beta-Hydroxysteroid Dehydrogenase Type 13 Inhibitors And Methods Of Use Thereof

(71) Applicant: ENANTA PHARMACEUTICALS, INC., Watertown, MA (US)

(72) Inventors: Yaohui NIE, West Roxbury, MA (US); Manuel ROQUETA-RIVERA, Waltham, MA (US); Jordan BUTTS, Waltham, MA (US); Mary CHAU, Waltham, MA (US); Kelsey GARLICK, Waltham, MA (US)

(21) Appl. No.: 18/905,716

(22) Filed: Oct. 3, 2024

Related U.S. Application Data
(60) Provisional application No. 63/542,483, filed on Oct. 4, 2023.

(51) Int. Cl. A61K 31/426; A61K 31/196; A61K 31/343; A61K 31/404; A61K 31/415; A61K 31/4375; A61K 31/538; A61P 11/00

(52) U.S. Cl. CPC A61K 31/426; A61K 31/196; A61K 31/343; A61K 31/404; A61K 31/415; A61K 31/4375; A61K 31/538; A61P 11/00

(57) ABSTRACT

The present invention provides methods for treating a fibrotic disease using a compound of Formula (I),

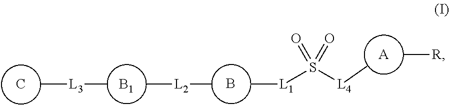

or a pharmaceutically acceptable salt thereof, where the variables are as defined herein. The invention further provides pharmaceutical compositions comprising a compound of Formula (I) which are useful in the methods of the invention.

### RELATED APPLICATION

This application claims the benefit of U.S. Provisional Application No. 63/542,483, filed on Oct. 4, 2023. The entire teachings of the above application are incorporated herein by reference.

### TECHNICAL FIELD

The present invention relates generally to compounds and pharmaceutical compositions useful as 17β-HSD13 inhibitors. Specifically, the present invention relates to compounds useful as inhibitors of 17β-HSD13 and methods for their preparation and use.

### BACKGROUND OF THE INVENTION

17-Beta-hydroxysteroid dehydrogenases (17β-HSDs) are NADP or NAD dependent oxidoreductases that catalyze oxidation/reduction reactions of 17β-hydroxysteroids or 17-ketosteroids, respectively. For example, 17β-HSDs can catalyze the interconversion of androstenedione with testosterone, estrone with estradiol, or dehydroepiandrosterone (DHEA) with androstenediol. Of the fifteen 17β-HSDs that have been identified, all but one (17β-HSD type 5) are short-chain dehydrogenases/reductases (SDRs) (J. M. Day, et al., Endocrine-Related Cancer 2008, 15, 665-692).

More specifically, 17-Beta-hydroxysteroid dehydrogenase type 13 (17β-HSD13) is encoded by the HSD17B13 gene. Further studies have shown that a 17β-HSD13 loss-of-function variant has been associated with a significantly reduced risk of NAFLD, cirrhosis associated with nonalcoholic steatohepatitis (NASH), alcoholic liver disease, alcoholic cirrhosis, hepatocellular carcinoma (HCC), NASH disease severity, ballooning degeneration, lobular inflammation, and fibrosis (N. S. Abul-Husn, et al., N. Engl. J. Med. 2018, 378, 1096-1106; C. J. Pirola, et al., J. Lipid Res. 2019, 60, 176-185).

Fibrosis can affect various organs and tissues in the body, such as the lungs (pulmonary fibrosis), liver (cirrhosis), kidney (renal fibrosis), heart (cardiac fibrosis), and skin (scleroderma), among others.

Pulmonary fibrosis is one of the most critical fibrotic diseases to treat. Pulmonary fibrosis is defined as the progressive replacement of alveolar tissue with fibrotic scar that threatens alveolar gas exchange and reduces lung compliance. The resulting increased work of breathing and hypoxemia lead to progressive respiratory failure and eventual death. There are two main types of pulmonary fibrosis: idiopathic pulmonary fibrosis (IPF) and secondary pulmonary fibrosis. Idiopathic pulmonary fibrosis refers to cases where the specific cause of the condition is unknown, whereas secondary pulmonary fibrosis is associated with known causes, such as certain occupational exposures, connective tissue diseases, medication side effects, or other underlying medical conditions. The clinical and economic burden of pulmonary fibrosis is sizeable: the worldwide incidence of IPF is increasing and attributable medical costs for patients with IPF in the United States alone have been estimated at nearly 2 billion dollars (Hutchinson J, Fogarty A, Hubbard R, McKeever T. Global incidence and mortality of idiopathic pulmonary fibrosis: a systematic review. Eur Respir J. 2015; 46:795-806). Despite decades of research, only a handful of therapies are available. Although these therapies slow disease progression, the response is variable, and lung transplantation represents the only option for temporary cure. Also, both pirfenidone and nintedanib have been shown in clinical trials to slow the decline in lung function, decrease the rate of disease progression, and improve overall quality of life for some individuals with IPF. It's important to note that not all patients respond the same way to these medications, and individual responses can vary. Accordingly, there is an urgent need to provide molecular that can be used to diagnosis and therapy for pulmonary fibrosis disease.

There is a need for therapeutic agents and methods for the treatment and prevention of fibrotic diseases, particularly pulmonary fibrosis.

### SUMMARY OF THE INVENTION

The present invention relates to compositions and methods for treating a fibrotic disease.

In one aspect, the invention provides a method of treating a fibrotic disease in a subject in need thereof, comprising administering to the subject a therapeutically effective amount of a compound of Formula I,

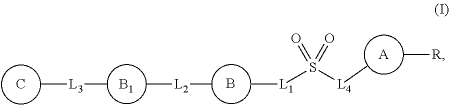

or a pharmaceutically acceptable salt thereof, wherein:
L₁ and L₄ are each independently absent or NR₁;
R₁ is hydrogen, optionally substituted methyl, or optionally substituted acetyl;
\textcircled{B} is absent or selected from the group consisting of optionally substituted aryl, optionally substituted heteroaryl, optionally substituted arylalkyl, optionally substituted heteroarylalkyl, optionally substituted —C₃-C₁₂ cycloalkyl, optionally substituted 3- to 12-membered heterocycloalkyl, and optionally substituted —C₃-C₁₂ cycloalkenyl;
Alternatively, L₁ is NR₁, R₁ is connected to \textcircled{B}, and R₁ and the nitrogen atom to which it is attached form a heterocyclic ring;
L₂ is absent or selected from the groups consisting of —NR₂—, —O—, optionally substituted —C₁-C₆ alkyl, optionally substituted —C₂-C₆ alkenyl and optionally substituted —C₂-C₆ alkynyl;
R₂ is hydrogen, optionally substituted —C₁-C₆ alkyl, optionally substituted —C₃-C₁₂ cycloalkyl, and optionally substituted 3- to 12-membered heterocycloalkyl;
\textcircled{B₁} is absent or selected from the groups consisting of optionally substituted aryl, optionally substituted heteroaryl, optionally substituted —C₃-C₁₂ cycloalkyl, optionally substituted 3- to 12-membered heterocycloalkyl, and optionally substituted —C₃-C₁₂ cycloalkenyl;
L₃ is absent or selected from the group consisting of optionally substituted —C₁-C₆ alkyl, optionally substituted —C₂-C₆ alkenyl, optionally substituted —C₂-C₆ alkynyl, optional substituted-aliphatic which contains 1-6 carbon atoms and at least one other atom selected from oxygen, nitrogen and sulfur atom, optionally substituted —C₃-C₁₂ cycloalkyl, optionally substituted 3- to 12-membered heterocycloalkyl, —O—, —NR₂—, —C(O)—, —OC(O)—, —C(O)O—, —NR₂C(O)—, —C(O)NR₂—, —S(O)₂—, —S(O)₂NR₂—, and —NR₂S(O)₂—, wherein R₂ is as previously defined;
\textcircled{C} is absent or selected from the groups consisting of optionally substituted aryl, optionally substituted heteroaryl, optionally substituted arylalkyl, optionally substituted heteroarylalkyl optionally substituted —C₃-C₁₂ cycloalkyl, optionally substituted 3- to 12-membered heterocycloalkyl, and optionally substituted —C₃-C₁₂ cycloalkenyl;
\textcircled{A} is optionally substituted aryl, optionally substituted heteroaryl, optionally substituted arylalkyl, optionally substituted heteroarylalkyl, optionally substituted —C₃-C₁₂ cycloalkyl, optionally substituted 3- to 12-membered heterocycloalkyl; or optionally substituted —C₃-C₁₂ cycloalkenyl;
Alternatively, L₄ is NR₁, R₁ is connected to \textcircled{A}, and R₁ and the nitrogen atom to which it is attached form a heterocyclic ring;
R is absent or selected from the group consisting of hydroxy, protected hydroxy, —O-(hydroxy prodrug group), tetrazolyl, 1-methyltetrazolyl, —CH₂CO₂R₃, —CO₂R₃, —C(O)N(R₃)O(R₃), —C(O)NR₄R₅, —P(O)(OR₆)₂, —C(O)NR₇S(O)₂R₈, —S(O)₂R₈, —CH₂OH, —CH₂OC(O)NR₄R₅, and —CH₂OC(O)NR₇S(O)₂R₈;
Each R₃ is independently selected from the groups consisting of hydrogen, optionally substituted —C₁-C₈ alkyl, and optionally substituted —C₃-C₈ cycloalkyl;
R₄ and R₅ are each independently selected from the group consisting of hydrogen, optionally substituted —C₁-C₈ alkyl, and optionally substituted —C₃-C₈ cycloalkyl; alternatively, R₄ and R₅ are taken together with the nitrogen atom to which they are attached to form a heterocyclic ring;
R₆ is selected from the group consisting of hydrogen, optionally substituted —C₁-C₈ alkyl, optionally substituted —C₃-C₈ cycloalkyl, optionally substituted 3- to 8-membered heterocycloalkyl, optionally substituted aryl, and optionally substituted heteroaryl;
R₇ is selected from the group consisting of hydrogen, optionally substituted —C₁-C₆ alkyl, and optionally substituted —C₃-C₆ cycloalkyl;
R₈ is selected from the group consisting of NR₄R₅ optionally substituted —C₁-C₈ alkyl, optionally substituted —C₃-C₈ cycloalkyl, optionally substituted 3- to 8-membered heterocycloalkyl, optionally substituted aryl, and optionally substituted heteroaryl; provided that at least one of \textcircled{B}, \textcircled{B₁}, and \textcircled{C} is not absent.

The present invention further provides a pharmaceutical composition comprising a compound of Formula (I), or a pharmaceutically acceptable salt thereof, and a pharmaceutically acceptable carrier or excipient, which is useful for treating a fibrotic disease.

### BRIEF DESCRIPTION OF THE DRAWINGS

FIG. 1 is a graph showing the time course of HSD17B13 protein abundance after bleomycin induced lung fibrosis.

FIG. 2 shows lung samples from the bleomycin-induced lung fibrosis model following staining for HSD17B13 and SCGB1A1.

FIG. 3 is a western blot showing elevated HSD17B13 protein presence in fibrotic human lung tissue.

FIG. 4 shows immunohistochemical localization of HSD17B13 in normal human lung tissue within bronchiole but not alveoli.

FIG. 5 shows immunohistochemical detection and quantification of HSD17B13 and SCGB1A1 comparing human lung tissue from normal and idiopathic pulmonary fibrosis donors.

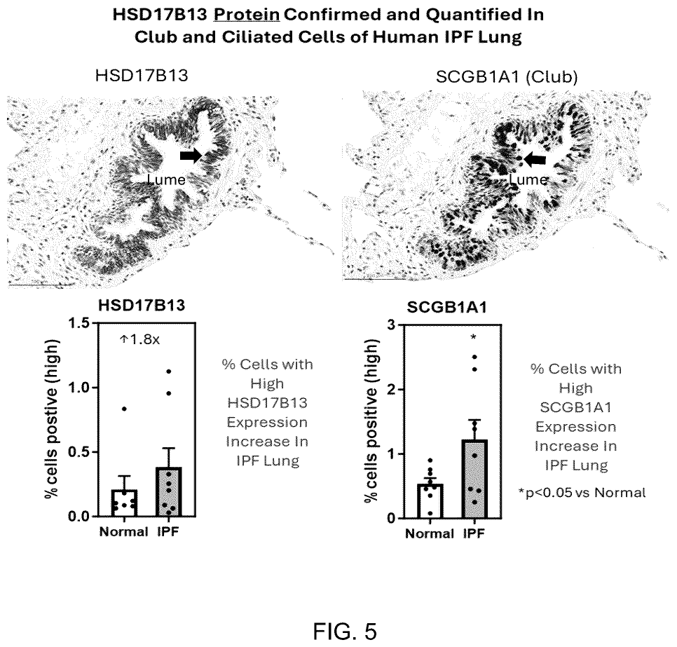

FIG. 6 presents graphs showing the effect of Compound 812 at 1 uM (grey line) versus vehicle treatment (black line) on the expression of lung epithelial markers (SCGB1A1, MUC5B) and fibrotic related genes (TGFB2, ACTA2) during the differentiation of primary human small airway epithelial cells (HSAEC) in an air-liquid interphase culture.

FIG. 7 is a graph showing the effects of compounds 790 and 812 on gene and secreted protein markers of fibrosis in precision cut lung slices (PCLS) from IPF donors (n=5).

FIG. 8 is a graph showing the effects of compounds 790 and 812 on gene and secreted protein markers of fibrosis in precision cut lung slices (PCLS) from fibrotic cocktail (FC)-treated normal donors. Five IPF PCLS donors were tested and efficacy by compounds was observed in all cases.

FIG. 9 is a Western blot showing abundance of HSD17B13 protein in the H441 lung epithelial cell line.

FIG. 10 is a graph showing the effects of HSD17B13 inhibitors on enzymatic activity.

FIG. 11 is a graph showing the effects of HSD17B13 inhibitors on Collagen and COL1A1 mRNA in H441 cells.

### DETAILED DESCRIPTION OF THE INVENTION

The present invention provides methods and compositions for treating a fibrotic disease as described above.

In certain embodiments of the methods and compositions of the invention, L₁ is —NR₁—.

In certain embodiments of the methods and compositions of the invention, L₁ is —NH—.

In certain embodiments of the methods and compositions of the invention, L₄ is —NR₁—.

In certain embodiments of the methods and compositions of the invention, L₄ is —NH—.

In certain embodiments of the methods and compositions of the invention, L₁ is —NH— and L₄ is absent.

In certain embodiments of the methods and compositions of the invention, L₄ is —NH—, and L₁ is absent.

In certain embodiments of the methods and compositions of the invention, L₁ is —NH—, and L₄ is —NH—.

In certain embodiments of the methods and compositions of the invention, \textcircled{A} is optionally substituted aryl.

In certain embodiments of the methods and compositions of the invention, \textcircled{A} is optionally substituted heteroaryl.

In certain embodiments of the methods and compositions of the invention, \textcircled{A} is optionally substituted phenyl.

In certain embodiments of the methods and compositions of the invention, \textcircled{A} is

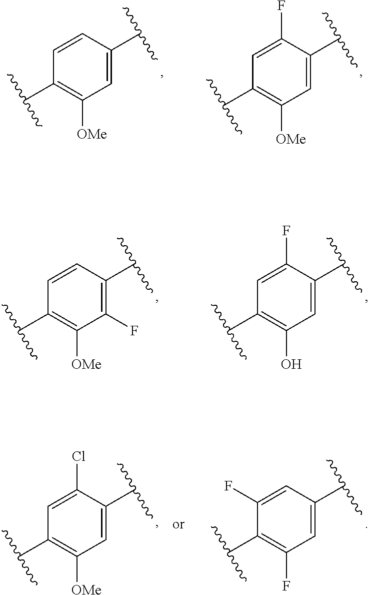 [SMILES: *c1ccc(*)c(OC)c1 ; *c1cc(OC)c(*)cc1F ; *c1ccc(*)c(OC)c1F ; *c1cc(F)c(*)cc1O ; *c1cc(OC)c(*)cc1Cl ; *c1cc(F)c(*)c(F)c1]

In certain embodiments of the methods and compositions of the invention, R is —CO₂H.

In certain embodiments of the methods and compositions of the invention, R is optionally substituted tetrazolyl, such as 5-tetrazolyl or 1-methyl-5-tetrazolyl.

In certain embodiments of the methods and compositions of the invention, R is —CH₂OH.

In certain embodiments of the methods and compositions of the invention, R is —CH₂OC(O)NR₄R₅, or —CH₂OC(O)NR₇S(O)₂R₈, and R₄, R₅, R₇, and R₈ are as previously defined.

In certain embodiments of the methods and compositions of the invention, R is —P(O)(OR₆)₂ or —C(O)NR₇S(O)₂R₈, and —R₆, R₇, and R₈ are as previously defined.

In certain embodiments of the methods and compositions of the invention, \textcircled{B} is optionally substituted aryl or optionally substituted heteroaryl.

In certain embodiments of the methods and compositions of the invention, \textcircled{B} is selected from the groups set forth below by removal of two hydrogen atoms, and \textcircled{B} is optionally substituted:

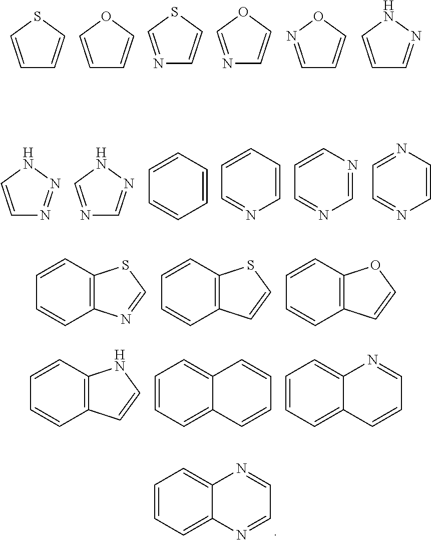

In certain embodiments of the methods and compositions of the invention is selected from the groups set forth below, and \textcircled{B} is optionally substituted:

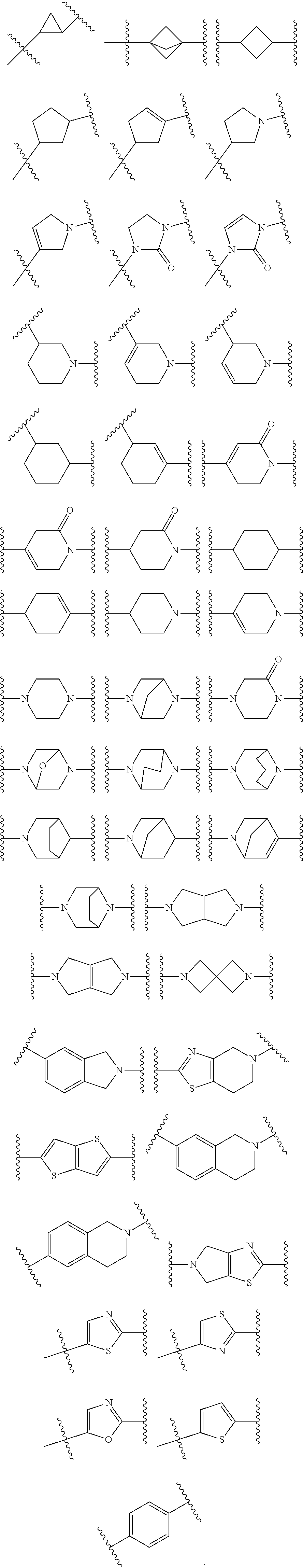

In certain embodiments of the methods and compositions of the invention, L₂ is absent.

In certain embodiments of the methods and compositions of the invention, L₂ is —NR₂—, and R₂ is as previously defined.

In certain embodiments of the methods and compositions of the invention, L₂ is —O—, —NH— or

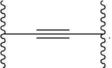 [SMILES: *C#C*]

In certain embodiments of the methods and compositions of the invention, L₂ is optionally substituted —C₁-C₆-alkyl.

In certain embodiments of the methods and compositions of the invention, L₂ is —C₂-C₆-alkynyl, such as ethynyl.

In certain embodiments of the methods and compositions of the invention, L₃ is absent.

In certain embodiments of the methods and compositions of the invention, L₃ is optionally substituted —C₁-C₆-alkyl.

In certain embodiments of the methods and compositions of the invention, L₃ is —O— or —NR₂—, and R₂ is as previously defined.

In certain embodiments, the present invention relates to methods and compositions of the invention, and pharmaceutically acceptable salts thereof, wherein L₃ is —C(O)—, —OC(O)—, —C(O)O—, —S(O)₂—, —S(O)₂NR₂—, or —NR₂S(O)₂—.

In certain embodiments of the methods and compositions of the invention, L₃ is optionally substituted —C₁-C₆ alkyl, optionally substituted —C₂-C₈ heteroalkyl, optionally substituted —C₁-C₆ alkenyl, or optionally substituted —C₃-C₈ heteroalkenyl.

In certain embodiments of the methods and compositions of the invention, \textcircled{C} is absent and L₃ is selected from the groups below:

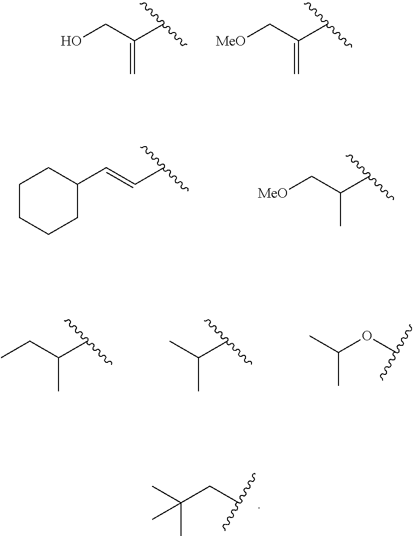

In certain embodiments of the methods and compositions of the invention, L₂ is absent, and L₃ is absent.

In certain embodiments of the methods and compositions of the invention, \textcircled{B₁} is selected from the groups set forth below by removal of two hydrogen atoms, and \textcircled{B₁} is optionally substituted:

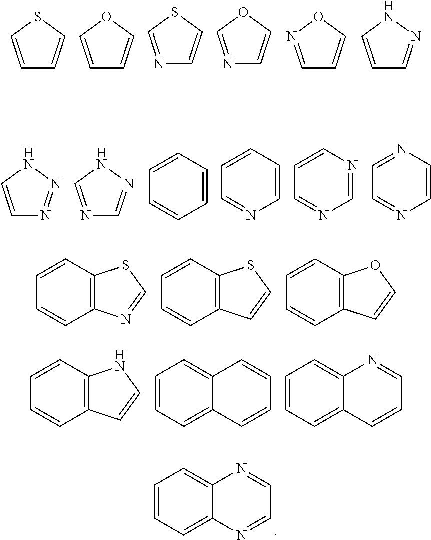

In certain embodiments of the methods and compositions of the invention, \textcircled{B₁} is selected from the groups set forth below, and \textcircled{B₁} is optionally substituted:

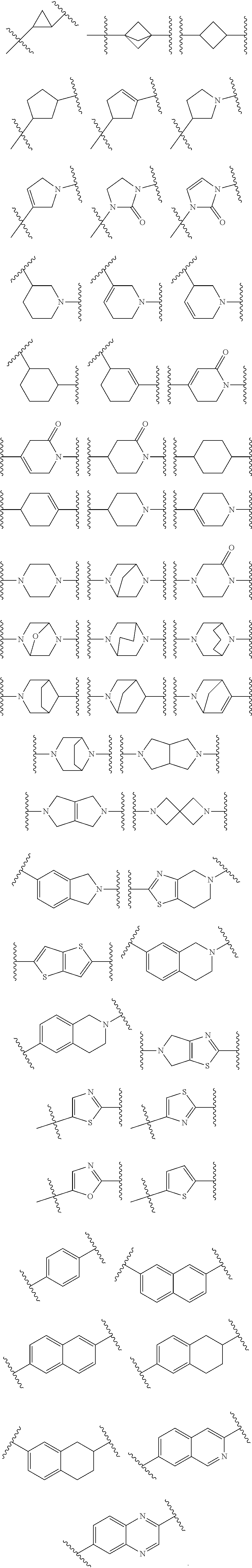

In certain embodiments of the methods and compositions of the invention, \textcircled{C} is absent.

In certain embodiments of the methods and compositions of the invention, \textcircled{C} is optionally substituted aryl. Preferably, \textcircled{C} is optionally substituted phenyl.

In certain embodiments of the methods and compositions of the invention, \textcircled{C} is optionally substituted heteroaryl.

In certain embodiments of the methods and compositions of the invention, \textcircled{C} is optionally substituted —C₃-C₁₂ cycloalkyl, such as, but not limited to, optionally substituted cyclobutyl, optionally substituted cyclopentyl, optionally substituted cyclohexyl.

In certain embodiments of the methods and compositions of the invention, \textcircled{C} is optionally substituted —C₄-C₁₂ cycloalkenyl, such as, but not limited to, optionally substituted cyclopentenyl, optionally substituted cyclohexenyl.

In certain embodiments of the methods and compositions of the invention, \textcircled{C} is optionally substituted 3 to 12 membered heterocycloalkyl, such as, but not limited to, optionally substituted pyrrolidinyl or optionally substituted piperidinyl.

In certain embodiments of the methods and compositions of the invention, \textcircled{C} is selected from the groups set forth below by removal of one hydrogen atoms, and \textcircled{C} is optionally substituted:

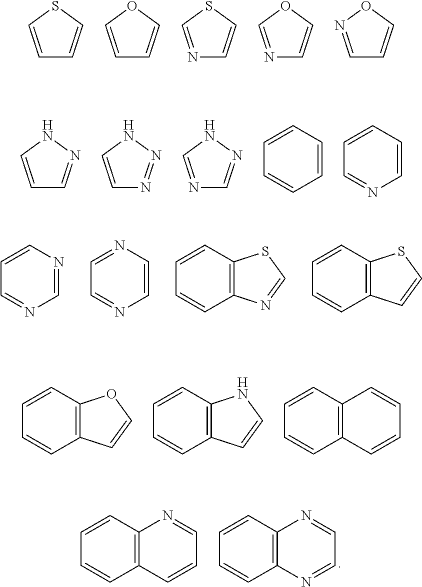

In certain embodiments of the methods and compositions of the invention, \textcircled{C} is selected from the groups set forth below, and \textcircled{C} is optionally substituted:

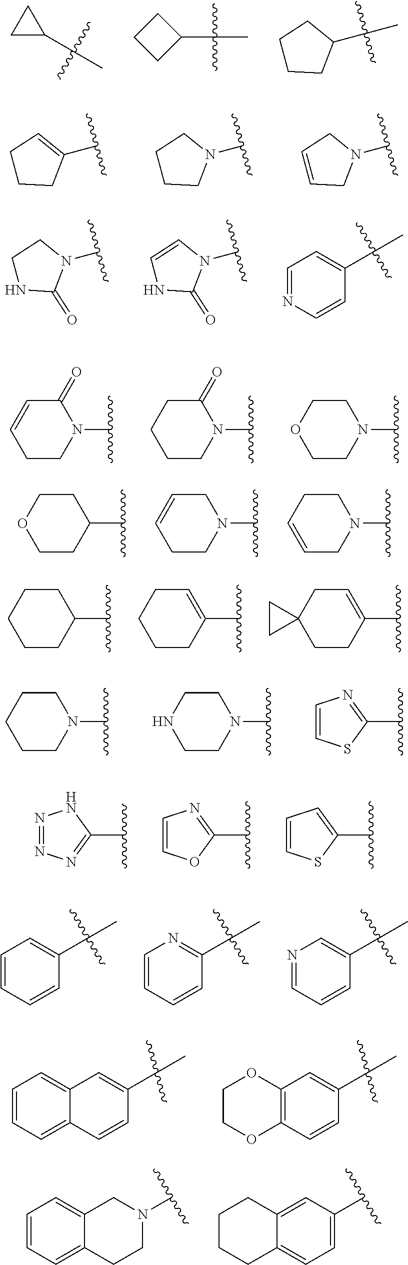

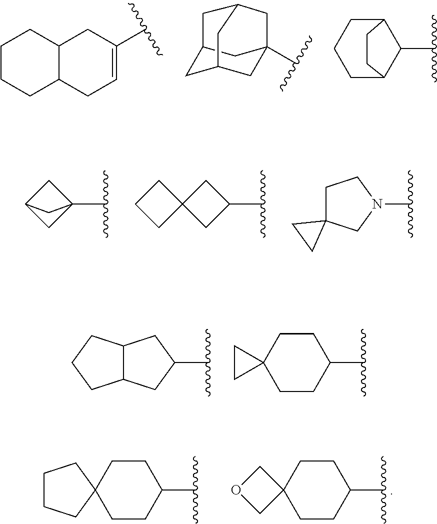

In certain embodiments of the methods and compositions of the invention, the compound of Formula (I) is represented by Formula (Ia), or a pharmaceutically acceptable salt thereof:

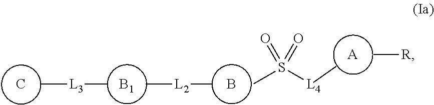

wherein \textcircled{A}, \textcircled{B}, \textcircled{B₁}, \textcircled{C}, L₂, L₃, L₄, and R, are as previously defined.

In certain embodiments of the methods and compositions of the invention, the compound of Formula (I) is represented by Formula (Tb), or a pharmaceutically acceptable salt thereof:

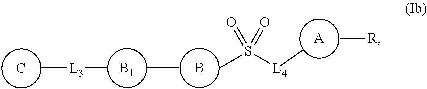

wherein \textcircled{A}, \textcircled{B}, \textcircled{B₁}, \textcircled{C}, L₃, L₄, and R, are as previously defined.

In certain embodiments of the methods and compositions of the invention, the compound of Formula (I) is represented by Formula (Ic), or a pharmaceutically acceptable salt thereof:

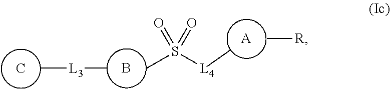

wherein \textcircled{A}, \textcircled{B}, \textcircled{C}, L₃, L₄, and R, are as previously defined.

In certain embodiments of the methods and compositions of the invention, the compound of Formula (I) is represented by Formula (Id), or a pharmaceutically acceptable salt thereof:

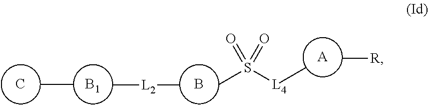

wherein \textcircled{A}, \textcircled{B}, \textcircled{B₁}, \textcircled{C}, L₂, L₄, and R, are as previously defined.

In certain embodiments of the methods and compositions of the invention, the compound of Formula (I) is represented by Formula (Ie), or a pharmaceutically acceptable salt thereof:

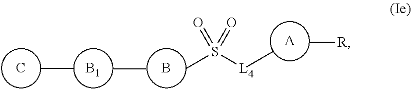

wherein \textcircled{A}, \textcircled{B}, \textcircled{B₁}, \textcircled{C}, L₄, and R, are as previously defined.

In another embodiment, the compound of Formula (I) is represented by one of Formulae (II-1) to (II-6), or a pharmaceutically acceptable salt thereof:

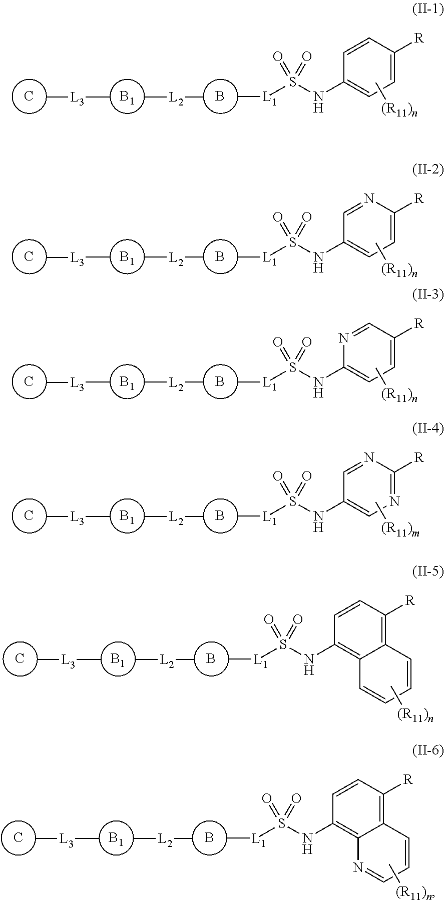

wherein each R₁₁ is independently selected from the group consisting of halogen, hydroxy, —NH₂, —NHMe, —NMe₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; n is 0, 1, 2, or 3; m is 0, 1 or 2; and, \textcircled{B}, \textcircled{B₁}, \textcircled{C}, L₁, L₂, L₃, and R are as previously defined.

In another embodiment, the compound of Formula (I) is represented by one of Formulae (III-1) to (III-6), or a pharmaceutically acceptable salt thereof:

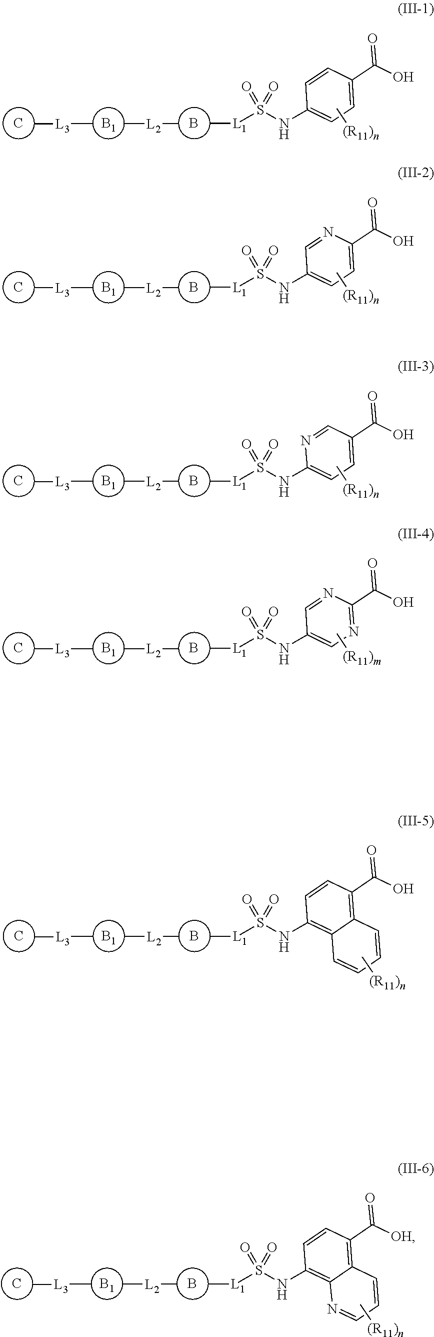

wherein R₁₁, m, n, \textcircled{B}, \textcircled{B₁}, \textcircled{C}, L₁, L₂, and L₃ are as previously defined.

In another embodiment, the compound of Formula (I) is represented by one of Formulae (III-1a) to (III-1f), or a pharmaceutically acceptable salt thereof:

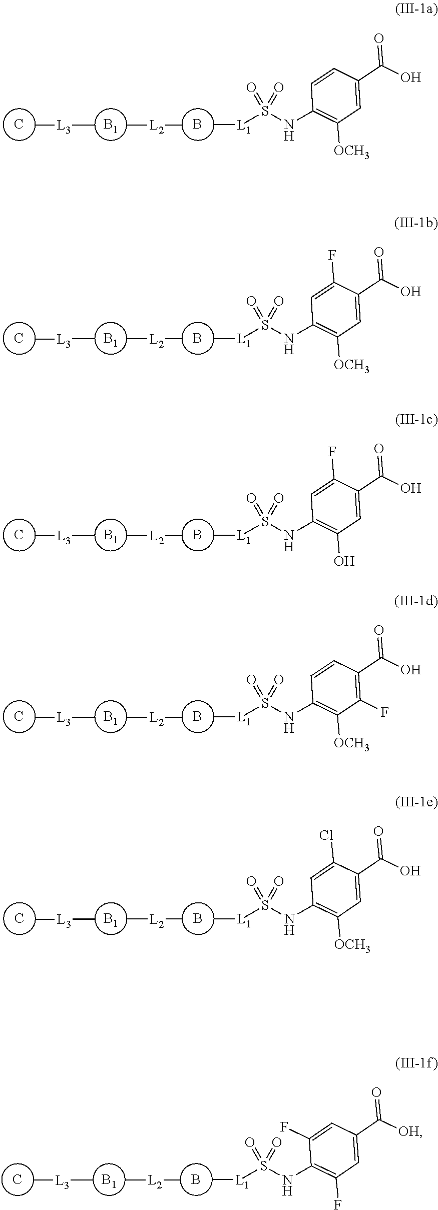

wherein \textcircled{B}, \textcircled{B₁}, \textcircled{C}, L₁, L₂, and L₃ are as previously defined.

In another embodiment, the compound of Formula (I) is represented by one of Formulae (IV-1) to (IV-6), or a pharmaceutically acceptable salt thereof:

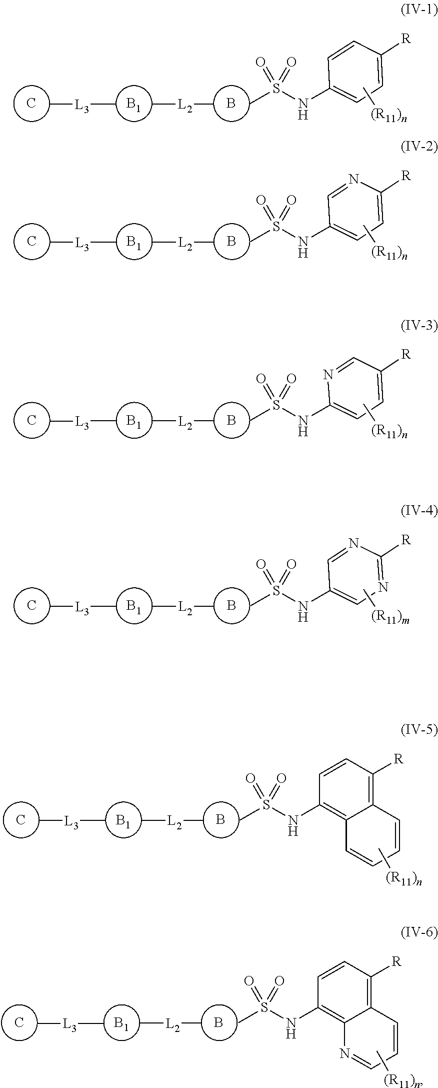

wherein R₁, m, n, \textcircled{B}, \textcircled{B₁}, \textcircled{C}, L₂, L₃, and R are as previously defined.

In another embodiment, the compound of Formula (I) is represented by one of Formulae (V-1) to (V-10), or a pharmaceutically acceptable salt thereof:

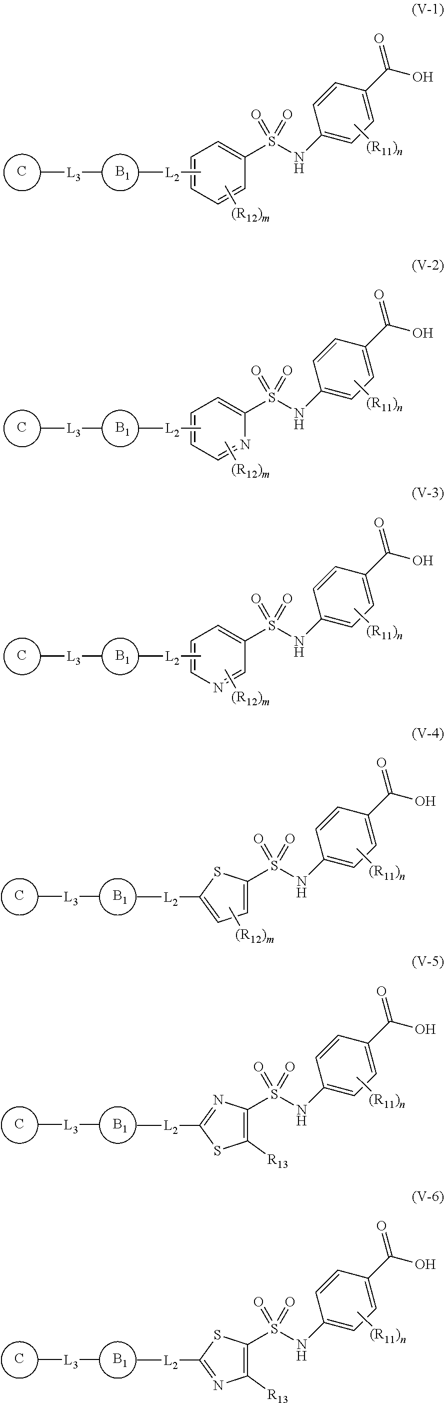

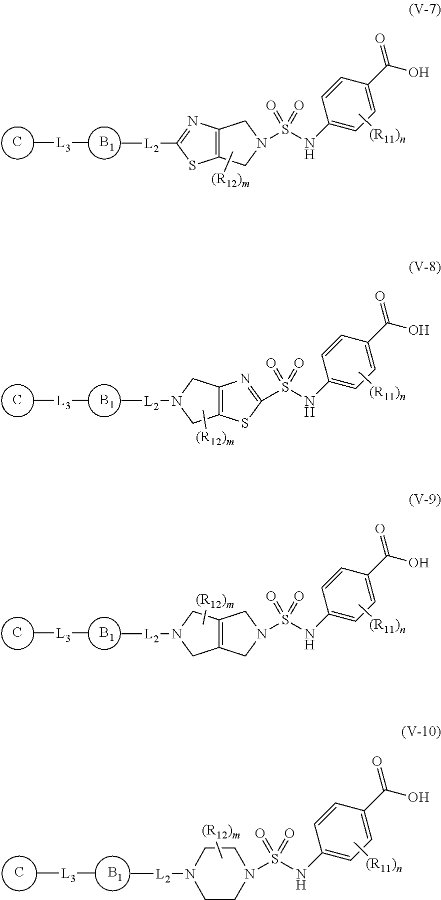

wherein each R₁₂ is independently selected from the group consisting of halogen, hydroxy, —NH₂, —NHMe, —NMe₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; R₁₃ is independently selected from the group consisting of hydrogen, halogen, hydroxy, —NH₂, —NHMe, —NMe₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; and R₁, m, n, \textcircled{B₁}, \textcircled{C}, L₂, and L₃ are as previously defined.

In another embodiment, the compound of Formula (I) is represented by one of Formulae (VI-1) to (VI-6), or a pharmaceutically acceptable salt thereof:

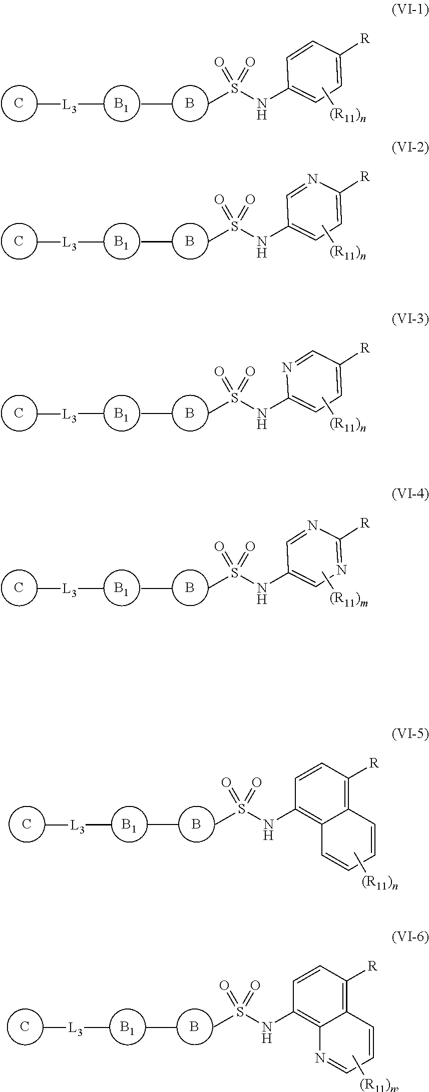

wherein R₁, m, n, \textcircled{B}, \textcircled{B₁}, \textcircled{C}, L₃, and R are as previously defined.

In another embodiment, the compound of Formula (I) is represented by one of Formulae (VII-1) to (VII-10), or a pharmaceutically acceptable salt thereof:

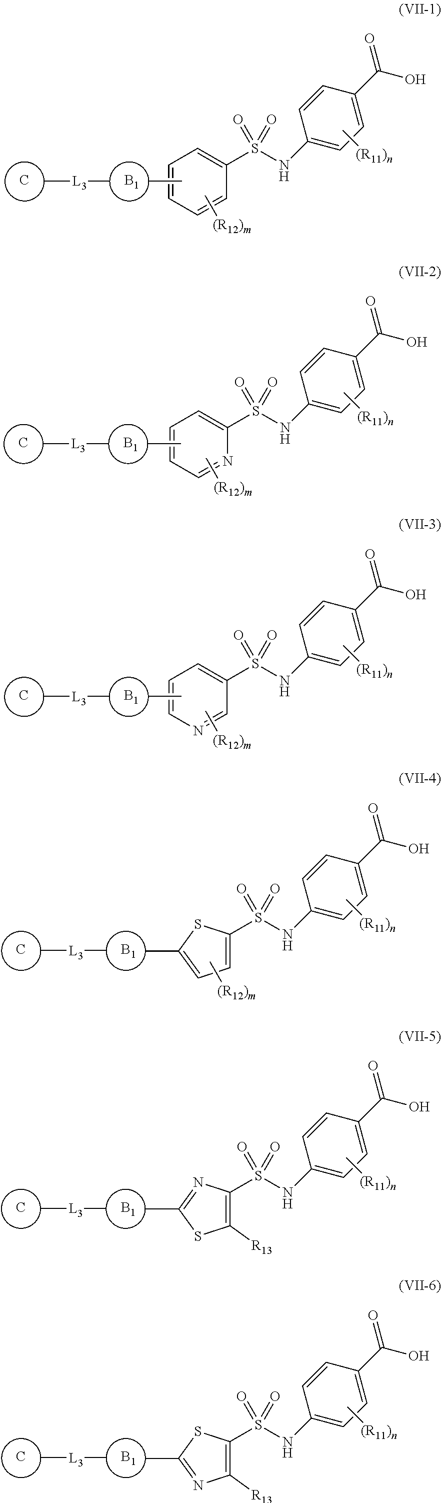

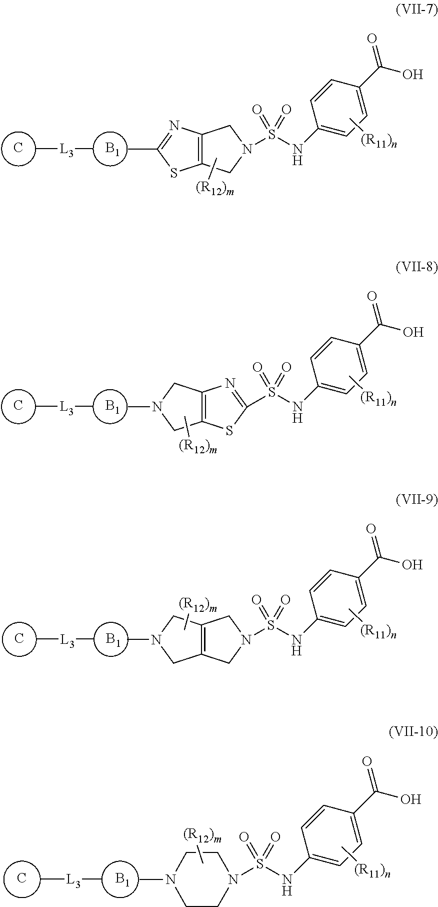

wherein R₁₁, R₁₂, R₁₃, m, n, \textcircled{B₁}, \textcircled{C}, and L₃ are as previously defined.

In another embodiment, the compound of Formula (I) is represented by one of Formulae (VIII-1) to (VIII-10), or a pharmaceutically acceptable salt thereof:

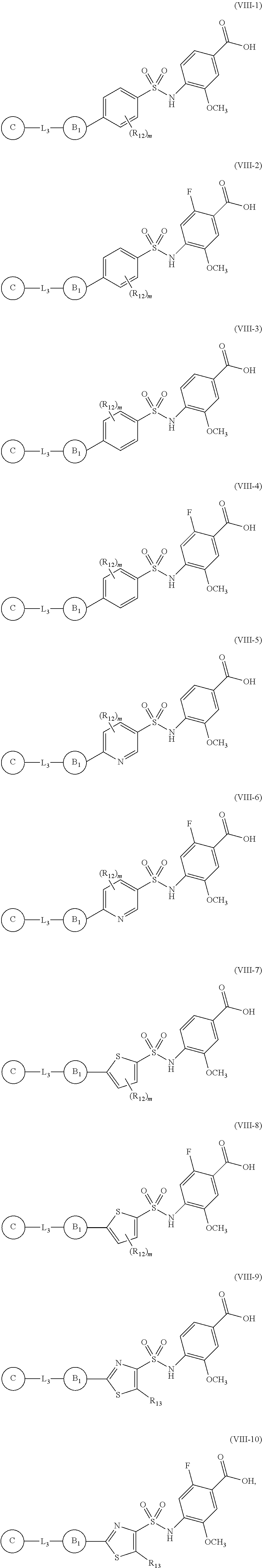

wherein R₁₂, R₁₃, m, \textcircled{B₁}, \textcircled{C}, and L₃ are as previously defined.

In certain embodiments of the methods and compositions of the invention, the compound of Formula (I) is represented by one of Formulae (VIII-1) to (VIII-10), or a pharmaceutically acceptable salt thereof, wherein \textcircled{B₁} is selected from the groups below, and \textcircled{B₁} is optionally substituted:

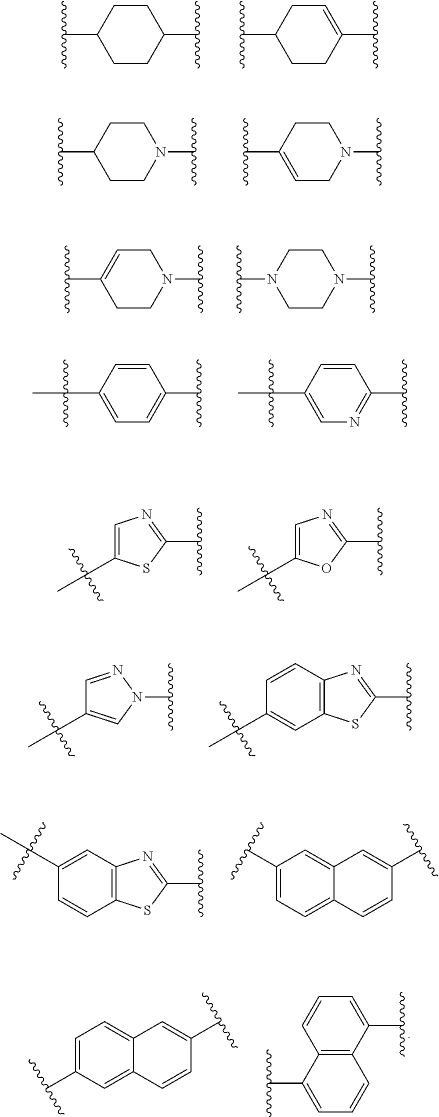

In certain embodiments of the methods and compositions of the invention, the compound of Formula (I) is represented by one of Formulae (VIII-1) to (VIII-10), or a pharmaceutically acceptable salt thereof, wherein L₃ is —C(O)—, —OC(O)—, or —C(O)O—, \textcircled{B₁} is selected from the groups below, and \textcircled{B₁} is optionally substituted:

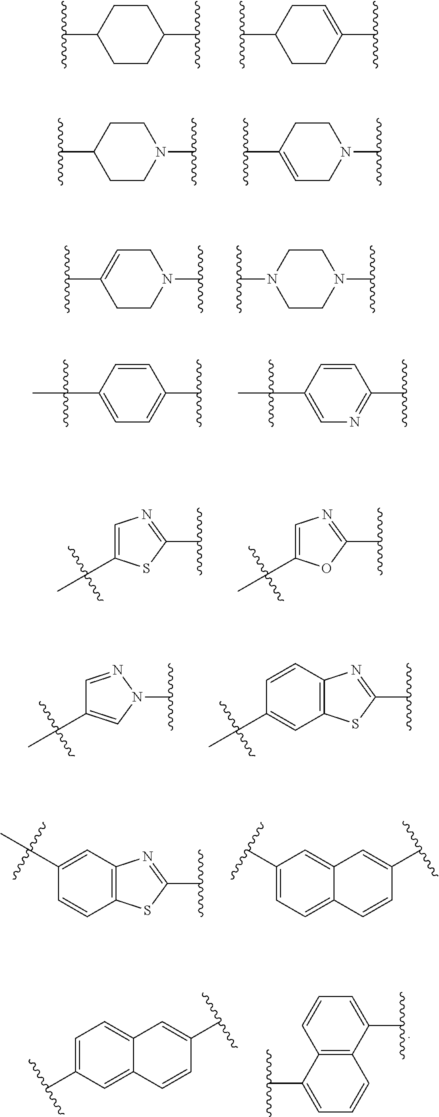

In another embodiment, the compound of Formula (I) is represented by one of Formulae (IX-1) to (IX-6), or a pharmaceutically acceptable salt thereof:

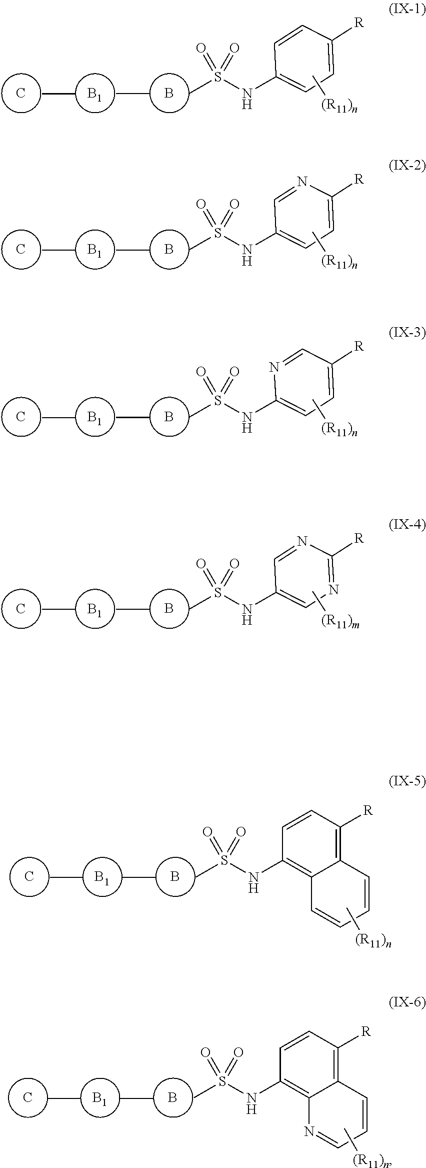

wherein R₁, m, n, \textcircled{B}, \textcircled{B₁}, \textcircled{C}, and R are as previously defined.

In another embodiment, the compound of Formula (I) is represented by one of Formulae (X-1) to (X-12), or a pharmaceutically acceptable salt thereof:

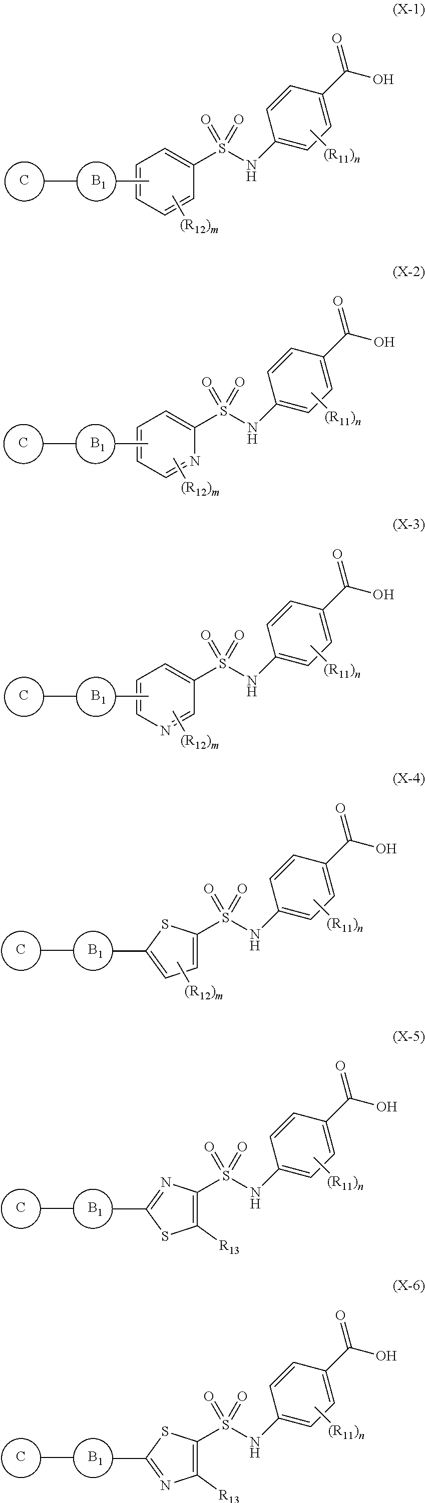

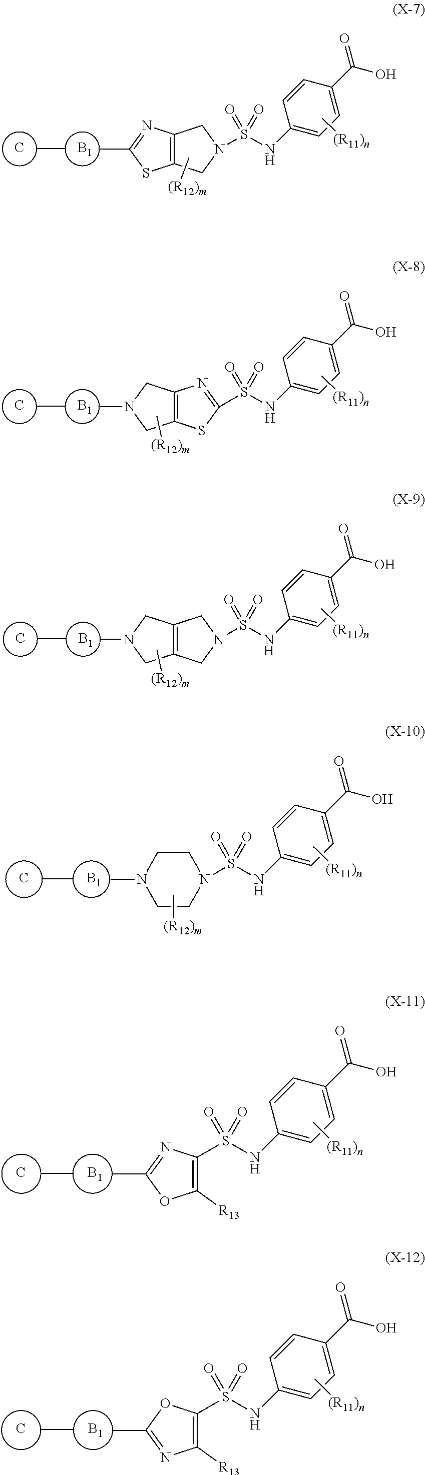

wherein R₁, R₁₂, R₁₃, m, n, \textcircled{B₁} and \textcircled{C} are as previously defined.

In another embodiment, the compound of Formula (I) is represented by one of Formulae (XI-1) to (XI-22), or a pharmaceutically acceptable salt thereof:

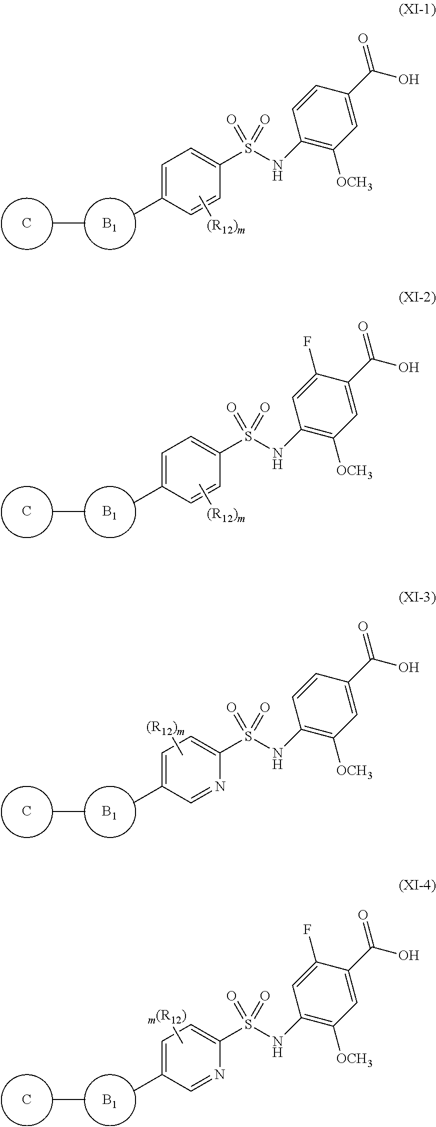

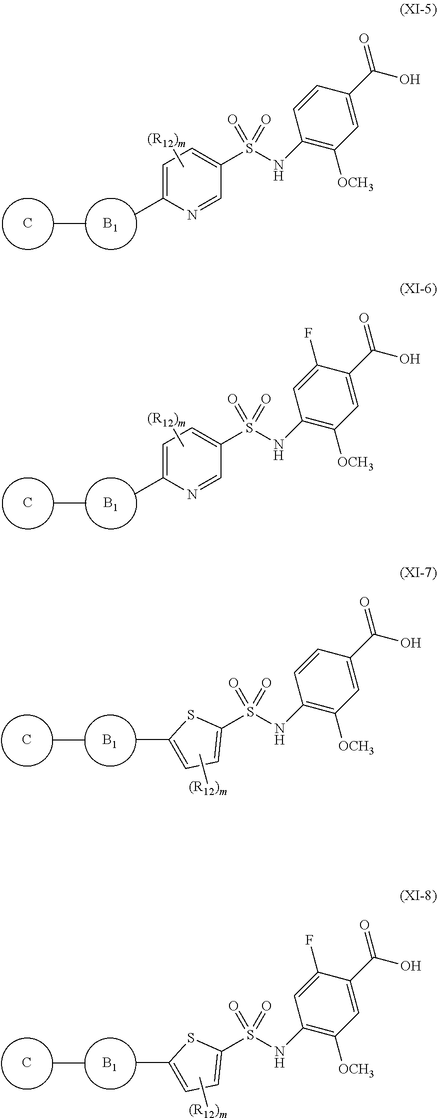

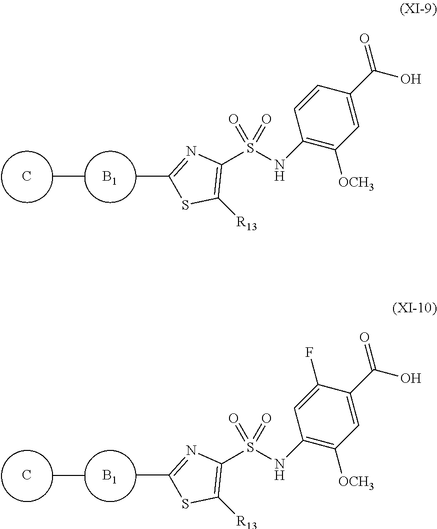

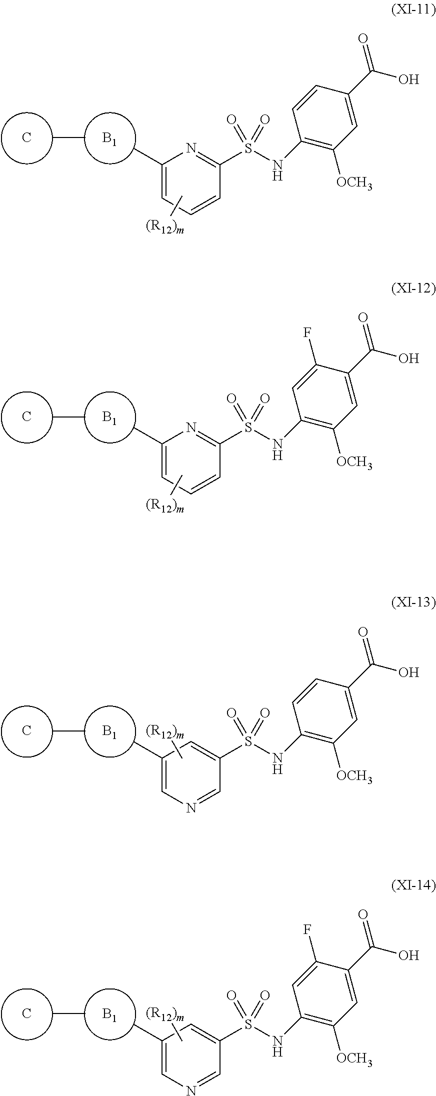

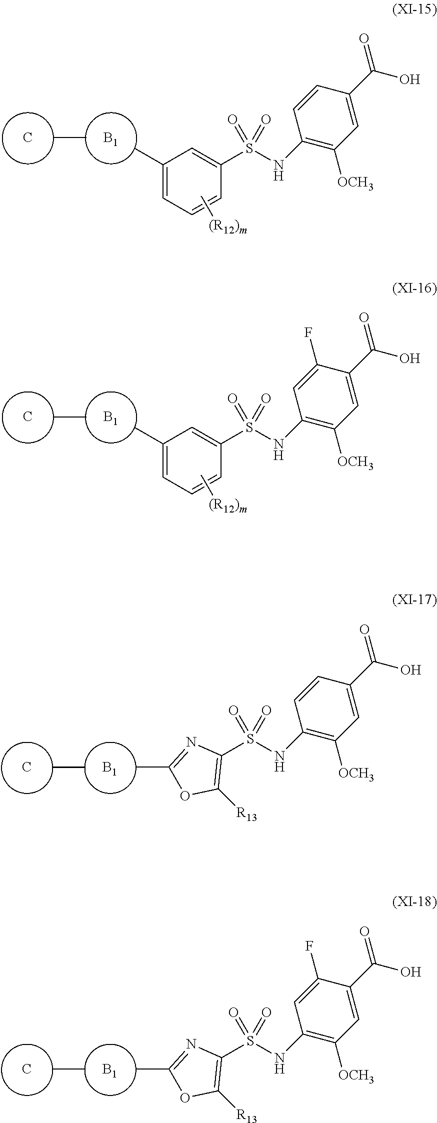

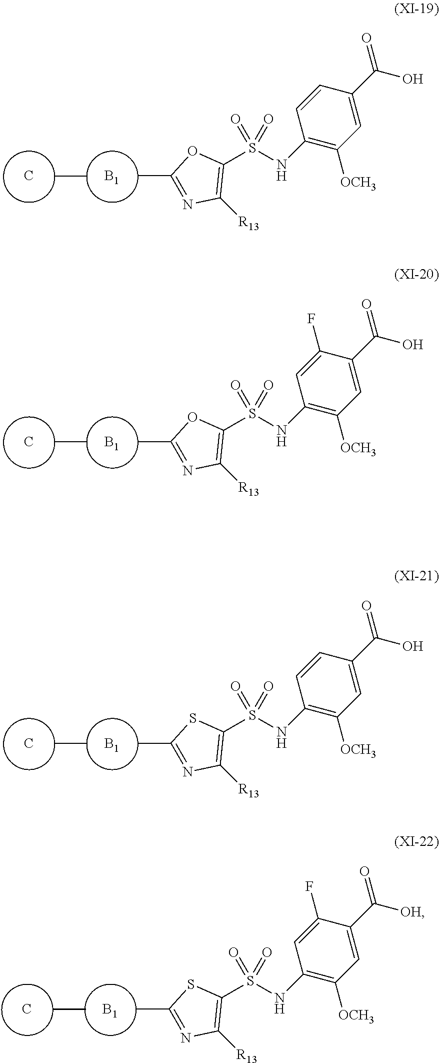

wherein R₁₂, R₁₃, m, \textcircled{B₁} and \textcircled{C} are as previously defined.

In certain embodiments of the methods and compositions of the invention, the compound of Formula (I) is represented by one of Formulae (XI-1) to (XI-22), or a pharmaceutically acceptable salt thereof, where \textcircled{B₁} is selected from the groups below, and \textcircled{B₁} is optionally substituted:

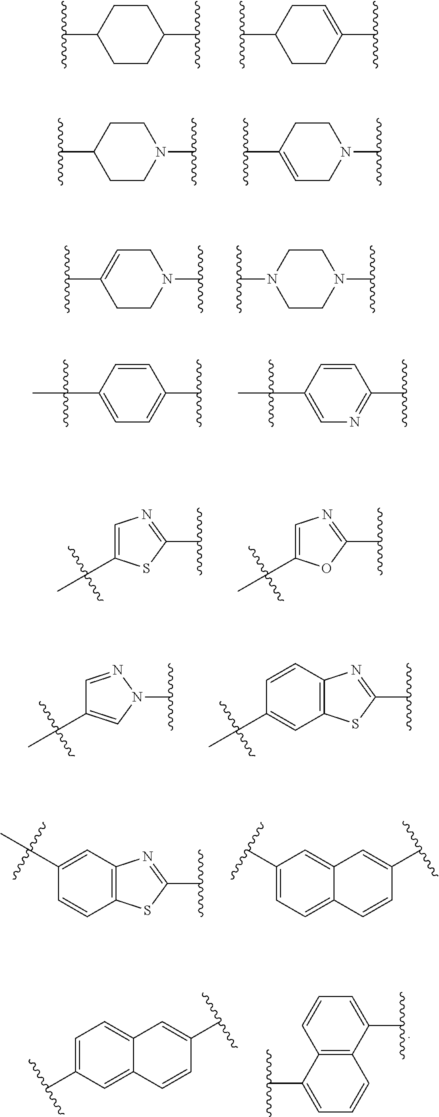

In another embodiment, the compound of Formula (I) is represented by one of Formulae (XII-1) to (XII-6), or a pharmaceutically acceptable salt thereof:

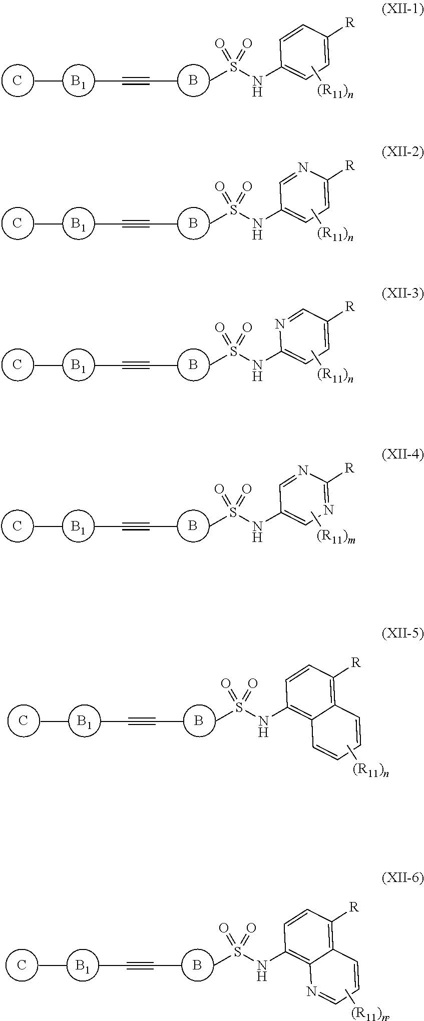

wherein R₁, m, n, \textcircled{B}, \textcircled{B₁}, \textcircled{C}, and R are as previously defined.

In another embodiment, the compound of Formula (I) is represented by one of Formulae (XIII-1) to (XIII-7), or a pharmaceutically acceptable salt thereof:

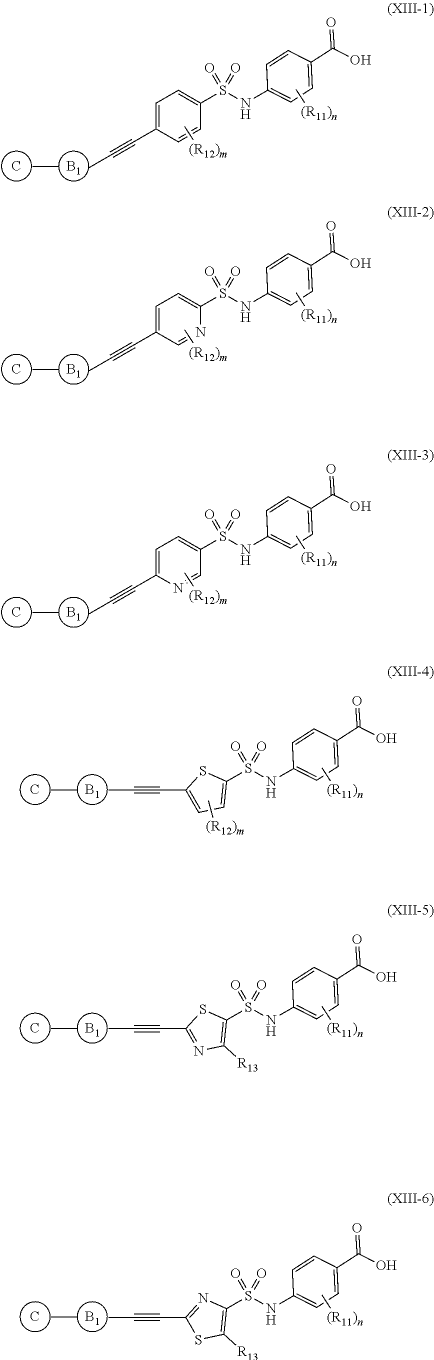

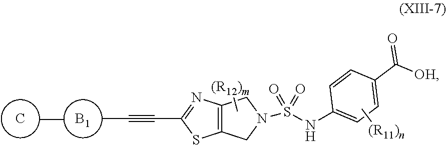

wherein R₁₁, R₁₂, R₁₃, m, n, \textcircled{B₁} and \textcircled{C} are as previously defined.

In another embodiment, the compound of Formula (I) is represented by one of Formulae (XIV-1a) to (XIV-7a), Formulae (XIV-1b) to (XIV-7b), or a pharmaceutically acceptable salt thereof:

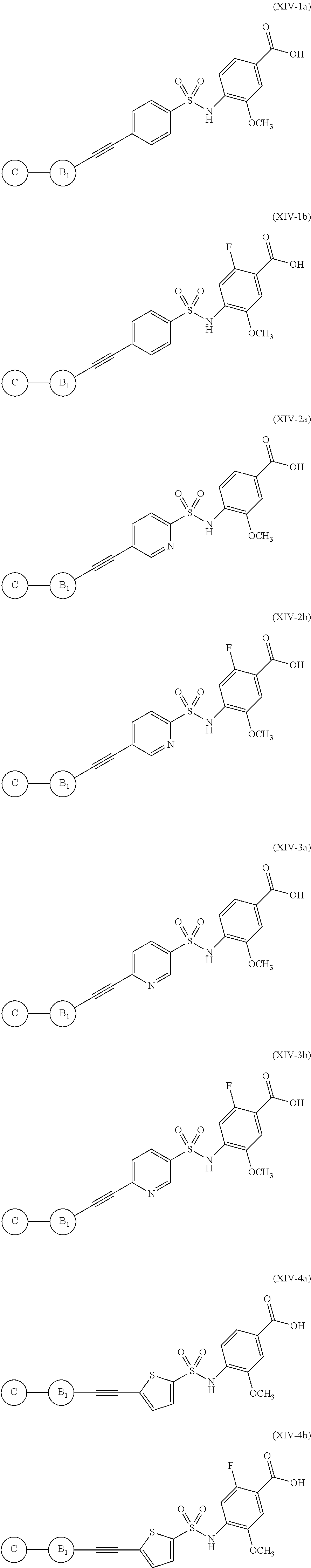

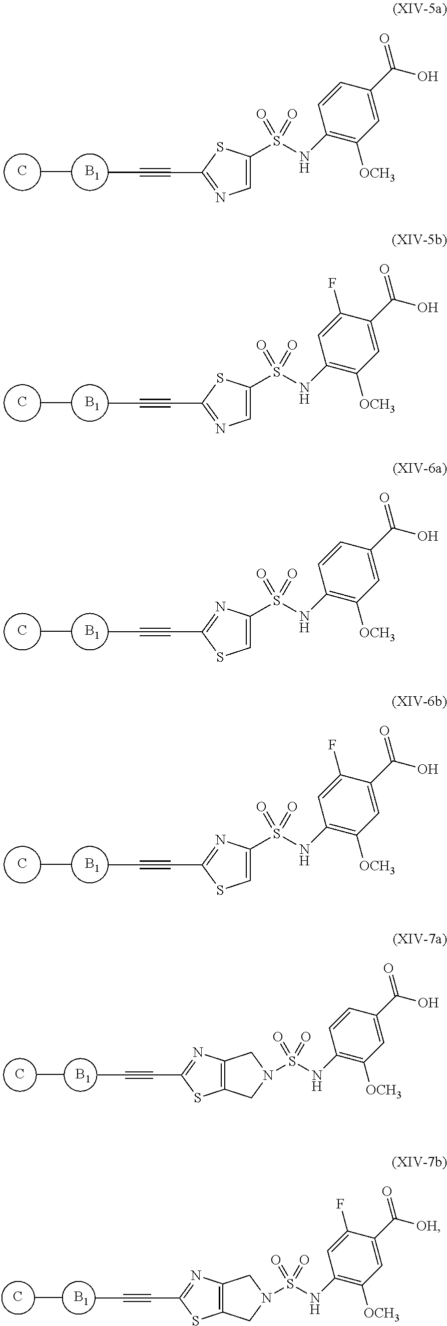

wherein \textcircled{B₁} and \textcircled{C} are as previously defined.

In certain embodiments of the methods and compositions of the invention, the compound of Formula (I) is represented by Formulae (XIV-1a) to (XIV-7a), Formulae (XIV-1b) to (XIV-7b), or a pharmaceutically acceptable salt thereof, where \textcircled{B₁} is selected from the groups below, and \textcircled{B₁} is optionally substituted:

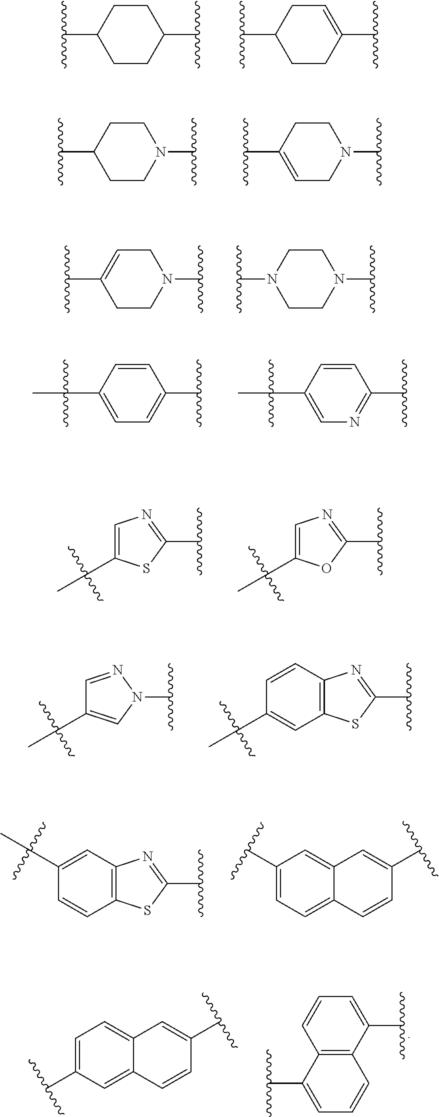

In another embodiment, the compound of Formula (I) is represented by one of Formulae (XV-1) to (XV-4), or a pharmaceutically acceptable salt thereof:

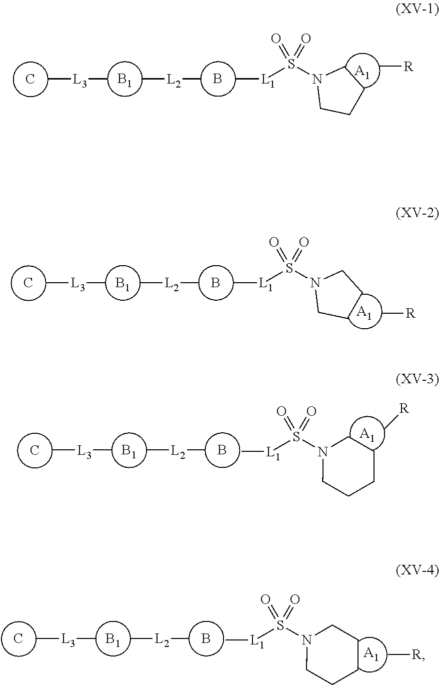

wherein \textcircled{A₁} is optionally substituted aryl, optionally substituted heteroaryl, optionally substituted —C₃-C₁₂ cycloalkyl, optionally substituted 3- to 12-membered heterocycloalkyl; or optionally substituted —C₃-C₁₂ cycloalkenyl; and \textcircled{B}, \textcircled{B₁}, \textcircled{C}, L₁, L₂, L₃, and R are as previously defined.

In another embodiment, the compound of Formula (I) is represented by one of Formulae (XVI-1) to (XVI-8), or a pharmaceutically acceptable salt thereof:

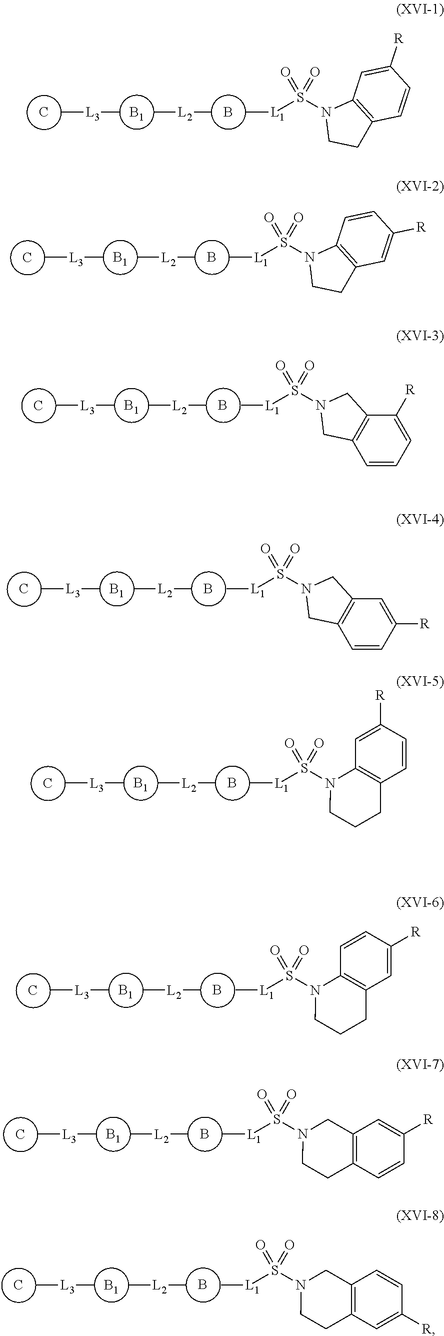

wherein \textcircled{B}, \textcircled{B₁}, \textcircled{C}, L₁, L₂, L₃, and R are as previously defined.

In another embodiment, the compound of Formula (I) is represented by one of Formulae (XVII-1) to (XVII-8), or a pharmaceutically acceptable salt thereof:

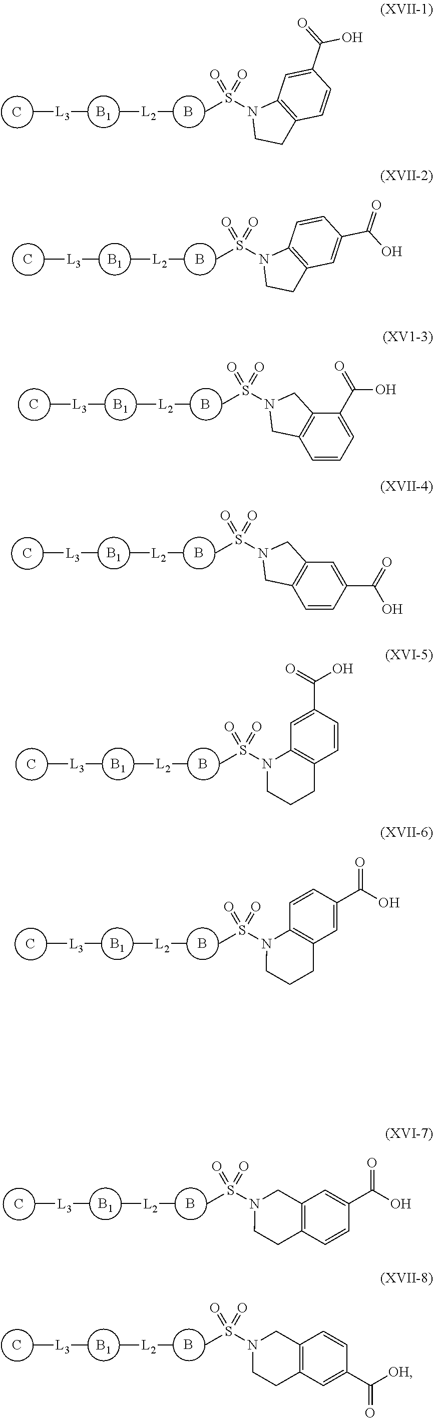

wherein \textcircled{B}, \textcircled{B₁}, \textcircled{C}, L₂, and L₃ are as previously defined.

In another embodiment, the compound of Formula (I) is represented by one of Formulae (XVIII-1) to (XVIII-2), or a pharmaceutically acceptable salt thereof:

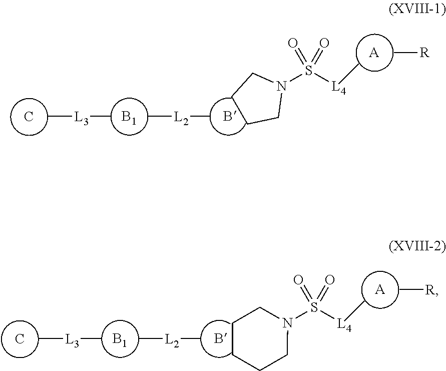

wherein \textcircled{B′} is optionally substituted aryl, optionally substituted heteroaryl, optionally substituted —C₃-C₁₂ cycloalkyl, optionally substituted 3- to 12-membered heterocycloalkyl, or optionally substituted —C₃-C₁₂ cycloalkenyl; and \textcircled{A}, \textcircled{B₁}, \textcircled{C}, L₂, L₃, L₄, and R are as previously defined.

In another embodiment, the compound of Formula (I) is represented by one of Formulae (XVIII-1) to (XVIII-2), or a pharmaceutically acceptable salt thereof, wherein \textcircled{C} is selected from the groups set forth below, and \textcircled{C} is optionally substituted:

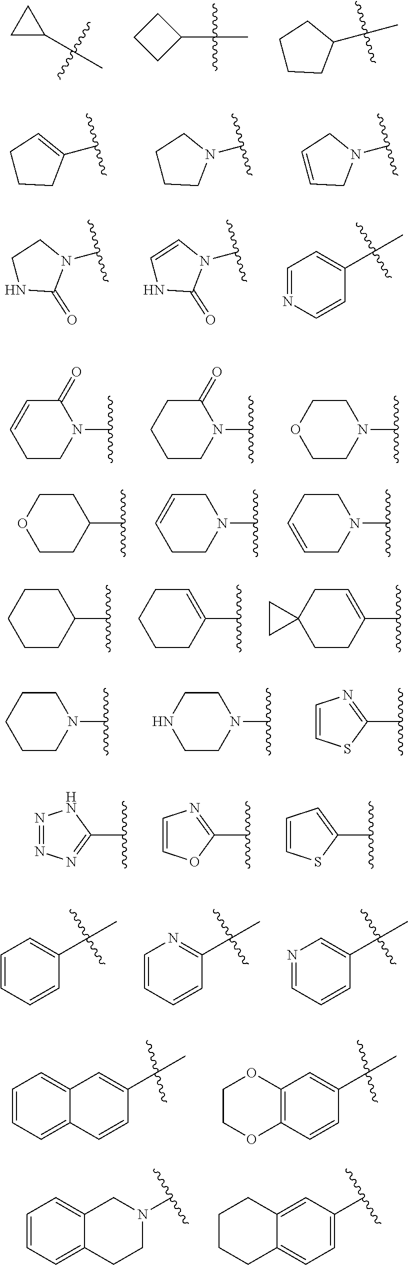

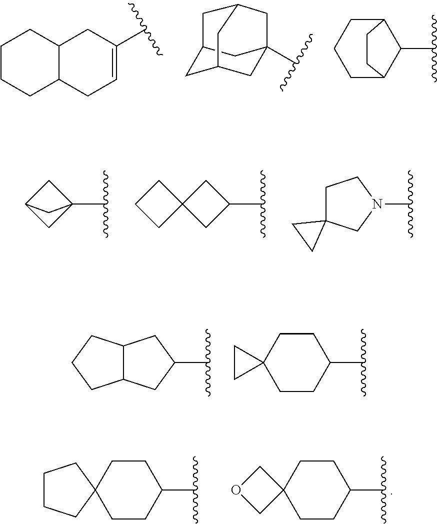

In another embodiment, the compound of Formula (I) is represented by one of Formulae (XIX-1) to (XIX-10), or a pharmaceutically acceptable salt thereof:

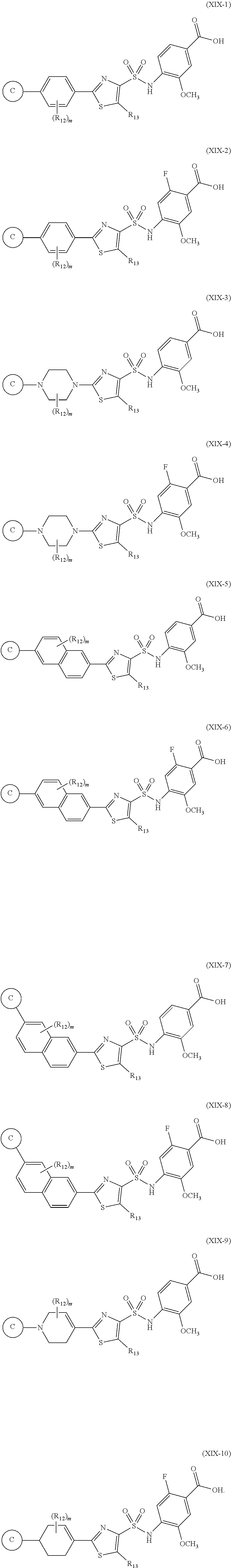

wherein R₁₂, R₁₃, m, and \textcircled{C} are as previously defined, preferably R₁₃ is hydrogen.

In one embodiment, the compound of Formula (I) is selected from the compounds set forth below:

| Compound # | Structure |
|---|---|
| 1 | 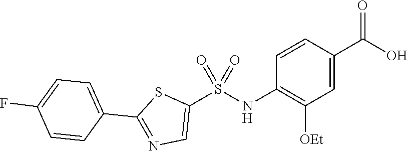 [SMILES: CCOc1cc(C(=O)O)ccc1NS(=O)(=O)c1cnc(-c2ccc(F)cc2)s1] |
| 3 | 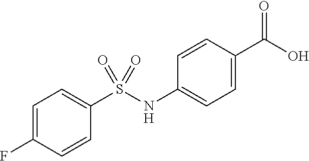 [SMILES: O=C(O)c1ccc(NS(=O)(=O)c2ccc(F)cc2)cc1] |
| 4 | 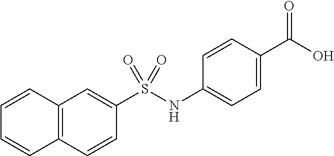 [SMILES: O=C(O)c1ccc(NS(=O)(=O)c2ccc3ccccc3c2)cc1] |
| 5 | 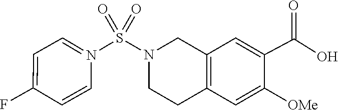 |
| 6 | 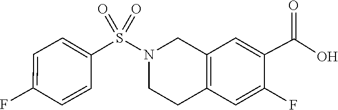 [SMILES: O=C(O)c1cc2c(cc1F)CCN(S(=O)(=O)c1ccc(F)cc1)C2] |
| 7 | 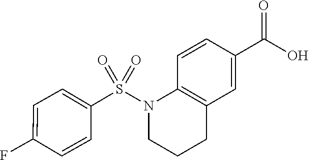 [SMILES: O=C(O)c1ccc2c(c1)CCCN2S(=O)(=O)c1ccc(F)cc1] |
| 8 | 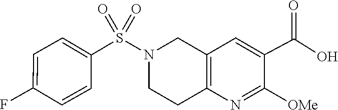 [SMILES: COc1nc2c(cc1C(=O)O)CN(S(=O)(=O)c1ccc(F)cc1)CC2] |
| 9 | 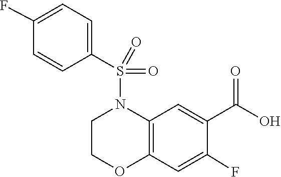 [SMILES: O=C(O)c1cc2c(cc1F)OCCN2S(=O)(=O)c1ccc(F)cc1] |
| 10 | 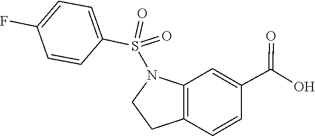 |
| 11 | 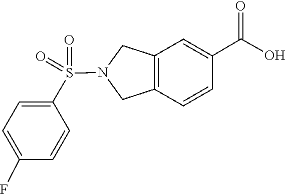 [SMILES: O=C(O)c1ccc2c(c1)CN(S(=O)(=O)c1ccc(F)cc1)C2] |
| 12 | 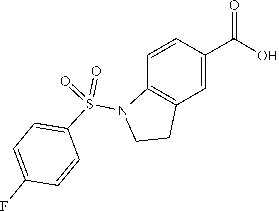 [SMILES: O=C(O)c1ccc2c(c1)CCN2S(=O)(=O)c1ccc(F)cc1] |
| 13 | 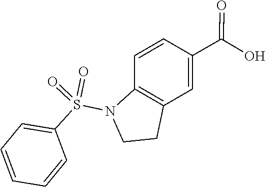 [SMILES: O=C(O)c1ccc2c(c1)CCN2S(=O)(=O)c1ccccc1] |
| 14 | 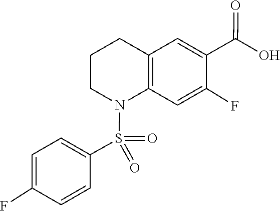 [SMILES: O=C(O)c1cc2c(cc1F)N(S(=O)(=O)c1ccc(F)cc1)CCC2] |
| 15 | 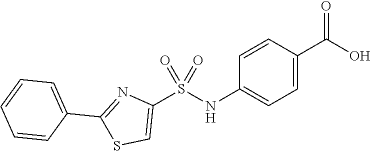 [SMILES: O=C(O)c1ccc(NS(=O)(=O)c2csc(-c3ccccc3)n2)cc1] |
| 16 | 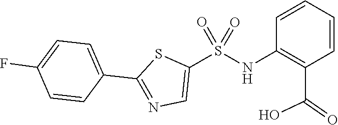 [SMILES: O=C(O)c1ccccc1NS(=O)(=O)c1cnc(-c2ccc(F)cc2)s1] |
| 17 | 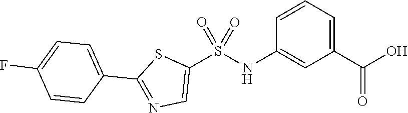 [SMILES: O=C(O)c1cccc(NS(=O)(=O)c2cnc(-c3ccc(F)cc3)s2)c1] |
| 18 | 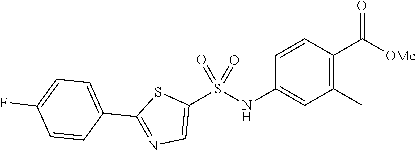 [SMILES: COC(=O)c1ccc(NS(=O)(=O)c2cnc(-c3ccc(F)cc3)s2)cc1C] |
| 19 | 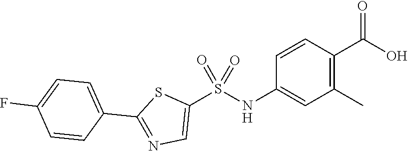 [SMILES: Cc1cc(NS(=O)(=O)c2cnc(-c3ccc(F)cc3)s2)ccc1C(=O)O] |
| 20 | 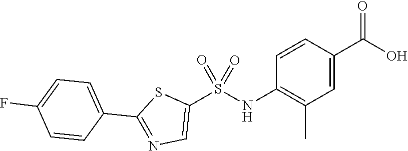 [SMILES: Cc1cc(C(=O)O)ccc1NS(=O)(=O)c1cnc(-c2ccc(F)cc2)s1] |
| 21 | 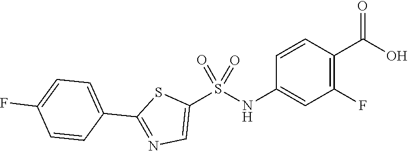 [SMILES: O=C(O)c1ccc(NS(=O)(=O)c2cnc(-c3ccc(F)cc3)s2)cc1F] |
| 22 | 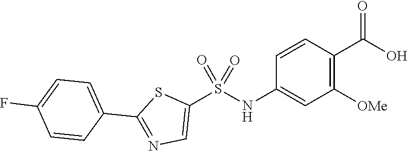 [SMILES: COc1cc(NS(=O)(=O)c2cnc(-c3ccc(F)cc3)s2)ccc1C(=O)O] |
| 23 | 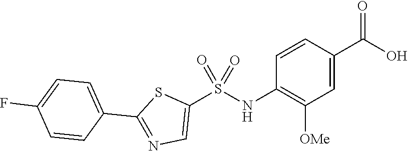 [SMILES: COc1cc(C(=O)O)ccc1NS(=O)(=O)c1cnc(-c2ccc(F)cc2)s1] |
| 24 | 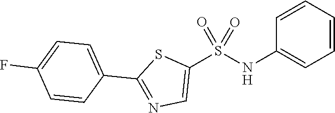 [SMILES: O=S(=O)(Nc1ccccc1)c1cnc(-c2ccc(F)cc2)s1] |
| 25 | 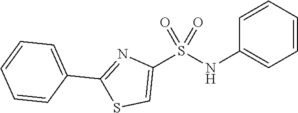 [SMILES: O=S(=O)(Nc1ccccc1)c1csc(-c2ccccc2)n1] |
| 26 | 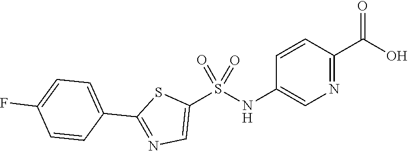 [SMILES: O=C(O)c1ccc(NS(=O)(=O)c2cnc(-c3ccc(F)cc3)s2)cn1] |
| 27 | 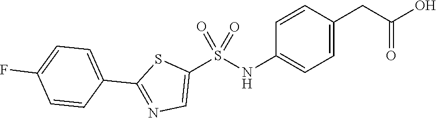 [SMILES: O=C(O)Cc1ccc(NS(=O)(=O)c2cnc(-c3ccc(F)cc3)s2)cc1] |
| 28 | 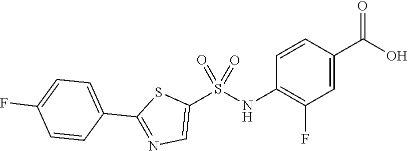 [SMILES: O=C(O)c1ccc(NS(=O)(=O)c2cnc(-c3ccc(F)cc3)s2)c(F)c1] |
| 29 | 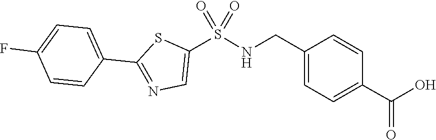 [SMILES: O=C(O)c1ccc(CNS(=O)(=O)c2cnc(-c3ccc(F)cc3)s2)cc1] |
| 30 | 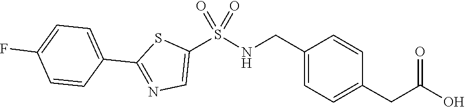 [SMILES: O=C(O)Cc1ccc(CNS(=O)(=O)c2cnc(-c3ccc(F)cc3)s2)cc1] |
| 31 | 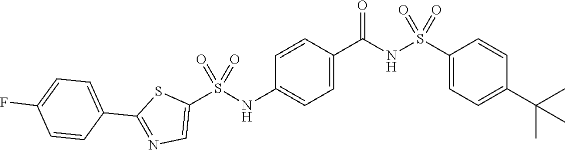 [SMILES: CC(C)(C)c1ccc(S(=O)(=O)NC(=O)c2ccc(NS(=O)(=O)c3cnc(-c4ccc(F)cc4)s3)cc2)cc1] |
| 32 | 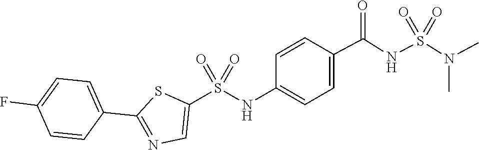 [SMILES: CN(C)S(=O)(=O)NC(=O)c1ccc(NS(=O)(=O)c2cnc(-c3ccc(F)cc3)s2)cc1] |
| 33 | 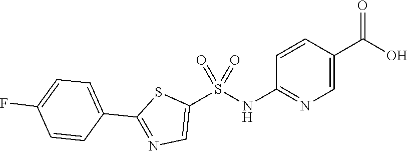 [SMILES: O=C(O)c1ccc(NS(=O)(=O)c2cnc(-c3ccc(F)cc3)s2)nc1] |
| 34 | 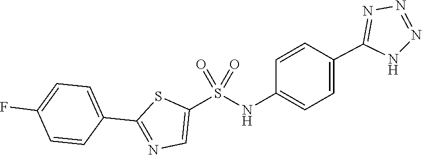 [SMILES: O=S(=O)(Nc1ccc(-c2nnn[nH]2)cc1)c1cnc(-c2ccc(F)cc2)s1] |
| 35 | 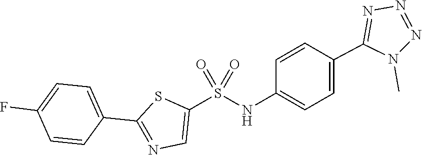 [SMILES: Cn1nnnc1-c1ccc(NS(=O)(=O)c2cnc(-c3ccc(F)cc3)s2)cc1] |
| 36 | 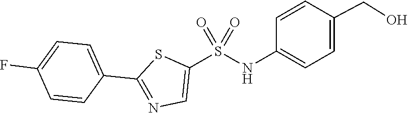 [SMILES: O=S(=O)(Nc1ccc(CO)cc1)c1cnc(-c2ccc(F)cc2)s1] |
| 37 | 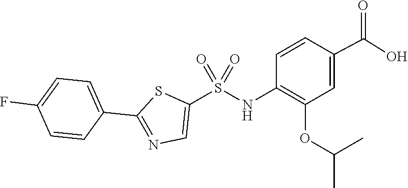 [SMILES: CC(C)Oc1cc(C(=O)O)ccc1NS(=O)(=O)c1cnc(-c2ccc(F)cc2)s1] |
| 38 | 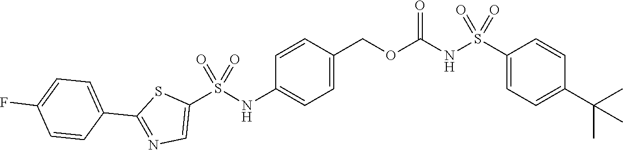 [SMILES: CC(C)(C)c1ccc(S(=O)(=O)NC(=O)OCc2ccc(NS(=O)(=O)c3cnc(-c4ccc(F)cc4)s3)cc2)cc1] |
| 39 | 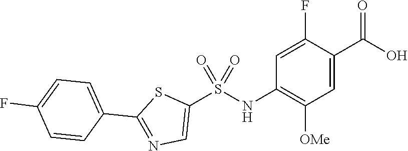 [SMILES: COc1cc(C(=O)O)c(F)cc1NS(=O)(=O)c1cnc(-c2ccc(F)cc2)s1] |
| 40 | 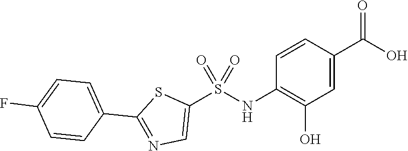 [SMILES: O=C(O)c1ccc(NS(=O)(=O)c2cnc(-c3ccc(F)cc3)s2)c(O)c1] |
| 41 | 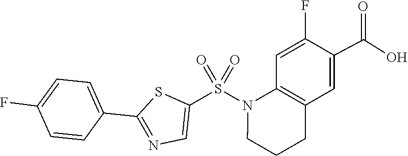 [SMILES: O=C(O)c1cc2c(cc1F)N(S(=O)(=O)c1cnc(-c3ccc(F)cc3)s1)CCC2] |
| 42 | 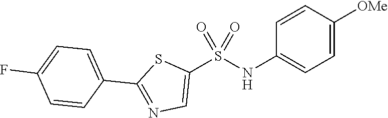 [SMILES: COc1ccc(NS(=O)(=O)c2cnc(-c3ccc(F)cc3)s2)cc1] |
| 43 | 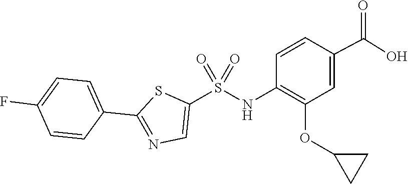 [SMILES: O=C(O)c1ccc(NS(=O)(=O)c2cnc(-c3ccc(F)cc3)s2)c(OC2CC2)c1] |
| 44 | 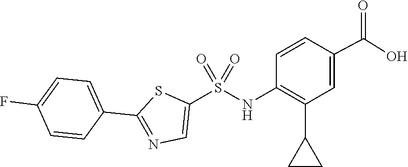 [SMILES: O=C(O)c1ccc(NS(=O)(=O)c2cnc(-c3ccc(F)cc3)s2)c(C2CC2)c1] |
| 45 | 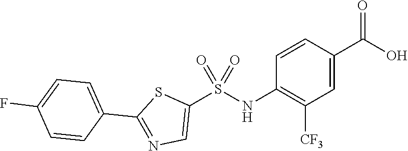 [SMILES: O=C(O)c1ccc(NS(=O)(=O)c2cnc(-c3ccc(F)cc3)s2)c(C(F)(F)F)c1] |
| 46 | 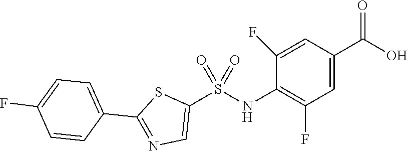 [SMILES: O=C(O)c1cc(F)c(NS(=O)(=O)c2cnc(-c3ccc(F)cc3)s2)c(F)c1] |
| 47 | 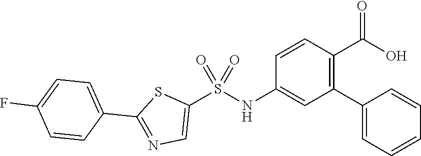 [SMILES: O=C(O)c1ccc(NS(=O)(=O)c2cnc(-c3ccc(F)cc3)s2)cc1-c1ccccc1] |
| 48 | 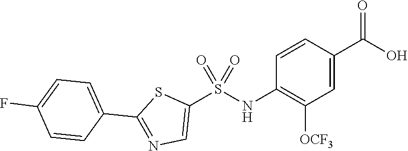 [SMILES: O=C(O)c1ccc(NS(=O)(=O)c2cnc(-c3ccc(F)cc3)s2)c(OC(F)(F)F)c1] |
| 49 | 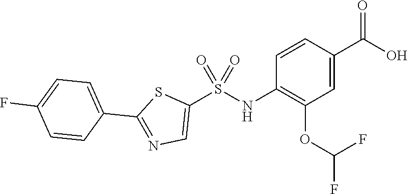 [SMILES: O=C(O)c1ccc(NS(=O)(=O)c2cnc(-c3ccc(F)cc3)s2)c(OC(F)F)c1] |
| 50 | 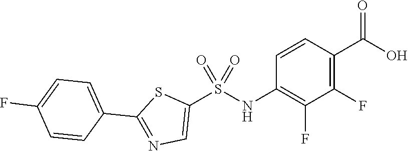 [SMILES: O=C(O)c1ccc(NS(=O)(=O)c2cnc(-c3ccc(F)cc3)s2)c(F)c1F] |
| 51 | 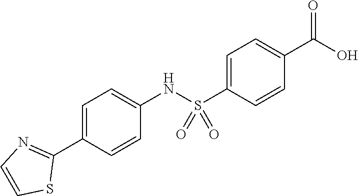 [SMILES: O=C(O)c1ccc(S(=O)(=O)Nc2ccc(-c3nccs3)cc2)cc1] |
| 52 | 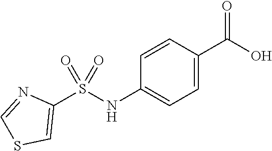 [SMILES: O=C(O)c1ccc(NS(=O)(=O)c2cscn2)cc1] |
| 53 | 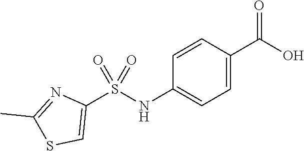 [SMILES: Cc1nc(S(=O)(=O)Nc2ccc(C(=O)O)cc2)cs1] |
| 54 | 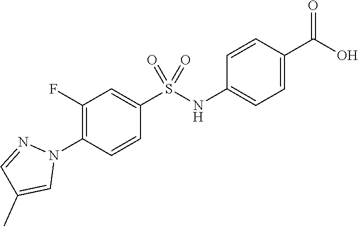 [SMILES: Cc1cnn(-c2ccc(S(=O)(=O)Nc3ccc(C(=O)O)cc3)cc2F)c1] |
| 55 | 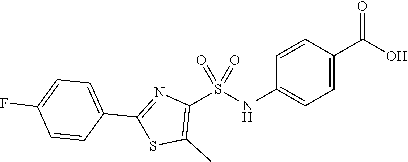 [SMILES: Cc1sc(-c2ccc(F)cc2)nc1S(=O)(=O)Nc1ccc(C(=O)O)cc1] |
| 56 | 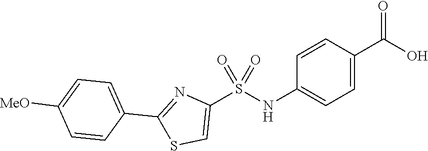 [SMILES: COc1ccc(-c2nc(S(=O)(=O)Nc3ccc(C(=O)O)cc3)cs2)cc1] |
| 57 | 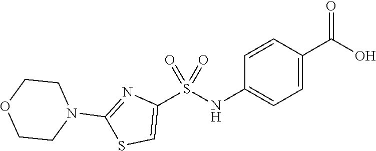 [SMILES: O=C(O)c1ccc(NS(=O)(=O)c2csc(N3CCOCC3)n2)cc1] |
| 58 | 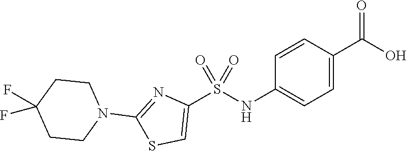 [SMILES: O=C(O)c1ccc(NS(=O)(=O)c2csc(N3CCC(F)(F)CC3)n2)cc1] |
| 59 | 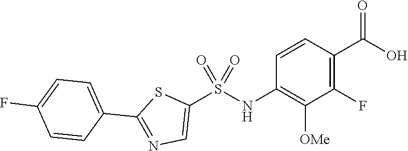 [SMILES: COc1c(NS(=O)(=O)c2cnc(-c3ccc(F)cc3)s2)ccc(C(=O)O)c1F] |
| 60 | 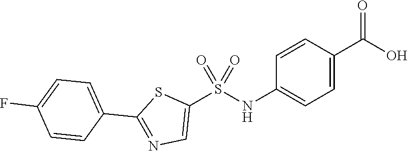 [SMILES: O=C(O)c1ccc(NS(=O)(=O)c2cnc(-c3ccc(F)cc3)s2)cc1] |
| 61 | 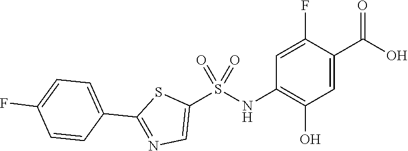 [SMILES: O=C(O)c1cc(O)c(NS(=O)(=O)c2cnc(-c3ccc(F)cc3)s2)cc1F] |
| 62 | 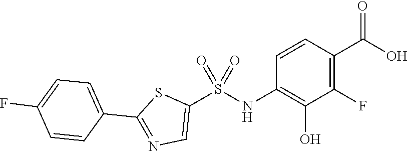 [SMILES: O=C(O)c1ccc(NS(=O)(=O)c2cnc(-c3ccc(F)cc3)s2)c(O)c1F] |
| 63 | 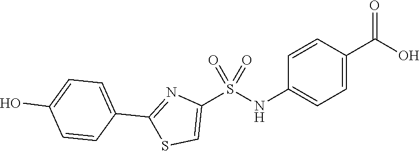 [SMILES: O=C(O)c1ccc(NS(=O)(=O)c2csc(-c3ccc(O)cc3)n2)cc1] |
| 64 | 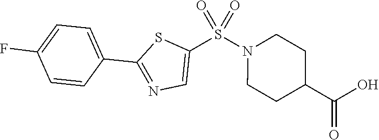 [SMILES: O=C(O)C1CCN(S(=O)(=O)c2cnc(-c3ccc(F)cc3)s2)CC1] |
| 65 | 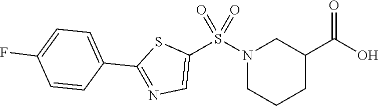 [SMILES: O=C(O)C1CCCN(S(=O)(=O)c2cnc(-c3ccc(F)cc3)s2)C1] |
| 66 | 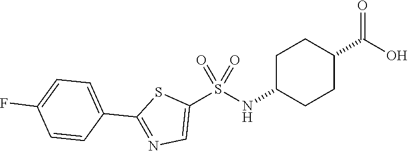 [SMILES: O=C(O)[C@H]1CC[C@@H](NS(=O)(=O)c2cnc(-c3ccc(F)cc3)s2)CC1] |
| 67 | 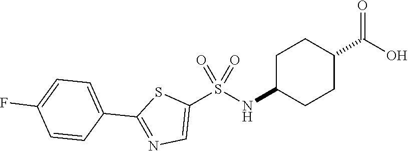 [SMILES: O=C(O)[C@H]1CC[C@H](NS(=O)(=O)c2cnc(-c3ccc(F)cc3)s2)CC1] |
| 68 | 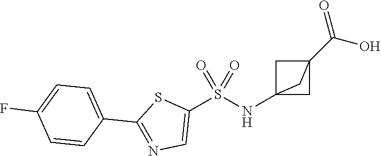 [SMILES: O=C(O)C12CC(NS(=O)(=O)c3cnc(-c4ccc(F)cc4)s3)(C1)C2] |
| 69 | 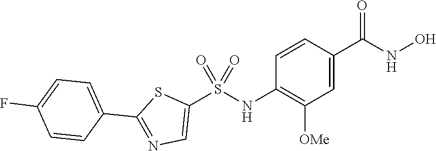 [SMILES: COc1cc(C(=O)NO)ccc1NS(=O)(=O)c1cnc(-c2ccc(F)cc2)s1] |
| 70 | 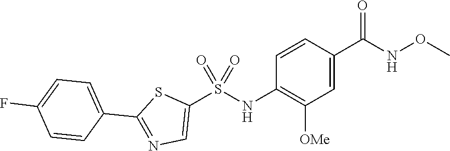 [SMILES: CONC(=O)c1ccc(NS(=O)(=O)c2cnc(-c3ccc(F)cc3)s2)c(OC)c1] |
| 71 | 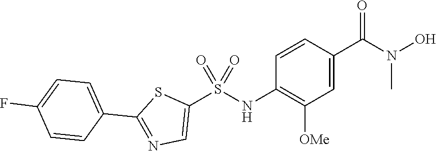 [SMILES: COc1cc(C(=O)N(C)O)ccc1NS(=O)(=O)c1cnc(-c2ccc(F)cc2)s1] |
| 72 | 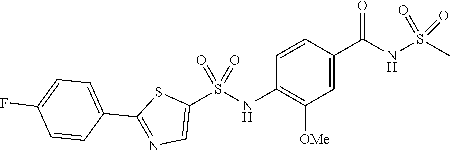 [SMILES: COc1cc(C(=O)NS(C)(=O)=O)ccc1NS(=O)(=O)c1cnc(-c2ccc(F)cc2)s1] |
| 73 | 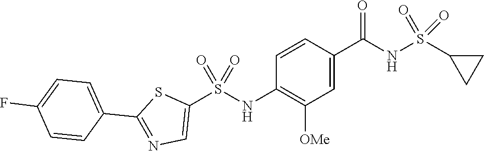 [SMILES: COc1cc(C(=O)NS(=O)(=O)C2CC2)ccc1NS(=O)(=O)c1cnc(-c2ccc(F)cc2)s1] |
| 74 | 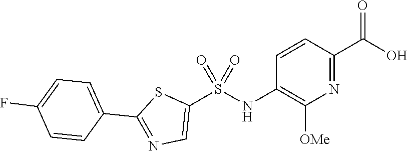 [SMILES: COc1nc(C(=O)O)ccc1NS(=O)(=O)c1cnc(-c2ccc(F)cc2)s1] |
| 75 | 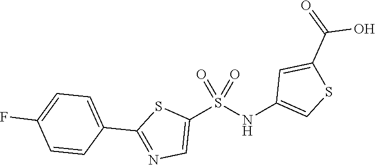 [SMILES: O=C(O)c1cc(NS(=O)(=O)c2cnc(-c3ccc(F)cc3)s2)cs1] |
| 76 | 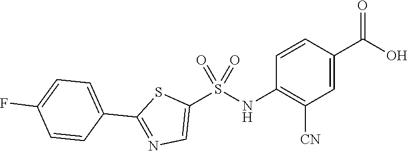 [SMILES: N#Cc1cc(C(=O)O)ccc1NS(=O)(=O)c1cnc(-c2ccc(F)cc2)s1] |
| 77 | 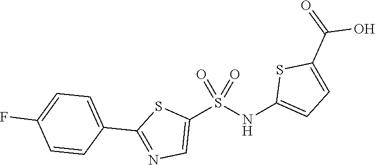 [SMILES: O=C(O)c1ccc(NS(=O)(=O)c2cnc(-c3ccc(F)cc3)s2)s1] |
| 78 | 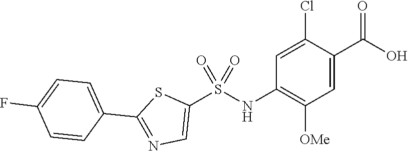 [SMILES: COc1cc(C(=O)O)c(Cl)cc1NS(=O)(=O)c1cnc(-c2ccc(F)cc2)s1] |
| 79 | 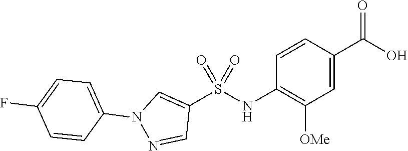 [SMILES: COc1cc(C(=O)O)ccc1NS(=O)(=O)c1cnn(-c2ccc(F)cc2)c1] |
| 80 | 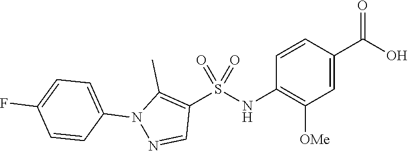 [SMILES: COc1cc(C(=O)O)ccc1NS(=O)(=O)c1cnn(-c2ccc(F)cc2)c1C] |
| 81 | 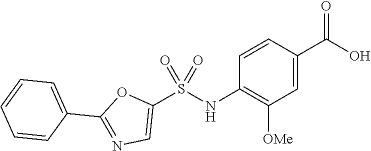 [SMILES: COc1cc(C(=O)O)ccc1NS(=O)(=O)c1cnc(-c2ccccc2)o1] |
| 82 | 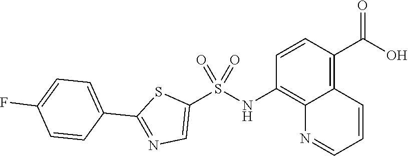 [SMILES: O=C(O)c1ccc(NS(=O)(=O)c2cnc(-c3ccc(F)cc3)s2)c2ncccc12] |
| 83 | 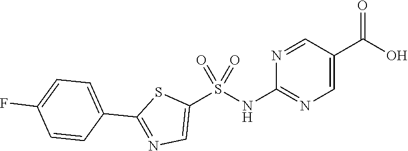 [SMILES: O=C(O)c1cnc(NS(=O)(=O)c2cnc(-c3ccc(F)cc3)s2)nc1] |
| 84 | 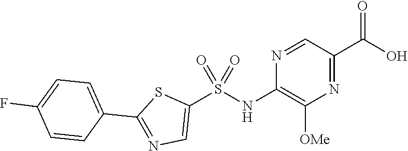 [SMILES: COc1nc(C(=O)O)cnc1NS(=O)(=O)c1cnc(-c2ccc(F)cc2)s1] |
| 85 | 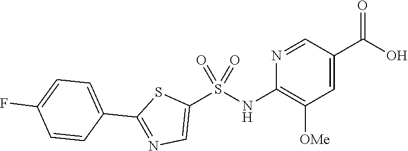 [SMILES: COc1cc(C(=O)O)cnc1NS(=O)(=O)c1cnc(-c2ccc(F)cc2)s1] |
| 86 | 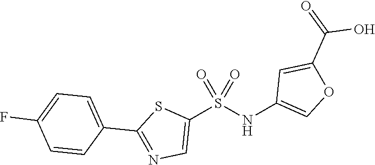 [SMILES: O=C(O)c1cc(NS(=O)(=O)c2cnc(-c3ccc(F)cc3)s2)co1] |
| 87 | 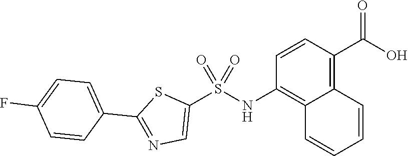 [SMILES: O=C(O)c1ccc(NS(=O)(=O)c2cnc(-c3ccc(F)cc3)s2)c2ccccc12] |
| 88 | 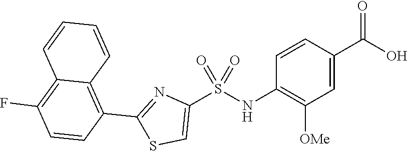 [SMILES: COc1cc(C(=O)O)ccc1NS(=O)(=O)c1csc(-c2ccc(F)c3ccccc23)n1] |
| 89 | 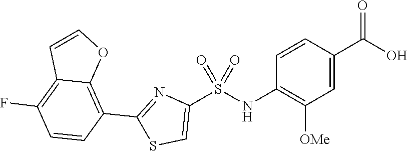 [SMILES: COc1cc(C(=O)O)ccc1NS(=O)(=O)c1csc(-c2ccc(F)c3ccoc23)n1] |
| 90 | 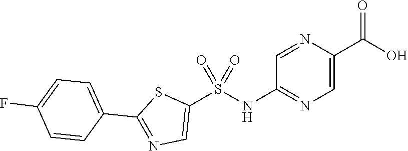 [SMILES: O=C(O)c1cnc(NS(=O)(=O)c2cnc(-c3ccc(F)cc3)s2)cn1] |
| 91 | 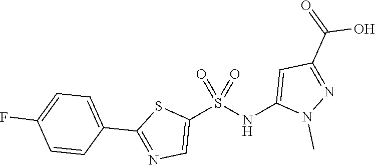 [SMILES: Cn1nc(C(=O)O)cc1NS(=O)(=O)c1cnc(-c2ccc(F)cc2)s1] |
| 92 | 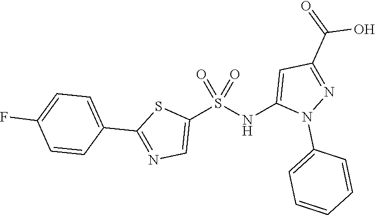 [SMILES: O=C(O)c1cc(NS(=O)(=O)c2cnc(-c3ccc(F)cc3)s2)n(-c2ccccc2)n1] |
| 93 | 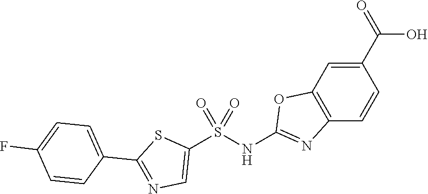 [SMILES: O=C(O)c1ccc2nc(NS(=O)(=O)c3cnc(-c4ccc(F)cc4)s3)oc2c1] |
| 94 | 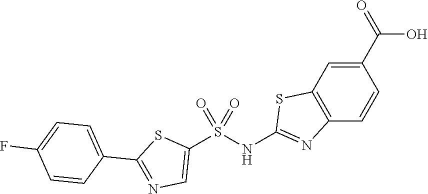 [SMILES: O=C(O)c1ccc2nc(NS(=O)(=O)c3cnc(-c4ccc(F)cc4)s3)sc2c1] |
| 95 | 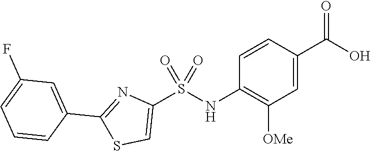 [SMILES: COc1cc(C(=O)O)ccc1NS(=O)(=O)c1csc(-c2cccc(F)c2)n1] |
| 96 | 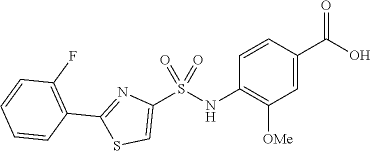 [SMILES: COc1cc(C(=O)O)ccc1NS(=O)(=O)c1csc(-c2ccccc2F)n1] |
| 97 | 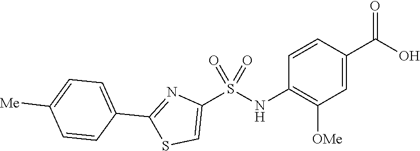 [SMILES: COc1cc(C(=O)O)ccc1NS(=O)(=O)c1csc(-c2ccc(C)cc2)n1] |
| 98 | 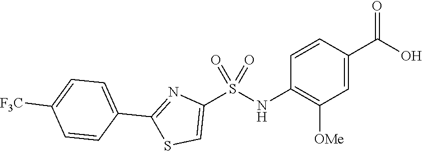 [SMILES: COc1cc(C(=O)O)ccc1NS(=O)(=O)c1csc(-c2ccc(C(F)(F)F)cc2)n1] |
| 99 | 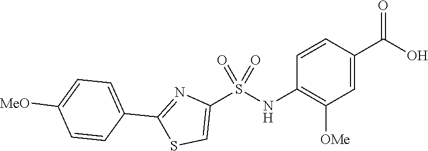 [SMILES: COc1ccc(-c2nc(S(=O)(=O)Nc3ccc(C(=O)O)cc3OC)cs2)cc1] |
| 100 | 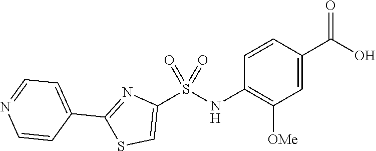 [SMILES: COc1cc(C(=O)O)ccc1NS(=O)(=O)c1csc(-c2ccncc2)n1] |
| 101 | 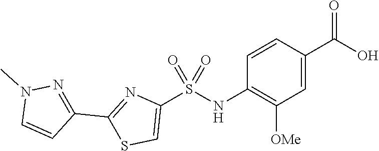 [SMILES: COc1cc(C(=O)O)ccc1NS(=O)(=O)c1csc(-c2ccn(C)n2)n1] |
| 102 | 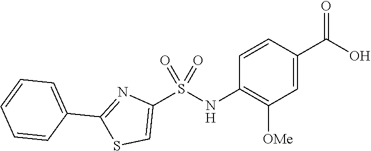 [SMILES: COc1cc(C(=O)O)ccc1NS(=O)(=O)c1csc(-c2ccccc2)n1] |
| 103 | 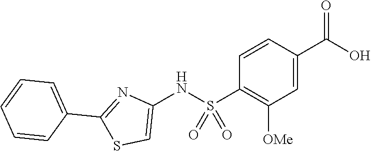 [SMILES: COc1cc(C(=O)O)ccc1S(=O)(=O)Nc1csc(-c2ccccc2)n1] |
| 104 | 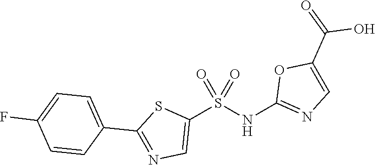 [SMILES: O=C(O)c1cnc(NS(=O)(=O)c2cnc(-c3ccc(F)cc3)s2)o1] |
| 105 | 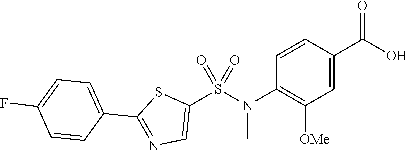 [SMILES: COc1cc(C(=O)O)ccc1N(C)S(=O)(=O)c1cnc(-c2ccc(F)cc2)s1] |
| 106 | 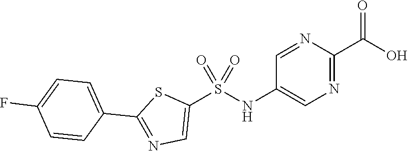 [SMILES: O=C(O)c1ncc(NS(=O)(=O)c2cnc(-c3ccc(F)cc3)s2)cn1] |
| 107 | 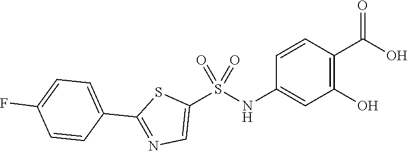 [SMILES: O=C(O)c1ccc(NS(=O)(=O)c2cnc(-c3ccc(F)cc3)s2)cc1O] |
| 108 | 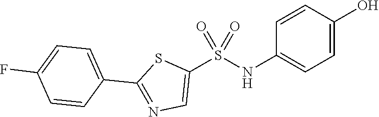 [SMILES: O=S(=O)(Nc1ccc(O)cc1)c1cnc(-c2ccc(F)cc2)s1] |
| 109 | 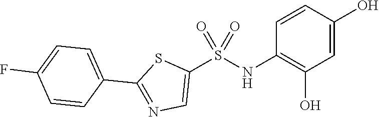 [SMILES: O=S(=O)(Nc1ccc(O)cc1O)c1cnc(-c2ccc(F)cc2)s1] |
| 110 | 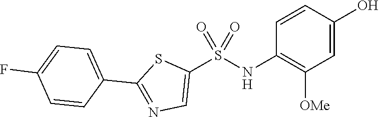 [SMILES: COc1cc(O)ccc1NS(=O)(=O)c1cnc(-c2ccc(F)cc2)s1] |
| 111 | 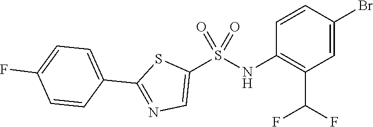 [SMILES: O=S(=O)(Nc1ccc(Br)cc1C(F)F)c1cnc(-c2ccc(F)cc2)s1] |
| 112 | 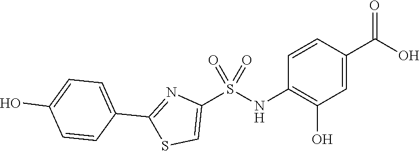 [SMILES: O=C(O)c1ccc(NS(=O)(=O)c2csc(-c3ccc(O)cc3)n2)c(O)c1] |
| 113 | 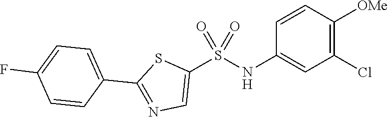 [SMILES: COc1ccc(NS(=O)(=O)c2cnc(-c3ccc(F)cc3)s2)cc1Cl] |
| 114 | 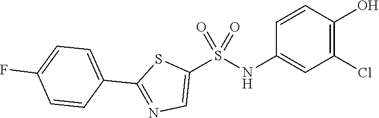 [SMILES: O=S(=O)(Nc1ccc(O)c(Cl)c1)c1cnc(-c2ccc(F)cc2)s1] |
| 115 | 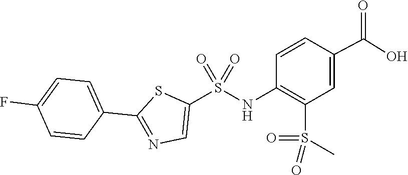 [SMILES: CS(=O)(=O)c1cc(C(=O)O)ccc1NS(=O)(=O)c1cnc(-c2ccc(F)cc2)s1] |
| 116 | 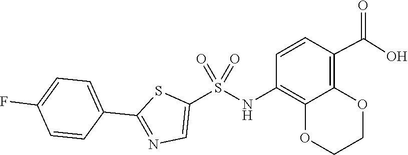 [SMILES: O=C(O)c1ccc(NS(=O)(=O)c2cnc(-c3ccc(F)cc3)s2)c2c1OCCO2] |
| 117 | 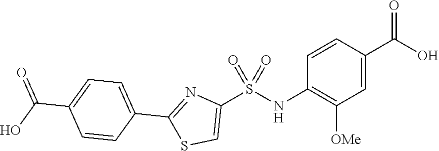 [SMILES: COc1cc(C(=O)O)ccc1NS(=O)(=O)c1csc(-c2ccc(C(=O)O)cc2)n1] |
| 118 | 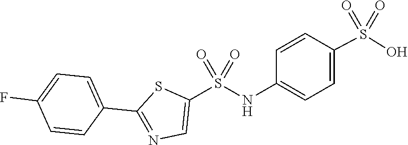 [SMILES: O=S(=O)(O)c1ccc(NS(=O)(=O)c2cnc(-c3ccc(F)cc3)s2)cc1] |
| 119 | 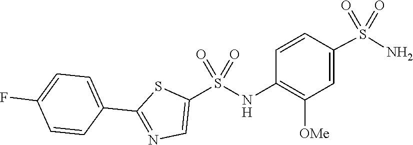 [SMILES: COc1cc(S(N)(=O)=O)ccc1NS(=O)(=O)c1cnc(-c2ccc(F)cc2)s1] |
| 120 | 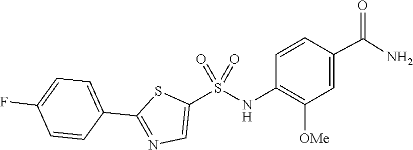 [SMILES: COc1cc(C(N)=O)ccc1NS(=O)(=O)c1cnc(-c2ccc(F)cc2)s1] |
| 121 | 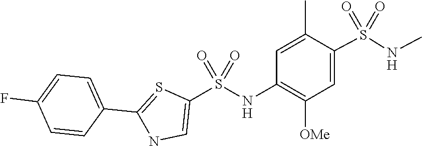 |
| 122 | 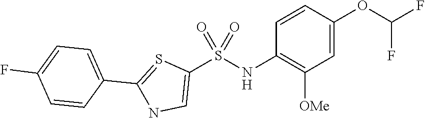 |
| 123 | 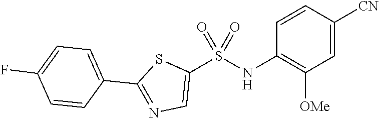 [SMILES: COc1cc(C#N)ccc1NS(=O)(=O)c1cnc(-c2ccc(F)cc2)s1] |
| 124 | 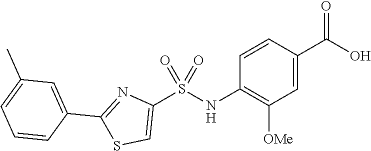 [SMILES: COc1cc(C(=O)O)ccc1NS(=O)(=O)c1csc(-c2cccc(C)c2)n1] |
| 125 | 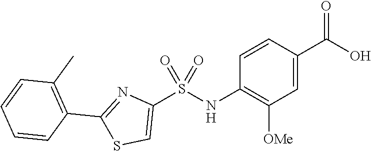 [SMILES: COc1cc(C(=O)O)ccc1NS(=O)(=O)c1csc(-c2ccccc2C)n1] |
| 126 | 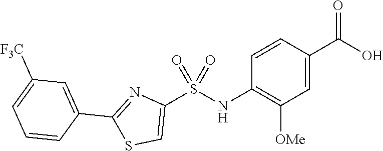 [SMILES: COc1cc(C(=O)O)ccc1NS(=O)(=O)c1csc(-c2cccc(C(F)(F)F)c2)n1] |
| 127 | 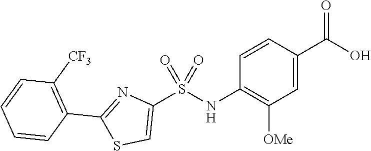 [SMILES: COc1cc(C(=O)O)ccc1NS(=O)(=O)c1csc(-c2ccccc2C(F)(F)F)n1] |
| 128 | 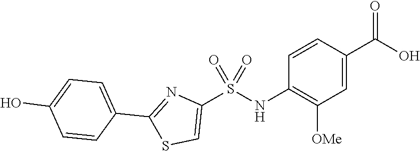 [SMILES: COc1cc(C(=O)O)ccc1NS(=O)(=O)c1csc(-c2ccc(O)cc2)n1] |
| 129 | 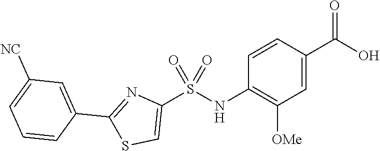 [SMILES: COc1cc(C(=O)O)ccc1NS(=O)(=O)c1csc(-c2cccc(C#N)c2)n1] |
| 130 | 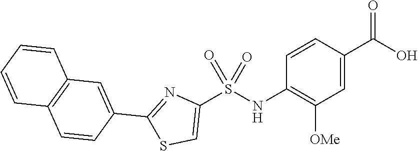 [SMILES: COc1cc(C(=O)O)ccc1NS(=O)(=O)c1csc(-c2ccc3ccccc3c2)n1] |
| 131 | 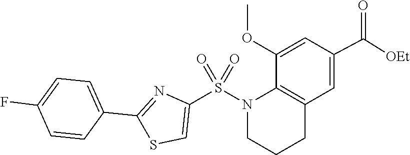 [SMILES: CCOC(=O)c1cc2c(c(OC)c1)N(S(=O)(=O)c1csc(-c3ccc(F)cc3)n1)CCC2] |
| 132 | 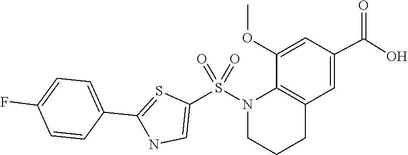 |
| 133 | 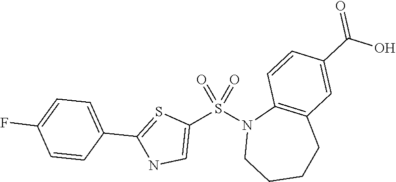 |
| 134 | 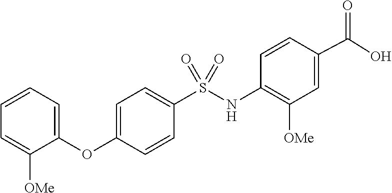 [SMILES: COc1cc(C(=O)O)ccc1NS(=O)(=O)c1ccc(Oc2ccccc2OC)cc1] |
| 135 | 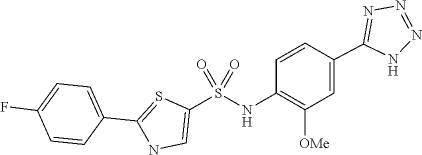 |
| 136 | 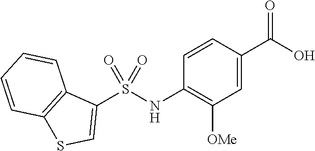 [SMILES: COc1cc(C(=O)O)ccc1NS(=O)(=O)c1csc2ccccc12] |
| 137 | 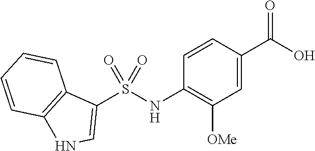 [SMILES: COc1cc(C(=O)O)ccc1NS(=O)(=O)c1c[nH]c2ccccc12] |
| 138 | 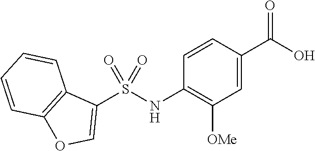 [SMILES: COc1cc(C(=O)O)ccc1NS(=O)(=O)c1coc2ccccc12] |
| 139 | 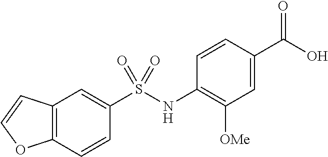 [SMILES: COc1cc(C(=O)O)ccc1NS(=O)(=O)c1ccc2occc2c1] |
| 140 | 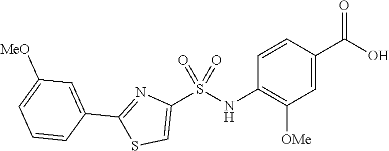 [SMILES: COc1cccc(-c2nc(S(=O)(=O)Nc3ccc(C(=O)O)cc3OC)cs2)c1] |
| 141 | 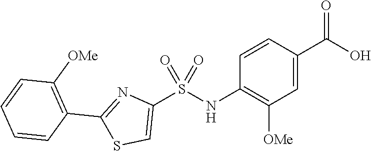 [SMILES: COc1cc(C(=O)O)ccc1NS(=O)(=O)c1csc(-c2ccccc2OC)n1] |
| 142 | 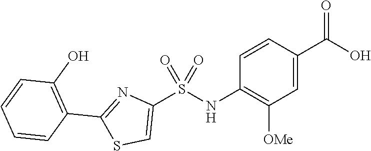 [SMILES: COc1cc(C(=O)O)ccc1NS(=O)(=O)c1csc(-c2ccccc2O)n1] |
| 143 | 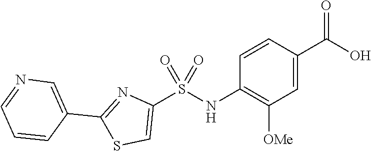 [SMILES: COc1cc(C(=O)O)ccc1NS(=O)(=O)c1csc(-c2cccnc2)n1] |
| 144 | 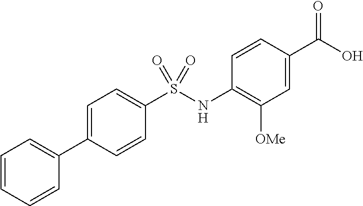 [SMILES: COc1cc(C(=O)O)ccc1NS(=O)(=O)c1ccc(-c2ccccc2)cc1] |
| 145 | 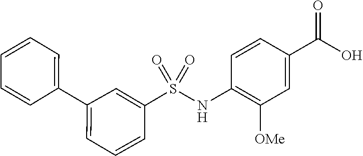 [SMILES: COc1cc(C(=O)O)ccc1NS(=O)(=O)c1cccc(-c2ccccc2)c1] |
| 146 | 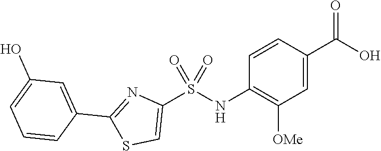 [SMILES: COc1cc(C(=O)O)ccc1NS(=O)(=O)c1csc(-c2cccc(O)c2)n1] |
| 147 | 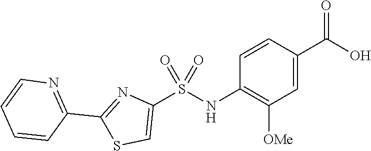 [SMILES: COc1cc(C(=O)O)ccc1NS(=O)(=O)c1csc(-c2ccccn2)n1] |
| 148 | 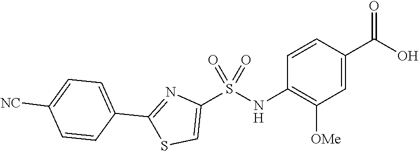 [SMILES: COc1cc(C(=O)O)ccc1NS(=O)(=O)c1csc(-c2ccc(C#N)cc2)n1] |
| 149 | 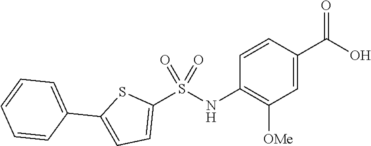 [SMILES: COc1cc(C(=O)O)ccc1NS(=O)(=O)c1ccc(-c2ccccc2)s1] |
| 150 | 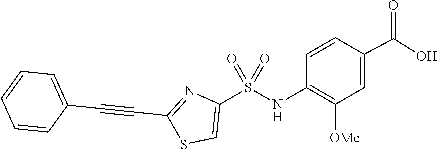 [SMILES: COc1cc(C(=O)O)ccc1NS(=O)(=O)c1csc(C#Cc2ccccc2)n1] |
| 151 | 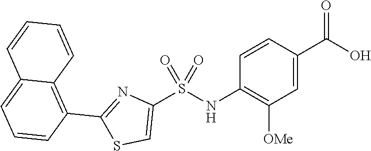 [SMILES: COc1cc(C(=O)O)ccc1NS(=O)(=O)c1csc(-c2cccc3ccccc23)n1] |
| 152 | 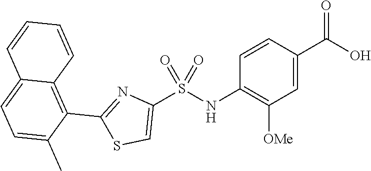 [SMILES: COc1cc(C(=O)O)ccc1NS(=O)(=O)c1csc(-c2c(C)ccc3ccccc23)n1] |
| 153 | 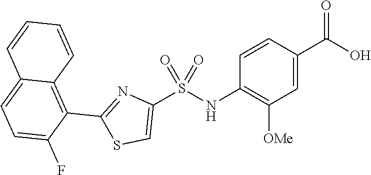 [SMILES: COc1cc(C(=O)O)ccc1NS(=O)(=O)c1csc(-c2c(F)ccc3ccccc23)n1] |
| 154 | 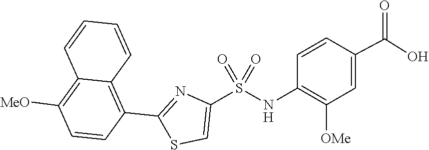 [SMILES: COc1cc(C(=O)O)ccc1NS(=O)(=O)c1csc(-c2ccc(OC)c3ccccc23)n1] |
| 155 | 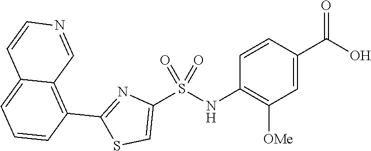 [SMILES: COc1cc(C(=O)O)ccc1NS(=O)(=O)c1csc(-c2cccc3ccncc23)n1] |
| 156 | 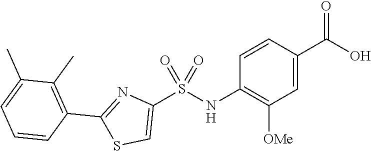 [SMILES: COc1cc(C(=O)O)ccc1NS(=O)(=O)c1csc(-c2cccc(C)c2C)n1] |
| 157 | 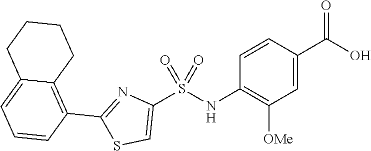 [SMILES: COc1cc(C(=O)O)ccc1NS(=O)(=O)c1csc(-c2cccc3c2CCCC3)n1] |
| 158 | 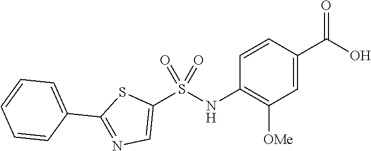 [SMILES: COc1cc(C(=O)O)ccc1NS(=O)(=O)c1cnc(-c2ccccc2)s1] |
| 159 | 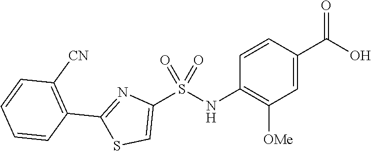 [SMILES: COc1cc(C(=O)O)ccc1NS(=O)(=O)c1csc(-c2ccccc2C#N)n1] |
| 160 | 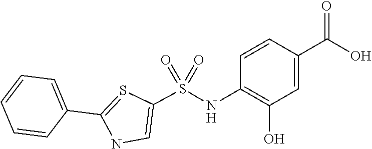 |
| 161 | 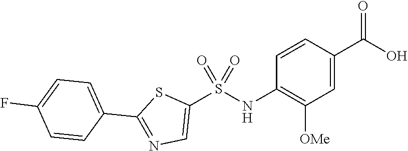 [SMILES: COc1cc(C(=O)O)ccc1NS(=O)(=O)c1cnc(-c2ccc(F)cc2)s1] |
| 162 | 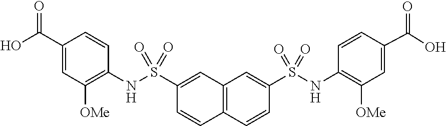 [SMILES: COc1cc(C(=O)O)ccc1NS(=O)(=O)c1ccc2ccc(S(=O)(=O)Nc3ccc(C(=O)O)cc3OC)cc2c1] |
| 163 | 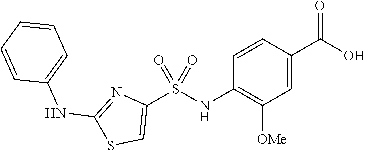 [SMILES: COc1cc(C(=O)O)ccc1NS(=O)(=O)c1csc(Nc2ccccc2)n1] |
| 164 | 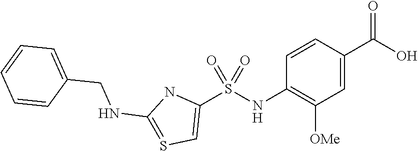 |
| 165 | 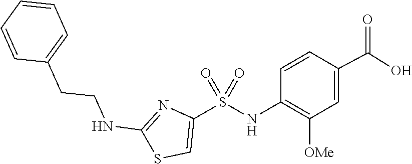 [SMILES: COc1cc(C(=O)O)ccc1NS(=O)(=O)c1csc(NCCc2ccccc2)n1] |
| 166 | 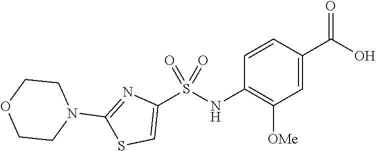 [SMILES: COc1cc(C(=O)O)ccc1NS(=O)(=O)c1csc(N2CCOCC2)n1] |
| 167 | 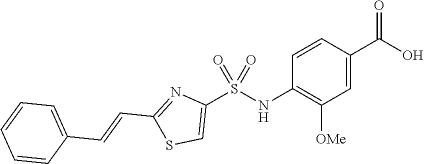 [SMILES: COc1cc(C(=O)O)ccc1NS(=O)(=O)c1csc(/C=C/c2ccccc2)n1] |
| 168 | 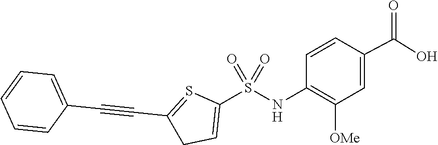 |
| 169 | 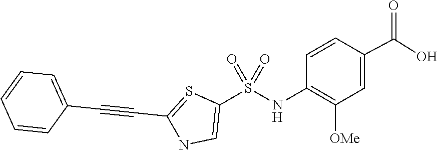 |
| 170 | 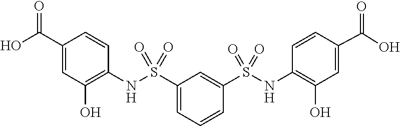 [SMILES: O=C(O)c1ccc(NS(=O)(=O)c2cccc(S(=O)(=O)Nc3ccc(C(=O)O)cc3O)c2)c(O)c1] |
| 171 | 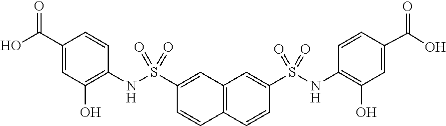 [SMILES: O=C(O)c1ccc(NS(=O)(=O)c2ccc3ccc(S(=O)(=O)Nc4ccc(C(=O)O)cc4O)cc3c2)c(O)c1] |
| 172 | 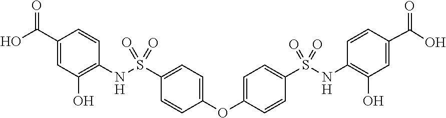 [SMILES: O=C(O)c1ccc(NS(=O)(=O)c2ccc(Oc3ccc(S(=O)(=O)Nc4ccc(C(=O)O)cc4O)cc3)cc2)c(O)c1] |
| 173 | 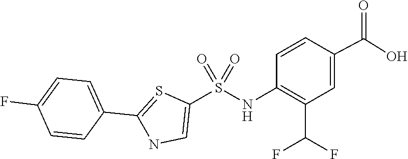 |
| 174 | 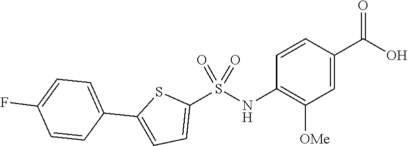 [SMILES: COc1cc(C(=O)O)ccc1NS(=O)(=O)c1ccc(-c2ccc(F)cc2)s1] |
| 175 | 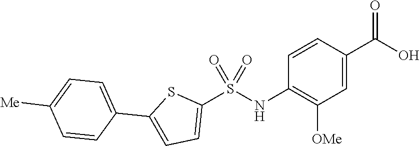 [SMILES: COc1cc(C(=O)O)ccc1NS(=O)(=O)c1ccc(-c2ccc(C)cc2)s1] |
| 176 | 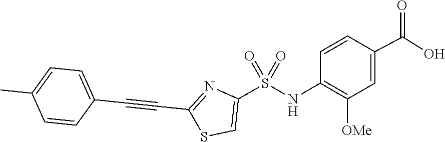 [SMILES: COc1cc(C(=O)O)ccc1NS(=O)(=O)c1csc(C#Cc2ccc(C)cc2)n1] |
| 177 | 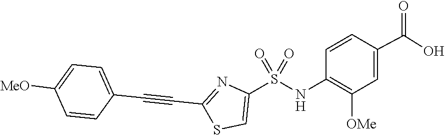 [SMILES: COc1ccc(C#Cc2nc(S(=O)(=O)Nc3ccc(C(=O)O)cc3OC)cs2)cc1] |
| 178 | 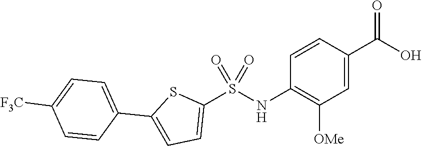 [SMILES: COc1cc(C(=O)O)ccc1NS(=O)(=O)c1ccc(-c2ccc(C(F)(F)F)cc2)s1] |
| 179 | 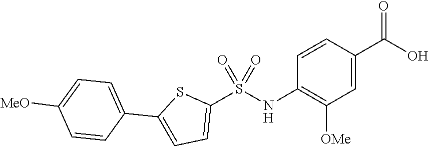 [SMILES: COc1ccc(-c2ccc(S(=O)(=O)Nc3ccc(C(=O)O)cc3OC)s2)cc1] |
| 180 | 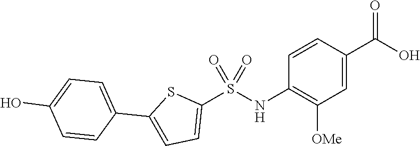 [SMILES: COc1cc(C(=O)O)ccc1NS(=O)(=O)c1ccc(-c2ccc(O)cc2)s1] |
| 181 | 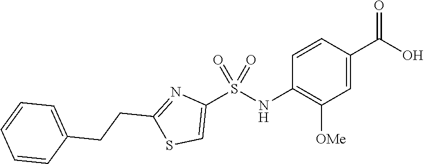 [SMILES: COc1cc(C(=O)O)ccc1NS(=O)(=O)c1csc(CCc2ccccc2)n1] |
| 182 | 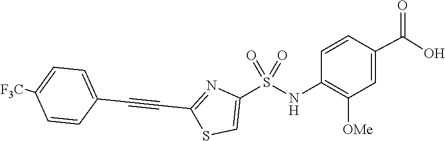 [SMILES: COc1cc(C(=O)O)ccc1NS(=O)(=O)c1csc(C#Cc2ccc(C(F)(F)F)cc2)n1] |
| 183 | 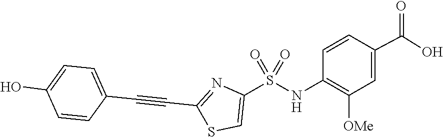 [SMILES: COc1cc(C(=O)O)ccc1NS(=O)(=O)c1csc(C#Cc2ccc(O)cc2)n1] |
| 184 | 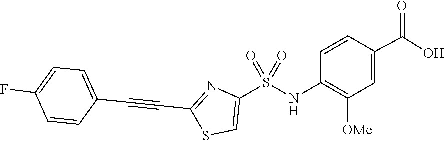 [SMILES: COc1cc(C(=O)O)ccc1NS(=O)(=O)c1csc(C#Cc2ccc(F)cc2)n1] |
| 185 | 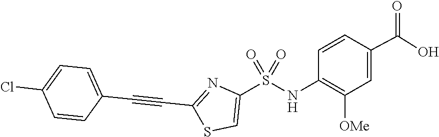 [SMILES: COc1cc(C(=O)O)ccc1NS(=O)(=O)c1csc(C#Cc2ccc(Cl)cc2)n1] |
| 186 | 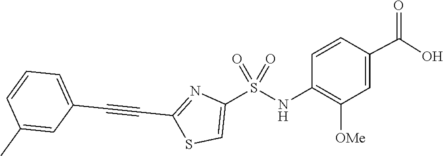 [SMILES: COc1cc(C(=O)O)ccc1NS(=O)(=O)c1csc(C#Cc2cccc(C)c2)n1] |
| 187 | 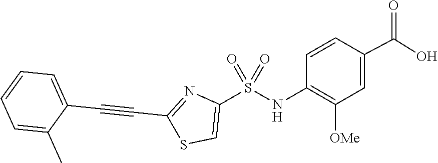 [SMILES: COc1cc(C(=O)O)ccc1NS(=O)(=O)c1csc(C#Cc2ccccc2C)n1] |
| 188 | 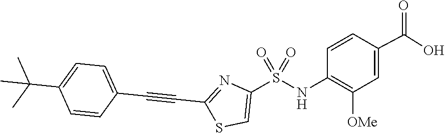 [SMILES: COc1cc(C(=O)O)ccc1NS(=O)(=O)c1csc(C#Cc2ccc(C(C)(C)C)cc2)n1] |
| 189 | 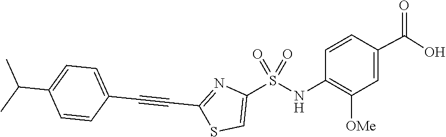 [SMILES: COc1cc(C(=O)O)ccc1NS(=O)(=O)c1csc(C#Cc2ccc(C(C)C)cc2)n1] |
| 190 | 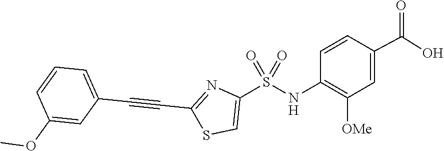 [SMILES: COc1cccc(C#Cc2nc(S(=O)(=O)Nc3ccc(C(=O)O)cc3OC)cs2)c1] |
| 191 | 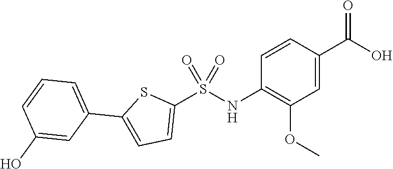 [SMILES: COc1cc(C(=O)O)ccc1NS(=O)(=O)c1ccc(-c2cccc(O)c2)s1] |
| 192 | 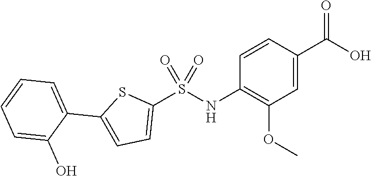 [SMILES: COc1cc(C(=O)O)ccc1NS(=O)(=O)c1ccc(-c2ccccc2O)s1] |
| 193 | 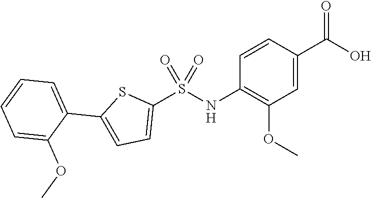 [SMILES: COc1cc(C(=O)O)ccc1NS(=O)(=O)c1ccc(-c2ccccc2OC)s1] |
| 194 | 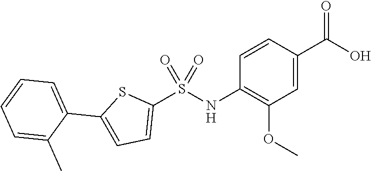 [SMILES: COc1cc(C(=O)O)ccc1NS(=O)(=O)c1ccc(-c2ccccc2C)s1] |
| 195 | 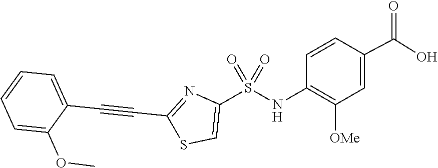 [SMILES: COc1ccccc1C#Cc1nc(S(=O)(=O)Nc2ccc(C(=O)O)cc2OC)cs1] |
| 196 | 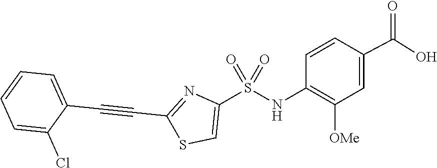 [SMILES: COc1cc(C(=O)O)ccc1NS(=O)(=O)c1csc(C#Cc2ccccc2Cl)n1] |
| 197 | 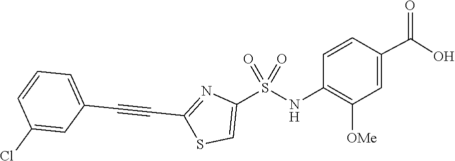 [SMILES: COc1cc(C(=O)O)ccc1NS(=O)(=O)c1csc(C#Cc2cccc(Cl)c2)n1] |
| 198 | 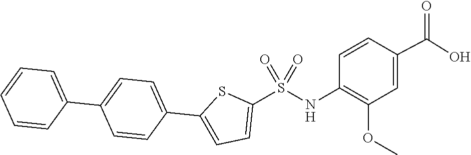 [SMILES: COc1cc(C(=O)O)ccc1NS(=O)(=O)c1ccc(-c2ccc(-c3ccccc3)cc2)s1] |
| 199 | 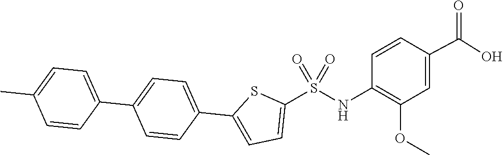 [SMILES: COc1cc(C(=O)O)ccc1NS(=O)(=O)c1ccc(-c2ccc(-c3ccc(C)cc3)cc2)s1] |
| 200 | 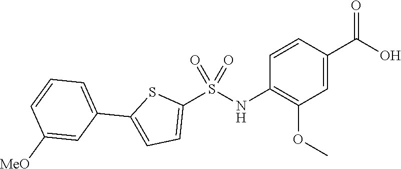 [SMILES: COc1cccc(-c2ccc(S(=O)(=O)Nc3ccc(C(=O)O)cc3OC)s2)c1] |
| 201 | 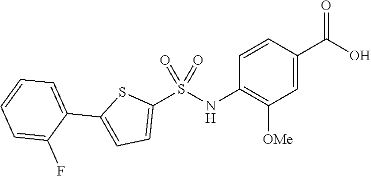 [SMILES: COc1cc(C(=O)O)ccc1NS(=O)(=O)c1ccc(-c2ccccc2F)s1] |
| 202 | 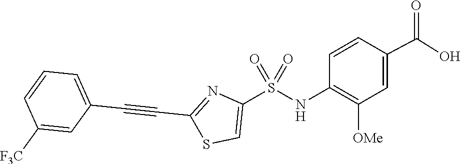 [SMILES: COc1cc(C(=O)O)ccc1NS(=O)(=O)c1csc(C#Cc2cccc(C(F)(F)F)c2)n1] |
| 203 | 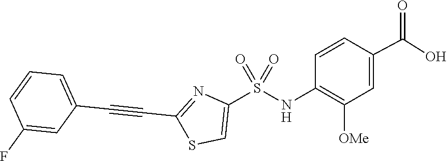 [SMILES: COc1cc(C(=O)O)ccc1NS(=O)(=O)c1csc(C#Cc2cccc(F)c2)n1] |
| 204 | 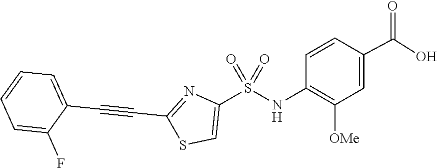 [SMILES: COc1cc(C(=O)O)ccc1NS(=O)(=O)c1csc(C#Cc2ccccc2F)n1] |
| 205 | 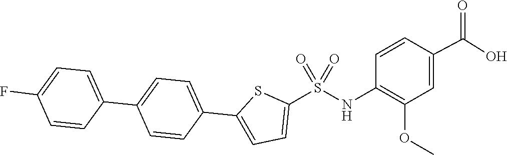 [SMILES: COc1cc(C(=O)O)ccc1NS(=O)(=O)c1ccc(-c2ccc(-c3ccc(F)cc3)cc2)s1] |
| 206 | 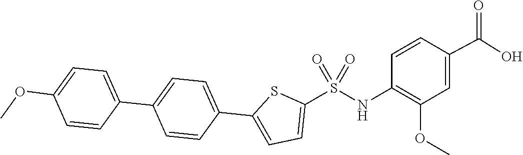 [SMILES: COc1ccc(-c2ccc(-c3ccc(S(=O)(=O)Nc4ccc(C(=O)O)cc4OC)s3)cc2)cc1] |
| 207 | 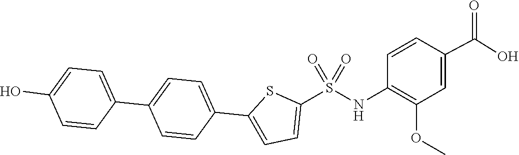 [SMILES: COc1cc(C(=O)O)ccc1NS(=O)(=O)c1ccc(-c2ccc(-c3ccc(O)cc3)cc2)s1] |
| 208 | 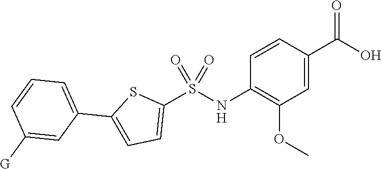 |
| 209 | 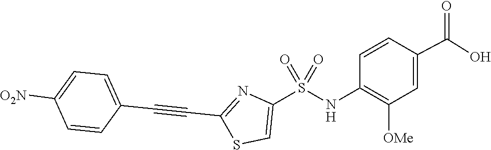 [SMILES: COc1cc(C(=O)O)ccc1NS(=O)(=O)c1csc(C#Cc2ccc([N+](=O)[O-])cc2)n1] |
| 210 | 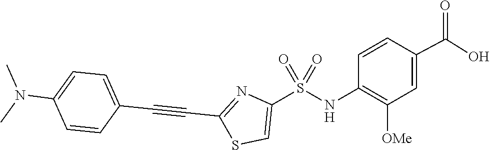 [SMILES: COc1cc(C(=O)O)ccc1NS(=O)(=O)c1csc(C#Cc2ccc(N(C)C)cc2)n1] |
| 211 | 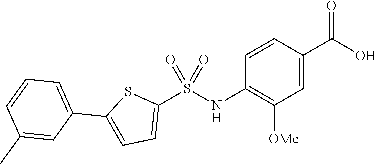 [SMILES: COc1cc(C(=O)O)ccc1NS(=O)(=O)c1ccc(-c2cccc(C)c2)s1] |
| 212 | 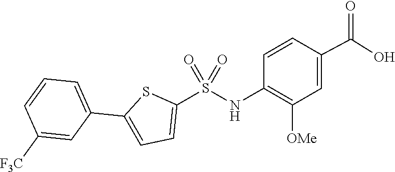 [SMILES: COc1cc(C(=O)O)ccc1NS(=O)(=O)c1ccc(-c2cccc(C(F)(F)F)c2)s1] |
| 213 | 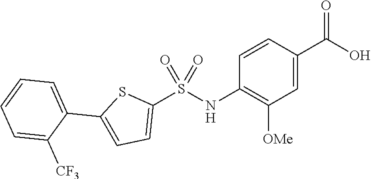 [SMILES: COc1cc(C(=O)O)ccc1NS(=O)(=O)c1ccc(-c2ccccc2C(F)(F)F)s1] |
| 214 | 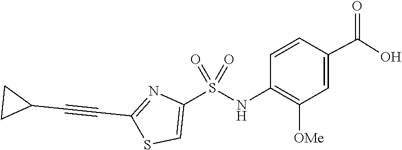 [SMILES: COc1cc(C(=O)O)ccc1NS(=O)(=O)c1csc(C#CC2CC2)n1] |
| 215 | 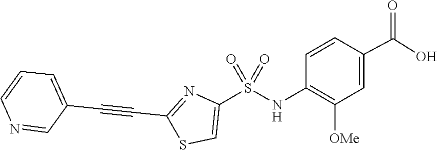 [SMILES: COc1cc(C(=O)O)ccc1NS(=O)(=O)c1csc(C#Cc2cccnc2)n1] |
| 216 | 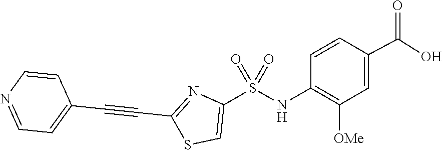 [SMILES: COc1cc(C(=O)O)ccc1NS(=O)(=O)c1csc(C#Cc2ccncc2)n1] |
| 217 | 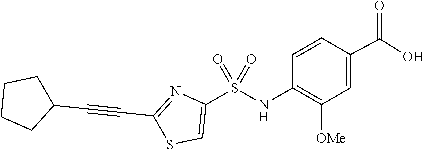 [SMILES: COc1cc(C(=O)O)ccc1NS(=O)(=O)c1csc(C#CC2CCCC2)n1] |
| 218 | 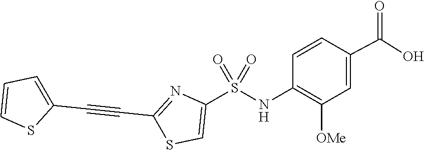 [SMILES: COc1cc(C(=O)O)ccc1NS(=O)(=O)c1csc(C#Cc2cccs2)n1] |
| 219 | 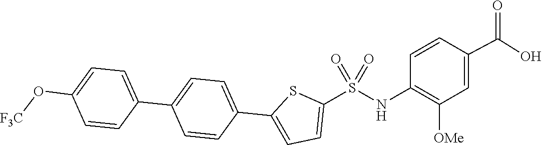 [SMILES: COc1cc(C(=O)O)ccc1NS(=O)(=O)c1ccc(-c2ccc(-c3ccc(OC(F)(F)F)cc3)cc2)s1] |
| 220 | 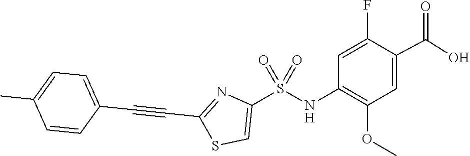 [SMILES: COc1cc(C(=O)O)c(F)cc1NS(=O)(=O)c1csc(C#Cc2ccc(C)cc2)n1] |
| 221 | 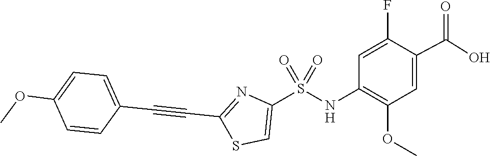 [SMILES: COc1ccc(C#Cc2nc(S(=O)(=O)Nc3cc(F)c(C(=O)O)cc3OC)cs2)cc1] |
| 222 | 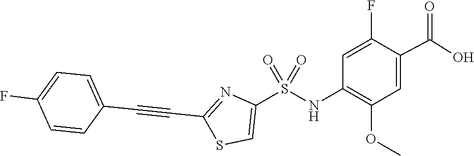 [SMILES: COc1cc(C(=O)O)c(F)cc1NS(=O)(=O)c1csc(C#Cc2ccc(F)cc2)n1] |
| 223 | 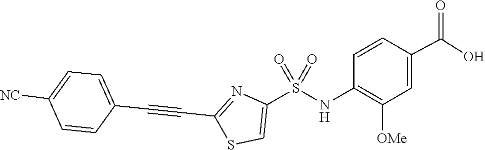 [SMILES: COc1cc(C(=O)O)ccc1NS(=O)(=O)c1csc(C#Cc2ccc(C#N)cc2)n1] |
| 224 | 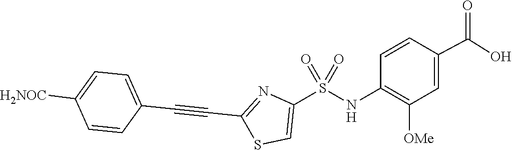 [SMILES: COc1cc(C(=O)O)ccc1NS(=O)(=O)c1csc(C#Cc2ccc(C(N)=O)cc2)n1] |
| 225 | 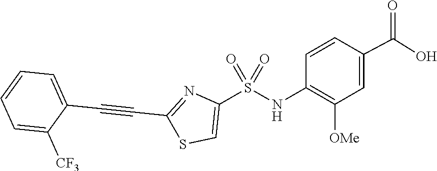 [SMILES: COc1cc(C(=O)O)ccc1NS(=O)(=O)c1csc(C#Cc2ccccc2C(F)(F)F)n1] |
| 226 | 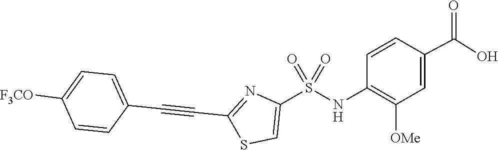 [SMILES: COc1cc(C(=O)O)ccc1NS(=O)(=O)c1csc(C#Cc2ccc(OC(F)(F)F)cc2)n1] |
| 227 | 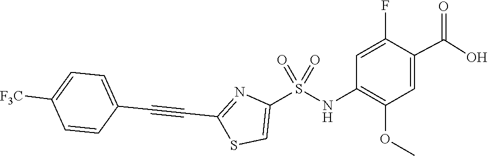 [SMILES: COc1cc(C(=O)O)c(F)cc1NS(=O)(=O)c1csc(C#Cc2ccc(C(F)(F)F)cc2)n1] |
| 228 | 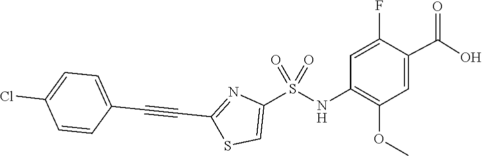 [SMILES: COc1cc(C(=O)O)c(F)cc1NS(=O)(=O)c1csc(C#Cc2ccc(Cl)cc2)n1] |
| 229 | 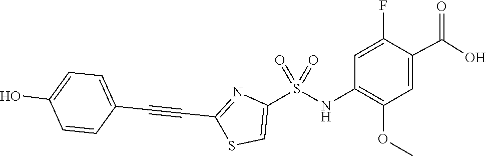 [SMILES: COc1cc(C(=O)O)c(F)cc1NS(=O)(=O)c1csc(C#Cc2ccc(O)cc2)n1] |
| 230 | 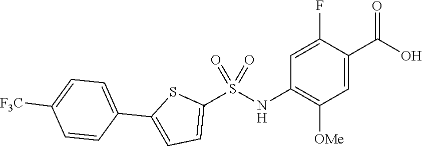 [SMILES: COc1cc(C(=O)O)c(F)cc1NS(=O)(=O)c1ccc(-c2ccc(C(F)(F)F)cc2)s1] |
| 231 | 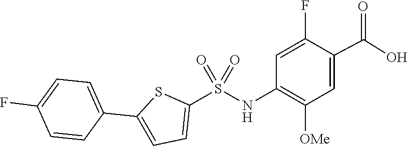 [SMILES: COc1cc(C(=O)O)c(F)cc1NS(=O)(=O)c1ccc(-c2ccc(F)cc2)s1] |
| 232 | 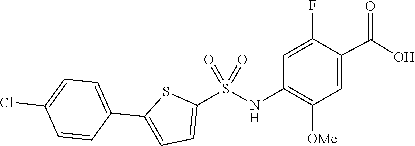 [SMILES: COc1cc(C(=O)O)c(F)cc1NS(=O)(=O)c1ccc(-c2ccc(Cl)cc2)s1] |
| 233 | 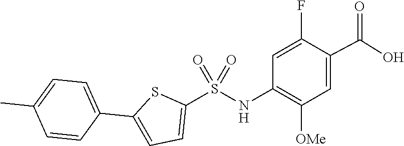 [SMILES: COc1cc(C(=O)O)c(F)cc1NS(=O)(=O)c1ccc(-c2ccc(C)cc2)s1] |
| 234 | 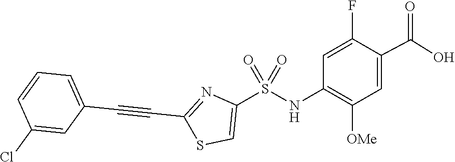 [SMILES: COc1cc(C(=O)O)c(F)cc1NS(=O)(=O)c1csc(C#Cc2cccc(Cl)c2)n1] |
| 235 | 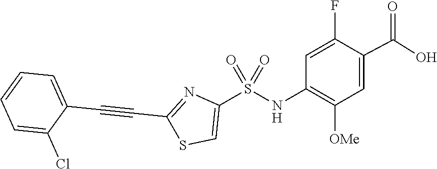 [SMILES: COc1cc(C(=O)O)c(F)cc1NS(=O)(=O)c1csc(C#Cc2ccccc2Cl)n1] |
| 236 | 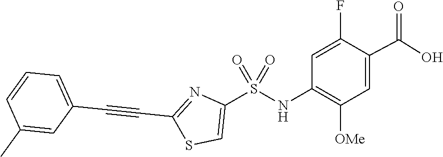 [SMILES: COc1cc(C(=O)O)c(F)cc1NS(=O)(=O)c1csc(C#Cc2cccc(C)c2)n1] |
| 237 | 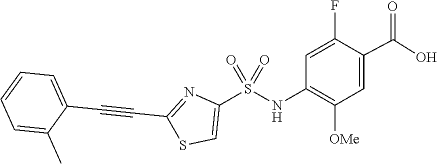 [SMILES: COc1cc(C(=O)O)c(F)cc1NS(=O)(=O)c1csc(C#Cc2ccccc2C)n1] |
| 238 | 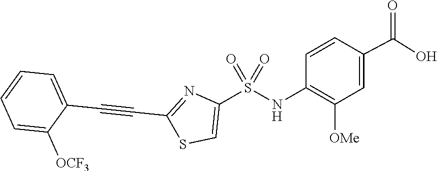 [SMILES: COc1cc(C(=O)O)ccc1NS(=O)(=O)c1csc(C#Cc2ccccc2OC(F)(F)F)n1] |
| 239 | 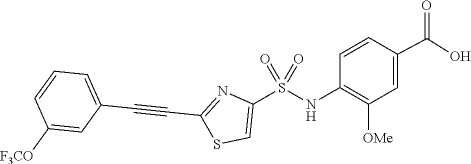 [SMILES: COc1cc(C(=O)O)ccc1NS(=O)(=O)c1csc(C#Cc2cccc(OC(F)(F)F)c2)n1] |
| 240 | 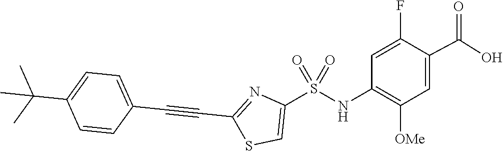 [SMILES: COc1cc(C(=O)O)c(F)cc1NS(=O)(=O)c1csc(C#Cc2ccc(C(C)(C)C)cc2)n1] |
| 241 | 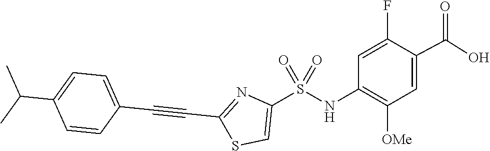 [SMILES: COc1cc(C(=O)O)c(F)cc1NS(=O)(=O)c1csc(C#Cc2ccc(C(C)C)cc2)n1] |
| 242 | 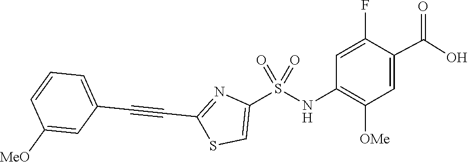 [SMILES: COc1cccc(C#Cc2nc(S(=O)(=O)Nc3cc(F)c(C(=O)O)cc3OC)cs2)c1] |
| 243 | 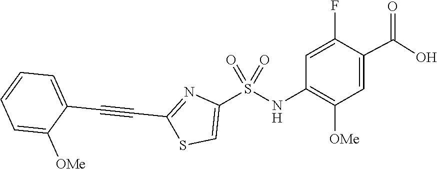 [SMILES: COc1ccccc1C#Cc1nc(S(=O)(=O)Nc2cc(F)c(C(=O)O)cc2OC)cs1] |
| 244 | 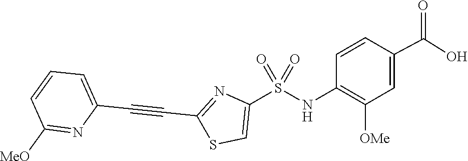 [SMILES: COc1cccc(C#Cc2nc(S(=O)(=O)Nc3ccc(C(=O)O)cc3OC)cs2)n1] |
| 245 | 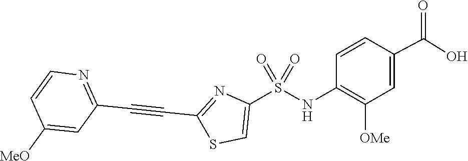 [SMILES: COc1ccnc(C#Cc2nc(S(=O)(=O)Nc3ccc(C(=O)O)cc3OC)cs2)c1] |
| 246 | 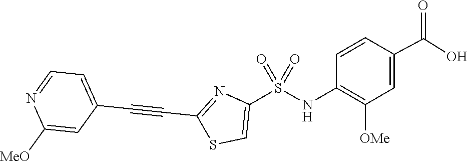 [SMILES: COc1cc(C#Cc2nc(S(=O)(=O)Nc3ccc(C(=O)O)cc3OC)cs2)ccn1] |
| 247 | 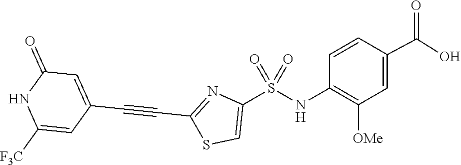 [SMILES: COc1cc(C(=O)O)ccc1NS(=O)(=O)c1csc(C#Cc2cc(C(F)(F)F)[nH]c(=O)c2)n1] |
| 248 | 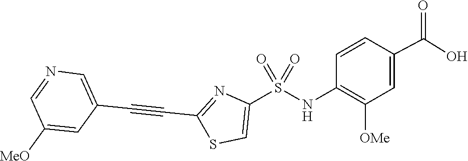 [SMILES: COc1cncc(C#Cc2nc(S(=O)(=O)Nc3ccc(C(=O)O)cc3OC)cs2)c1] |
| 249 | 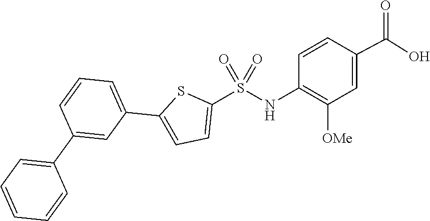 [SMILES: COc1cc(C(=O)O)ccc1NS(=O)(=O)c1ccc(-c2cccc(-c3ccccc3)c2)s1] |
| 250 | 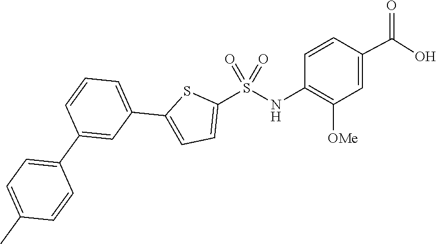 [SMILES: COc1cc(C(=O)O)ccc1NS(=O)(=O)c1ccc(-c2cccc(-c3ccc(C)cc3)c2)s1] |
| 251 | 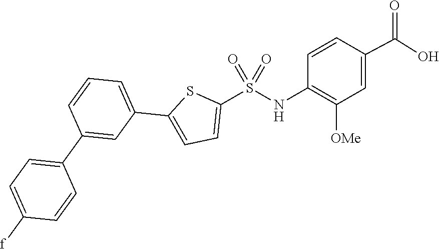 |
| 252 | 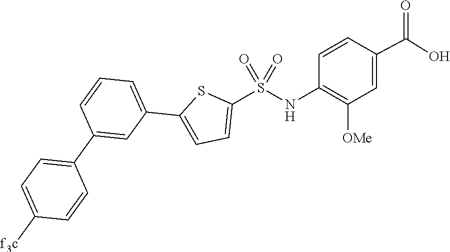 |
| 253 | 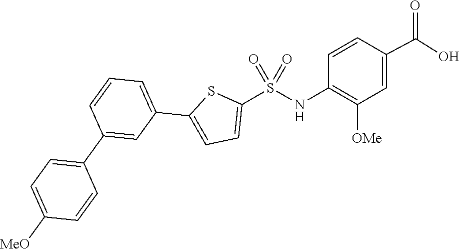 [SMILES: COc1ccc(-c2cccc(-c3ccc(S(=O)(=O)Nc4ccc(C(=O)O)cc4OC)s3)c2)cc1] |
| 254 | 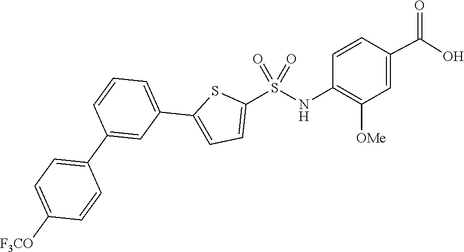 [SMILES: COc1cc(C(=O)O)ccc1NS(=O)(=O)c1ccc(-c2cccc(-c3ccc(OC(F)(F)F)cc3)c2)s1] |
| 255 | 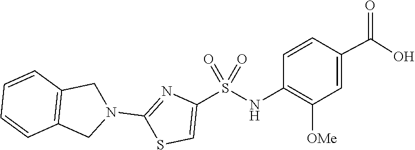 [SMILES: COc1cc(C(=O)O)ccc1NS(=O)(=O)c1csc(N2Cc3ccccc3C2)n1] |
| 256 | 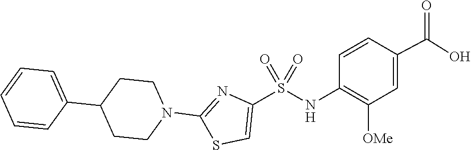 [SMILES: COc1cc(C(=O)O)ccc1NS(=O)(=O)c1csc(N2CCC(c3ccccc3)CC2)n1] |
| 257 | 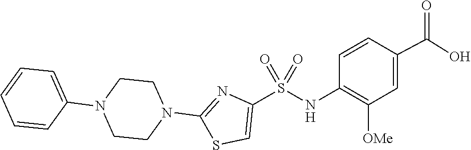 [SMILES: COc1cc(C(=O)O)ccc1NS(=O)(=O)c1csc(N2CCN(c3ccccc3)CC2)n1] |
| 258 | 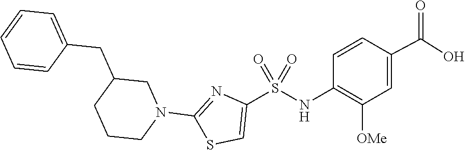 [SMILES: COc1cc(C(=O)O)ccc1NS(=O)(=O)c1csc(N2CCCC(Cc3ccccc3)C2)n1] |
| 259 | 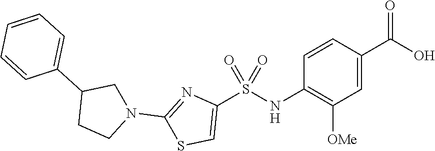 [SMILES: COc1cc(C(=O)O)ccc1NS(=O)(=O)c1csc(N2CCC(c3ccccc3)C2)n1] |
| 260 | 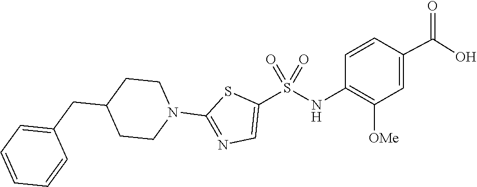 [SMILES: COc1cc(C(=O)O)ccc1NS(=O)(=O)c1cnc(N2CCC(Cc3ccccc3)CC2)s1] |
| 261 | 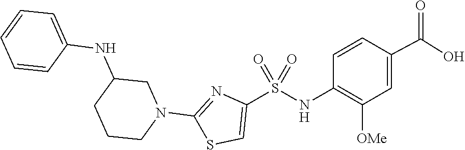 [SMILES: COc1cc(C(=O)O)ccc1NS(=O)(=O)c1csc(N2CCCC(Nc3ccccc3)C2)n1] |
| 262 | 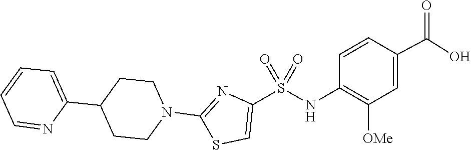 [SMILES: COc1cc(C(=O)O)ccc1NS(=O)(=O)c1csc(N2CCC(c3ccccn3)CC2)n1] |
| 263 | 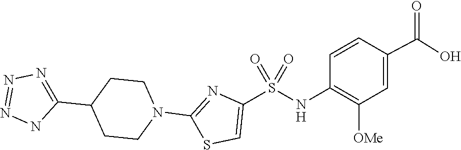 [SMILES: COc1cc(C(=O)O)ccc1NS(=O)(=O)c1csc(N2CCC(c3nnn[nH]3)CC2)n1] |
| 264 | 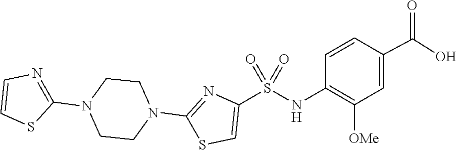 [SMILES: COc1cc(C(=O)O)ccc1NS(=O)(=O)c1csc(N2CCN(c3nccs3)CC2)n1] |
| 265 | 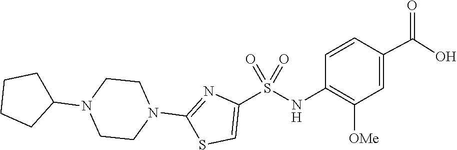 [SMILES: COc1cc(C(=O)O)ccc1NS(=O)(=O)c1csc(N2CCN(C3CCCC3)CC2)n1] |
| 266 | 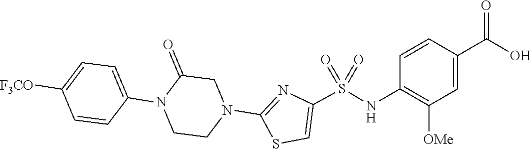 [SMILES: COc1cc(C(=O)O)ccc1NS(=O)(=O)c1csc(N2CCN(c3ccc(OC(F)(F)F)cc3)C(=O)C2)n1] |
| 267 | 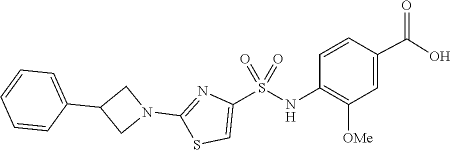 [SMILES: COc1cc(C(=O)O)ccc1NS(=O)(=O)c1csc(N2CC(c3ccccc3)C2)n1] |
| 268 | 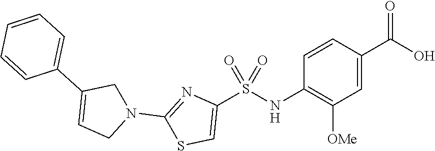 [SMILES: COc1cc(C(=O)O)ccc1NS(=O)(=O)c1csc(N2CC=C(c3ccccc3)C2)n1] |
| 269 | 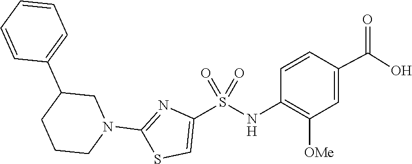 [SMILES: COc1cc(C(=O)O)ccc1NS(=O)(=O)c1csc(N2CCCC(c3ccccc3)C2)n1] |
| 270 | 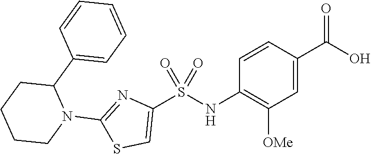 [SMILES: COc1cc(C(=O)O)ccc1NS(=O)(=O)c1csc(N2CCCCC2c2ccccc2)n1] |
| 271 | 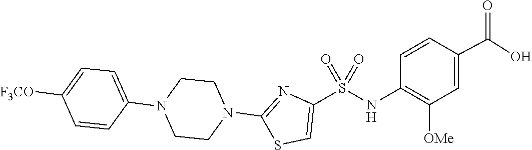 [SMILES: COc1cc(C(=O)O)ccc1NS(=O)(=O)c1csc(N2CCN(c3ccc(OC(F)(F)F)cc3)CC2)n1] |
| 272 | 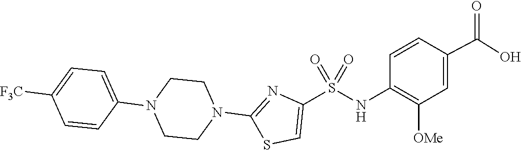 [SMILES: COc1cc(C(=O)O)ccc1NS(=O)(=O)c1csc(N2CCN(c3ccc(C(F)(F)F)cc3)CC2)n1] |
| 273 | 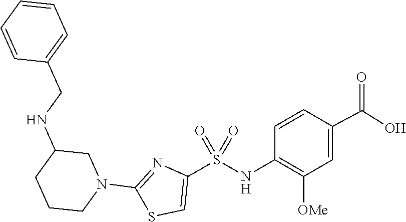 [SMILES: COc1cc(C(=O)O)ccc1NS(=O)(=O)c1csc(N2CCCC(NCc3ccccc3)C2)n1] |
| 274 | 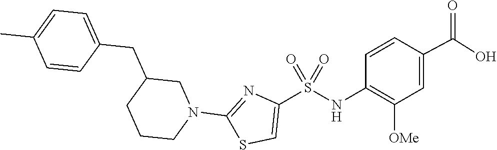 [SMILES: COc1cc(C(=O)O)ccc1NS(=O)(=O)c1csc(N2CCCC(Cc3ccc(C)cc3)C2)n1] |
| 275 | 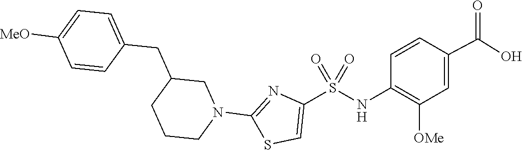 [SMILES: COc1ccc(CC2CCCN(c3nc(S(=O)(=O)Nc4ccc(C(=O)O)cc4OC)cs3)C2)cc1] |
| 276 | 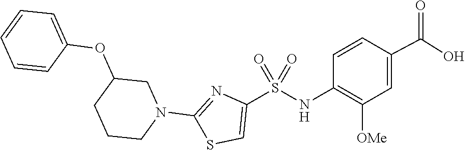 [SMILES: COc1cc(C(=O)O)ccc1NS(=O)(=O)c1csc(N2CCCC(Oc3ccccc3)C2)n1] |
| 277 | 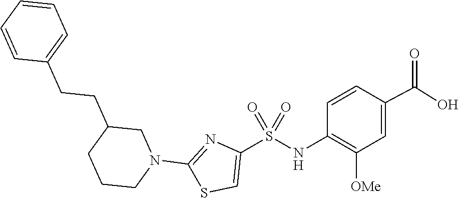 [SMILES: COc1cc(C(=O)O)ccc1NS(=O)(=O)c1csc(N2CCCC(CCc3ccccc3)C2)n1] |
| 278 | 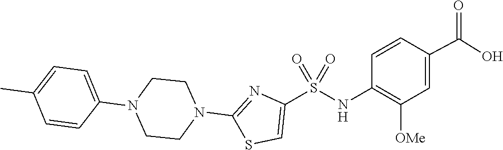 [SMILES: COc1cc(C(=O)O)ccc1NS(=O)(=O)c1csc(N2CCN(c3ccc(C)cc3)CC2)n1] |
| 279 | 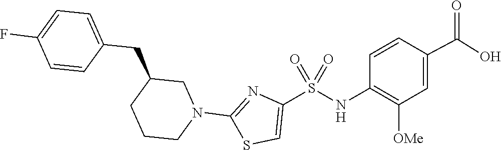 [SMILES: COc1cc(C(=O)O)ccc1NS(=O)(=O)c1csc(N2CCC[C@@H](Cc3ccc(F)cc3)C2)n1] |
| 280 | 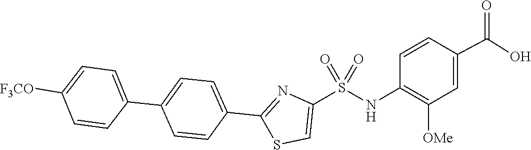 [SMILES: COc1cc(C(=O)O)ccc1NS(=O)(=O)c1csc(-c2ccc(-c3ccc(OC(F)(F)F)cc3)cc2)n1] |
| 281 | 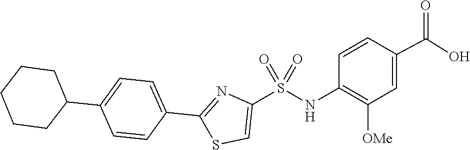 [SMILES: COc1cc(C(=O)O)ccc1NS(=O)(=O)c1csc(-c2ccc(C3CCCCC3)cc2)n1] |
| 282 | 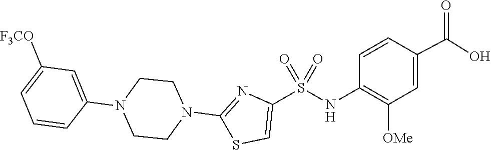 [SMILES: COc1cc(C(=O)O)ccc1NS(=O)(=O)c1csc(N2CCN(c3cccc(OC(F)(F)F)c3)CC2)n1] |
| 283 | 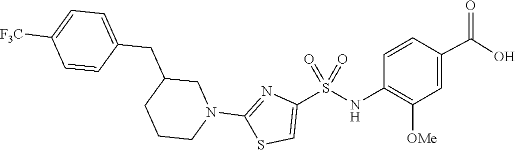 [SMILES: COc1cc(C(=O)O)ccc1NS(=O)(=O)c1csc(N2CCCC(Cc3ccc(C(F)(F)F)cc3)C2)n1] |
| 284 | 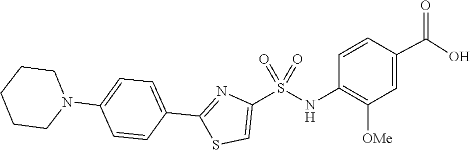 [SMILES: COc1cc(C(=O)O)ccc1NS(=O)(=O)c1csc(-c2ccc(N3CCCCC3)cc2)n1] |
| 285 | 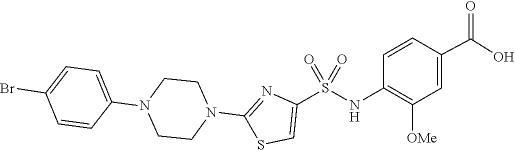 [SMILES: COc1cc(C(=O)O)ccc1NS(=O)(=O)c1csc(N2CCN(c3ccc(Br)cc3)CC2)n1] |
| 286 | 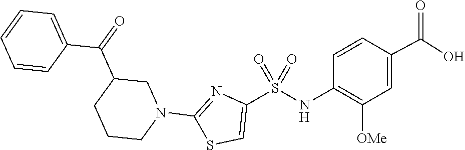 [SMILES: COc1cc(C(=O)O)ccc1NS(=O)(=O)c1csc(N2CCCC(C(=O)c3ccccc3)C2)n1] |
| 287 | 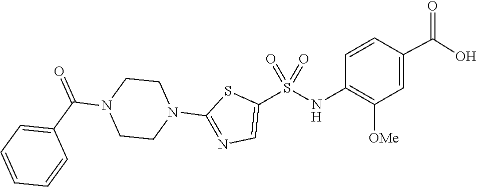 [SMILES: COc1cc(C(=O)O)ccc1NS(=O)(=O)c1cnc(N2CCN(C(=O)c3ccccc3)CC2)s1] |
| 288 | 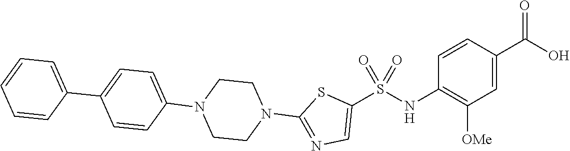 [SMILES: COc1cc(C(=O)O)ccc1NS(=O)(=O)c1cnc(N2CCN(c3ccc(-c4ccccc4)cc3)CC2)s1] |
| 289 | 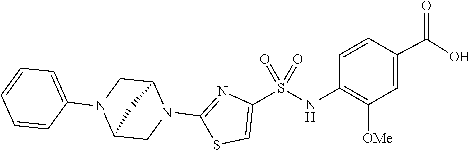 [SMILES: COc1cc(C(=O)O)ccc1NS(=O)(=O)c1csc(N2C[C@@H]3C[C@H]2CN3c2ccccc2)n1] |
| 290 | 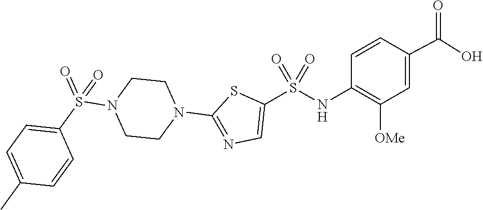 [SMILES: COc1cc(C(=O)O)ccc1NS(=O)(=O)c1cnc(N2CCN(S(=O)(=O)c3ccc(C)cc3)CC2)s1] |
| 291 | 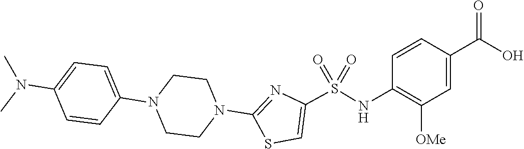 [SMILES: COc1cc(C(=O)O)ccc1NS(=O)(=O)c1csc(N2CCN(c3ccc(N(C)C)cc3)CC2)n1] |
| 292 | 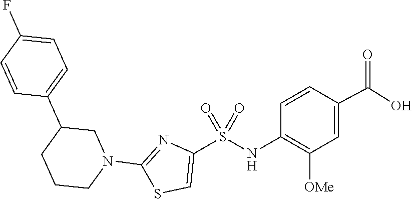 [SMILES: COc1cc(C(=O)O)ccc1NS(=O)(=O)c1csc(N2CCCC(c3ccc(F)cc3)C2)n1] |
| 293 | 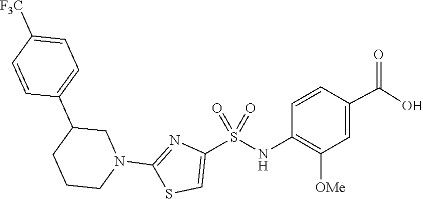 [SMILES: COc1cc(C(=O)O)ccc1NS(=O)(=O)c1csc(N2CCCC(c3ccc(C(F)(F)F)cc3)C2)n1] |
| 294 | 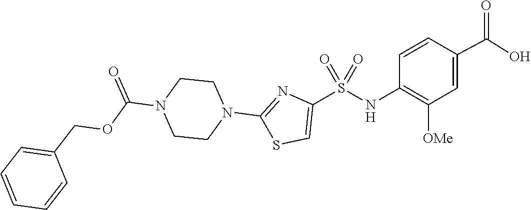 [SMILES: COc1cc(C(=O)O)ccc1NS(=O)(=O)c1csc(N2CCN(C(=O)OCc3ccccc3)CC2)n1] |
| 295 | 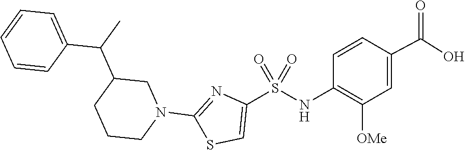 [SMILES: COc1cc(C(=O)O)ccc1NS(=O)(=O)c1csc(N2CCCC(C(C)c3ccccc3)C2)n1] |
| 296 | 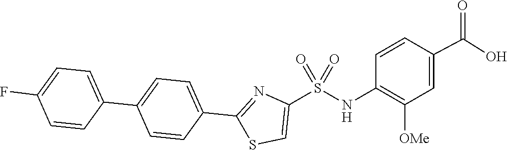 [SMILES: COc1cc(C(=O)O)ccc1NS(=O)(=O)c1csc(-c2ccc(-c3ccc(F)cc3)cc2)n1] |
| 297 | 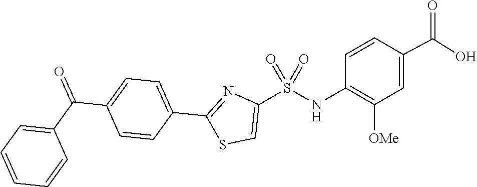 [SMILES: COc1cc(C(=O)O)ccc1NS(=O)(=O)c1csc(-c2ccc(C(=O)c3ccccc3)cc2)n1] |
| 298 | 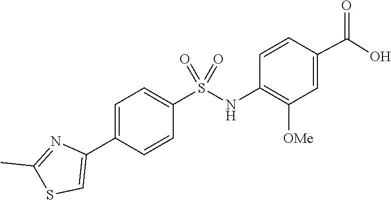 [SMILES: COc1cc(C(=O)O)ccc1NS(=O)(=O)c1ccc(-c2csc(C)n2)cc1] |
| 299 | 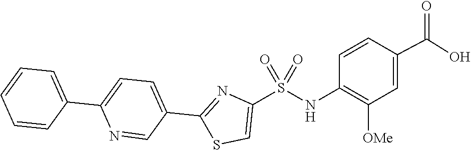 [SMILES: COc1cc(C(=O)O)ccc1NS(=O)(=O)c1csc(-c2ccc(-c3ccccc3)nc2)n1] |
| 300 | 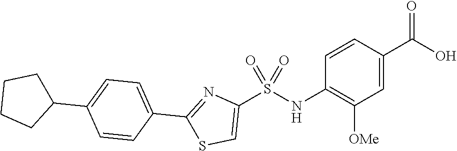 [SMILES: COc1cc(C(=O)O)ccc1NS(=O)(=O)c1csc(-c2ccc(C3CCCC3)cc2)n1] |
| 301 | 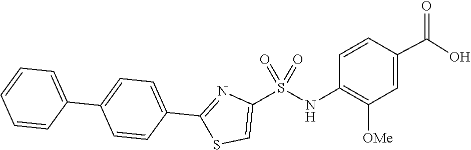 [SMILES: COc1cc(C(=O)O)ccc1NS(=O)(=O)c1csc(-c2ccc(-c3ccccc3)cc2)n1] |
| 302 | 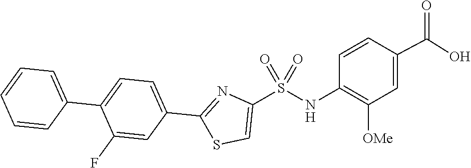 [SMILES: COc1cc(C(=O)O)ccc1NS(=O)(=O)c1csc(-c2ccc(-c3ccccc3)c(F)c2)n1] |
| 303 | 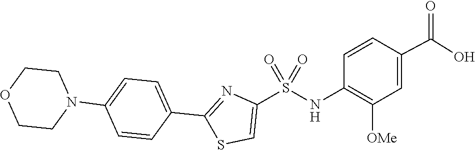 [SMILES: COc1cc(C(=O)O)ccc1NS(=O)(=O)c1csc(-c2ccc(N3CCOCC3)cc2)n1] |
| 304 | 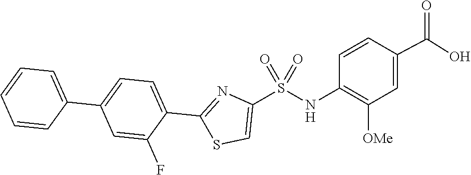 [SMILES: COc1cc(C(=O)O)ccc1NS(=O)(=O)c1csc(-c2ccc(-c3ccccc3)cc2F)n1] |
| 305 | 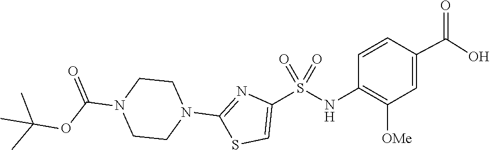 [SMILES: COc1cc(C(=O)O)ccc1NS(=O)(=O)c1csc(N2CCN(C(=O)OC(C)(C)C)CC2)n1] |
| 306 | 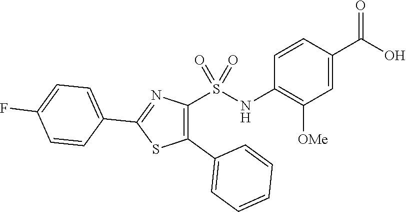 [SMILES: COc1cc(C(=O)O)ccc1NS(=O)(=O)c1nc(-c2ccc(F)cc2)sc1-c1ccccc1] |
| 307 | 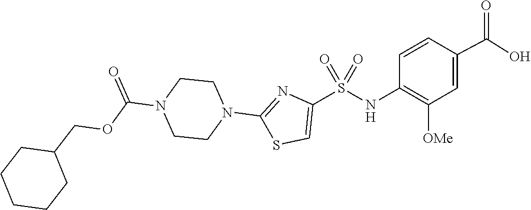 [SMILES: COc1cc(C(=O)O)ccc1NS(=O)(=O)c1csc(N2CCN(C(=O)OCC3CCCCC3)CC2)n1] |
| 308 | 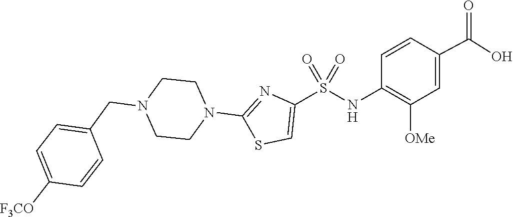 [SMILES: COc1cc(C(=O)O)ccc1NS(=O)(=O)c1csc(N2CCN(Cc3ccc(OC(F)(F)F)cc3)CC2)n1] |
| 309 | 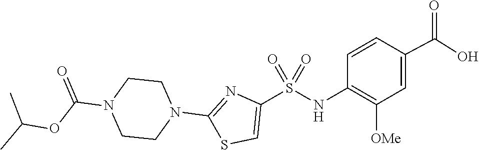 [SMILES: COc1cc(C(=O)O)ccc1NS(=O)(=O)c1csc(N2CCN(C(=O)OC(C)C)CC2)n1] |
| 310 | 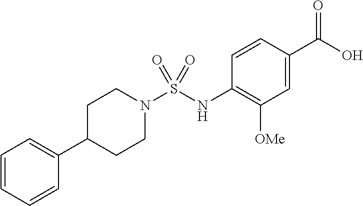 [SMILES: COc1cc(C(=O)O)ccc1NS(=O)(=O)N1CCC(c2ccccc2)CC1] |
| 311 | 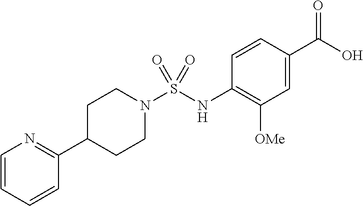 [SMILES: COc1cc(C(=O)O)ccc1NS(=O)(=O)N1CCC(c2ccccn2)CC1] |
| 312 | 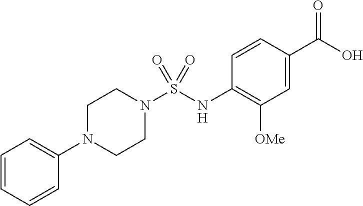 [SMILES: COc1cc(C(=O)O)ccc1NS(=O)(=O)N1CCN(c2ccccc2)CC1] |
| 313 | 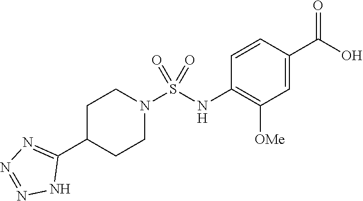 [SMILES: COc1cc(C(=O)O)ccc1NS(=O)(=O)N1CCC(c2nnn[nH]2)CC1] |
| 314 | 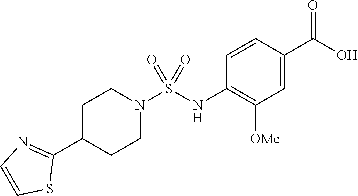 [SMILES: COc1cc(C(=O)O)ccc1NS(=O)(=O)N1CCC(c2nccs2)CC1] |
| 315 | 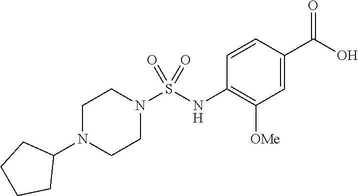 [SMILES: COc1cc(C(=O)O)ccc1NS(=O)(=O)N1CCN(C2CCCC2)CC1] |
| 316 | 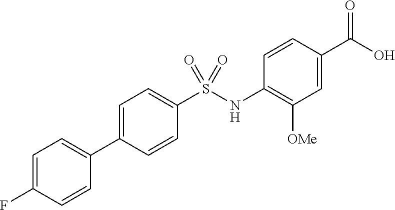 [SMILES: COc1cc(C(=O)O)ccc1NS(=O)(=O)c1ccc(-c2ccc(F)cc2)cc1] |
| 317 | 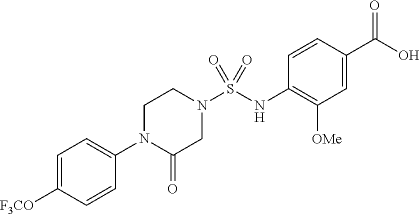 [SMILES: COc1cc(C(=O)O)ccc1NS(=O)(=O)N1CCN(c2ccc(OC(F)(F)F)cc2)C(=O)C1] |
| 318 | 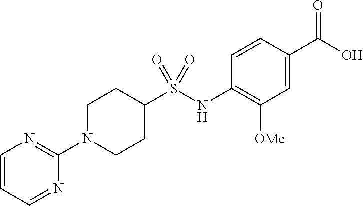 [SMILES: COc1cc(C(=O)O)ccc1NS(=O)(=O)C1CCN(c2ncccn2)CC1] |
| 319 | 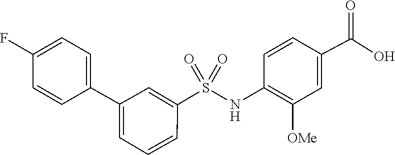 [SMILES: COc1cc(C(=O)O)ccc1NS(=O)(=O)c1cccc(-c2ccc(F)cc2)c1] |
| 320 | 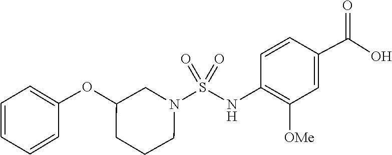 [SMILES: COc1cc(C(=O)O)ccc1NS(=O)(=O)N1CCCC(Oc2ccccc2)C1] |
| 321 | 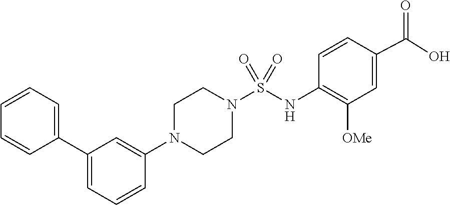 [SMILES: COc1cc(C(=O)O)ccc1NS(=O)(=O)N1CCN(c2cccc(-c3ccccc3)c2)CC1] |
| 322 | 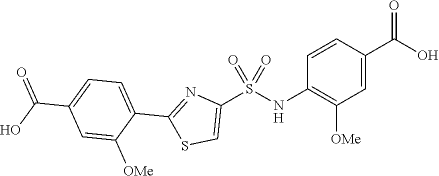 [SMILES: COc1cc(C(=O)O)ccc1NS(=O)(=O)c1csc(-c2ccc(C(=O)O)cc2OC)n1] |
| 323 | 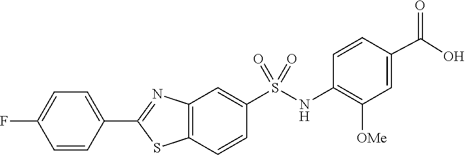 [SMILES: COc1cc(C(=O)O)ccc1NS(=O)(=O)c1ccc2sc(-c3ccc(F)cc3)nc2c1] |
| 324 | 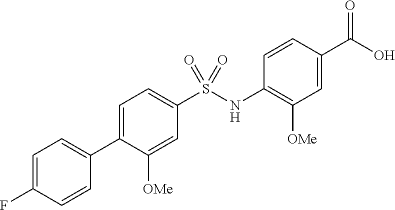 [SMILES: COc1cc(C(=O)O)ccc1NS(=O)(=O)c1ccc(-c2ccc(F)cc2)c(OC)c1] |
| 325 | 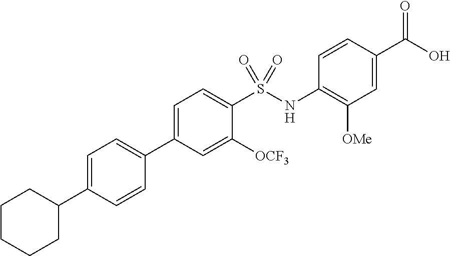 [SMILES: COc1cc(C(=O)O)ccc1NS(=O)(=O)c1ccc(-c2ccc(C3CCCCC3)cc2)cc1OC(F)(F)F] |
| 326 | 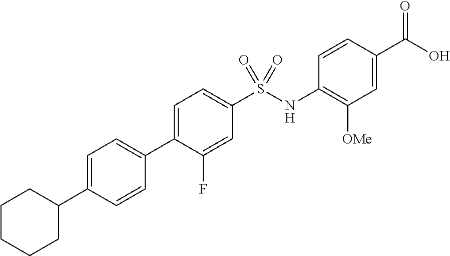 [SMILES: COc1cc(C(=O)O)ccc1NS(=O)(=O)c1ccc(-c2ccc(C3CCCCC3)cc2)c(F)c1] |
| 327 | 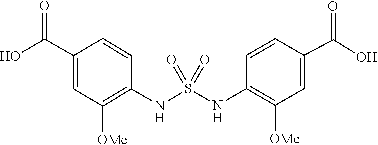 [SMILES: COc1cc(C(=O)O)ccc1NS(=O)(=O)Nc1ccc(C(=O)O)cc1OC] |
| 328 | 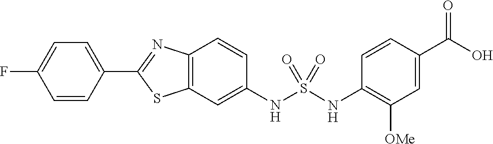 [SMILES: COc1cc(C(=O)O)ccc1NS(=O)(=O)Nc1ccc2nc(-c3ccc(F)cc3)sc2c1] |
| 329 | 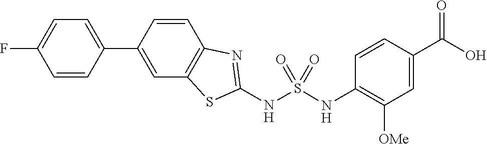 [SMILES: COc1cc(C(=O)O)ccc1NS(=O)(=O)Nc1nc2ccc(-c3ccc(F)cc3)cc2s1] |
| 330 | 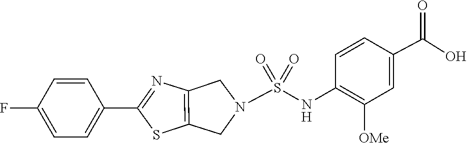 |
| 331 | 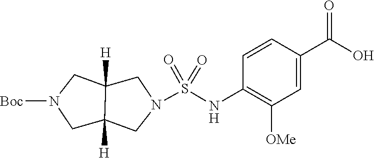 [SMILES: COc1cc(C(=O)O)ccc1NS(=O)(=O)N1C[C@H]2CN(C(=O)OC(C)(C)C)C[C@H]2C1] |
| 332 | 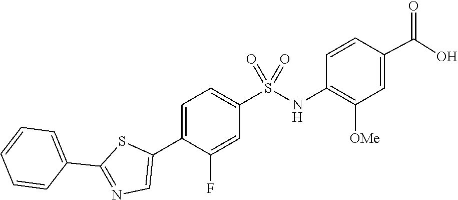 [SMILES: COc1cc(C(=O)O)ccc1NS(=O)(=O)c1ccc(-c2cnc(-c3ccccc3)s2)c(F)c1] |
| 333 | 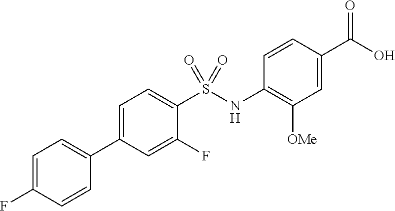 [SMILES: COc1cc(C(=O)O)ccc1NS(=O)(=O)c1ccc(-c2ccc(F)cc2)cc1F] |
| 334 | 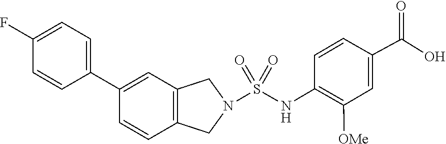 [SMILES: COc1cc(C(=O)O)ccc1NS(=O)(=O)N1Cc2ccc(-c3ccc(F)cc3)cc2C1] |
| 335 | 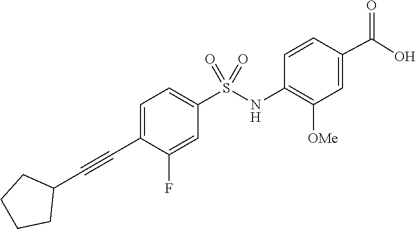 [SMILES: COc1cc(C(=O)O)ccc1NS(=O)(=O)c1ccc(C#CC2CCCC2)c(F)c1] |
| 336 | 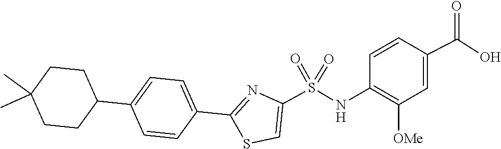 [SMILES: COc1cc(C(=O)O)ccc1NS(=O)(=O)c1csc(-c2ccc(C3CCC(C)(C)CC3)cc2)n1] |
| 337 | 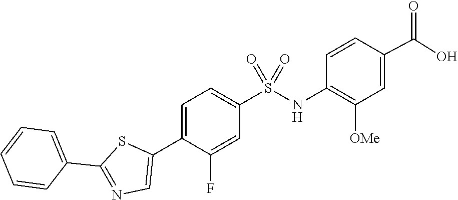 [SMILES: COc1cc(C(=O)O)ccc1NS(=O)(=O)c1ccc(-c2cnc(-c3ccccc3)s2)c(F)c1] |
| 338 | 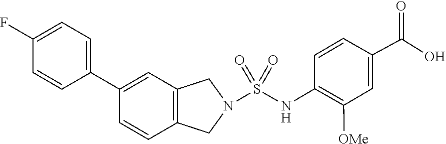 [SMILES: COc1cc(C(=O)O)ccc1NS(=O)(=O)N1Cc2ccc(-c3ccc(F)cc3)cc2C1] |
| 339 | 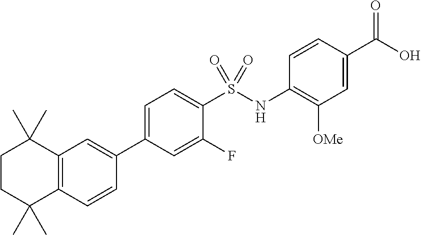 [SMILES: COc1cc(C(=O)O)ccc1NS(=O)(=O)c1ccc(-c2ccc3c(c2)C(C)(C)CCC3(C)C)cc1F] |
| 340 | 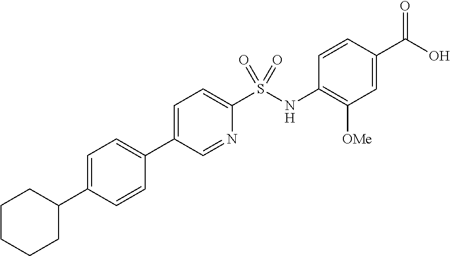 [SMILES: COc1cc(C(=O)O)ccc1NS(=O)(=O)c1ccc(-c2ccc(C3CCCCC3)cc2)cn1] |
| 341 | 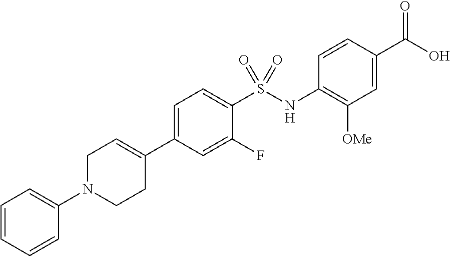 [SMILES: COc1cc(C(=O)O)ccc1NS(=O)(=O)c1ccc(C2=CCN(c3ccccc3)CC2)cc1F] |
| 342 | 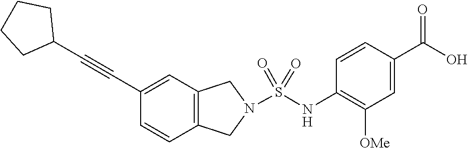 [SMILES: COc1cc(C(=O)O)ccc1NS(=O)(=O)N1Cc2ccc(C#CC3CCCC3)cc2C1] |
| 343 | 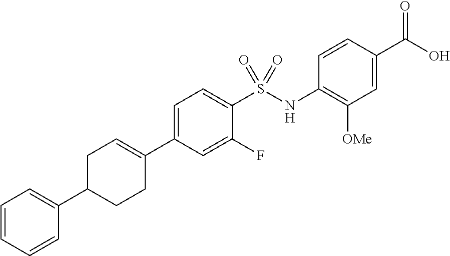 [SMILES: COc1cc(C(=O)O)ccc1NS(=O)(=O)c1ccc(C2=CCC(c3ccccc3)CC2)cc1F] |
| 344 | 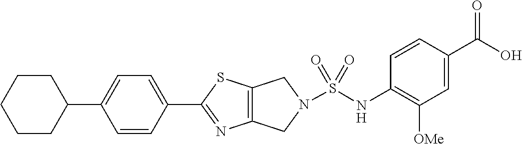 [SMILES: COc1cc(C(=O)O)ccc1NS(=O)(=O)N1Cc2nc(-c3ccc(C4CCCCC4)cc3)sc2C1] |
| 345 | 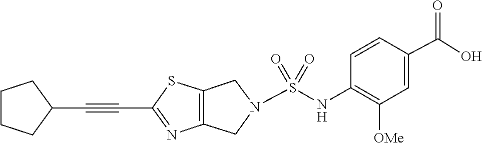 |
| 346 | 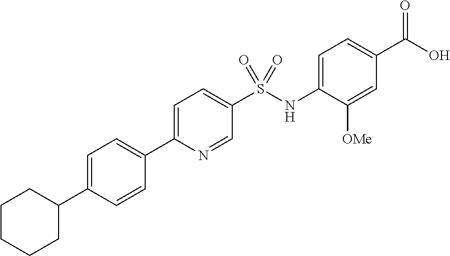 [SMILES: COc1cc(C(=O)O)ccc1NS(=O)(=O)c1ccc(-c2ccc(C3CCCCC3)cc2)nc1] |
| 347 | 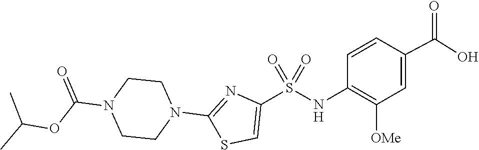 [SMILES: COc1cc(C(=O)O)ccc1NS(=O)(=O)c1csc(N2CCN(C(=O)OC(C)C)CC2)n1] |
| 348 | 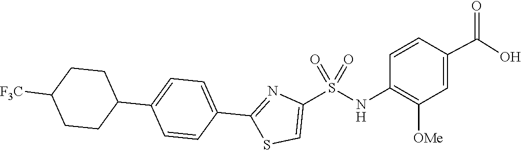 [SMILES: COc1cc(C(=O)O)ccc1NS(=O)(=O)c1csc(-c2ccc(C3CCC(C(F)(F)F)CC3)cc2)n1] |
| 349 | 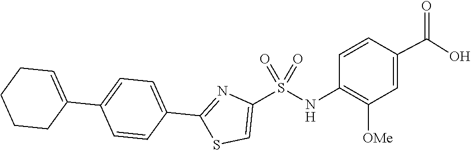 [SMILES: COc1cc(C(=O)O)ccc1NS(=O)(=O)c1csc(-c2ccc(C3=CCCCC3)cc2)n1] |
| 350 | 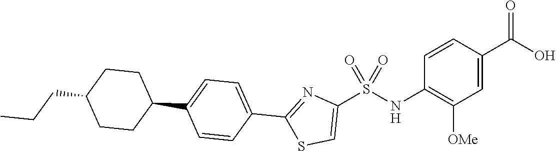 [SMILES: CCC[C@H]1CC[C@H](c2ccc(-c3nc(S(=O)(=O)Nc4ccc(C(=O)O)cc4OC)cs3)cc2)CC1] |
| 351 | 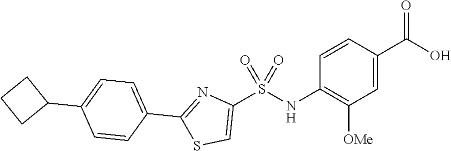 [SMILES: COc1cc(C(=O)O)ccc1NS(=O)(=O)c1csc(-c2ccc(C3CCC3)cc2)n1] |
| 352 | 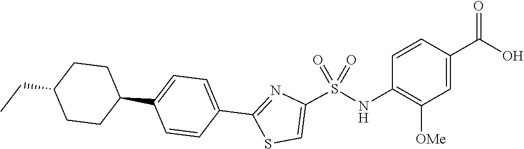 [SMILES: CC[C@H]1CC[C@H](c2ccc(-c3nc(S(=O)(=O)Nc4ccc(C(=O)O)cc4OC)cs3)cc2)CC1] |
| 353 | 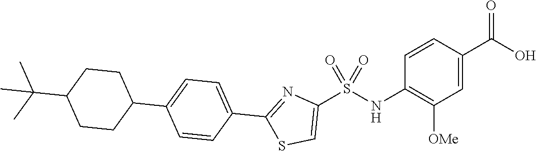 [SMILES: COc1cc(C(=O)O)ccc1NS(=O)(=O)c1csc(-c2ccc(C3CCC(C(C)(C)C)CC3)cc2)n1] |
| 354 | 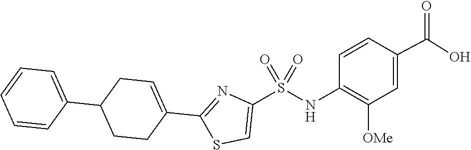 [SMILES: COc1cc(C(=O)O)ccc1NS(=O)(=O)c1csc(C2=CCC(c3ccccc3)CC2)n1] |
| 355 | 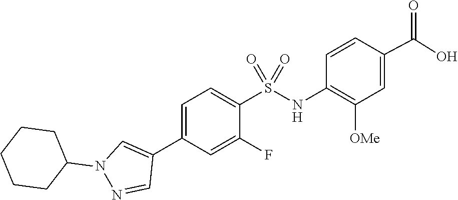 [SMILES: COc1cc(C(=O)O)ccc1NS(=O)(=O)c1ccc(-c2cnn(C3CCCCC3)c2)cc1F] |
| 356 | 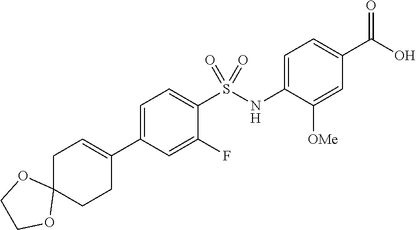 [SMILES: COc1cc(C(=O)O)ccc1NS(=O)(=O)c1ccc(C2=CCC3(CC2)OCCO3)cc1F] |
| 357 | 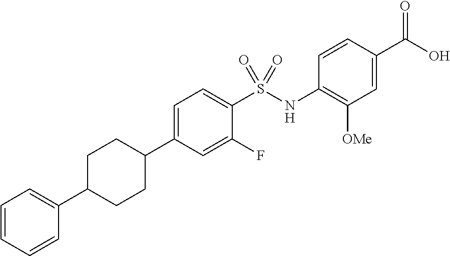 [SMILES: COc1cc(C(=O)O)ccc1NS(=O)(=O)c1ccc(C2CCC(c3ccccc3)CC2)cc1F] |
| 358 | 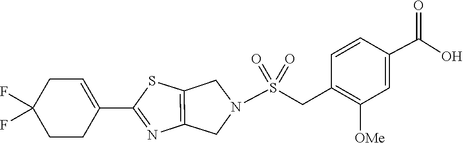 |
| 359 | 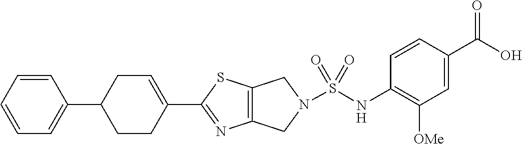 [SMILES: COc1cc(C(=O)O)ccc1NS(=O)(=O)N1Cc2nc(C3=CCC(c4ccccc4)CC3)sc2C1] |
| 360 | 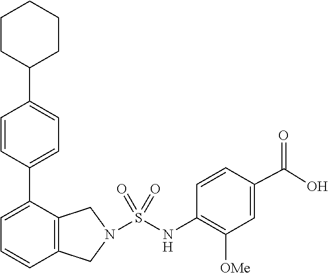 [SMILES: COc1cc(C(=O)O)ccc1NS(=O)(=O)N1Cc2cccc(-c3ccc(C4CCCCC4)cc3)c2C1] |
| 361 | 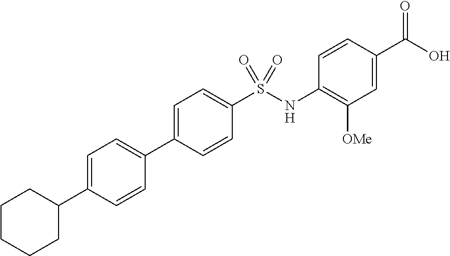 [SMILES: COc1cc(C(=O)O)ccc1NS(=O)(=O)c1ccc(-c2ccc(C3CCCCC3)cc2)cc1] |
| 362 | 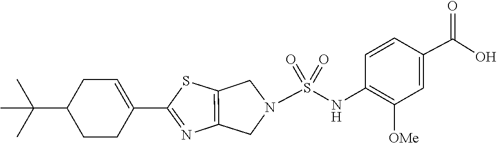 |
| 363 | 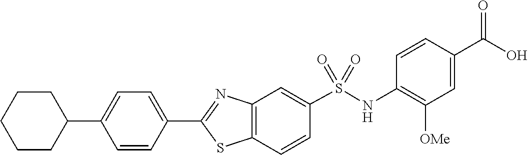 [SMILES: COc1cc(C(=O)O)ccc1NS(=O)(=O)c1ccc2sc(-c3ccc(C4CCCCC4)cc3)nc2c1] |
| 364 | 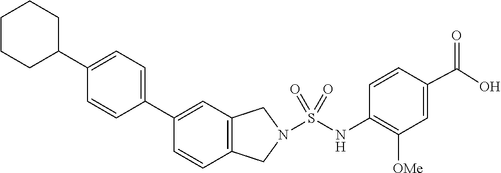 [SMILES: COc1cc(C(=O)O)ccc1NS(=O)(=O)N1Cc2ccc(-c3ccc(C4CCCCC4)cc3)cc2C1] |
| 365 | 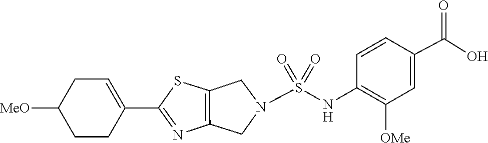 |
| 366 | 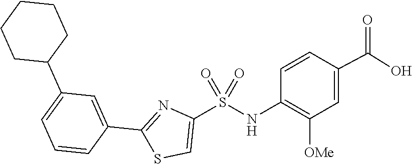 [SMILES: COc1cc(C(=O)O)ccc1NS(=O)(=O)c1csc(-c2cccc(C3CCCCC3)c2)n1] |
| 367 | 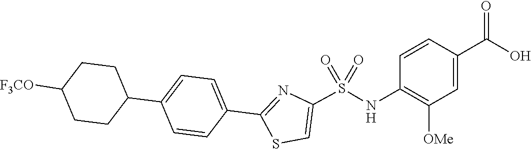 [SMILES: COc1cc(C(=O)O)ccc1NS(=O)(=O)c1csc(-c2ccc(C3CCC(OC(F)(F)F)CC3)cc2)n1] |
| 368 | 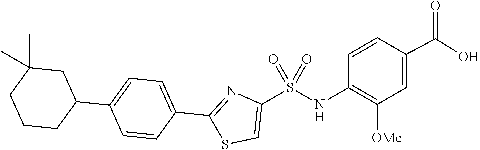 [SMILES: COc1cc(C(=O)O)ccc1NS(=O)(=O)c1csc(-c2ccc(C3CCCC(C)(C)C3)cc2)n1] |
| 369 | 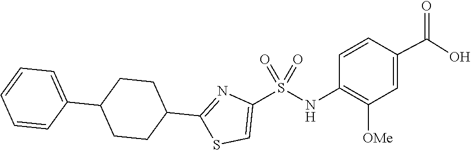 [SMILES: COc1cc(C(=O)O)ccc1NS(=O)(=O)c1csc(C2CCC(c3ccccc3)CC2)n1] |
| 370 | 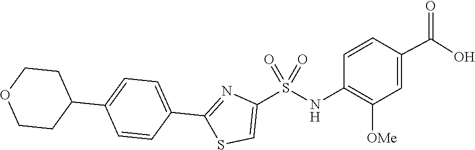 [SMILES: COc1cc(C(=O)O)ccc1NS(=O)(=O)c1csc(-c2ccc(C3CCOCC3)cc2)n1] |
| 371 | 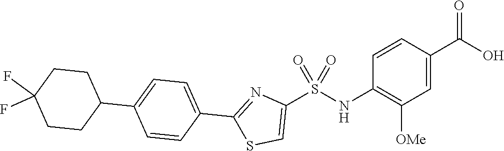 [SMILES: COc1cc(C(=O)O)ccc1NS(=O)(=O)c1csc(-c2ccc(C3CCC(F)(F)CC3)cc2)n1] |
| 372 | 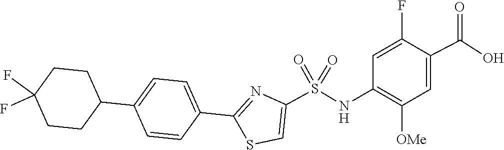 [SMILES: COc1cc(C(=O)O)c(F)cc1NS(=O)(=O)c1csc(-c2ccc(C3CCC(F)(F)CC3)cc2)n1] |
| 373 | 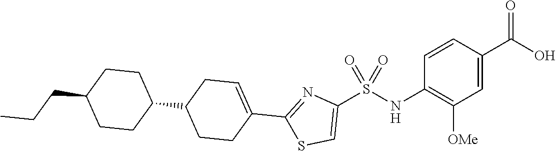 [SMILES: CCC[C@H]1CC[C@H](C2CC=C(c3nc(S(=O)(=O)Nc4ccc(C(=O)O)cc4OC)cs3)CC2)CC1] |
| 374 | 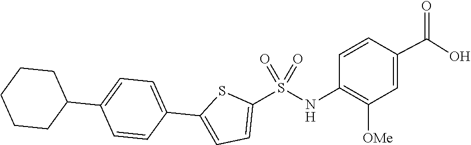 [SMILES: COc1cc(C(=O)O)ccc1NS(=O)(=O)c1ccc(-c2ccc(C3CCCCC3)cc2)s1] |
| 375 | 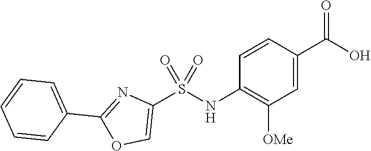 [SMILES: COc1cc(C(=O)O)ccc1NS(=O)(=O)c1coc(-c2ccccc2)n1] |
| 376 | 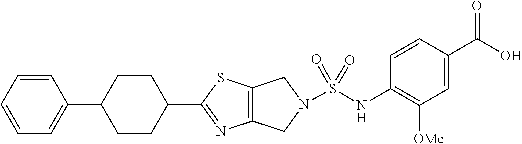 [SMILES: COc1cc(C(=O)O)ccc1NS(=O)(=O)N1Cc2nc(C3CCC(c4ccccc4)CC3)sc2C1] |
| 377 | 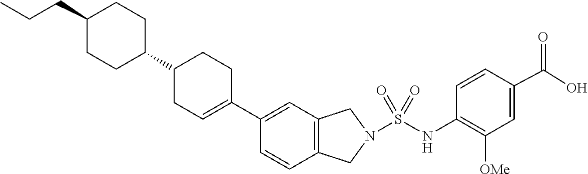 [SMILES: CCC[C@H]1CC[C@H](C2CC=C(c3ccc4c(c3)CN(S(=O)(=O)Nc3ccc(C(=O)O)cc3OC)C4)CC2)CC1] |
| 378 | 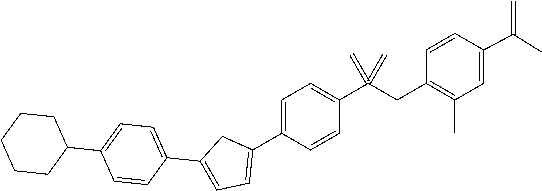 |
| 379 | 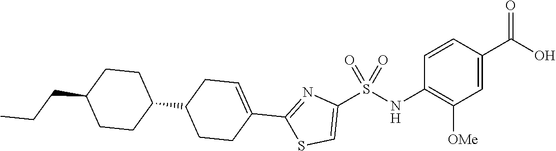 [SMILES: CCC[C@H]1CC[C@H](C2CC=C(c3nc(S(=O)(=O)Nc4ccc(C(=O)O)cc4OC)cs3)CC2)CC1] |
| 380 | 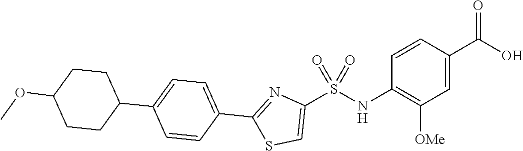 [SMILES: COc1cc(C(=O)O)ccc1NS(=O)(=O)c1csc(-c2ccc(C3CCC(OC)CC3)cc2)n1] |
| 381 | 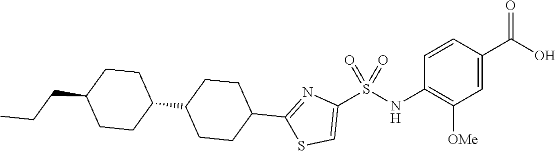 [SMILES: CCC[C@H]1CC[C@H](C2CCC(c3nc(S(=O)(=O)Nc4ccc(C(=O)O)cc4OC)cs3)CC2)CC1] |
| 382 | 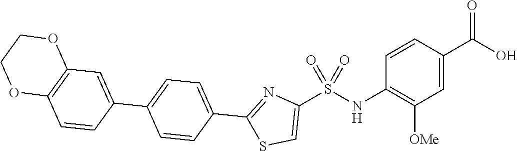 [SMILES: COc1cc(C(=O)O)ccc1NS(=O)(=O)c1csc(-c2ccc(-c3ccc4c(c3)OCCO4)cc2)n1] |
| 383 | 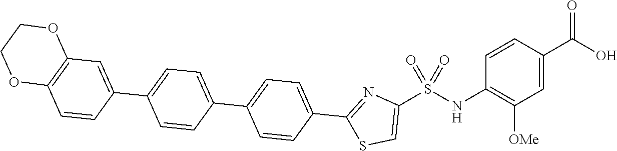 [SMILES: COc1cc(C(=O)O)ccc1NS(=O)(=O)c1csc(-c2ccc(-c3ccc(-c4ccc5c(c4)OCCO5)cc3)cc2)n1] |
| 384 | 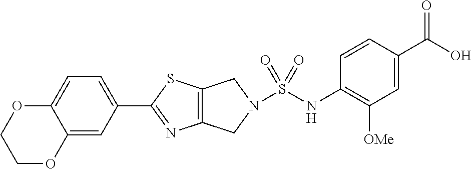 [SMILES: COc1cc(C(=O)O)ccc1NS(=O)(=O)N1Cc2nc(-c3ccc4c(c3)OCCO4)sc2C1] |
| 385 | 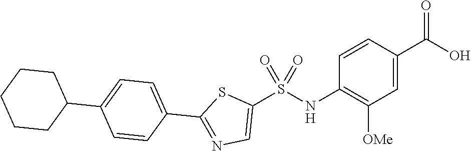 [SMILES: COc1cc(C(=O)O)ccc1NS(=O)(=O)c1cnc(-c2ccc(C3CCCCC3)cc2)s1] |
| 386 | 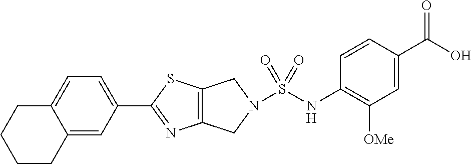 [SMILES: COc1cc(C(=O)O)ccc1NS(=O)(=O)N1Cc2nc(-c3ccc4c(c3)CCCC4)sc2C1] |
| 387 | 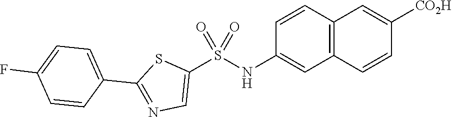 [SMILES: O=C(O)c1ccc2cc(NS(=O)(=O)c3cnc(-c4ccc(F)cc4)s3)ccc2c1] |
| 388 | 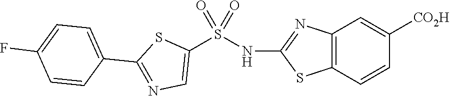 [SMILES: O=C(O)c1ccc2sc(NS(=O)(=O)c3cnc(-c4ccc(F)cc4)s3)nc2c1] |
| 389 | 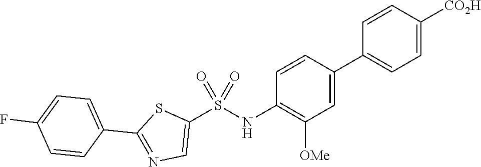 [SMILES: COc1cc(-c2ccc(C(=O)O)cc2)ccc1NS(=O)(=O)c1cnc(-c2ccc(F)cc2)s1] |
| 390 | 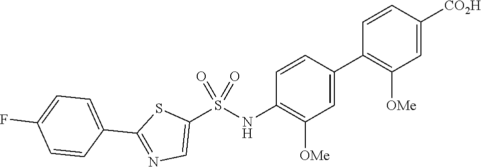 [SMILES: COc1cc(-c2ccc(C(=O)O)cc2OC)ccc1NS(=O)(=O)c1cnc(-c2ccc(F)cc2)s1] |
| 391 | 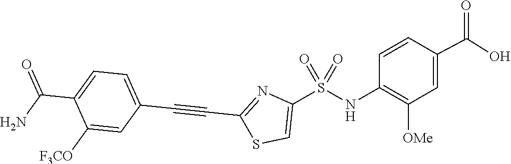 [SMILES: COc1cc(C(=O)O)ccc1NS(=O)(=O)c1csc(C#Cc2ccc(C(N)=O)c(OC(F)(F)F)c2)n1] |
| 392 | 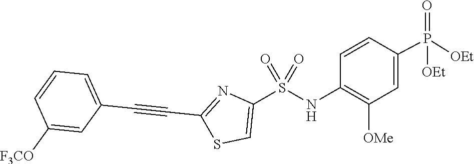 [SMILES: CCOP(=O)(OCC)c1ccc(NS(=O)(=O)c2csc(C#Cc3cccc(OC(F)(F)F)c3)n2)c(OC)c1] |
| 393 | 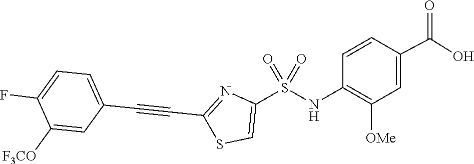 [SMILES: COc1cc(C(=O)O)ccc1NS(=O)(=O)c1csc(C#Cc2ccc(F)c(OC(F)(F)F)c2)n1] |
| 394 | 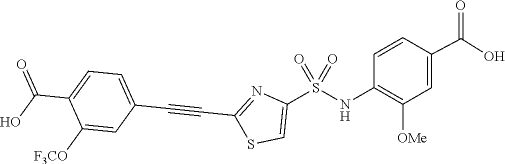 [SMILES: COc1cc(C(=O)O)ccc1NS(=O)(=O)c1csc(C#Cc2ccc(C(=O)O)c(OC(F)(F)F)c2)n1] |
| 395 | 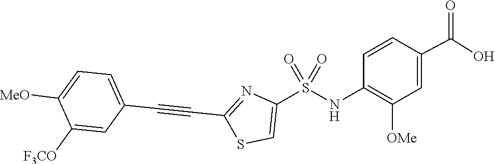 [SMILES: COc1cc(C(=O)O)ccc1NS(=O)(=O)c1csc(C#Cc2ccc(OC)c(OC(F)(F)F)c2)n1] |
| 396 | 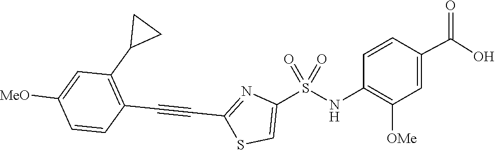 [SMILES: COc1ccc(C#Cc2nc(S(=O)(=O)Nc3ccc(C(=O)O)cc3OC)cs2)c(C2CC2)c1] |
| 397 | 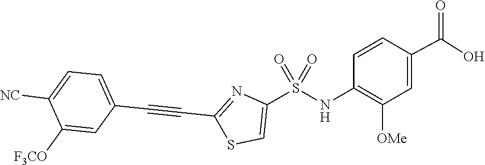 [SMILES: COc1cc(C(=O)O)ccc1NS(=O)(=O)c1csc(C#Cc2ccc(C#N)c(OC(F)(F)F)c2)n1] |
| 398 | 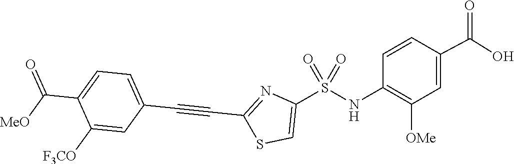 [SMILES: COC(=O)c1ccc(C#Cc2nc(S(=O)(=O)Nc3ccc(C(=O)O)cc3OC)cs2)cc1OC(F)(F)F] |
| 399 | 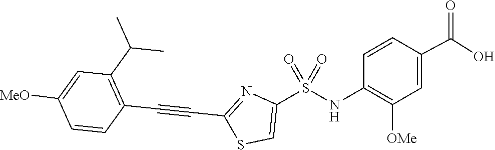 [SMILES: COc1ccc(C#Cc2nc(S(=O)(=O)Nc3ccc(C(=O)O)cc3OC)cs2)c(C(C)C)c1] |
| 400 | 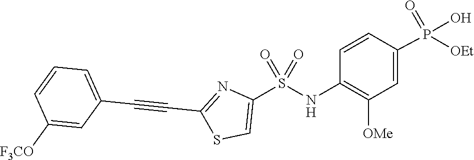 [SMILES: CCOP(=O)(O)c1ccc(NS(=O)(=O)c2csc(C#Cc3cccc(OC(F)(F)F)c3)n2)c(OC)c1] |
| 401 | 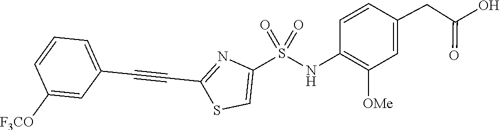 [SMILES: COc1cc(CC(=O)O)ccc1NS(=O)(=O)c1csc(C#Cc2cccc(OC(F)(F)F)c2)n1] |
| 402 | 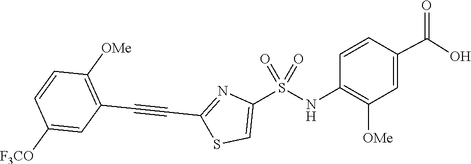 [SMILES: COc1ccc(OC(F)(F)F)cc1C#Cc1nc(S(=O)(=O)Nc2ccc(C(=O)O)cc2OC)cs1] |
| 403 | 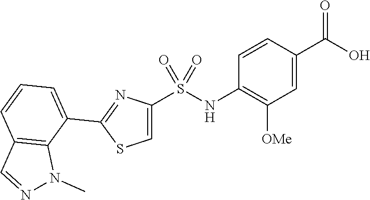 [SMILES: COc1cc(C(=O)O)ccc1NS(=O)(=O)c1csc(-c2cccc3cnn(C)c23)n1] |
| 404 | 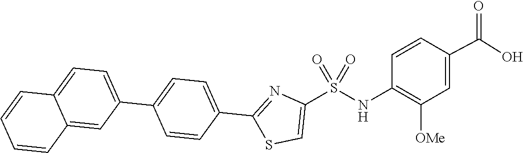 [SMILES: COc1cc(C(=O)O)ccc1NS(=O)(=O)c1csc(-c2ccc(-c3ccc4ccccc4c3)cc2)n1] |
| 405 | 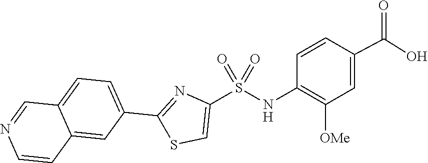 [SMILES: COc1cc(C(=O)O)ccc1NS(=O)(=O)c1csc(-c2ccc3cnccc3c2)n1] |
| 406 | 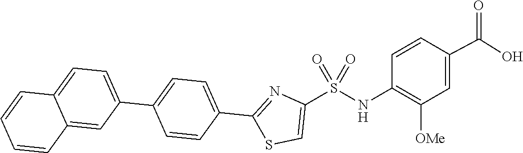 [SMILES: COc1cc(C(=O)O)ccc1NS(=O)(=O)c1csc(-c2ccc(-c3ccc4ccccc4c3)cc2)n1] |
| 407 | 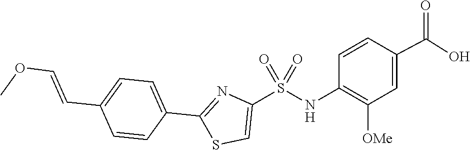 [SMILES: CO/C=C/c1ccc(-c2nc(S(=O)(=O)Nc3ccc(C(=O)O)cc3OC)cs2)cc1] |
| 408 | 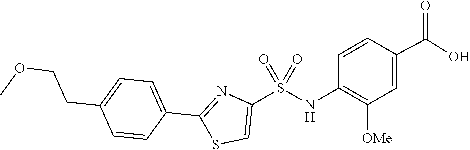 [SMILES: COCCc1ccc(-c2nc(S(=O)(=O)Nc3ccc(C(=O)O)cc3OC)cs2)cc1] |
| 409 | 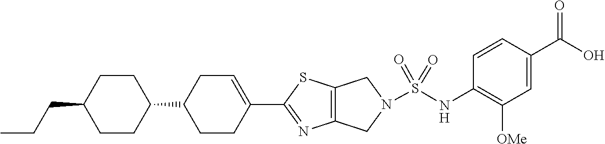 [SMILES: CCC[C@H]1CC[C@H](C2CC=C(c3nc4c(s3)CN(S(=O)(=O)Nc3ccc(C(=O)O)cc3OC)C4)CC2)CC1] |
| 410 | 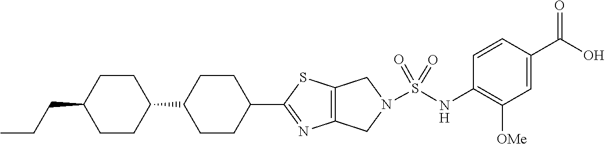 [SMILES: CCC[C@H]1CC[C@H](C2CCC(c3nc4c(s3)CN(S(=O)(=O)Nc3ccc(C(=O)O)cc3OC)C4)CC2)CC1] |
| 411 | 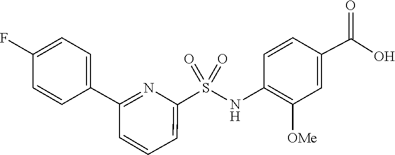 [SMILES: COc1cc(C(=O)O)ccc1NS(=O)(=O)c1cccc(-c2ccc(F)cc2)n1] |
| 412 | 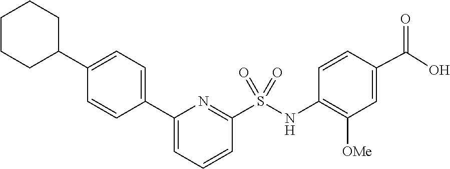 [SMILES: COc1cc(C(=O)O)ccc1NS(=O)(=O)c1cccc(-c2ccc(C3CCCCC3)cc2)n1] |
| 413 | 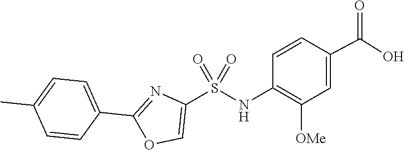 [SMILES: COc1cc(C(=O)O)ccc1NS(=O)(=O)c1coc(-c2ccc(C)cc2)n1] |
| 414 | 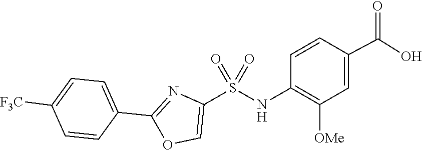 [SMILES: COc1cc(C(=O)O)ccc1NS(=O)(=O)c1coc(-c2ccc(C(F)(F)F)cc2)n1] |
| 415 | 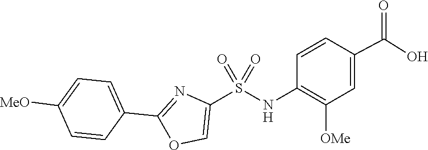 [SMILES: COc1ccc(-c2nc(S(=O)(=O)Nc3ccc(C(=O)O)cc3OC)co2)cc1] |
| 416 | 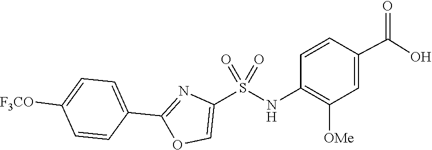 [SMILES: COc1cc(C(=O)O)ccc1NS(=O)(=O)c1coc(-c2ccc(OC(F)(F)F)cc2)n1] |
| 417 | 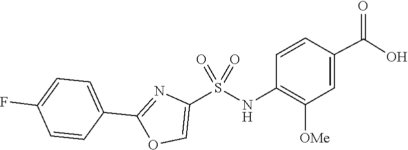 [SMILES: COc1cc(C(=O)O)ccc1NS(=O)(=O)c1coc(-c2ccc(F)cc2)n1] |
| 418 | 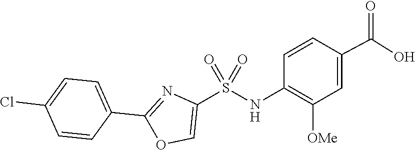 [SMILES: COc1cc(C(=O)O)ccc1NS(=O)(=O)c1coc(-c2ccc(Cl)cc2)n1] |
| 419 | 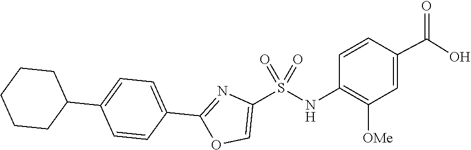 [SMILES: COc1cc(C(=O)O)ccc1NS(=O)(=O)c1coc(-c2ccc(C3CCCCC3)cc2)n1] |
| 420 | 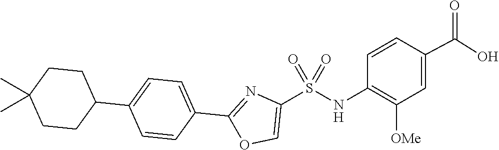 [SMILES: COc1cc(C(=O)O)ccc1NS(=O)(=O)c1coc(-c2ccc(C3CCC(C)(C)CC3)cc2)n1] |
| 421 | 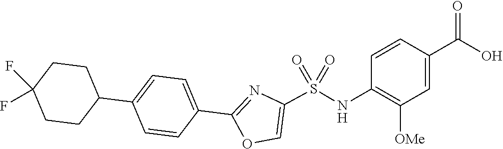 [SMILES: COc1cc(C(=O)O)ccc1NS(=O)(=O)c1coc(-c2ccc(C3CCC(F)(F)CC3)cc2)n1] |
| 422 | 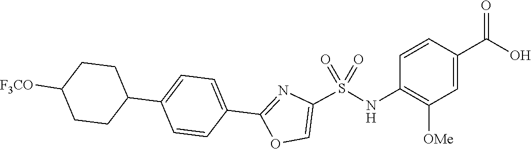 [SMILES: COc1cc(C(=O)O)ccc1NS(=O)(=O)c1coc(-c2ccc(C3CCC(OC(F)(F)F)CC3)cc2)n1] |
| 423 | 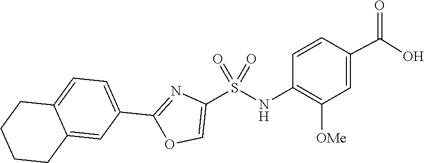 [SMILES: COc1cc(C(=O)O)ccc1NS(=O)(=O)c1coc(-c2ccc3c(c2)CCCC3)n1] |
| 424 | 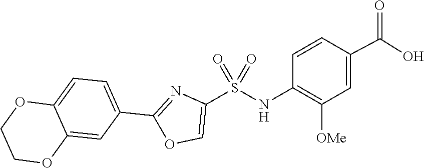 [SMILES: COc1cc(C(=O)O)ccc1NS(=O)(=O)c1coc(-c2ccc3c(c2)OCCO3)n1] |
| 425 | 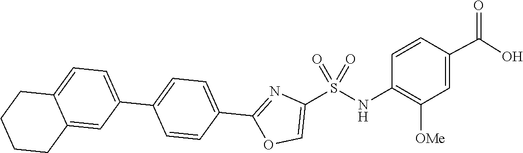 [SMILES: COc1cc(C(=O)O)ccc1NS(=O)(=O)c1coc(-c2ccc(-c3ccc4c(c3)CCCC4)cc2)n1] |
| 426 | 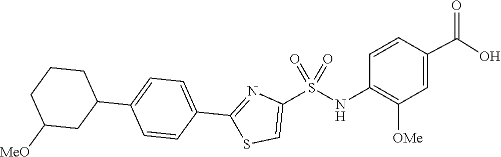 [SMILES: COc1cc(C(=O)O)ccc1NS(=O)(=O)c1csc(-c2ccc(C3CCCC(OC)C3)cc2)n1] |
| 427 | 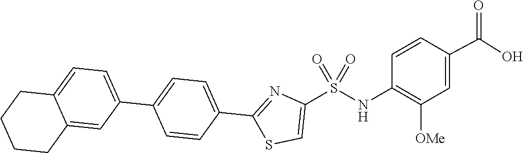 [SMILES: COc1cc(C(=O)O)ccc1NS(=O)(=O)c1csc(-c2ccc(-c3ccc4c(c3)CCCC4)cc2)n1] |
| 428 | 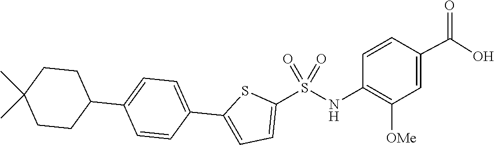 [SMILES: COc1cc(C(=O)O)ccc1NS(=O)(=O)c1ccc(-c2ccc(C3CCC(C)(C)CC3)cc2)s1] |
| 429 | 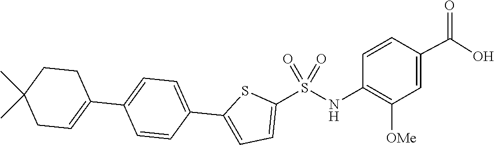 [SMILES: COc1cc(C(=O)O)ccc1NS(=O)(=O)c1ccc(-c2ccc(C3=CCC(C)(C)CC3)cc2)s1] |
| 430 | 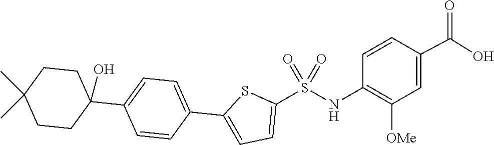 [SMILES: COc1cc(C(=O)O)ccc1NS(=O)(=O)c1ccc(-c2ccc(C3(O)CCC(C)(C)CC3)cc2)s1] |
| 431 | 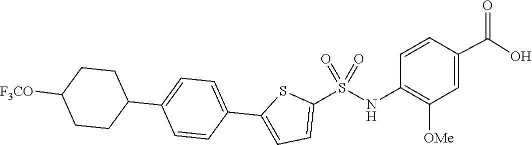 [SMILES: COc1cc(C(=O)O)ccc1NS(=O)(=O)c1ccc(-c2ccc(C3CCC(OC(F)(F)F)CC3)cc2)s1] |
| 432 | 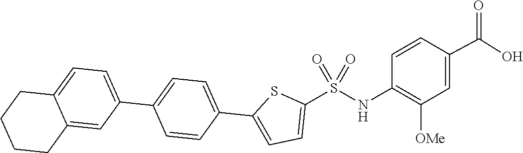 [SMILES: COc1cc(C(=O)O)ccc1NS(=O)(=O)c1ccc(-c2ccc(-c3ccc4c(c3)CCCC4)cc2)s1] |
| 433 | 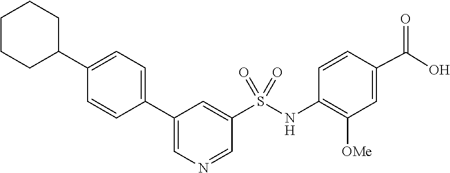 [SMILES: COc1cc(C(=O)O)ccc1NS(=O)(=O)c1cncc(-c2ccc(C3CCCCC3)cc2)c1] |
| 434 | 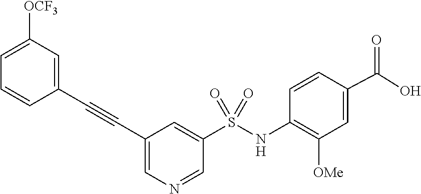 [SMILES: COc1cc(C(=O)O)ccc1NS(=O)(=O)c1cncc(C#Cc2cccc(OC(F)(F)F)c2)c1] |
| 435 | 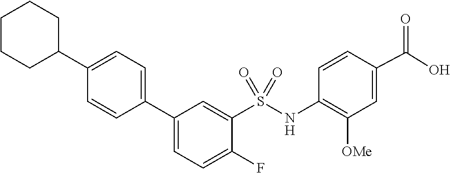 [SMILES: COc1cc(C(=O)O)ccc1NS(=O)(=O)c1cc(-c2ccc(C3CCCCC3)cc2)ccc1F] |
| 436 | 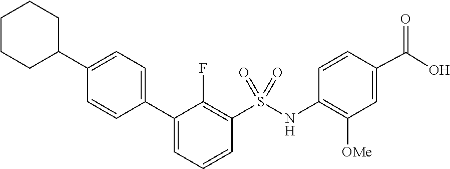 [SMILES: COc1cc(C(=O)O)ccc1NS(=O)(=O)c1cccc(-c2ccc(C3CCCCC3)cc2)c1F] |
| 437 | 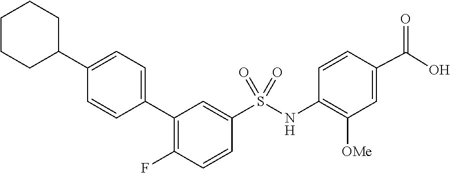 [SMILES: COc1cc(C(=O)O)ccc1NS(=O)(=O)c1ccc(F)c(-c2ccc(C3CCCCC3)cc2)c1] |
| 438 | 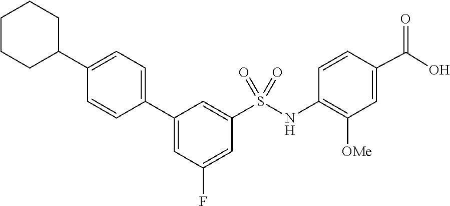 [SMILES: COc1cc(C(=O)O)ccc1NS(=O)(=O)c1cc(F)cc(-c2ccc(C3CCCCC3)cc2)c1] |
| 439 | 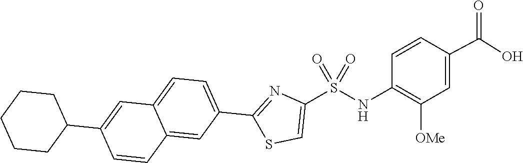 [SMILES: COc1cc(C(=O)O)ccc1NS(=O)(=O)c1csc(-c2ccc3cc(C4CCCCC4)ccc3c2)n1] |
| 440 | 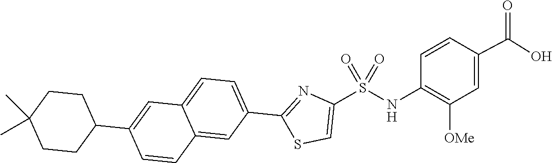 [SMILES: COc1cc(C(=O)O)ccc1NS(=O)(=O)c1csc(-c2ccc3cc(C4CCC(C)(C)CC4)ccc3c2)n1] |
| 441 | 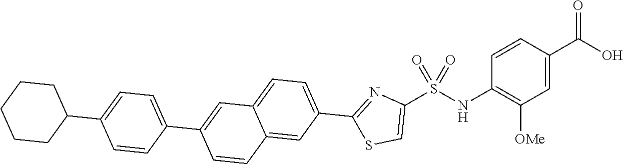 [SMILES: COc1cc(C(=O)O)ccc1NS(=O)(=O)c1csc(-c2ccc3cc(-c4ccc(C5CCCCC5)cc4)ccc3c2)n1] |
| 442 | 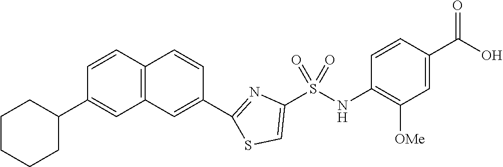 [SMILES: COc1cc(C(=O)O)ccc1NS(=O)(=O)c1csc(-c2ccc3ccc(C4CCCCC4)cc3c2)n1] |
| 443 | 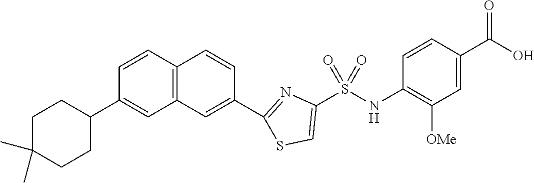 [SMILES: COc1cc(C(=O)O)ccc1NS(=O)(=O)c1csc(-c2ccc3ccc(C4CCC(C)(C)CC4)cc3c2)n1] |
| 444 | 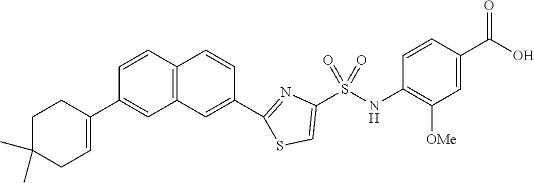 [SMILES: COc1cc(C(=O)O)ccc1NS(=O)(=O)c1csc(-c2ccc3ccc(C4=CCC(C)(C)CC4)cc3c2)n1] |
| 445 | 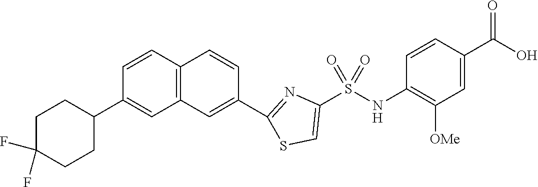 [SMILES: COc1cc(C(=O)O)ccc1NS(=O)(=O)c1csc(-c2ccc3ccc(C4CCC(F)(F)CC4)cc3c2)n1] |
| 446 | 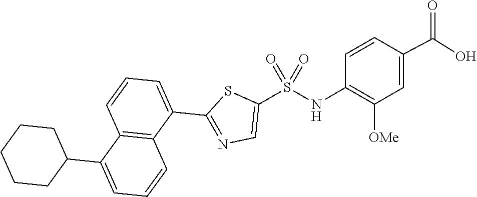 [SMILES: COc1cc(C(=O)O)ccc1NS(=O)(=O)c1cnc(-c2cccc3c(C4CCCCC4)cccc23)s1] |
| 447 | 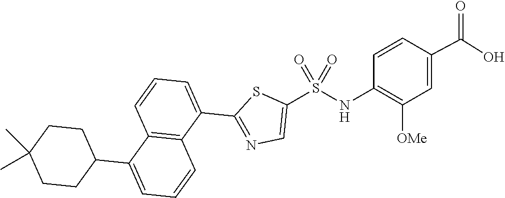 [SMILES: COc1cc(C(=O)O)ccc1NS(=O)(=O)c1cnc(-c2cccc3c(C4CCC(C)(C)CC4)cccc23)s1] |
| 448 | 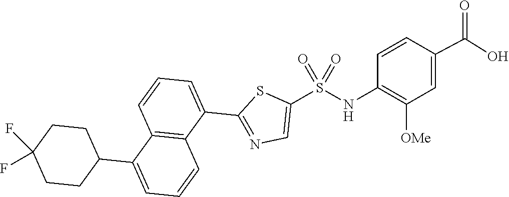 [SMILES: COc1cc(C(=O)O)ccc1NS(=O)(=O)c1cnc(-c2cccc3c(C4CCC(F)(F)CC4)cccc23)s1] |
| 449 | 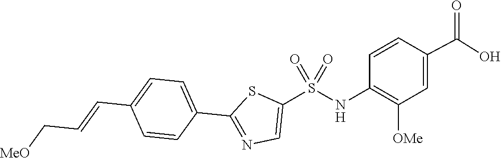 [SMILES: COC/C=C/c1ccc(-c2ncc(S(=O)(=O)Nc3ccc(C(=O)O)cc3OC)s2)cc1] |
| 450 | 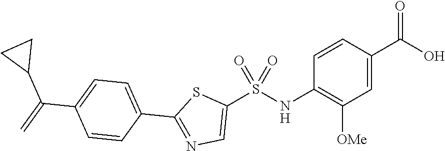 [SMILES: C=C(c1ccc(-c2ncc(S(=O)(=O)Nc3ccc(C(=O)O)cc3OC)s2)cc1)C1CC1] |
| 451 | 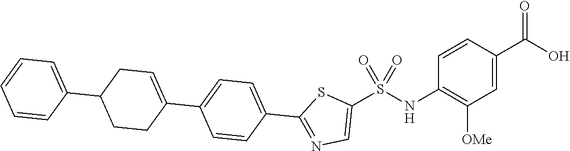 [SMILES: COc1cc(C(=O)O)ccc1NS(=O)(=O)c1cnc(-c2ccc(C3=CCC(c4ccccc4)CC3)cc2)s1] |
| 452 | 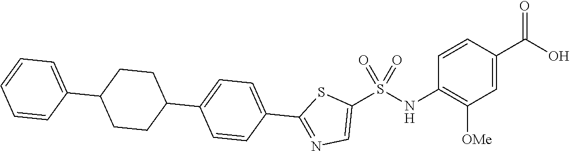 [SMILES: COc1cc(C(=O)O)ccc1NS(=O)(=O)c1cnc(-c2ccc(C3CCC(c4ccccc4)CC3)cc2)s1] |
| 453 | 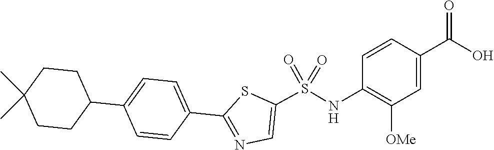 [SMILES: COc1cc(C(=O)O)ccc1NS(=O)(=O)c1cnc(-c2ccc(C3CCC(C)(C)CC3)cc2)s1] |
| 454 | 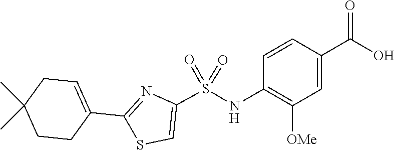 [SMILES: COc1cc(C(=O)O)ccc1NS(=O)(=O)c1csc(C2=CCC(C)(C)CC2)n1] |
| 455 | 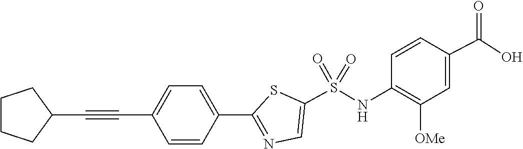 [SMILES: COc1cc(C(=O)O)ccc1NS(=O)(=O)c1cnc(-c2ccc(C#CC3CCCC3)cc2)s1] |
| 456 | 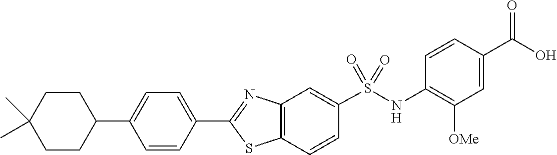 [SMILES: COc1cc(C(=O)O)ccc1NS(=O)(=O)c1ccc2sc(-c3ccc(C4CCC(C)(C)CC4)cc3)nc2c1] |
| 457 | 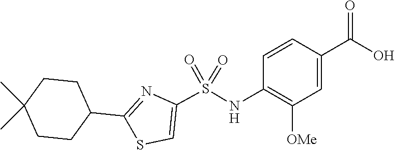 [SMILES: COc1cc(C(=O)O)ccc1NS(=O)(=O)c1csc(C2CCC(C)(C)CC2)n1] |
| 458 | 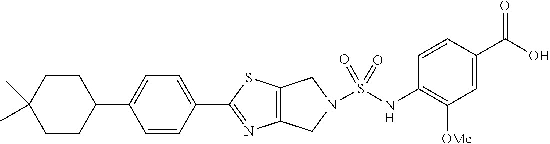 [SMILES: COc1cc(C(=O)O)ccc1NS(=O)(=O)N1Cc2nc(-c3ccc(C4CCC(C)(C)CC4)cc3)sc2C1] |
| 459 | 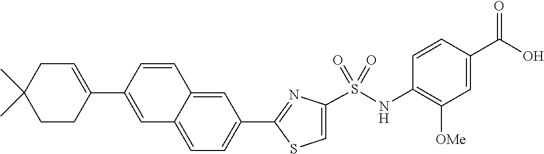 [SMILES: COc1cc(C(=O)O)ccc1NS(=O)(=O)c1csc(-c2ccc3cc(C4=CCC(C)(C)CC4)ccc3c2)n1] |
| 460 | 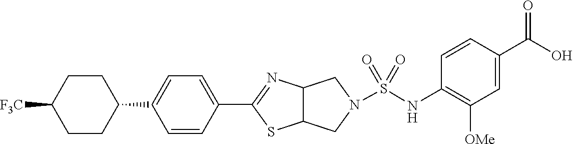 [SMILES: COc1cc(C(=O)O)ccc1NS(=O)(=O)N1CC2N=C(c3ccc([C@H]4CC[C@H](C(F)(F)F)CC4)cc3)SC2C1] |
| 461 | 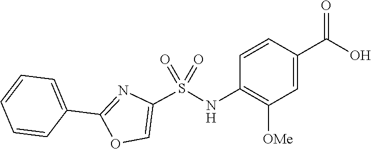 [SMILES: COc1cc(C(=O)O)ccc1NS(=O)(=O)c1coc(-c2ccccc2)n1] |
| 462 | 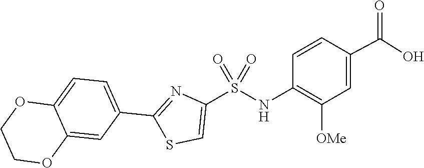 [SMILES: COc1cc(C(=O)O)ccc1NS(=O)(=O)c1csc(-c2ccc3c(c2)OCCO3)n1] |
| 463 | 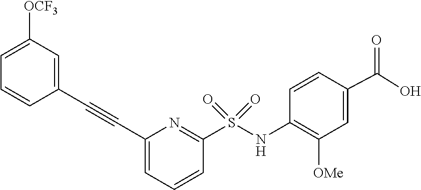 [SMILES: COc1cc(C(=O)O)ccc1NS(=O)(=O)c1cccc(C#Cc2cccc(OC(F)(F)F)c2)n1] |
| 464 | 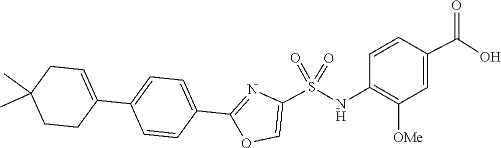 [SMILES: COc1cc(C(=O)O)ccc1NS(=O)(=O)c1coc(-c2ccc(C3=CCC(C)(C)CC3)cc2)n1] |
| 465 | 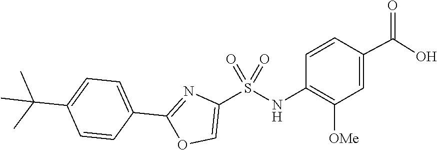 [SMILES: COc1cc(C(=O)O)ccc1NS(=O)(=O)c1coc(-c2ccc(C(C)(C)C)cc2)n1] |
| 466 | 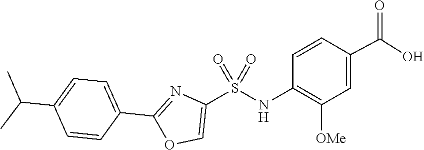 [SMILES: COc1cc(C(=O)O)ccc1NS(=O)(=O)c1coc(-c2ccc(C(C)C)cc2)n1] |
| 467 | 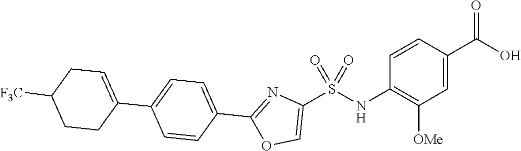 [SMILES: COc1cc(C(=O)O)ccc1NS(=O)(=O)c1coc(-c2ccc(C3=CCC(C(F)(F)F)CC3)cc2)n1] |
| 468 | 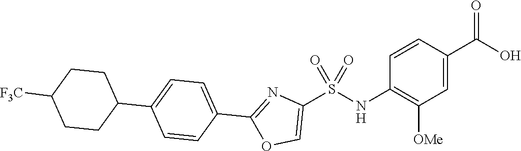 [SMILES: COc1cc(C(=O)O)ccc1NS(=O)(=O)c1coc(-c2ccc(C3CCC(C(F)(F)F)CC3)cc2)n1] |
| 469 | 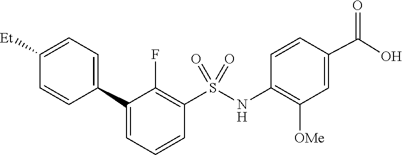 |
| 470 | 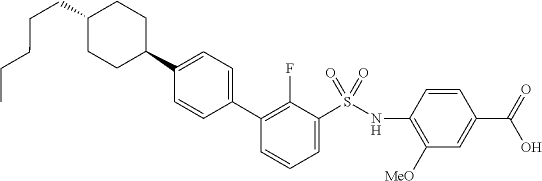 [SMILES: CCCCC[C@H]1CC[C@H](c2ccc(-c3cccc(S(=O)(=O)Nc4ccc(C(=O)O)cc4OC)c3F)cc2)CC1] |
| 471 | 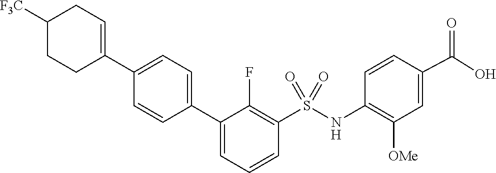 [SMILES: COc1cc(C(=O)O)ccc1NS(=O)(=O)c1cccc(-c2ccc(C3=CCC(C(F)(F)F)CC3)cc2)c1F] |
| 472 | 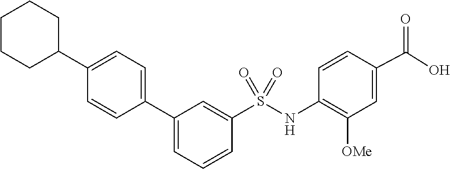 [SMILES: COc1cc(C(=O)O)ccc1NS(=O)(=O)c1cccc(-c2ccc(C3CCCCC3)cc2)c1] |
| 473 | 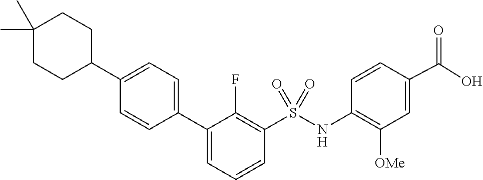 [SMILES: COc1cc(C(=O)O)ccc1NS(=O)(=O)c1cccc(-c2ccc(C3CCC(C)(C)CC3)cc2)c1F] |
| 474 | 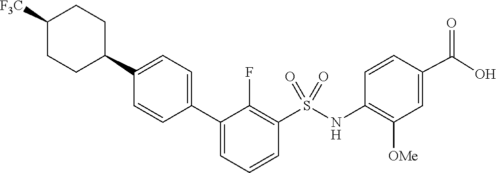 [SMILES: COc1cc(C(=O)O)ccc1NS(=O)(=O)c1cccc(-c2ccc([C@H]3CC[C@@H](C(F)(F)F)CC3)cc2)c1F] |
| 475 | 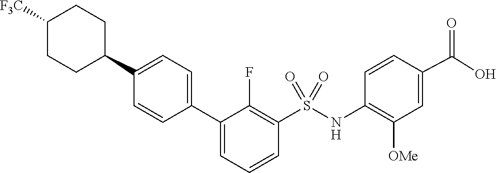 [SMILES: COc1cc(C(=O)O)ccc1NS(=O)(=O)c1cccc(-c2ccc([C@H]3CC[C@H](C(F)(F)F)CC3)cc2)c1F] |
| 476 | 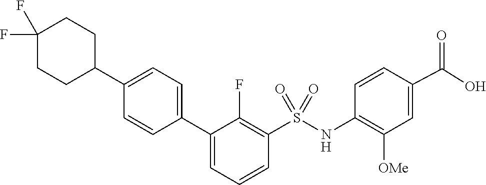 [SMILES: COc1cc(C(=O)O)ccc1NS(=O)(=O)c1cccc(-c2ccc(C3CCC(F)(F)CC3)cc2)c1F] |
| 477 | 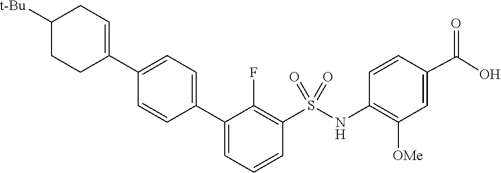 [SMILES: COc1cc(C(=O)O)ccc1NS(=O)(=O)c1cccc(-c2ccc(C3=CCC(C(C)(C)C)CC3)cc2)c1F] |
| 478 | 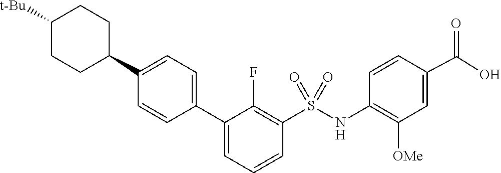 [SMILES: COc1cc(C(=O)O)ccc1NS(=O)(=O)c1cccc(-c2ccc([C@H]3CC[C@H](C(C)(C)C)CC3)cc2)c1F] |
| 479 | 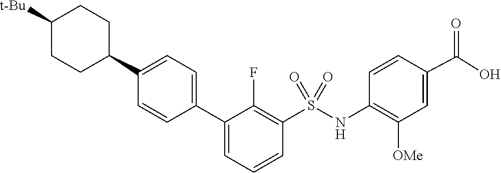 [SMILES: COc1cc(C(=O)O)ccc1NS(=O)(=O)c1cccc(-c2ccc([C@H]3CC[C@@H](C(C)(C)C)CC3)cc2)c1F] |
| 480 | 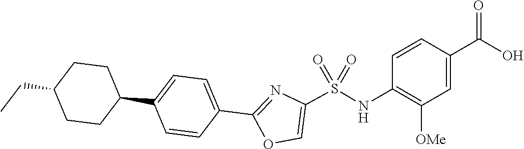 [SMILES: CC[C@H]1CC[C@H](c2ccc(-c3nc(S(=O)(=O)Nc4ccc(C(=O)O)cc4OC)co3)cc2)CC1] |
| 481 | 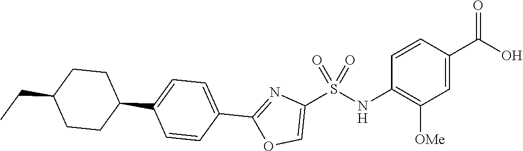 [SMILES: CC[C@H]1CC[C@@H](c2ccc(-c3nc(S(=O)(=O)Nc4ccc(C(=O)O)cc4OC)co3)cc2)CC1] |
| 482 | 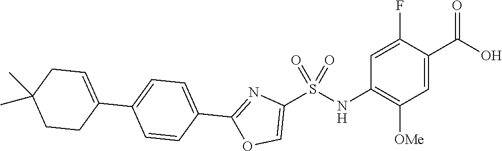 [SMILES: COc1cc(C(=O)O)c(F)cc1NS(=O)(=O)c1coc(-c2ccc(C3=CCC(C)(C)CC3)cc2)n1] |
| 483 | 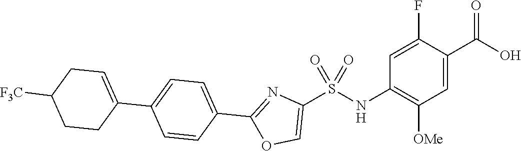 [SMILES: COc1cc(C(=O)O)c(F)cc1NS(=O)(=O)c1coc(-c2ccc(C3=CCC(C(F)(F)F)CC3)cc2)n1] |
| 484 | 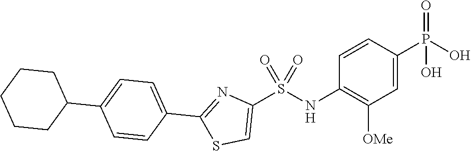 [SMILES: COc1cc(P(=O)(O)O)ccc1NS(=O)(=O)c1csc(-c2ccc(C3CCCCC3)cc2)n1] |
| 485 | 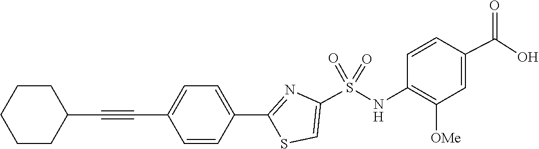 [SMILES: COc1cc(C(=O)O)ccc1NS(=O)(=O)c1csc(-c2ccc(C#CC3CCCCC3)cc2)n1] |
| 486 | 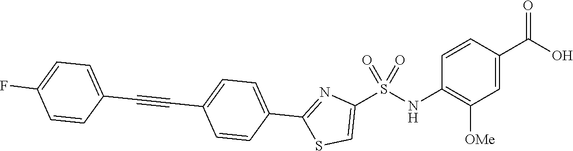 [SMILES: COc1cc(C(=O)O)ccc1NS(=O)(=O)c1csc(-c2ccc(C#Cc3ccc(F)cc3)cc2)n1] |
| 487 | 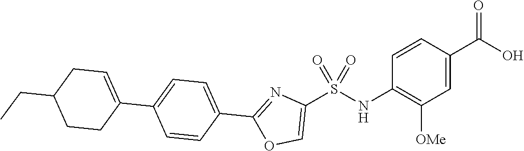 [SMILES: CCC1CC=C(c2ccc(-c3nc(S(=O)(=O)Nc4ccc(C(=O)O)cc4OC)co3)cc2)CC1] |
| 488 | 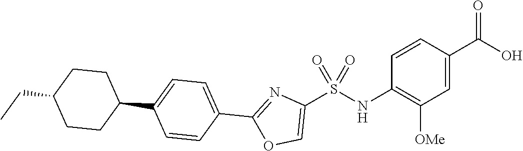 [SMILES: CC[C@H]1CC[C@H](c2ccc(-c3nc(S(=O)(=O)Nc4ccc(C(=O)O)cc4OC)co3)cc2)CC1] |
| 489 | 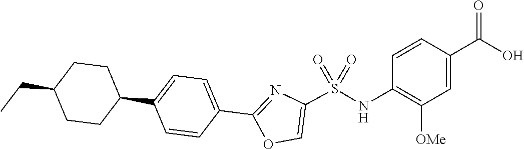 [SMILES: CC[C@H]1CC[C@@H](c2ccc(-c3nc(S(=O)(=O)Nc4ccc(C(=O)O)cc4OC)co3)cc2)CC1] |
| 490 | 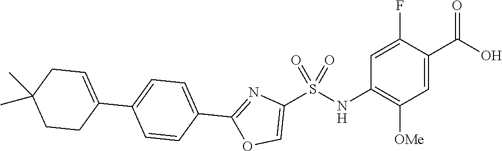 [SMILES: COc1cc(C(=O)O)c(F)cc1NS(=O)(=O)c1coc(-c2ccc(C3=CCC(C)(C)CC3)cc2)n1] |
| 491 | 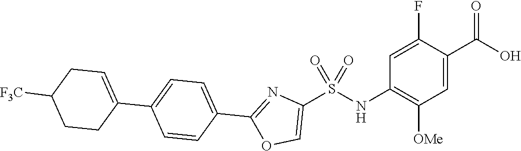 [SMILES: COc1cc(C(=O)O)c(F)cc1NS(=O)(=O)c1coc(-c2ccc(C3=CCC(C(F)(F)F)CC3)cc2)n1] |
| 492 | 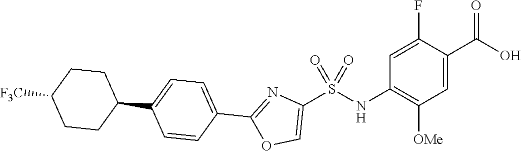 [SMILES: COc1cc(C(=O)O)c(F)cc1NS(=O)(=O)c1coc(-c2ccc([C@H]3CC[C@H](C(F)(F)F)CC3)cc2)n1] |
| 493 | 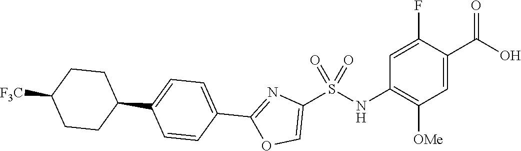 [SMILES: COc1cc(C(=O)O)c(F)cc1NS(=O)(=O)c1coc(-c2ccc([C@H]3CC[C@@H](C(F)(F)F)CC3)cc2)n1] |
| 494 | 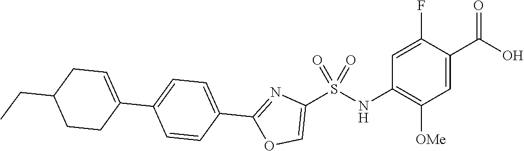 [SMILES: CCC1CC=C(c2ccc(-c3nc(S(=O)(=O)Nc4cc(F)c(C(=O)O)cc4OC)co3)cc2)CC1] |
| 495 | 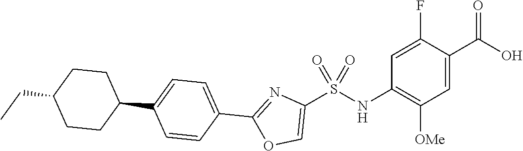 [SMILES: CC[C@H]1CC[C@H](c2ccc(-c3nc(S(=O)(=O)Nc4cc(F)c(C(=O)O)cc4OC)co3)cc2)CC1] |
| 496 | 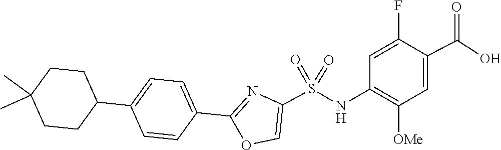 [SMILES: COc1cc(C(=O)O)c(F)cc1NS(=O)(=O)c1coc(-c2ccc(C3CCC(C)(C)CC3)cc2)n1] |
| 497 | 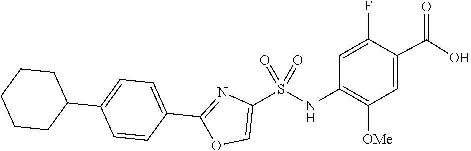 [SMILES: COc1cc(C(=O)O)c(F)cc1NS(=O)(=O)c1coc(-c2ccc(C3CCCCC3)cc2)n1] |
| 498 | 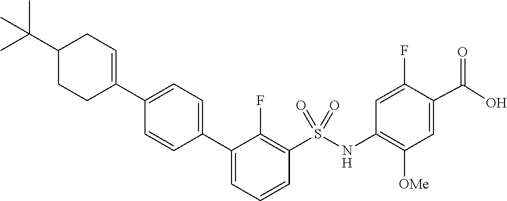 [SMILES: COc1cc(C(=O)O)c(F)cc1NS(=O)(=O)c1cccc(-c2ccc(C3=CCC(C(C)(C)C)CC3)cc2)c1F] |
| 499 | 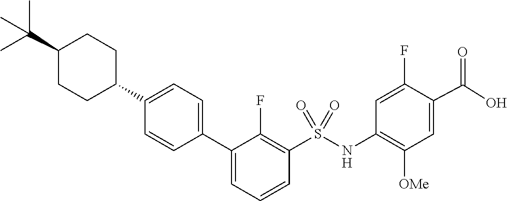 [SMILES: COc1cc(C(=O)O)c(F)cc1NS(=O)(=O)c1cccc(-c2ccc([C@H]3CC[C@H](C(C)(C)C)CC3)cc2)c1F] |
| 500 | 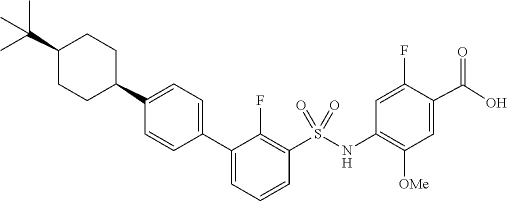 [SMILES: COc1cc(C(=O)O)c(F)cc1NS(=O)(=O)c1cccc(-c2ccc([C@H]3CC[C@@H](C(C)(C)C)CC3)cc2)c1F] |
| 501 | 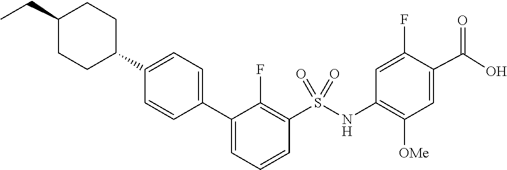 [SMILES: CC[C@H]1CC[C@H](c2ccc(-c3cccc(S(=O)(=O)Nc4cc(F)c(C(=O)O)cc4OC)c3F)cc2)CC1] |
| 502 | 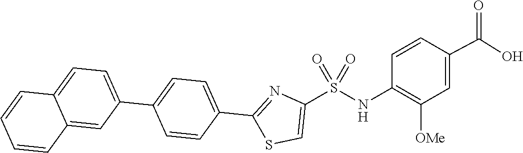 [SMILES: COc1cc(C(=O)O)ccc1NS(=O)(=O)c1csc(-c2ccc(-c3ccc4ccccc4c3)cc2)n1] |
| 503 | 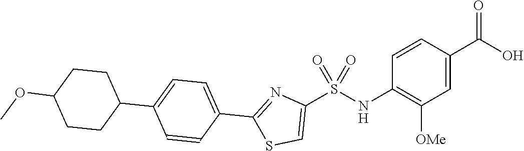 [SMILES: COc1cc(C(=O)O)ccc1NS(=O)(=O)c1csc(-c2ccc(C3CCC(OC)CC3)cc2)n1] |
| 504 | 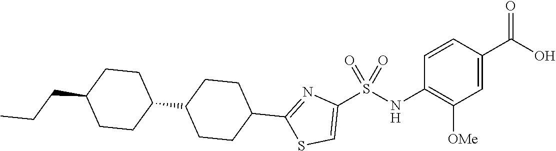 [SMILES: CCC[C@H]1CC[C@H](C2CCC(c3nc(S(=O)(=O)Nc4ccc(C(=O)O)cc4OC)cs3)CC2)CC1] |
| 505 | 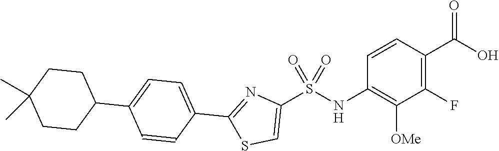 [SMILES: COc1c(NS(=O)(=O)c2csc(-c3ccc(C4CCC(C)(C)CC4)cc3)n2)ccc(C(=O)O)c1F] |
| 506 | 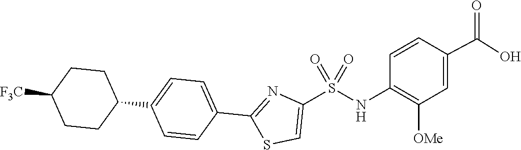 [SMILES: COc1cc(C(=O)O)ccc1NS(=O)(=O)c1csc(-c2ccc([C@H]3CC[C@H](C(F)(F)F)CC3)cc2)n1] |
| 507 | 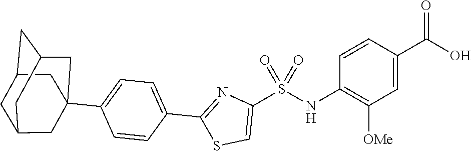 [SMILES: COc1cc(C(=O)O)ccc1NS(=O)(=O)c1csc(-c2ccc(C34CC5CC(CC(C5)C3)C4)cc2)n1] |
| 508 | 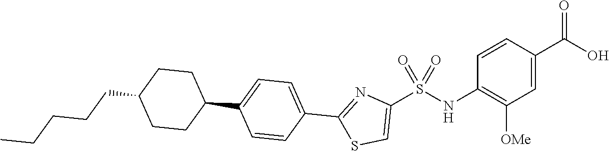 [SMILES: CCCCC[C@H]1CC[C@H](c2ccc(-c3nc(S(=O)(=O)Nc4ccc(C(=O)O)cc4OC)cs3)cc2)CC1] |
| 509 | 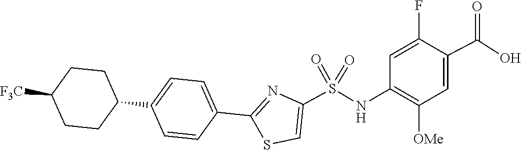 [SMILES: COc1cc(C(=O)O)c(F)cc1NS(=O)(=O)c1csc(-c2ccc([C@H]3CC[C@H](C(F)(F)F)CC3)cc2)n1] |
| 510 | 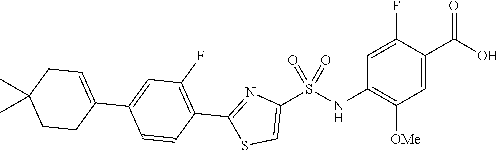 [SMILES: COc1cc(C(=O)O)c(F)cc1NS(=O)(=O)c1csc(-c2ccc(C3=CCC(C)(C)CC3)cc2F)n1] |
| 511 | 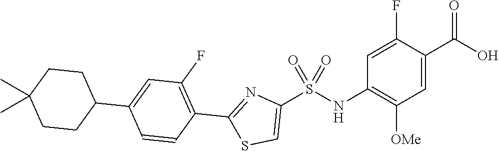 [SMILES: COc1cc(C(=O)O)c(F)cc1NS(=O)(=O)c1csc(-c2ccc(C3CCC(C)(C)CC3)cc2F)n1] |
| 512 | 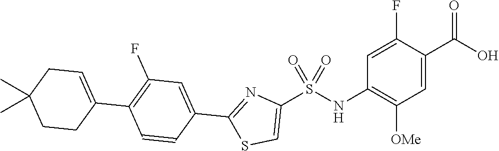 [SMILES: COc1cc(C(=O)O)c(F)cc1NS(=O)(=O)c1csc(-c2ccc(C3=CCC(C)(C)CC3)c(F)c2)n1] |
| 513 | 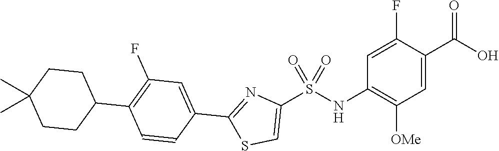 [SMILES: COc1cc(C(=O)O)c(F)cc1NS(=O)(=O)c1csc(-c2ccc(C3CCC(C)(C)CC3)c(F)c2)n1] |
| 514 | 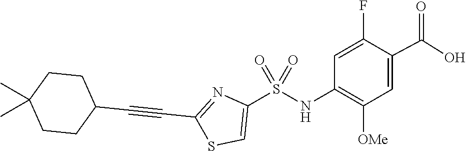 [SMILES: COc1cc(C(=O)O)c(F)cc1NS(=O)(=O)c1csc(C#CC2CCC(C)(C)CC2)n1] |
| 515 | 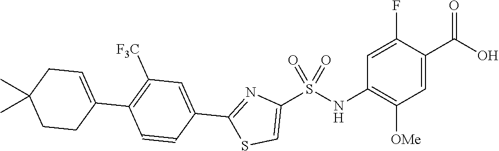 [SMILES: COc1cc(C(=O)O)c(F)cc1NS(=O)(=O)c1csc(-c2ccc(C3=CCC(C)(C)CC3)c(C(F)(F)F)c2)n1] |
| 516 | 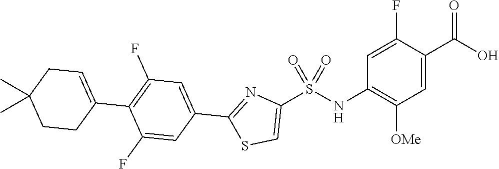 [SMILES: COc1cc(C(=O)O)c(F)cc1NS(=O)(=O)c1csc(-c2cc(F)c(C3=CCC(C)(C)CC3)c(F)c2)n1] |
| 517 | 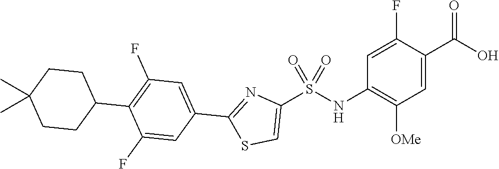 [SMILES: COc1cc(C(=O)O)c(F)cc1NS(=O)(=O)c1csc(-c2cc(F)c(C3CCC(C)(C)CC3)c(F)c2)n1] |
| 518 | 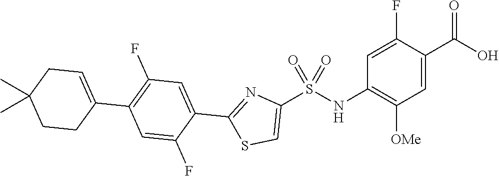 [SMILES: COc1cc(C(=O)O)c(F)cc1NS(=O)(=O)c1csc(-c2cc(F)c(C3=CCC(C)(C)CC3)cc2F)n1] |
| 519 | 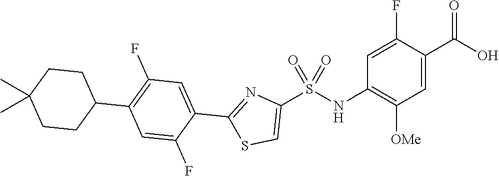 [SMILES: COc1cc(C(=O)O)c(F)cc1NS(=O)(=O)c1csc(-c2cc(F)c(C3CCC(C)(C)CC3)cc2F)n1] |
| 520 | 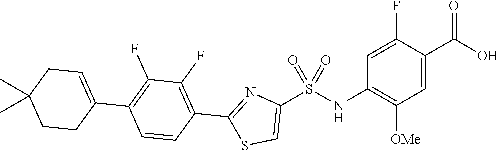 [SMILES: COc1cc(C(=O)O)c(F)cc1NS(=O)(=O)c1csc(-c2ccc(C3=CCC(C)(C)CC3)c(F)c2F)n1] |
| 521 | 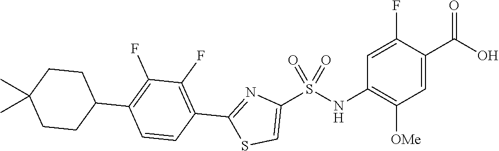 [SMILES: COc1cc(C(=O)O)c(F)cc1NS(=O)(=O)c1csc(-c2ccc(C3CCC(C)(C)CC3)c(F)c2F)n1] |
| 522 | 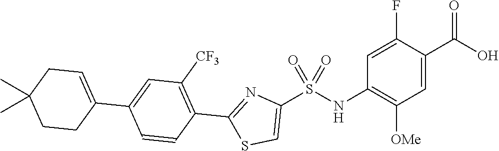 [SMILES: COc1cc(C(=O)O)c(F)cc1NS(=O)(=O)c1csc(-c2ccc(C3=CCC(C)(C)CC3)cc2C(F)(F)F)n1] |
| 523 | 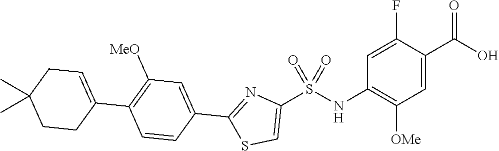 [SMILES: COc1cc(C(=O)O)c(F)cc1NS(=O)(=O)c1csc(-c2ccc(C3=CCC(C)(C)CC3)c(OC)c2)n1] |
| 524 | 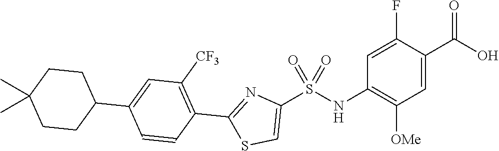 [SMILES: COc1cc(C(=O)O)c(F)cc1NS(=O)(=O)c1csc(-c2ccc(C3CCC(C)(C)CC3)cc2C(F)(F)F)n1] |
| 525 | 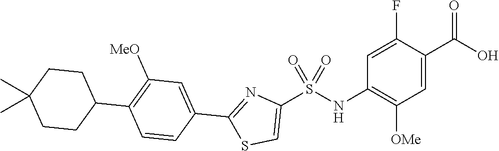 [SMILES: COc1cc(C(=O)O)c(F)cc1NS(=O)(=O)c1csc(-c2ccc(C3CCC(C)(C)CC3)c(OC)c2)n1] |
| 526 | 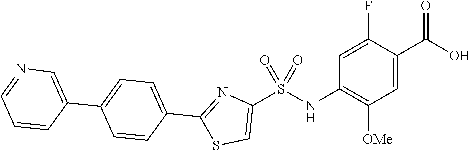 [SMILES: COc1cc(C(=O)O)c(F)cc1NS(=O)(=O)c1csc(-c2ccc(-c3cccnc3)cc2)n1] |
| 527 | 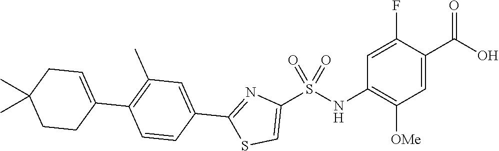 [SMILES: COc1cc(C(=O)O)c(F)cc1NS(=O)(=O)c1csc(-c2ccc(C3=CCC(C)(C)CC3)c(C)c2)n1] |
| 528 | 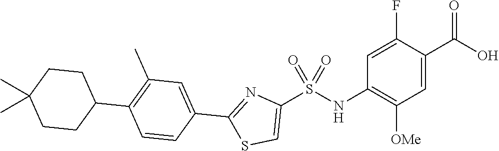 [SMILES: COc1cc(C(=O)O)c(F)cc1NS(=O)(=O)c1csc(-c2ccc(C3CCC(C)(C)CC3)c(C)c2)n1] |
| 529 | 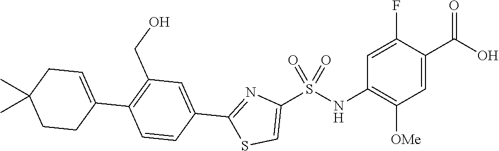 [SMILES: COc1cc(C(=O)O)c(F)cc1NS(=O)(=O)c1csc(-c2ccc(C3=CCC(C)(C)CC3)c(CO)c2)n1] |
| 530 | 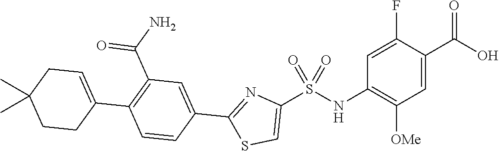 [SMILES: COc1cc(C(=O)O)c(F)cc1NS(=O)(=O)c1csc(-c2ccc(C3=CCC(C)(C)CC3)c(C(N)=O)c2)n1] |
| 531 | 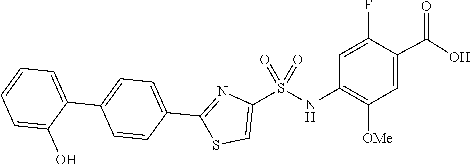 [SMILES: COc1cc(C(=O)O)c(F)cc1NS(=O)(=O)c1csc(-c2ccc(-c3ccccc3O)cc2)n1] |
| 532 | 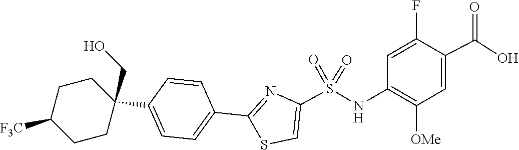 [SMILES: COc1cc(C(=O)O)c(F)cc1NS(=O)(=O)c1csc(-c2ccc([C@]3(CO)CC[C@@H](C(F)(F)F)CC3)cc2)n1] |
| 533 | 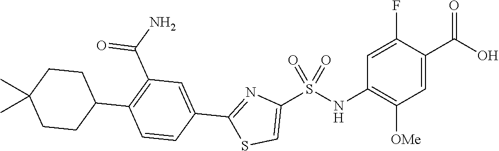 [SMILES: COc1cc(C(=O)O)c(F)cc1NS(=O)(=O)c1csc(-c2ccc(C3CCC(C)(C)CC3)c(C(N)=O)c2)n1] |
| 534 | 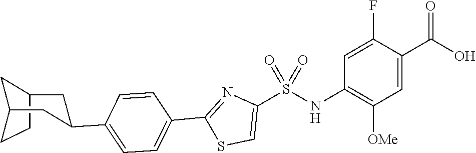 [SMILES: COc1cc(C(=O)O)c(F)cc1NS(=O)(=O)c1csc(-c2ccc(C3CC4CCC(C4)C3)cc2)n1] |
| 535 | 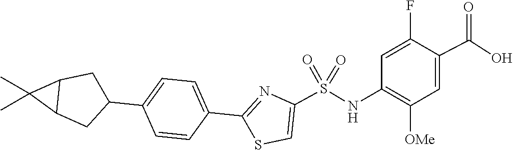 [SMILES: COc1cc(C(=O)O)c(F)cc1NS(=O)(=O)c1csc(-c2ccc(C3CC4C(C3)C4(C)C)cc2)n1] |
| 536 | 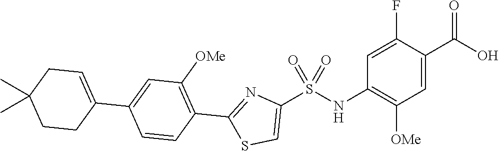 [SMILES: COc1cc(C(=O)O)c(F)cc1NS(=O)(=O)c1csc(-c2ccc(C3=CCC(C)(C)CC3)cc2OC)n1] |
| 537 | 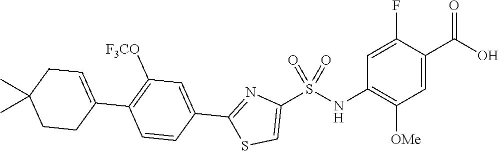 [SMILES: COc1cc(C(=O)O)c(F)cc1NS(=O)(=O)c1csc(-c2ccc(C3=CCC(C)(C)CC3)c(OC(F)(F)F)c2)n1] |
| 538 | 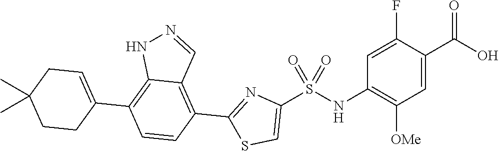 [SMILES: COc1cc(C(=O)O)c(F)cc1NS(=O)(=O)c1csc(-c2ccc(C3=CCC(C)(C)CC3)c3[nH]ncc23)n1] |
| 539 | 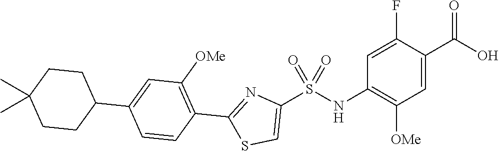 [SMILES: COc1cc(C(=O)O)c(F)cc1NS(=O)(=O)c1csc(-c2ccc(C3CCC(C)(C)CC3)cc2OC)n1] |
| 540 | 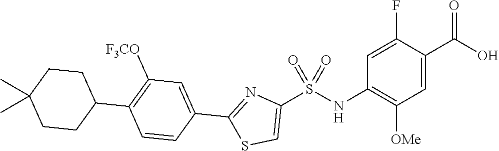 [SMILES: COc1cc(C(=O)O)c(F)cc1NS(=O)(=O)c1csc(-c2ccc(C3CCC(C)(C)CC3)c(OC(F)(F)F)c2)n1] |
| 541 | 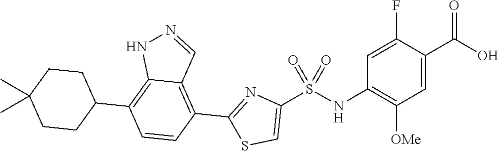 [SMILES: COc1cc(C(=O)O)c(F)cc1NS(=O)(=O)c1csc(-c2ccc(C3CCC(C)(C)CC3)c3[nH]ncc23)n1] |
| 542 | 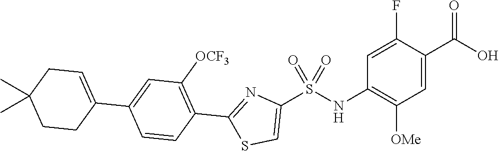 [SMILES: COc1cc(C(=O)O)c(F)cc1NS(=O)(=O)c1csc(-c2ccc(C3=CCC(C)(C)CC3)cc2OC(F)(F)F)n1] |
| 543 | 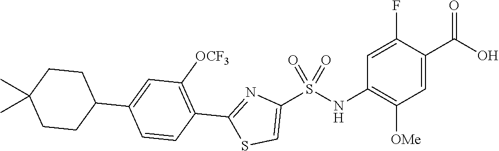 [SMILES: COc1cc(C(=O)O)c(F)cc1NS(=O)(=O)c1csc(-c2ccc(C3CCC(C)(C)CC3)cc2OC(F)(F)F)n1] |
| 544 | 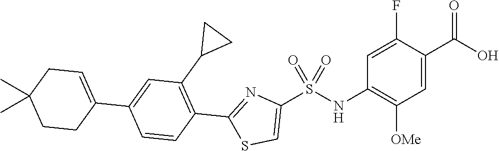 [SMILES: COc1cc(C(=O)O)c(F)cc1NS(=O)(=O)c1csc(-c2ccc(C3=CCC(C)(C)CC3)cc2C2CC2)n1] |
| 545 | 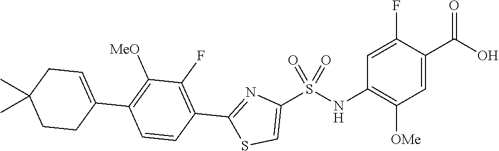 [SMILES: COc1cc(C(=O)O)c(F)cc1NS(=O)(=O)c1csc(-c2ccc(C3=CCC(C)(C)CC3)c(OC)c2F)n1] |
| 546 | 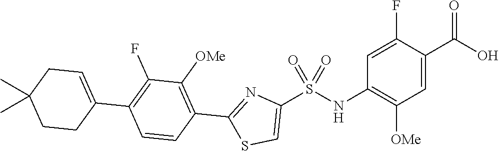 [SMILES: COc1cc(C(=O)O)c(F)cc1NS(=O)(=O)c1csc(-c2ccc(C3=CCC(C)(C)CC3)c(F)c2OC)n1] |
| 547 | 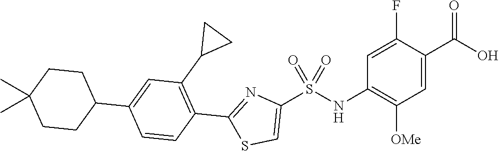 [SMILES: COc1cc(C(=O)O)c(F)cc1NS(=O)(=O)c1csc(-c2ccc(C3CCC(C)(C)CC3)cc2C2CC2)n1] |
| 548 | 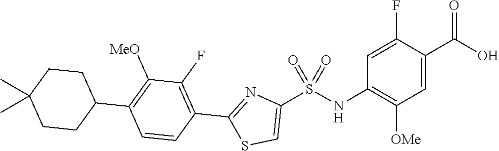 [SMILES: COc1cc(C(=O)O)c(F)cc1NS(=O)(=O)c1csc(-c2ccc(C3CCC(C)(C)CC3)c(OC)c2F)n1] |
| 549 | 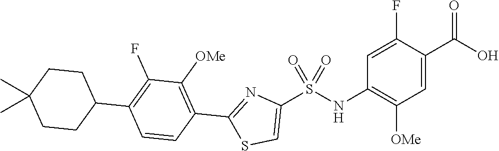 [SMILES: COc1cc(C(=O)O)c(F)cc1NS(=O)(=O)c1csc(-c2ccc(C3CCC(C)(C)CC3)c(F)c2OC)n1] |
| 550 | 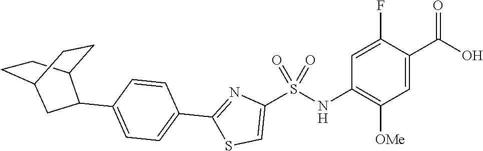 [SMILES: COc1cc(C(=O)O)c(F)cc1NS(=O)(=O)c1csc(-c2ccc(C3CC4CCC3CC4)cc2)n1] |
| 551 | 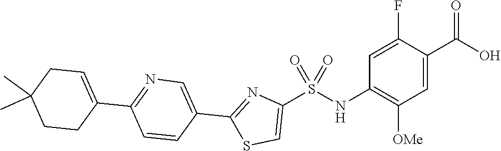 [SMILES: COc1cc(C(=O)O)c(F)cc1NS(=O)(=O)c1csc(-c2ccc(C3=CCC(C)(C)CC3)nc2)n1] |
| 552 | 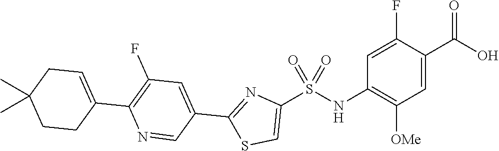 [SMILES: COc1cc(C(=O)O)c(F)cc1NS(=O)(=O)c1csc(-c2cnc(C3=CCC(C)(C)CC3)c(F)c2)n1] |
| 553 | 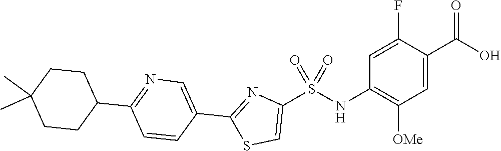 [SMILES: COc1cc(C(=O)O)c(F)cc1NS(=O)(=O)c1csc(-c2ccc(C3CCC(C)(C)CC3)nc2)n1] |
| 554 | 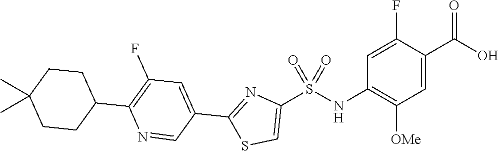 [SMILES: COc1cc(C(=O)O)c(F)cc1NS(=O)(=O)c1csc(-c2cnc(C3CCC(C)(C)CC3)c(F)c2)n1] |
| 555 | 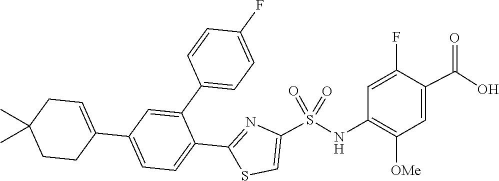 [SMILES: COc1cc(C(=O)O)c(F)cc1NS(=O)(=O)c1csc(-c2ccc(C3=CCC(C)(C)CC3)cc2-c2ccc(F)cc2)n1] |
| 556 | 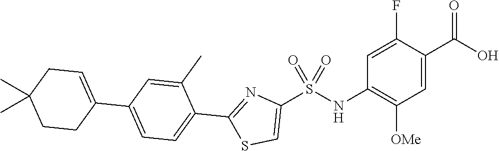 [SMILES: COc1cc(C(=O)O)c(F)cc1NS(=O)(=O)c1csc(-c2ccc(C3=CCC(C)(C)CC3)cc2C)n1] |
| 557 | 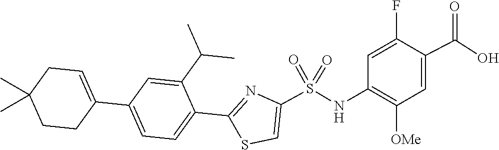 [SMILES: COc1cc(C(=O)O)c(F)cc1NS(=O)(=O)c1csc(-c2ccc(C3=CCC(C)(C)CC3)cc2C(C)C)n1] |
| 558 | 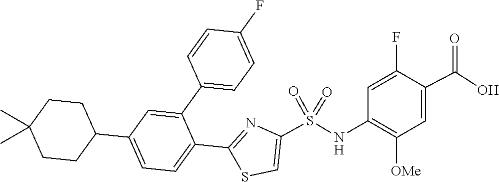 [SMILES: COc1cc(C(=O)O)c(F)cc1NS(=O)(=O)c1csc(-c2ccc(C3CCC(C)(C)CC3)cc2-c2ccc(F)cc2)n1] |
| 559 | 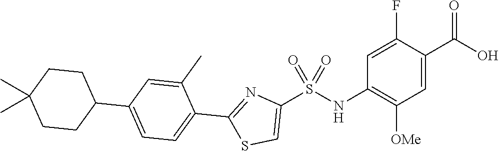 [SMILES: COc1cc(C(=O)O)c(F)cc1NS(=O)(=O)c1csc(-c2ccc(C3CCC(C)(C)CC3)cc2C)n1] |
| 560 | 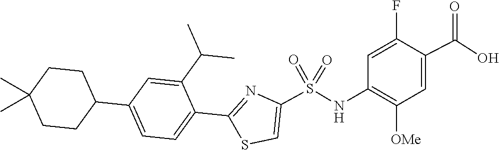 [SMILES: COc1cc(C(=O)O)c(F)cc1NS(=O)(=O)c1csc(-c2ccc(C3CCC(C)(C)CC3)cc2C(C)C)n1] |
| 561 | 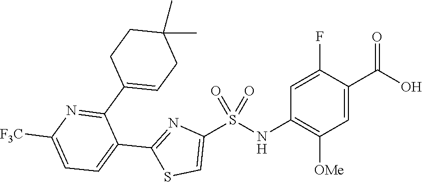 [SMILES: COc1cc(C(=O)O)c(F)cc1NS(=O)(=O)c1csc(-c2ccc(C(F)(F)F)nc2C2=CCC(C)(C)CC2)n1] |
| 562 | 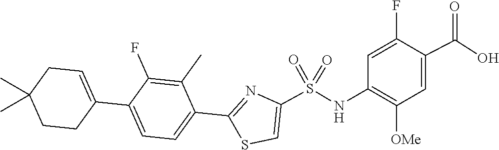 [SMILES: COc1cc(C(=O)O)c(F)cc1NS(=O)(=O)c1csc(-c2ccc(C3=CCC(C)(C)CC3)c(F)c2C)n1] |
| 563 | 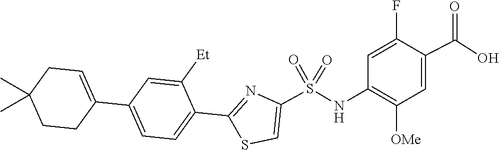 [SMILES: CCc1cc(C2=CCC(C)(C)CC2)ccc1-c1nc(S(=O)(=O)Nc2cc(F)c(C(=O)O)cc2OC)cs1] |
| 564 | 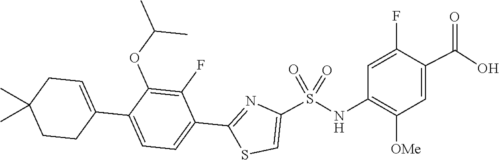 [SMILES: COc1cc(C(=O)O)c(F)cc1NS(=O)(=O)c1csc(-c2ccc(C3=CCC(C)(C)CC3)c(OC(C)C)c2F)n1] |
| 565 | 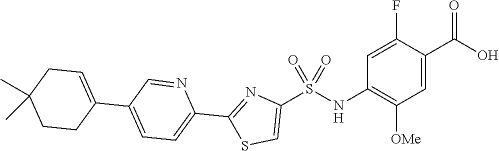 [SMILES: COc1cc(C(=O)O)c(F)cc1NS(=O)(=O)c1csc(-c2ccc(C3=CCC(C)(C)CC3)cn2)n1] |
| 566 | 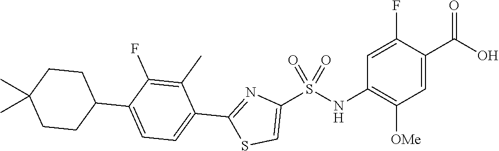 [SMILES: COc1cc(C(=O)O)c(F)cc1NS(=O)(=O)c1csc(-c2ccc(C3CCC(C)(C)CC3)c(F)c2C)n1] |
| 567 | 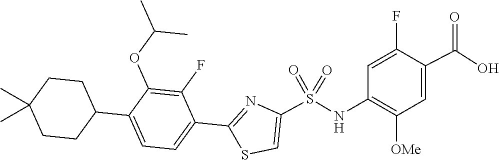 [SMILES: COc1cc(C(=O)O)c(F)cc1NS(=O)(=O)c1csc(-c2ccc(C3CCC(C)(C)CC3)c(OC(C)C)c2F)n1] |
| 568 | 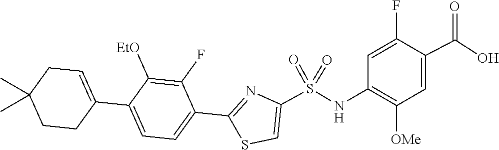 [SMILES: CCOc1c(C2=CCC(C)(C)CC2)ccc(-c2nc(S(=O)(=O)Nc3cc(F)c(C(=O)O)cc3OC)cs2)c1F] |
| 569 | 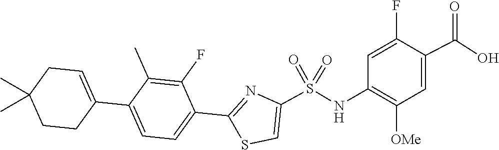 [SMILES: COc1cc(C(=O)O)c(F)cc1NS(=O)(=O)c1csc(-c2ccc(C3=CCC(C)(C)CC3)c(C)c2F)n1] |
| 570 | 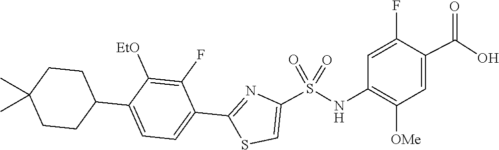 [SMILES: CCOc1c(C2CCC(C)(C)CC2)ccc(-c2nc(S(=O)(=O)Nc3cc(F)c(C(=O)O)cc3OC)cs2)c1F] |
| 571 | 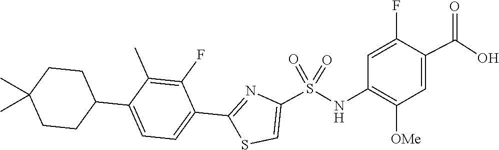 [SMILES: COc1cc(C(=O)O)c(F)cc1NS(=O)(=O)c1csc(-c2ccc(C3CCC(C)(C)CC3)c(C)c2F)n1] |
| 572 | 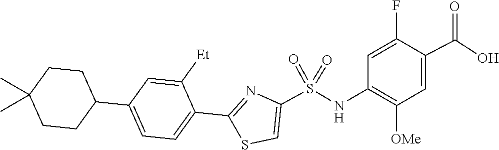 [SMILES: CCc1cc(C2CCC(C)(C)CC2)ccc1-c1nc(S(=O)(=O)Nc2cc(F)c(C(=O)O)cc2OC)cs1] |
| 573 | 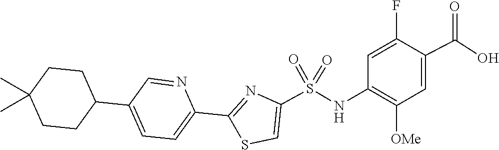 [SMILES: COc1cc(C(=O)O)c(F)cc1NS(=O)(=O)c1csc(-c2ccc(C3CCC(C)(C)CC3)cn2)n1] |
| 574 | 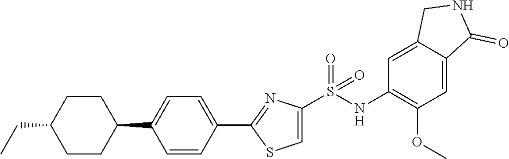 [SMILES: CC[C@H]1CC[C@H](c2ccc(-c3nc(S(=O)(=O)Nc4cc5c(cc4OC)C(=O)NC5)cs3)cc2)CC1] |
| 575 | 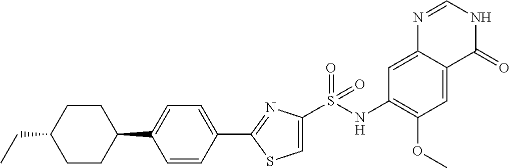 [SMILES: CC[C@H]1CC[C@H](c2ccc(-c3nc(S(=O)(=O)Nc4cc5nc[nH]c(=O)c5cc4OC)cs3)cc2)CC1] |
| 576 | 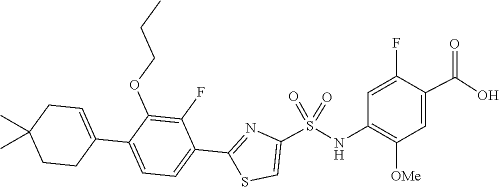 [SMILES: CCCOc1c(C2=CCC(C)(C)CC2)ccc(-c2nc(S(=O)(=O)Nc3cc(F)c(C(=O)O)cc3OC)cs2)c1F] |
| 577 | 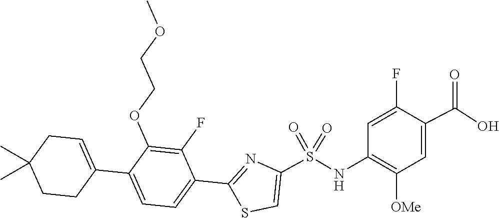 [SMILES: COCCOc1c(C2=CCC(C)(C)CC2)ccc(-c2nc(S(=O)(=O)Nc3cc(F)c(C(=O)O)cc3OC)cs2)c1F] |
| 578 | 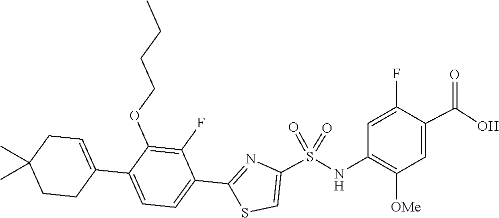 [SMILES: CCCCOc1c(C2=CCC(C)(C)CC2)ccc(-c2nc(S(=O)(=O)Nc3cc(F)c(C(=O)O)cc3OC)cs2)c1F] |
| 579 | 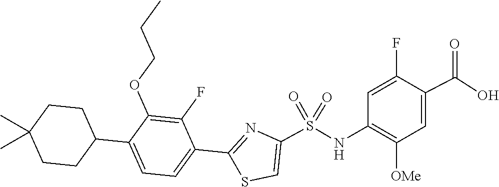 [SMILES: CCCOc1c(C2CCC(C)(C)CC2)ccc(-c2nc(S(=O)(=O)Nc3cc(F)c(C(=O)O)cc3OC)cs2)c1F] |
| 580 | 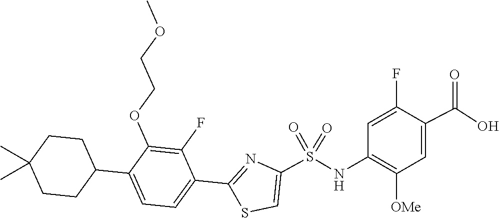 [SMILES: COCCOc1c(C2CCC(C)(C)CC2)ccc(-c2nc(S(=O)(=O)Nc3cc(F)c(C(=O)O)cc3OC)cs2)c1F] |
| 581 | 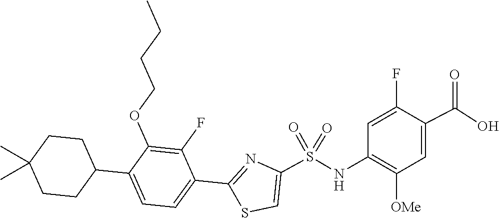 [SMILES: CCCCOc1c(C2CCC(C)(C)CC2)ccc(-c2nc(S(=O)(=O)Nc3cc(F)c(C(=O)O)cc3OC)cs2)c1F] |
| 582 | 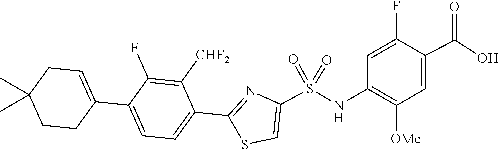 [SMILES: COc1cc(C(=O)O)c(F)cc1NS(=O)(=O)c1csc(-c2ccc(C3=CCC(C)(C)CC3)c(F)c2C(F)F)n1] |
| 583 | 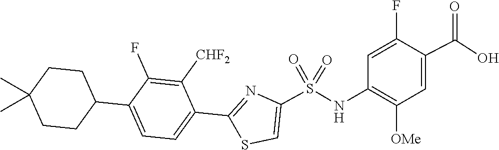 [SMILES: COc1cc(C(=O)O)c(F)cc1NS(=O)(=O)c1csc(-c2ccc(C3CCC(C)(C)CC3)c(F)c2C(F)F)n1] |
| 584 | 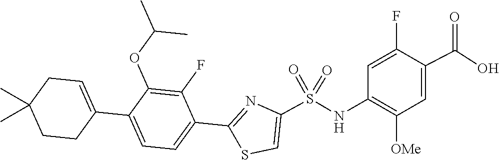 [SMILES: COc1cc(C(=O)O)c(F)cc1NS(=O)(=O)c1csc(-c2ccc(C3=CCC(C)(C)CC3)c(OC(C)C)c2F)n1] |
| 585 | 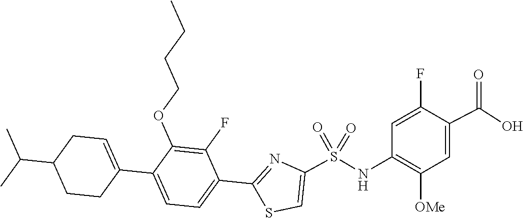 [SMILES: CCCCOc1c(C2=CCC(C(C)C)CC2)ccc(-c2nc(S(=O)(=O)Nc3cc(F)c(C(=O)O)cc3OC)cs2)c1F] |
| 586 | 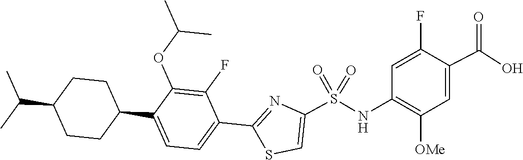 [SMILES: COc1cc(C(=O)O)c(F)cc1NS(=O)(=O)c1csc(-c2ccc([C@H]3CC[C@@H](C(C)C)CC3)c(OC(C)C)c2F)n1] |
| 587 | 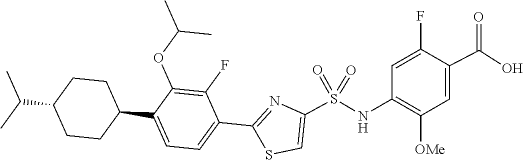 [SMILES: COc1cc(C(=O)O)c(F)cc1NS(=O)(=O)c1csc(-c2ccc([C@H]3CC[C@H](C(C)C)CC3)c(OC(C)C)c2F)n1] |
| 588 | 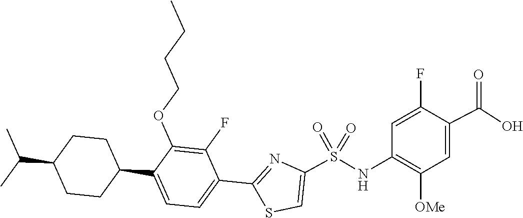 [SMILES: CCCCOc1c(F)c(-c2nc(S(=O)(=O)Nc3cc(F)c(C(=O)O)cc3OC)cs2)ccc1[C@H]1CC[C@@H](C(C)C)CC1] |
| 589 | 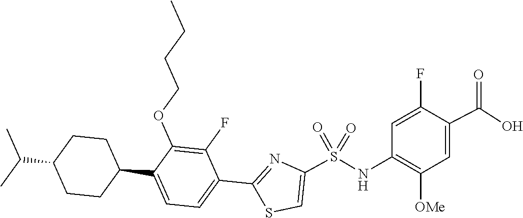 [SMILES: CCCCOc1c(F)c(-c2nc(S(=O)(=O)Nc3cc(F)c(C(=O)O)cc3OC)cs2)ccc1[C@H]1CC[C@H](C(C)C)CC1] |
| 590 | 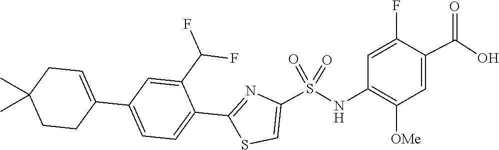 [SMILES: COc1cc(C(=O)O)c(F)cc1NS(=O)(=O)c1csc(-c2ccc(C3=CCC(C)(C)CC3)cc2C(F)F)n1] |
| 591 | 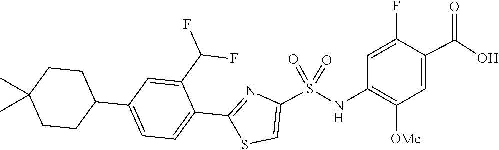 [SMILES: COc1cc(C(=O)O)c(F)cc1NS(=O)(=O)c1csc(-c2ccc(C3CCC(C)(C)CC3)cc2C(F)F)n1] |
| 592 | 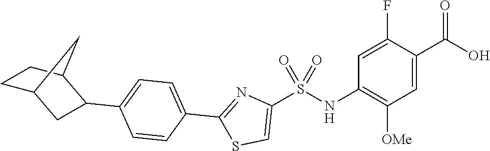 [SMILES: COc1cc(C(=O)O)c(F)cc1NS(=O)(=O)c1csc(-c2ccc(C3CC4CCC3C4)cc2)n1] |
| 593 | 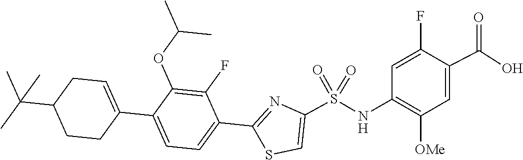 [SMILES: COc1cc(C(=O)O)c(F)cc1NS(=O)(=O)c1csc(-c2ccc(C3=CCC(C(C)(C)C)CC3)c(OC(C)C)c2F)n1] |
| 594 | 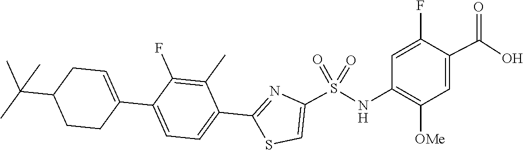 [SMILES: COc1cc(C(=O)O)c(F)cc1NS(=O)(=O)c1csc(-c2ccc(C3=CCC(C(C)(C)C)CC3)c(F)c2C)n1] |
| 595 | 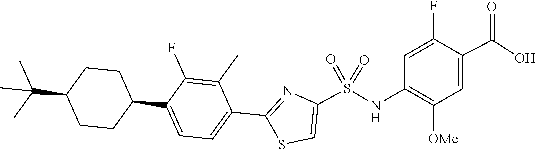 [SMILES: COc1cc(C(=O)O)c(F)cc1NS(=O)(=O)c1csc(-c2ccc([C@H]3CC[C@@H](C(C)(C)C)CC3)c(F)c2C)n1] |
| 596 | 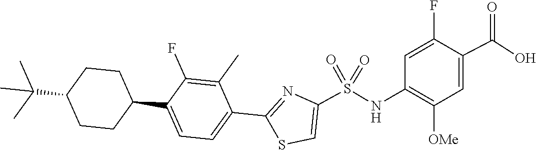 [SMILES: COc1cc(C(=O)O)c(F)cc1NS(=O)(=O)c1csc(-c2ccc([C@H]3CC[C@H](C(C)(C)C)CC3)c(F)c2C)n1] |
| 597 | 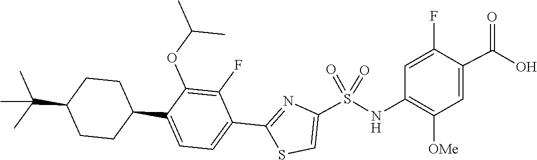 [SMILES: COc1cc(C(=O)O)c(F)cc1NS(=O)(=O)c1csc(-c2ccc([C@H]3CC[C@@H](C(C)(C)C)CC3)c(OC(C)C)c2F)n1] |
| 598 | 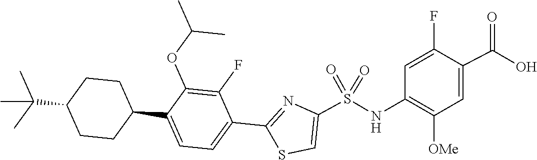 [SMILES: COc1cc(C(=O)O)c(F)cc1NS(=O)(=O)c1csc(-c2ccc([C@H]3CC[C@H](C(C)(C)C)CC3)c(OC(C)C)c2F)n1] |
| 599 | 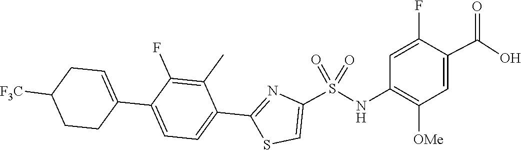 [SMILES: COc1cc(C(=O)O)c(F)cc1NS(=O)(=O)c1csc(-c2ccc(C3=CCC(C(F)(F)F)CC3)c(F)c2C)n1] |
| 600 | 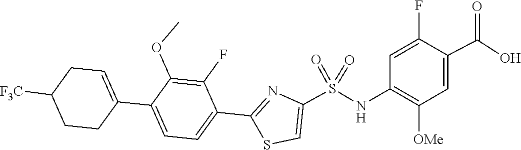 [SMILES: COc1cc(C(=O)O)c(F)cc1NS(=O)(=O)c1csc(-c2ccc(C3=CCC(C(F)(F)F)CC3)c(OC)c2F)n1] |
| 601 | 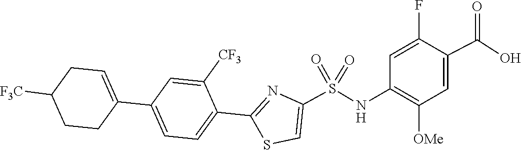 [SMILES: COc1cc(C(=O)O)c(F)cc1NS(=O)(=O)c1csc(-c2ccc(C3=CCC(C(F)(F)F)CC3)cc2C(F)(F)F)n1] |
| 602 | 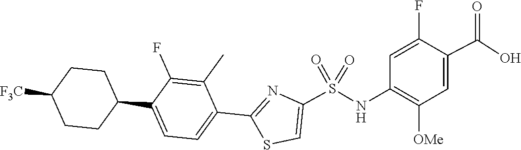 [SMILES: COc1cc(C(=O)O)c(F)cc1NS(=O)(=O)c1csc(-c2ccc([C@H]3CC[C@@H](C(F)(F)F)CC3)c(F)c2C)n1] |
| 603 | 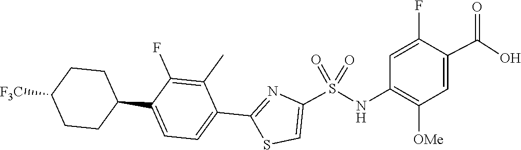 [SMILES: COc1cc(C(=O)O)c(F)cc1NS(=O)(=O)c1csc(-c2ccc([C@H]3CC[C@H](C(F)(F)F)CC3)c(F)c2C)n1] |
| 604 | 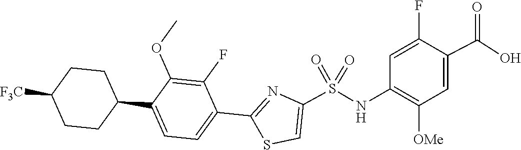 [SMILES: COc1cc(C(=O)O)c(F)cc1NS(=O)(=O)c1csc(-c2ccc([C@H]3CC[C@@H](C(F)(F)F)CC3)c(OC)c2F)n1] |
| 605 | 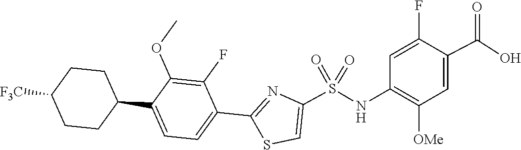 [SMILES: COc1cc(C(=O)O)c(F)cc1NS(=O)(=O)c1csc(-c2ccc([C@H]3CC[C@H](C(F)(F)F)CC3)c(OC)c2F)n1] |
| 606 | 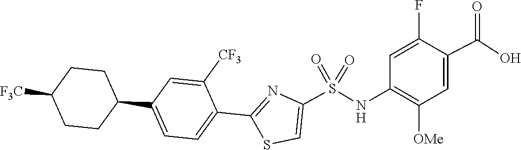 [SMILES: COc1cc(C(=O)O)c(F)cc1NS(=O)(=O)c1csc(-c2ccc([C@H]3CC[C@@H](C(F)(F)F)CC3)cc2C(F)(F)F)n1] |
| 607 | 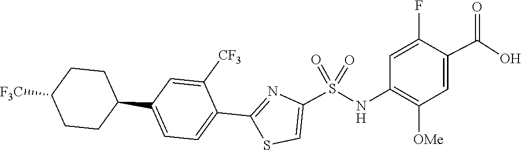 [SMILES: COc1cc(C(=O)O)c(F)cc1NS(=O)(=O)c1csc(-c2ccc([C@H]3CC[C@H](C(F)(F)F)CC3)cc2C(F)(F)F)n1] |
| 608 | 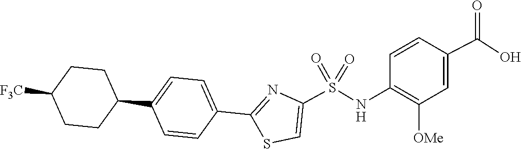 [SMILES: COc1cc(C(=O)O)ccc1NS(=O)(=O)c1csc(-c2ccc([C@H]3CC[C@@H](C(F)(F)F)CC3)cc2)n1] |
| 609 | 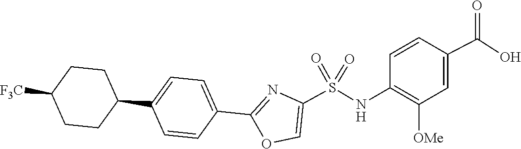 [SMILES: COc1cc(C(=O)O)ccc1NS(=O)(=O)c1coc(-c2ccc([C@H]3CC[C@@H](C(F)(F)F)CC3)cc2)n1] |
| 610 | 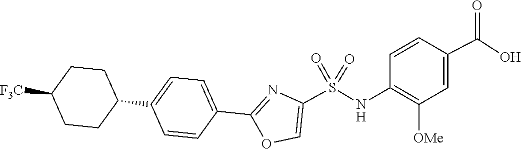 [SMILES: COc1cc(C(=O)O)ccc1NS(=O)(=O)c1coc(-c2ccc([C@H]3CC[C@H](C(F)(F)F)CC3)cc2)n1] |
| 611 | 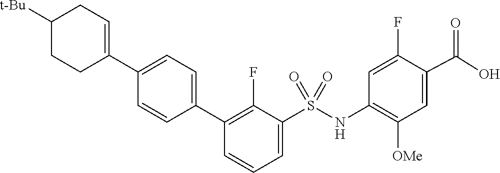 [SMILES: COc1cc(C(=O)O)c(F)cc1NS(=O)(=O)c1cccc(-c2ccc(C3=CCC(C(C)(C)C)CC3)cc2)c1F] |
| 612 | 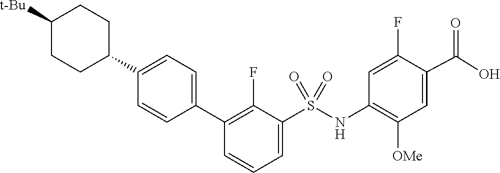 [SMILES: COc1cc(C(=O)O)c(F)cc1NS(=O)(=O)c1cccc(-c2ccc([C@H]3CC[C@H](C(C)(C)C)CC3)cc2)c1F] |
| 613 | 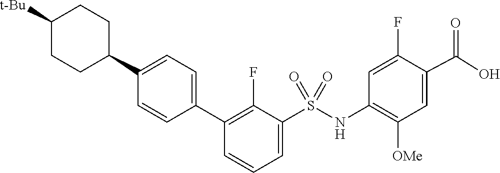 [SMILES: COc1cc(C(=O)O)c(F)cc1NS(=O)(=O)c1cccc(-c2ccc([C@H]3CC[C@@H](C(C)(C)C)CC3)cc2)c1F] |
| 614 | 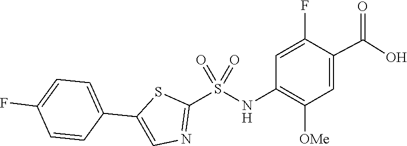 [SMILES: COc1cc(C(=O)O)c(F)cc1NS(=O)(=O)c1ncc(-c2ccc(F)cc2)s1] |
| 615 | 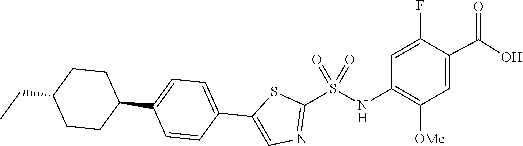 [SMILES: CC[C@H]1CC[C@H](c2ccc(-c3cnc(S(=O)(=O)Nc4cc(F)c(C(=O)O)cc4OC)s3)cc2)CC1] |
| 616 | 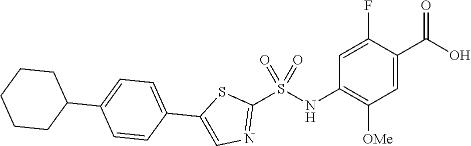 [SMILES: COc1cc(C(=O)O)c(F)cc1NS(=O)(=O)c1ncc(-c2ccc(C3CCCCC3)cc2)s1] |
| 617 | 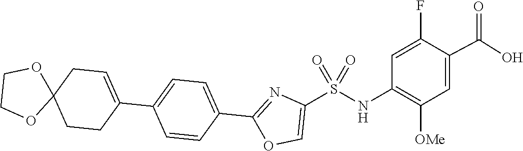 [SMILES: COc1cc(C(=O)O)c(F)cc1NS(=O)(=O)c1coc(-c2ccc(C3=CCC4(CC3)OCCO4)cc2)n1] |
| 618 | 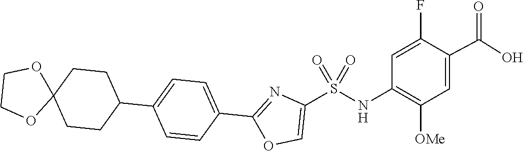 [SMILES: COc1cc(C(=O)O)c(F)cc1NS(=O)(=O)c1coc(-c2ccc(C3CCC4(CC3)OCCO4)cc2)n1] |
| 619 | 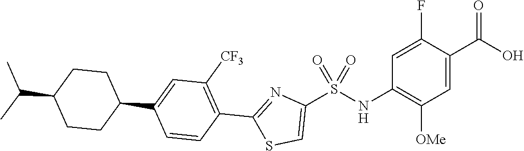 [SMILES: COc1cc(C(=O)O)c(F)cc1NS(=O)(=O)c1csc(-c2ccc([C@H]3CC[C@@H](C(C)C)CC3)cc2C(F)(F)F)n1] |
| 620 | 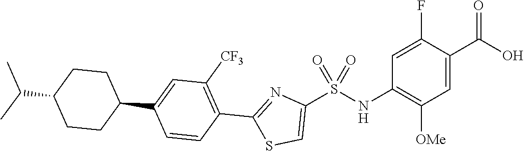 [SMILES: COc1cc(C(=O)O)c(F)cc1NS(=O)(=O)c1csc(-c2ccc([C@H]3CC[C@H](C(C)C)CC3)cc2C(F)(F)F)n1] |
| 621 | 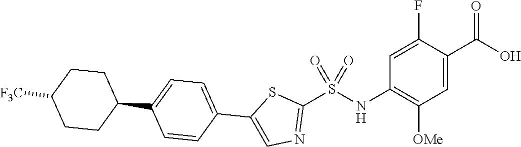 [SMILES: COc1cc(C(=O)O)c(F)cc1NS(=O)(=O)c1ncc(-c2ccc([C@H]3CC[C@H](C(F)(F)F)CC3)cc2)s1] |
| 622 | 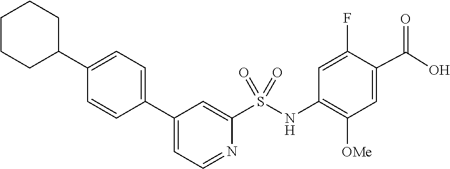 [SMILES: COc1cc(C(=O)O)c(F)cc1NS(=O)(=O)c1cc(-c2ccc(C3CCCCC3)cc2)ccn1] |
| 623 | 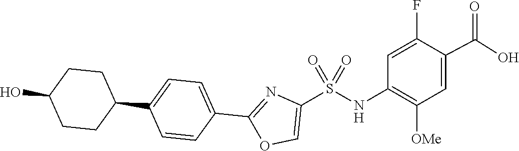 [SMILES: COc1cc(C(=O)O)c(F)cc1NS(=O)(=O)c1coc(-c2ccc([C@H]3CC[C@@H](O)CC3)cc2)n1] |
| 624 | 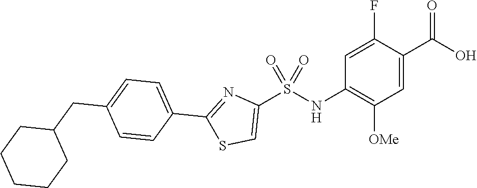 [SMILES: COc1cc(C(=O)O)c(F)cc1NS(=O)(=O)c1csc(-c2ccc(CC3CCCCC3)cc2)n1] |
| 625 | 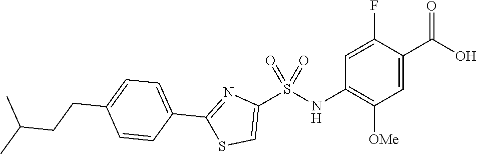 [SMILES: COc1cc(C(=O)O)c(F)cc1NS(=O)(=O)c1csc(-c2ccc(CCC(C)C)cc2)n1] |
| 626 | 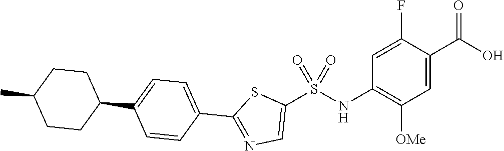 [SMILES: COc1cc(C(=O)O)c(F)cc1NS(=O)(=O)c1cnc(-c2ccc([C@H]3CC[C@@H](C)CC3)cc2)s1] |
| 627 | 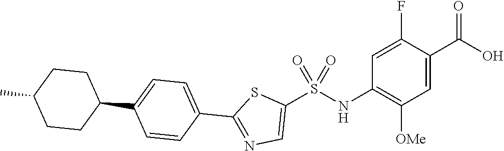 [SMILES: COc1cc(C(=O)O)c(F)cc1NS(=O)(=O)c1cnc(-c2ccc([C@H]3CC[C@H](C)CC3)cc2)s1] |
| 628 | 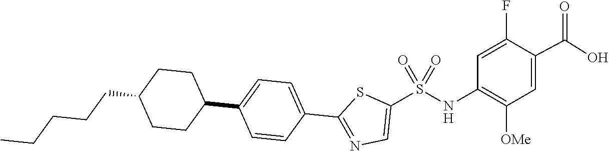 |
| 629 | 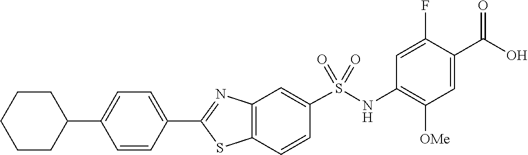 [SMILES: COc1cc(C(=O)O)c(F)cc1NS(=O)(=O)c1ccc2sc(-c3ccc(C4CCCCC4)cc3)nc2c1] |
| 630 | 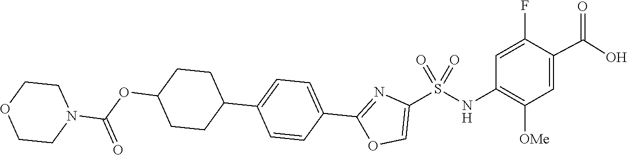 [SMILES: COc1cc(C(=O)O)c(F)cc1NS(=O)(=O)c1coc(-c2ccc(C3CCC(OC(=O)N4CCOCC4)CC3)cc2)n1] |
| 631 | 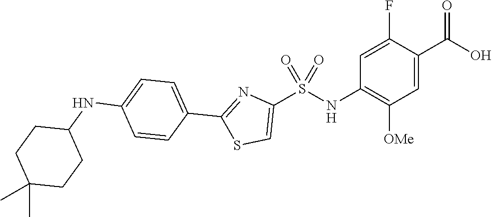 [SMILES: COc1cc(C(=O)O)c(F)cc1NS(=O)(=O)c1csc(-c2ccc(NC3CCC(C)(C)CC3)cc2)n1] |
| 632 | 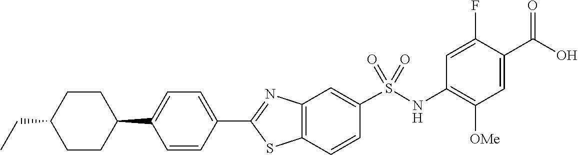 [SMILES: CC[C@H]1CC[C@H](c2ccc(-c3nc4cc(S(=O)(=O)Nc5cc(F)c(C(=O)O)cc5OC)ccc4s3)cc2)CC1] |
| 633 | 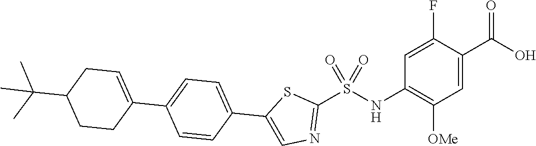 [SMILES: COc1cc(C(=O)O)c(F)cc1NS(=O)(=O)c1ncc(-c2ccc(C3=CCC(C(C)(C)C)CC3)cc2)s1] |
| 634 | 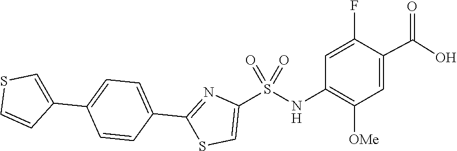 [SMILES: COc1cc(C(=O)O)c(F)cc1NS(=O)(=O)c1csc(-c2ccc(-c3ccsc3)cc2)n1] |
| 635 | 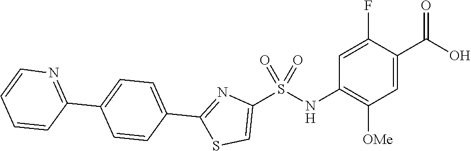 [SMILES: COc1cc(C(=O)O)c(F)cc1NS(=O)(=O)c1csc(-c2ccc(-c3ccccn3)cc2)n1] |
| 636 | 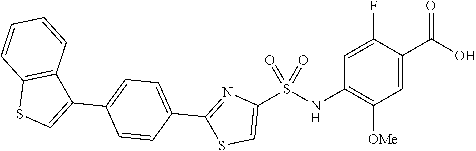 [SMILES: COc1cc(C(=O)O)c(F)cc1NS(=O)(=O)c1csc(-c2ccc(-c3csc4ccccc34)cc2)n1] |
| 637 | 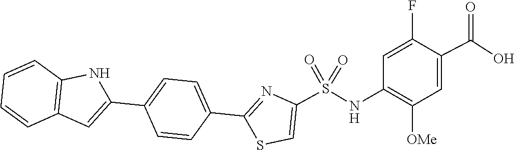 [SMILES: COc1cc(C(=O)O)c(F)cc1NS(=O)(=O)c1csc(-c2ccc(-c3cc4ccccc4[nH]3)cc2)n1] |
| 638 | 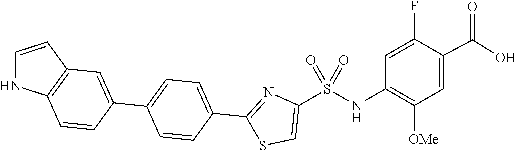 [SMILES: COc1cc(C(=O)O)c(F)cc1NS(=O)(=O)c1csc(-c2ccc(-c3ccc4[nH]ccc4c3)cc2)n1] |
| 639 | 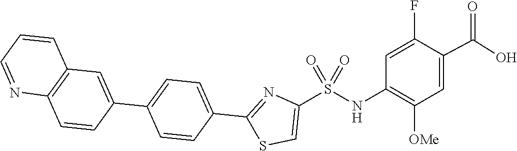 [SMILES: COc1cc(C(=O)O)c(F)cc1NS(=O)(=O)c1csc(-c2ccc(-c3ccc4ncccc4c3)cc2)n1] |
| 640 | 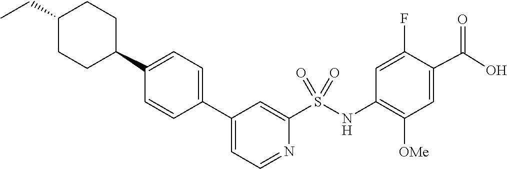 [SMILES: CC[C@H]1CC[C@H](c2ccc(-c3ccnc(S(=O)(=O)Nc4cc(F)c(C(=O)O)cc4OC)c3)cc2)CC1] |
| 641 | 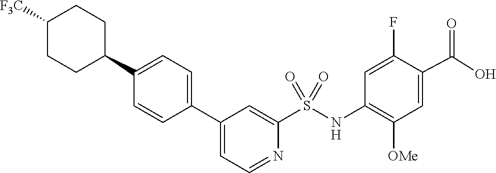 [SMILES: COc1cc(C(=O)O)c(F)cc1NS(=O)(=O)c1cc(-c2ccc([C@H]3CC[C@H](C(F)(F)F)CC3)cc2)ccn1] |
| 642 | 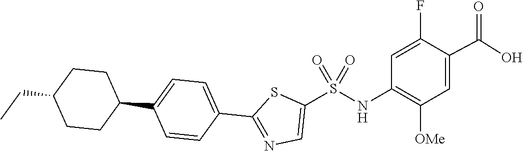 [SMILES: CC[C@H]1CC[C@H](c2ccc(-c3ncc(S(=O)(=O)Nc4cc(F)c(C(=O)O)cc4OC)s3)cc2)CC1] |
| 643 | 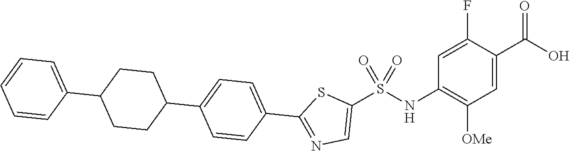 [SMILES: COc1cc(C(=O)O)c(F)cc1NS(=O)(=O)c1cnc(-c2ccc(C3CCC(c4ccccc4)CC3)cc2)s1] |
| 644 | 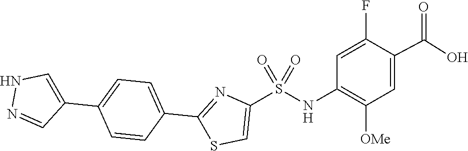 [SMILES: COc1cc(C(=O)O)c(F)cc1NS(=O)(=O)c1csc(-c2ccc(-c3cn[nH]c3)cc2)n1] |
| 645 | 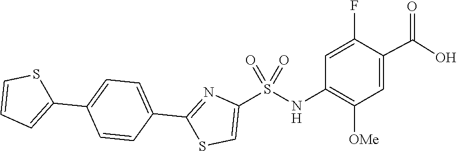 [SMILES: COc1cc(C(=O)O)c(F)cc1NS(=O)(=O)c1csc(-c2ccc(-c3cccs3)cc2)n1] |
| 646 | 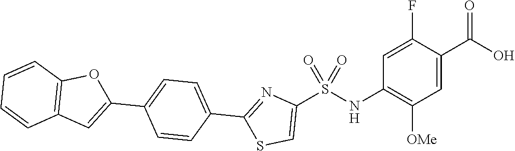 [SMILES: COc1cc(C(=O)O)c(F)cc1NS(=O)(=O)c1csc(-c2ccc(-c3cc4ccccc4o3)cc2)n1] |
| 647 | 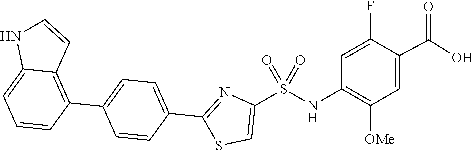 [SMILES: COc1cc(C(=O)O)c(F)cc1NS(=O)(=O)c1csc(-c2ccc(-c3cccc4[nH]ccc34)cc2)n1] |
| 648 | 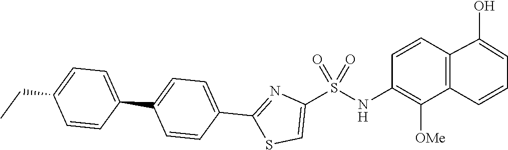 |
| 649 | 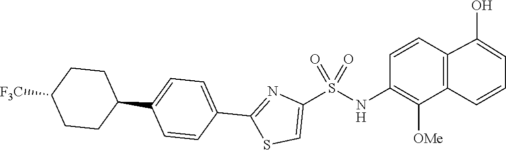 [SMILES: COc1c(NS(=O)(=O)c2csc(-c3ccc([C@H]4CC[C@H](C(F)(F)F)CC4)cc3)n2)ccc2c(O)cccc12] |
| 650 | 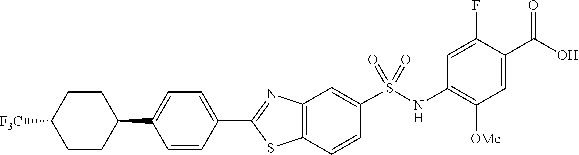 [SMILES: COc1cc(C(=O)O)c(F)cc1NS(=O)(=O)c1ccc2sc(-c3ccc([C@H]4CC[C@H](C(F)(F)F)CC4)cc3)nc2c1] |
| 651 | 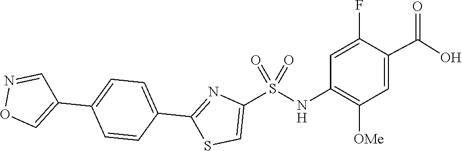 [SMILES: COc1cc(C(=O)O)c(F)cc1NS(=O)(=O)c1csc(-c2ccc(-c3cnoc3)cc2)n1] |
| 652 | 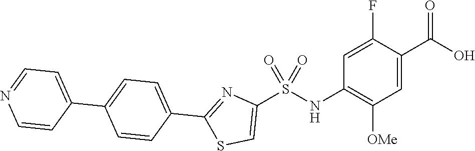 [SMILES: COc1cc(C(=O)O)c(F)cc1NS(=O)(=O)c1csc(-c2ccc(-c3ccncc3)cc2)n1] |
| 653 | 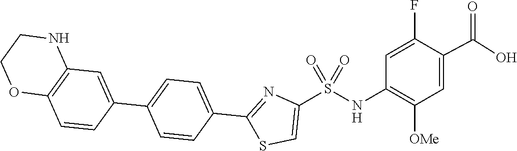 [SMILES: COc1cc(C(=O)O)c(F)cc1NS(=O)(=O)c1csc(-c2ccc(-c3ccc4c(c3)NCCO4)cc2)n1] |
| 654 | 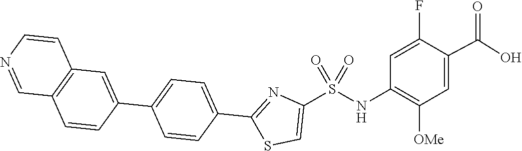 [SMILES: COc1cc(C(=O)O)c(F)cc1NS(=O)(=O)c1csc(-c2ccc(-c3ccc4cnccc4c3)cc2)n1] |
| 655 | 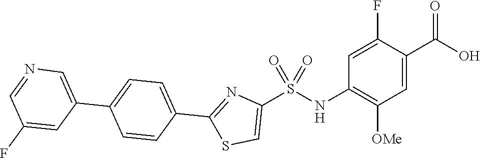 [SMILES: COc1cc(C(=O)O)c(F)cc1NS(=O)(=O)c1csc(-c2ccc(-c3cncc(F)c3)cc2)n1] |
| 656 | 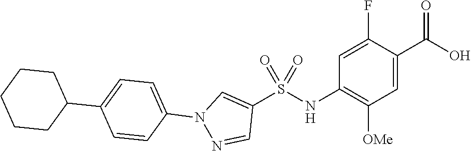 [SMILES: COc1cc(C(=O)O)c(F)cc1NS(=O)(=O)c1cnn(-c2ccc(C3CCCCC3)cc2)c1] |
| 657 | 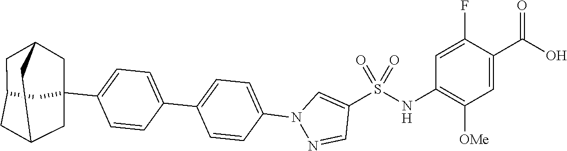 |
| 658 | 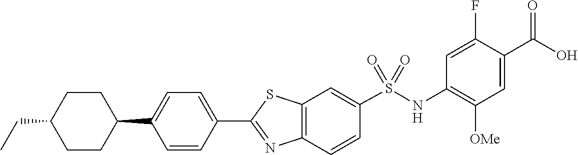 [SMILES: CC[C@H]1CC[C@H](c2ccc(-c3nc4ccc(S(=O)(=O)Nc5cc(F)c(C(=O)O)cc5OC)cc4s3)cc2)CC1] |
| 659 | 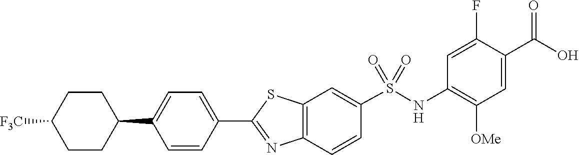 [SMILES: COc1cc(C(=O)O)c(F)cc1NS(=O)(=O)c1ccc2nc(-c3ccc([C@H]4CC[C@H](C(F)(F)F)CC4)cc3)sc2c1] |
| 660 | 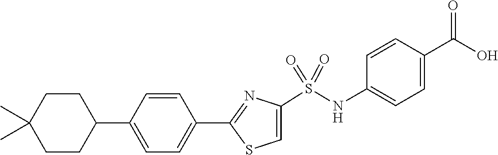 [SMILES: CC1(C)CCC(c2ccc(-c3nc(S(=O)(=O)Nc4ccc(C(=O)O)cc4)cs3)cc2)CC1] |
| 661 | 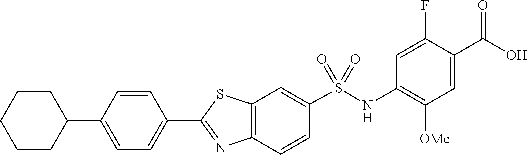 [SMILES: COc1cc(C(=O)O)c(F)cc1NS(=O)(=O)c1ccc2nc(-c3ccc(C4CCCCC4)cc3)sc2c1] |
| 662 | 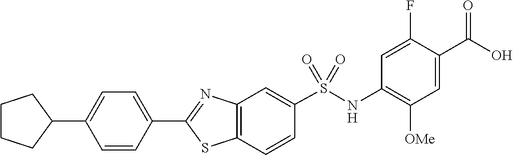 [SMILES: COc1cc(C(=O)O)c(F)cc1NS(=O)(=O)c1ccc2sc(-c3ccc(C4CCCC4)cc3)nc2c1] |
| 663 | 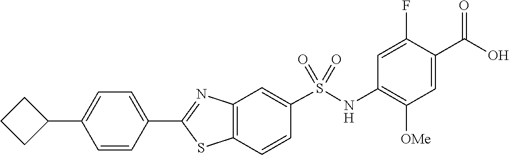 [SMILES: COc1cc(C(=O)O)c(F)cc1NS(=O)(=O)c1ccc2sc(-c3ccc(C4CCC4)cc3)nc2c1] |
| 664 | 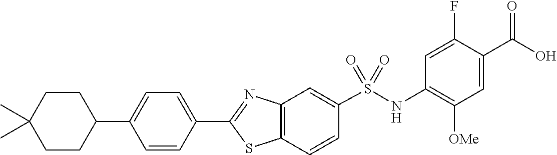 [SMILES: COc1cc(C(=O)O)c(F)cc1NS(=O)(=O)c1ccc2sc(-c3ccc(C4CCC(C)(C)CC4)cc3)nc2c1] |
| 665 | 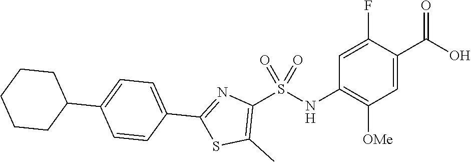 [SMILES: COc1cc(C(=O)O)c(F)cc1NS(=O)(=O)c1nc(-c2ccc(C3CCCCC3)cc2)sc1C] |
| 666 | 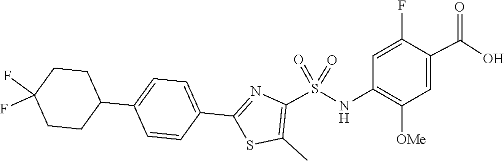 [SMILES: COc1cc(C(=O)O)c(F)cc1NS(=O)(=O)c1nc(-c2ccc(C3CCC(F)(F)CC3)cc2)sc1C] |
| 667 | 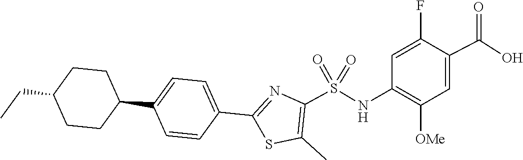 [SMILES: CC[C@H]1CC[C@H](c2ccc(-c3nc(S(=O)(=O)Nc4cc(F)c(C(=O)O)cc4OC)c(C)s3)cc2)CC1] |
| 668 | 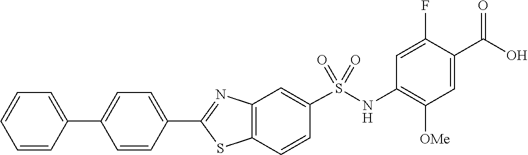 [SMILES: COc1cc(C(=O)O)c(F)cc1NS(=O)(=O)c1ccc2sc(-c3ccc(-c4ccccc4)cc3)nc2c1] |
| 669 | 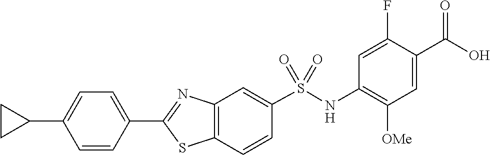 [SMILES: COc1cc(C(=O)O)c(F)cc1NS(=O)(=O)c1ccc2sc(-c3ccc(C4CC4)cc3)nc2c1] |
| 670 | 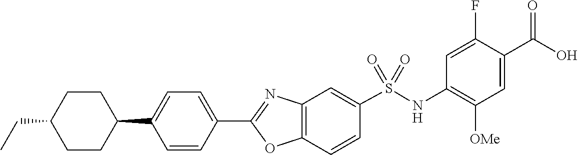 [SMILES: CC[C@H]1CC[C@H](c2ccc(-c3nc4cc(S(=O)(=O)Nc5cc(F)c(C(=O)O)cc5OC)ccc4o3)cc2)CC1] |
| 671 | 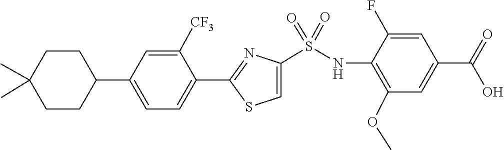 [SMILES: COc1cc(C(=O)O)cc(F)c1NS(=O)(=O)c1csc(-c2ccc(C3CCC(C)(C)CC3)cc2C(F)(F)F)n1] |
| 672 | 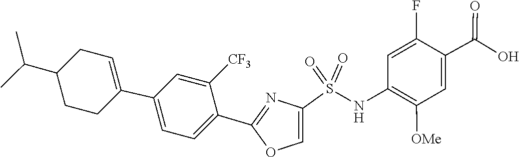 [SMILES: COc1cc(C(=O)O)c(F)cc1NS(=O)(=O)c1coc(-c2ccc(C3=CCC(C(C)C)CC3)cc2C(F)(F)F)n1] |
| 673 | 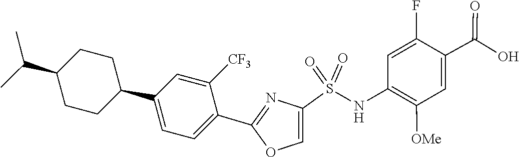 [SMILES: COc1cc(C(=O)O)c(F)cc1NS(=O)(=O)c1coc(-c2ccc([C@H]3CC[C@@H](C(C)C)CC3)cc2C(F)(F)F)n1] |
| 674 | 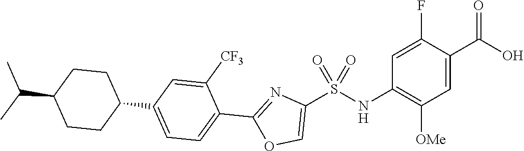 [SMILES: COc1cc(C(=O)O)c(F)cc1NS(=O)(=O)c1coc(-c2ccc([C@H]3CC[C@H](C(C)C)CC3)cc2C(F)(F)F)n1] |
| 675 | 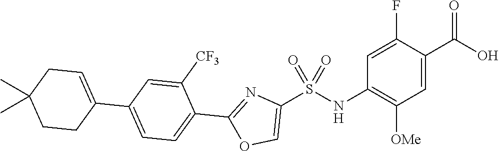 [SMILES: COc1cc(C(=O)O)c(F)cc1NS(=O)(=O)c1coc(-c2ccc(C3=CCC(C)(C)CC3)cc2C(F)(F)F)n1] |
| 676 | 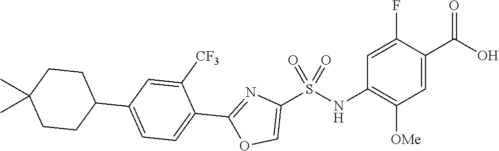 [SMILES: COc1cc(C(=O)O)c(F)cc1NS(=O)(=O)c1coc(-c2ccc(C3CCC(C)(C)CC3)cc2C(F)(F)F)n1] |
| 677 | 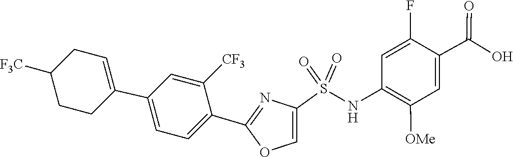 [SMILES: COc1cc(C(=O)O)c(F)cc1NS(=O)(=O)c1coc(-c2ccc(C3=CCC(C(F)(F)F)CC3)cc2C(F)(F)F)n1] |
| 678 | 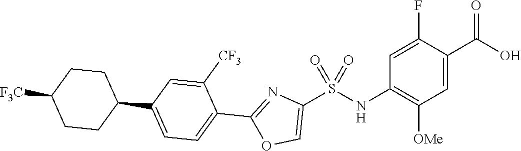 [SMILES: COc1cc(C(=O)O)c(F)cc1NS(=O)(=O)c1coc(-c2ccc([C@H]3CC[C@@H](C(F)(F)F)CC3)cc2C(F)(F)F)n1] |
| 679 | 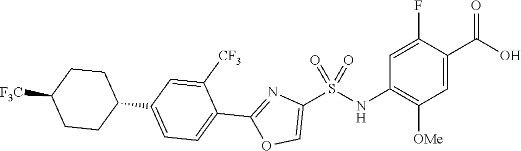 [SMILES: COc1cc(C(=O)O)c(F)cc1NS(=O)(=O)c1coc(-c2ccc([C@H]3CC[C@H](C(F)(F)F)CC3)cc2C(F)(F)F)n1] |
| 680 | 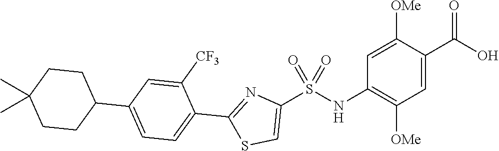 [SMILES: COc1cc(C(=O)O)c(OC)cc1NS(=O)(=O)c1csc(-c2ccc(C3CCC(C)(C)CC3)cc2C(F)(F)F)n1] |
| 681 | 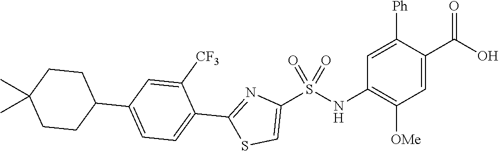 [SMILES: COc1cc(C(=O)O)c(-c2ccccc2)cc1NS(=O)(=O)c1csc(-c2ccc(C3CCC(C)(C)CC3)cc2C(F)(F)F)n1] |
| 682 | 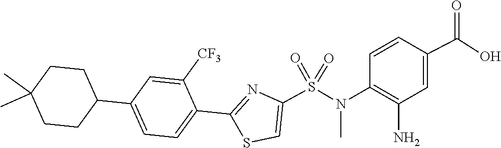 [SMILES: CN(c1ccc(C(=O)O)cc1N)S(=O)(=O)c1csc(-c2ccc(C3CCC(C)(C)CC3)cc2C(F)(F)F)n1] |
| 683 | 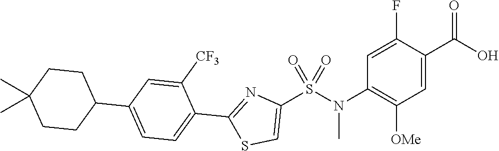 [SMILES: COc1cc(C(=O)O)c(F)cc1N(C)S(=O)(=O)c1csc(-c2ccc(C3CCC(C)(C)CC3)cc2C(F)(F)F)n1] |
| 684 | 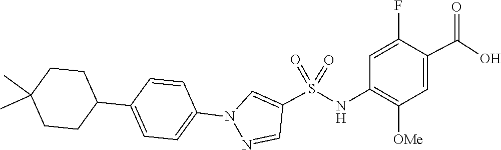 [SMILES: COc1cc(C(=O)O)c(F)cc1NS(=O)(=O)c1cnn(-c2ccc(C3CCC(C)(C)CC3)cc2)c1] |
| 685 | 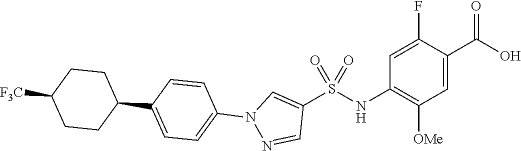 [SMILES: COc1cc(C(=O)O)c(F)cc1NS(=O)(=O)c1cnn(-c2ccc([C@H]3CC[C@@H](C(F)(F)F)CC3)cc2)c1] |
| 686 | 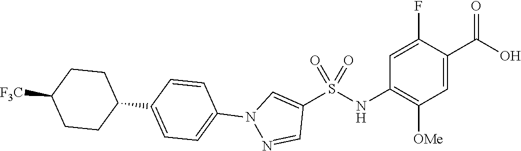 [SMILES: COc1cc(C(=O)O)c(F)cc1NS(=O)(=O)c1cnn(-c2ccc([C@H]3CC[C@H](C(F)(F)F)CC3)cc2)c1] |
| 687 | 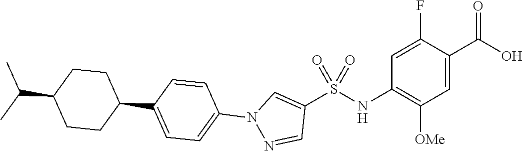 [SMILES: COc1cc(C(=O)O)c(F)cc1NS(=O)(=O)c1cnn(-c2ccc([C@H]3CC[C@@H](C(C)C)CC3)cc2)c1] |
| 688 | 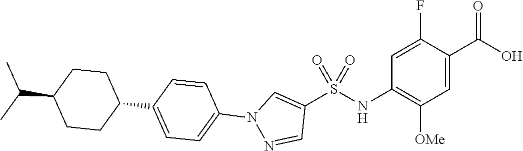 [SMILES: COc1cc(C(=O)O)c(F)cc1NS(=O)(=O)c1cnn(-c2ccc([C@H]3CC[C@H](C(C)C)CC3)cc2)c1] |
| 689 | 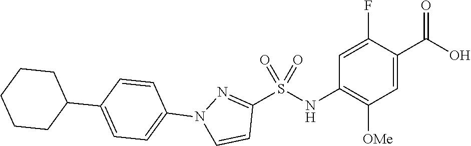 [SMILES: COc1cc(C(=O)O)c(F)cc1NS(=O)(=O)c1ccn(-c2ccc(C3CCCCC3)cc2)n1] |
| 690 | 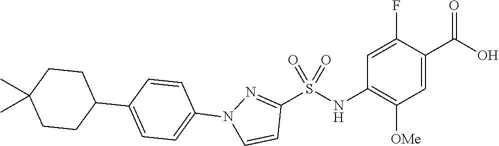 [SMILES: COc1cc(C(=O)O)c(F)cc1NS(=O)(=O)c1ccn(-c2ccc(C3CCC(C)(C)CC3)cc2)n1] |
| 691 | 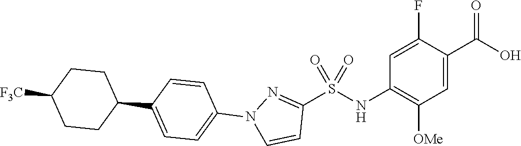 [SMILES: COc1cc(C(=O)O)c(F)cc1NS(=O)(=O)c1ccn(-c2ccc([C@H]3CC[C@@H](C(F)(F)F)CC3)cc2)n1] |
| 692 | 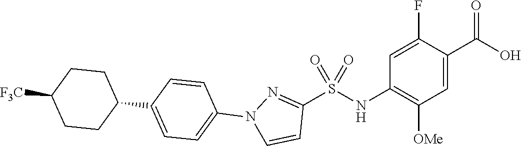 [SMILES: COc1cc(C(=O)O)c(F)cc1NS(=O)(=O)c1ccn(-c2ccc([C@H]3CC[C@H](C(F)(F)F)CC3)cc2)n1] |
| 693 | 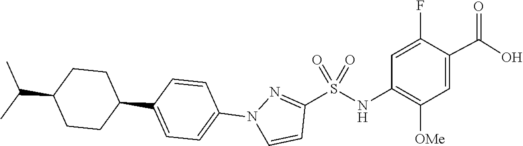 [SMILES: COc1cc(C(=O)O)c(F)cc1NS(=O)(=O)c1ccn(-c2ccc([C@H]3CC[C@@H](C(C)C)CC3)cc2)n1] |
| 694 | 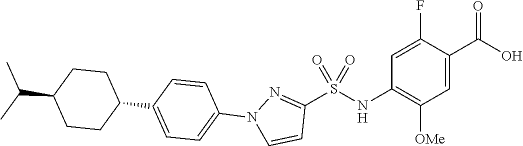 [SMILES: COc1cc(C(=O)O)c(F)cc1NS(=O)(=O)c1ccn(-c2ccc([C@H]3CC[C@H](C(C)C)CC3)cc2)n1] |
| 695 | 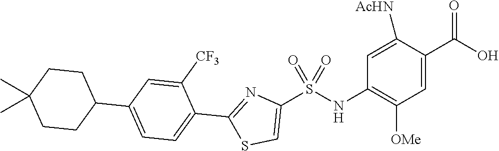 [SMILES: COc1cc(C(=O)O)c(NC(C)=O)cc1NS(=O)(=O)c1csc(-c2ccc(C3CCC(C)(C)CC3)cc2C(F)(F)F)n1] |
| 696 | 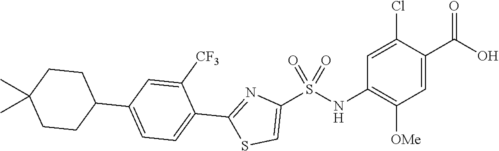 [SMILES: COc1cc(C(=O)O)c(Cl)cc1NS(=O)(=O)c1csc(-c2ccc(C3CCC(C)(C)CC3)cc2C(F)(F)F)n1] |
| 697 | 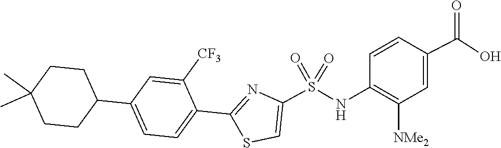 [SMILES: CN(C)c1cc(C(=O)O)ccc1NS(=O)(=O)c1csc(-c2ccc(C3CCC(C)(C)CC3)cc2C(F)(F)F)n1] |
| 698 | 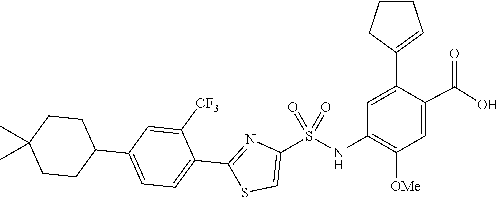 [SMILES: COc1cc(C(=O)O)c(C2=CCCC2)cc1NS(=O)(=O)c1csc(-c2ccc(C3CCC(C)(C)CC3)cc2C(F)(F)F)n1] |
| 699 | 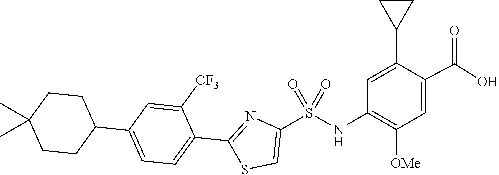 [SMILES: COc1cc(C(=O)O)c(C2CC2)cc1NS(=O)(=O)c1csc(-c2ccc(C3CCC(C)(C)CC3)cc2C(F)(F)F)n1] |
| 700 | 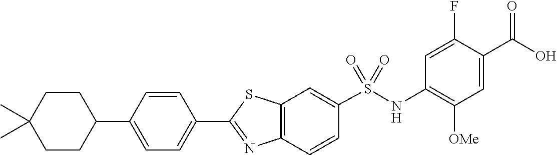 [SMILES: COc1cc(C(=O)O)c(F)cc1NS(=O)(=O)c1ccc2nc(-c3ccc(C4CCC(C)(C)CC4)cc3)sc2c1] |
| 701 | 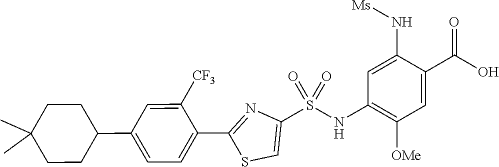 [SMILES: COc1cc(C(=O)O)c(NS(C)(=O)=O)cc1NS(=O)(=O)c1csc(-c2ccc(C3CCC(C)(C)CC3)cc2C(F)(F)F)n1] |
| 702 | 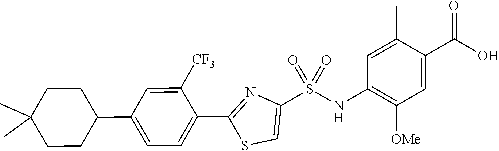 [SMILES: COc1cc(C(=O)O)c(C)cc1NS(=O)(=O)c1csc(-c2ccc(C3CCC(C)(C)CC3)cc2C(F)(F)F)n1] |
| 703 | 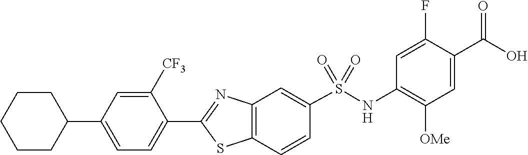 [SMILES: COc1cc(C(=O)O)c(F)cc1NS(=O)(=O)c1ccc2sc(-c3ccc(C4CCCCC4)cc3C(F)(F)F)nc2c1] |
| 704 | 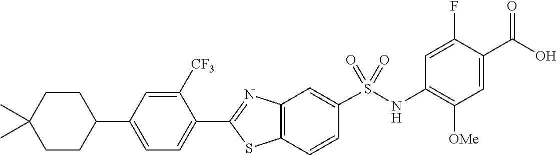 [SMILES: COc1cc(C(=O)O)c(F)cc1NS(=O)(=O)c1ccc2sc(-c3ccc(C4CCC(C)(C)CC4)cc3C(F)(F)F)nc2c1] |
| 705 | 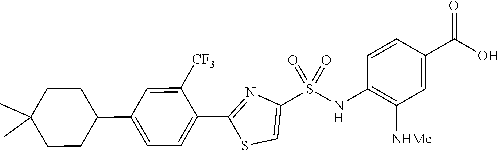 [SMILES: CNc1cc(C(=O)O)ccc1NS(=O)(=O)c1csc(-c2ccc(C3CCC(C)(C)CC3)cc2C(F)(F)F)n1] |
| 706 | 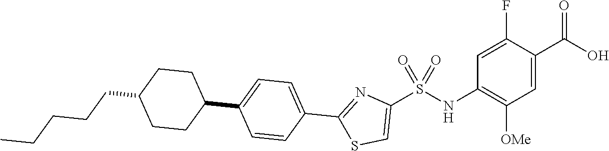 [SMILES: CCCCC[C@H]1CC[C@H](c2ccc(-c3nc(S(=O)(=O)Nc4cc(F)c(C(=O)O)cc4OC)cs3)cc2)CC1] |
| 707 | 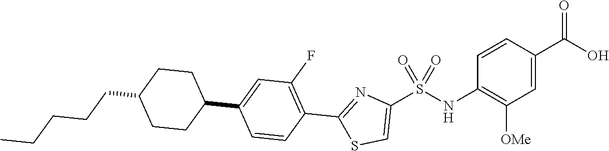 [SMILES: CCCCC[C@H]1CC[C@H](c2ccc(-c3nc(S(=O)(=O)Nc4ccc(C(=O)O)cc4OC)cs3)c(F)c2)CC1] |
| 708 | 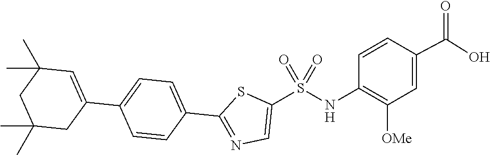 [SMILES: COc1cc(C(=O)O)ccc1NS(=O)(=O)c1cnc(-c2ccc(C3=CC(C)(C)CC(C)(C)C3)cc2)s1] |
| 709 | 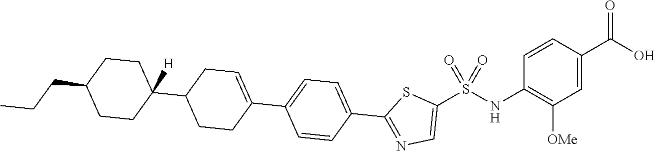 [SMILES: CCC[C@H]1CC[C@H](C2CC=C(c3ccc(-c4ncc(S(=O)(=O)Nc5ccc(C(=O)O)cc5OC)s4)cc3)CC2)CC1] |
| 710 | 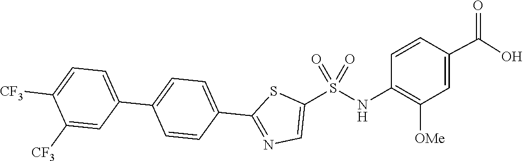 [SMILES: COc1cc(C(=O)O)ccc1NS(=O)(=O)c1cnc(-c2ccc(-c3ccc(C(F)(F)F)c(C(F)(F)F)c3)cc2)s1] |
| 711 | 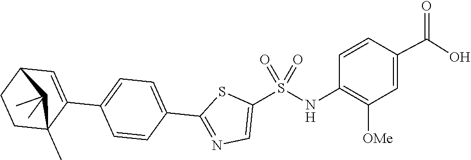 [SMILES: COc1cc(C(=O)O)ccc1NS(=O)(=O)c1cnc(-c2ccc(C3=C[C@H]4CC[C@]3(C)C4(C)C)cc2)s1] |
| 712 | 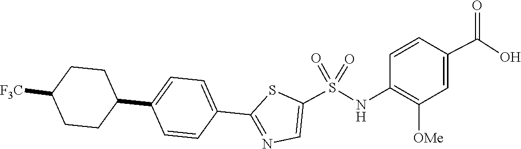 |
| 713 | 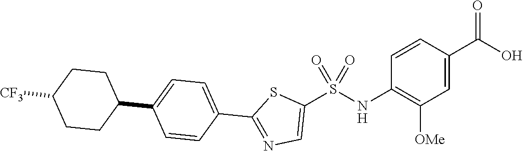 [SMILES: COc1cc(C(=O)O)ccc1NS(=O)(=O)c1cnc(-c2ccc([C@H]3CC[C@H](C(F)(F)F)CC3)cc2)s1] |
| 714 | 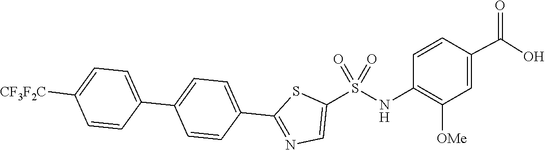 |
| 715 | 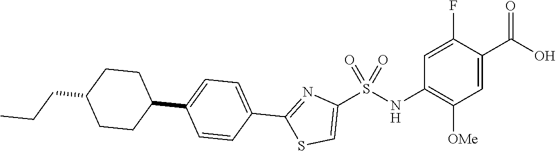 [SMILES: CCC[C@H]1CC[C@H](c2ccc(-c3nc(S(=O)(=O)Nc4cc(F)c(C(=O)O)cc4OC)cs3)cc2)CC1] |
| 716 | 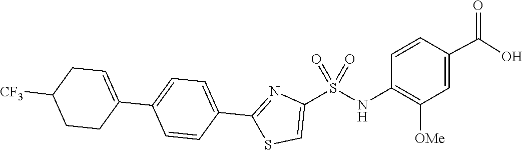 [SMILES: COc1cc(C(=O)O)ccc1NS(=O)(=O)c1csc(-c2ccc(C3=CCC(C(F)(F)F)CC3)cc2)n1] |
| 717 | 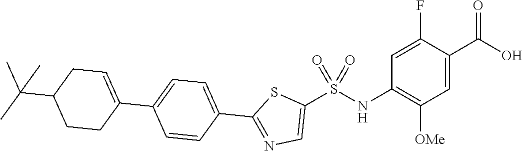 [SMILES: COc1cc(C(=O)O)c(F)cc1NS(=O)(=O)c1cnc(-c2ccc(C3=CCC(C(C)(C)C)CC3)cc2)s1] |
| 718 | 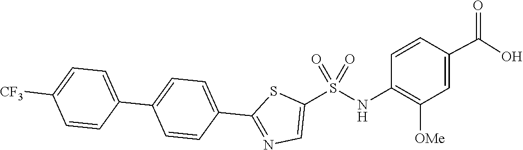 [SMILES: COc1cc(C(=O)O)ccc1NS(=O)(=O)c1cnc(-c2ccc(-c3ccc(C(F)(F)F)cc3)cc2)s1] |
| 719 | 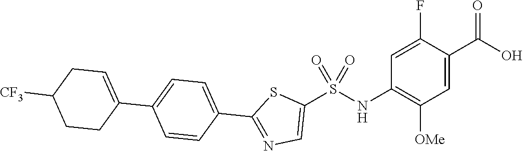 [SMILES: COc1cc(C(=O)O)c(F)cc1NS(=O)(=O)c1cnc(-c2ccc(C3=CCC(C(F)(F)F)CC3)cc2)s1] |
| 720 | 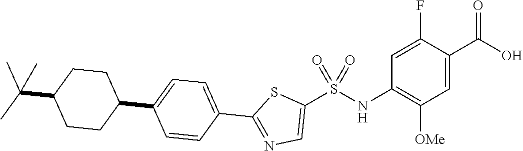 [SMILES: COc1cc(C(=O)O)c(F)cc1NS(=O)(=O)c1cnc(-c2ccc([C@H]3CC[C@@H](C(C)(C)C)CC3)cc2)s1] |
| 721 | 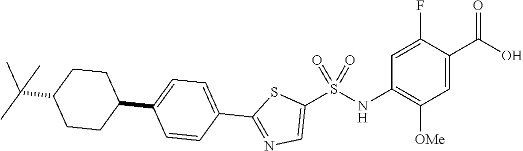 [SMILES: COc1cc(C(=O)O)c(F)cc1NS(=O)(=O)c1cnc(-c2ccc([C@H]3CC[C@H](C(C)(C)C)CC3)cc2)s1] |
| 722 | 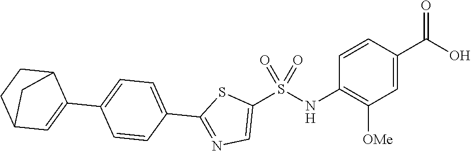 [SMILES: COc1cc(C(=O)O)ccc1NS(=O)(=O)c1cnc(-c2ccc(C3=CC4CCC3C4)cc2)s1] |
| 723 | 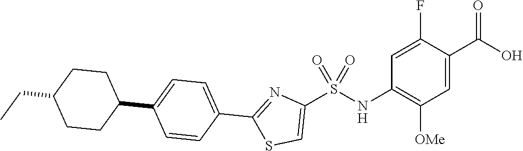 [SMILES: CC[C@H]1CC[C@H](c2ccc(-c3nc(S(=O)(=O)Nc4cc(F)c(C(=O)O)cc4OC)cs3)cc2)CC1] |
| 724 | 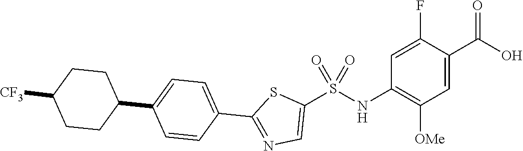 [SMILES: COc1cc(C(=O)O)c(F)cc1NS(=O)(=O)c1cnc(-c2ccc([C@H]3CC[C@@H](C(F)(F)F)CC3)cc2)s1] |
| 725 | 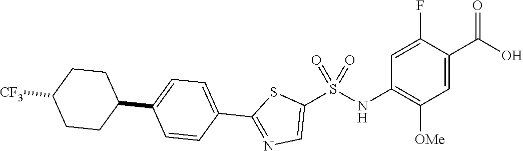 [SMILES: COc1cc(C(=O)O)c(F)cc1NS(=O)(=O)c1cnc(-c2ccc([C@H]3CC[C@H](C(F)(F)F)CC3)cc2)s1] |
| 726 | 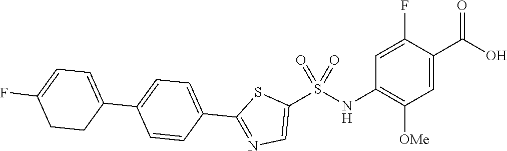 [SMILES: COc1cc(C(=O)O)c(F)cc1NS(=O)(=O)c1cnc(-c2ccc(C3=CC=C(F)CC3)cc2)s1] |
| 727 | 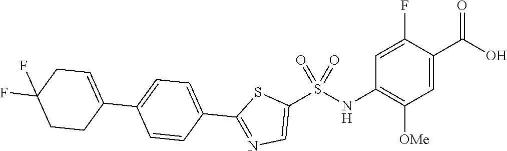 [SMILES: COc1cc(C(=O)O)c(F)cc1NS(=O)(=O)c1cnc(-c2ccc(C3=CCC(F)(F)CC3)cc2)s1] |
| 728 | 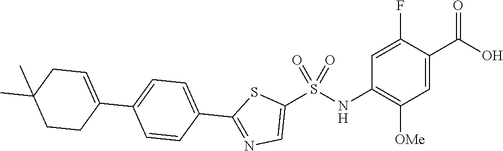 [SMILES: COc1cc(C(=O)O)c(F)cc1NS(=O)(=O)c1cnc(-c2ccc(C3=CCC(C)(C)CC3)cc2)s1] |
| 729 | 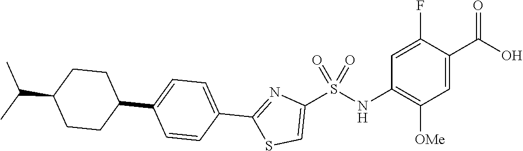 [SMILES: COc1cc(C(=O)O)c(F)cc1NS(=O)(=O)c1csc(-c2ccc([C@H]3CC[C@@H](C(C)C)CC3)cc2)n1] |
| 730 | 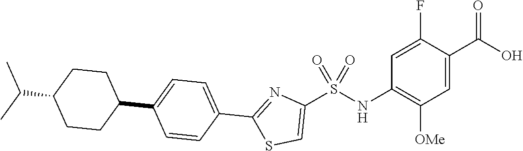 [SMILES: COc1cc(C(=O)O)c(F)cc1NS(=O)(=O)c1csc(-c2ccc([C@H]3CC[C@H](C(C)C)CC3)cc2)n1] |
| 731 | 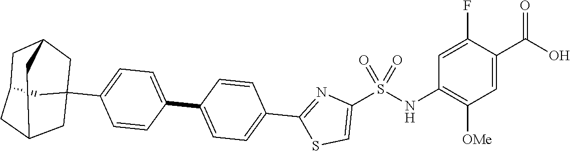 |
| 732 | 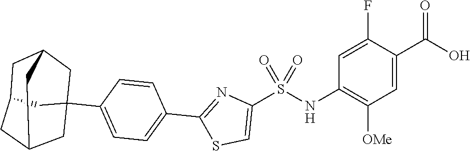 [SMILES: COc1cc(C(=O)O)c(F)cc1NS(=O)(=O)c1csc(-c2ccc(C34CC5C[C@H](C3)C[C@H](C5)C4)cc2)n1] |
| 733 | 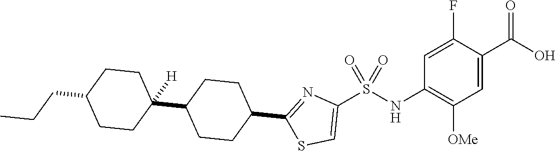 |
| 734 | 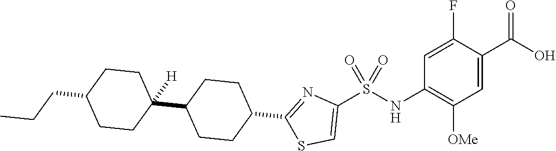 |
| 735 | 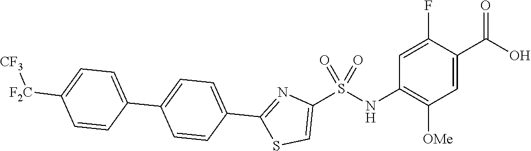 [SMILES: COc1cc(C(=O)O)c(F)cc1NS(=O)(=O)c1csc(-c2ccc(-c3ccc(C(F)(F)C(F)(F)F)cc3)cc2)n1] |
| 736 | 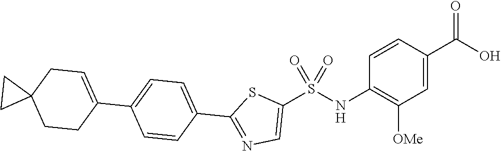 [SMILES: COc1cc(C(=O)O)ccc1NS(=O)(=O)c1cnc(-c2ccc(C3=CCC4(CC3)CC4)cc2)s1] |
| 737 | 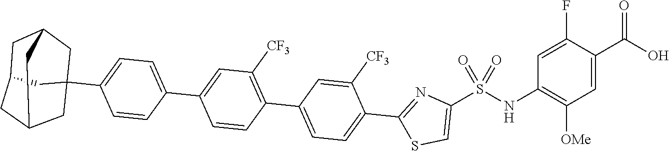 [SMILES: COc1cc(C(=O)O)c(F)cc1NS(=O)(=O)c1csc(-c2ccc(-c3ccc(-c4ccc(C56CC7C[C@H](C5)C[C@H](C7)C6)cc4)cc3C(F)(F)F)cc2C(F)(F)F)n1] |
| 738 | 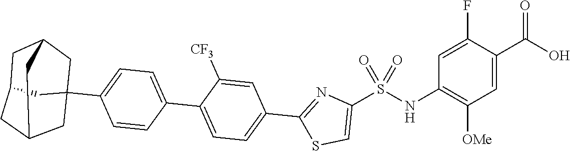 [SMILES: COc1cc(C(=O)O)c(F)cc1NS(=O)(=O)c1csc(-c2ccc(-c3ccc(C45CC6C[C@H](C4)C[C@H](C6)C5)cc3)c(C(F)(F)F)c2)n1] |
| 739 | 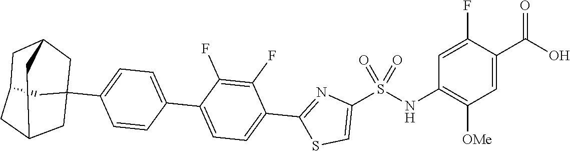 [SMILES: COc1cc(C(=O)O)c(F)cc1NS(=O)(=O)c1csc(-c2ccc(-c3ccc(C45CC6C[C@H](C4)C[C@H](C6)C5)cc3)c(F)c2F)n1] |
| 740 | 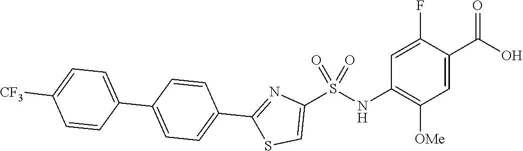 [SMILES: COc1cc(C(=O)O)c(F)cc1NS(=O)(=O)c1csc(-c2ccc(-c3ccc(C(F)(F)F)cc3)cc2)n1] |
| 741 | 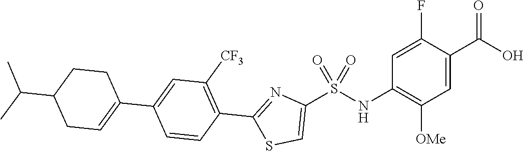 [SMILES: COc1cc(C(=O)O)c(F)cc1NS(=O)(=O)c1csc(-c2ccc(C3=CCC(C(C)C)CC3)cc2C(F)(F)F)n1] |
| 742 | 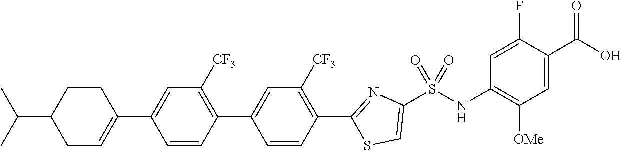 [SMILES: COc1cc(C(=O)O)c(F)cc1NS(=O)(=O)c1csc(-c2ccc(-c3ccc(C4=CCC(C(C)C)CC4)cc3C(F)(F)F)cc2C(F)(F)F)n1] |
| 743 | 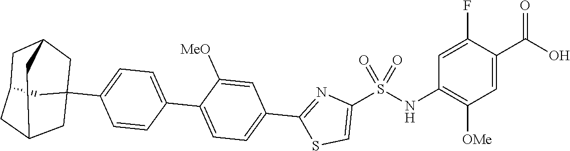 [SMILES: COc1cc(C(=O)O)c(F)cc1NS(=O)(=O)c1csc(-c2ccc(-c3ccc(C45CC6C[C@H](C4)C[C@H](C6)C5)cc3)c(OC)c2)n1] |
| 744 | 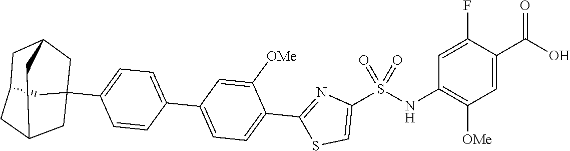 [SMILES: COc1cc(C(=O)O)c(F)cc1NS(=O)(=O)c1csc(-c2ccc(-c3ccc(C45CC6C[C@H](C4)C[C@H](C6)C5)cc3)cc2OC)n1] |
| 745 | 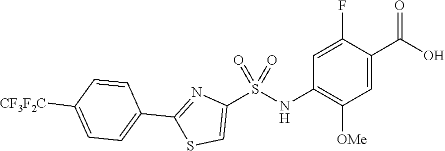 |
| 746 | 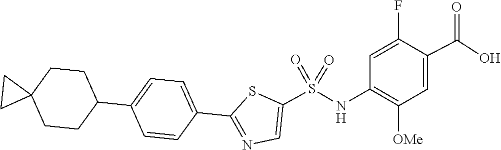 [SMILES: COc1cc(C(=O)O)c(F)cc1NS(=O)(=O)c1cnc(-c2ccc(C3CCC4(CC3)CC4)cc2)s1] |
| 747 | 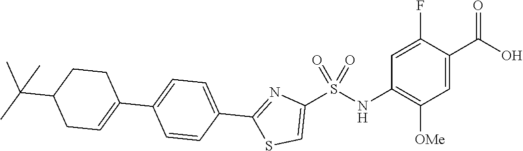 [SMILES: COc1cc(C(=O)O)c(F)cc1NS(=O)(=O)c1csc(-c2ccc(C3=CCC(C(C)(C)C)CC3)cc2)n1] |
| 748 | 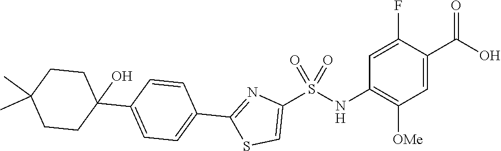 [SMILES: COc1cc(C(=O)O)c(F)cc1NS(=O)(=O)c1csc(-c2ccc(C3(O)CCC(C)(C)CC3)cc2)n1] |
| 749 | 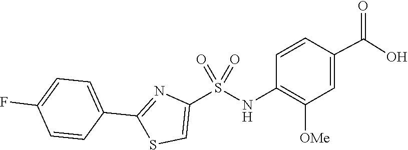 [SMILES: COc1cc(C(=O)O)ccc1NS(=O)(=O)c1csc(-c2ccc(F)cc2)n1] |
| 750 | 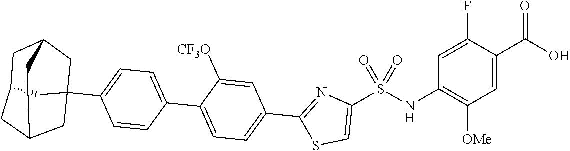 |
| 753 | 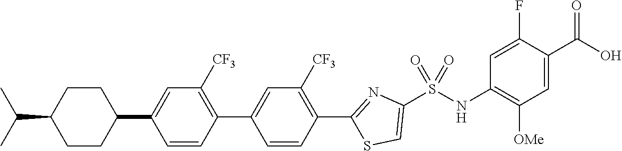 [SMILES: COc1cc(C(=O)O)c(F)cc1NS(=O)(=O)c1csc(-c2ccc(-c3ccc([C@H]4CC[C@@H](C(C)C)CC4)cc3C(F)(F)F)cc2C(F)(F)F)n1] |
| 754 | 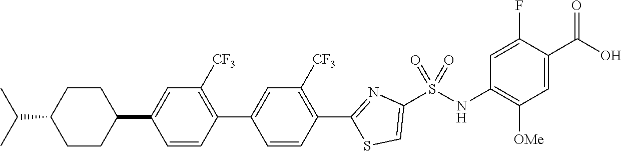 [SMILES: COc1cc(C(=O)O)c(F)cc1NS(=O)(=O)c1csc(-c2ccc(-c3ccc([C@H]4CC[C@H](C(C)C)CC4)cc3C(F)(F)F)cc2C(F)(F)F)n1] |
| 755 | 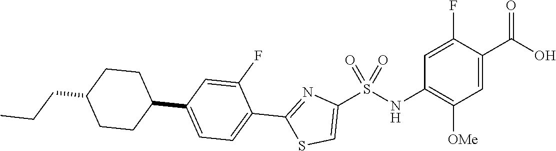 [SMILES: CCC[C@H]1CC[C@H](c2ccc(-c3nc(S(=O)(=O)Nc4cc(F)c(C(=O)O)cc4OC)cs3)c(F)c2)CC1] |
| 756 | 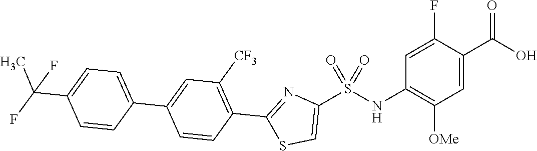 [SMILES: COc1cc(C(=O)O)c(F)cc1NS(=O)(=O)c1csc(-c2ccc(-c3ccc(C(C)(F)F)cc3)cc2C(F)(F)F)n1] |
| 757 | 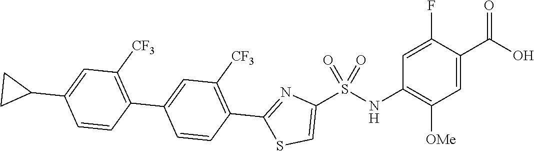 [SMILES: COc1cc(C(=O)O)c(F)cc1NS(=O)(=O)c1csc(-c2ccc(-c3ccc(C4CC4)cc3C(F)(F)F)cc2C(F)(F)F)n1] |
| 758 | 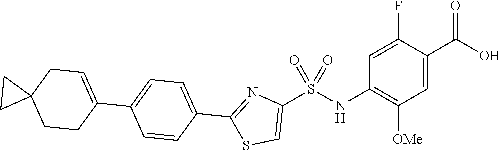 [SMILES: COc1cc(C(=O)O)c(F)cc1NS(=O)(=O)c1csc(-c2ccc(C3=CCC4(CC3)CC4)cc2)n1] |
| 759 | 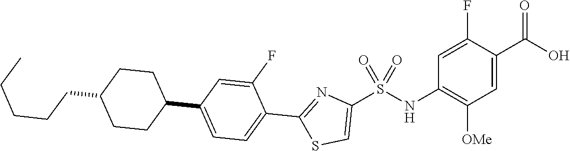 [SMILES: CCCCC[C@H]1CC[C@H](c2ccc(-c3nc(S(=O)(=O)Nc4cc(F)c(C(=O)O)cc4OC)cs3)c(F)c2)CC1] |
| 760 | 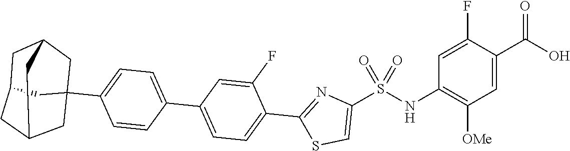 [SMILES: COc1cc(C(=O)O)c(F)cc1NS(=O)(=O)c1csc(-c2ccc(-c3ccc(C45CC6C[C@H](C4)C[C@H](C6)C5)cc3)cc2F)n1] |
| 761 | 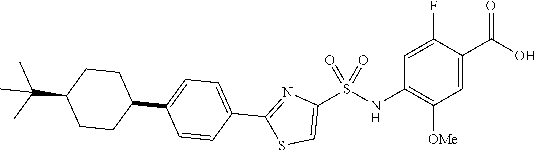 [SMILES: COc1cc(C(=O)O)c(F)cc1NS(=O)(=O)c1csc(-c2ccc([C@H]3CC[C@@H](C(C)(C)C)CC3)cc2)n1] |
| 762 | 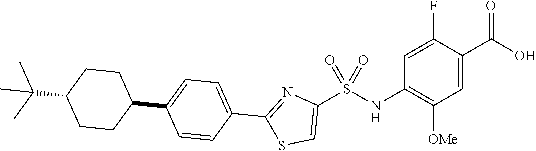 [SMILES: COc1cc(C(=O)O)c(F)cc1NS(=O)(=O)c1csc(-c2ccc([C@H]3CC[C@H](C(C)(C)C)CC3)cc2)n1] |
| 763 | 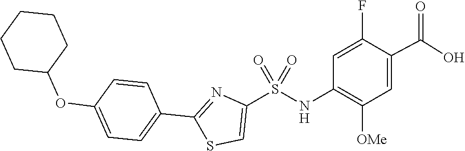 [SMILES: COc1cc(C(=O)O)c(F)cc1NS(=O)(=O)c1csc(-c2ccc(OC3CCCCC3)cc2)n1] |
| 764 | 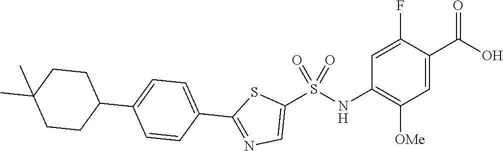 [SMILES: COc1cc(C(=O)O)c(F)cc1NS(=O)(=O)c1cnc(-c2ccc(C3CCC(C)(C)CC3)cc2)s1] |
| 765 | 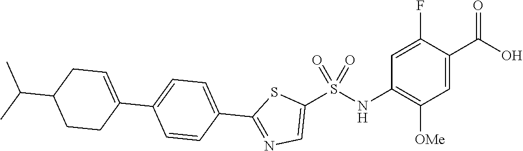 [SMILES: COc1cc(C(=O)O)c(F)cc1NS(=O)(=O)c1cnc(-c2ccc(C3=CCC(C(C)C)CC3)cc2)s1] |
| 766 | 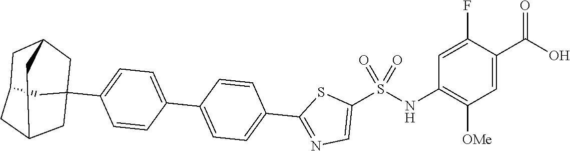 [SMILES: COc1cc(C(=O)O)c(F)cc1NS(=O)(=O)c1cnc(-c2ccc(-c3ccc(C45CC6C[C@H](C4)C[C@H](C6)C5)cc3)cc2)s1] |
| 767 | 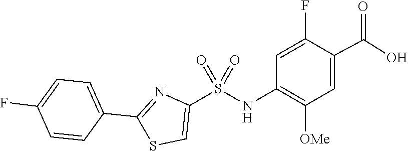 [SMILES: COc1cc(C(=O)O)c(F)cc1NS(=O)(=O)c1csc(-c2ccc(F)cc2)n1] |
| 768 | 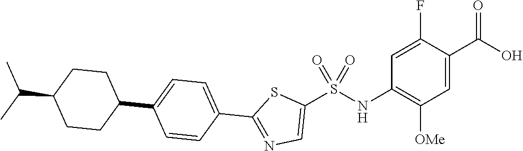 [SMILES: COc1cc(C(=O)O)c(F)cc1NS(=O)(=O)c1cnc(-c2ccc([C@H]3CC[C@@H](C(C)C)CC3)cc2)s1] |
| 769 | 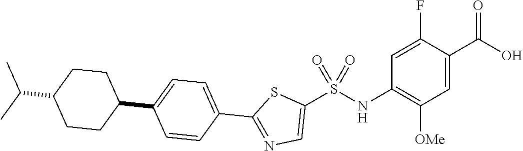 [SMILES: COc1cc(C(=O)O)c(F)cc1NS(=O)(=O)c1cnc(-c2ccc([C@H]3CC[C@H](C(C)C)CC3)cc2)s1] |
| 770 | 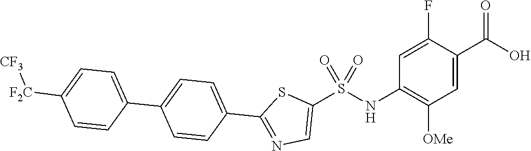 [SMILES: COc1cc(C(=O)O)c(F)cc1NS(=O)(=O)c1cnc(-c2ccc(-c3ccc(C(F)(F)C(F)(F)F)cc3)cc2)s1] |
| 771 | 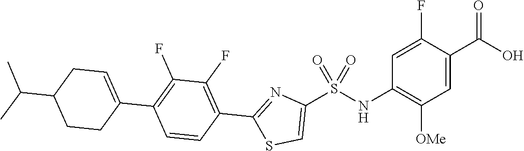 [SMILES: COc1cc(C(=O)O)c(F)cc1NS(=O)(=O)c1csc(-c2ccc(C3=CCC(C(C)C)CC3)c(F)c2F)n1] |
| 772 | 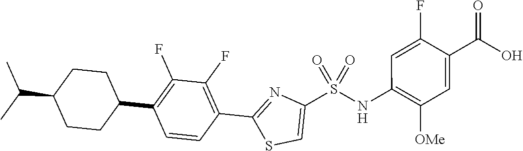 [SMILES: COc1cc(C(=O)O)c(F)cc1NS(=O)(=O)c1csc(-c2ccc([C@H]3CC[C@@H](C(C)C)CC3)c(F)c2F)n1] |
| 773 | 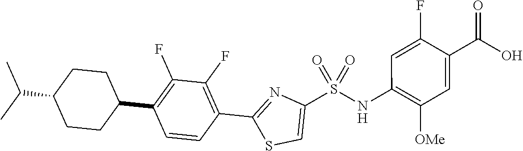 [SMILES: COc1cc(C(=O)O)c(F)cc1NS(=O)(=O)c1csc(-c2ccc([C@H]3CC[C@H](C(C)C)CC3)c(F)c2F)n1] |
| 774 | 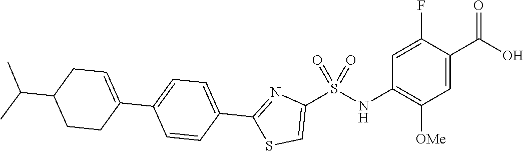 [SMILES: COc1cc(C(=O)O)c(F)cc1NS(=O)(=O)c1csc(-c2ccc(C3=CCC(C(C)C)CC3)cc2)n1] |
| 775 | 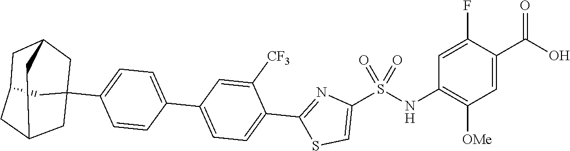 [SMILES: COc1cc(C(=O)O)c(F)cc1NS(=O)(=O)c1csc(-c2ccc(-c3ccc(C45CC6C[C@H](C4)C[C@H](C6)C5)cc3)cc2C(F)(F)F)n1] |
| 776 | 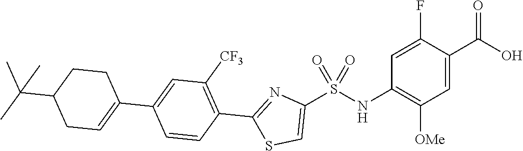 [SMILES: COc1cc(C(=O)O)c(F)cc1NS(=O)(=O)c1csc(-c2ccc(C3=CCC(C(C)(C)C)CC3)cc2C(F)(F)F)n1] |
| 777 | 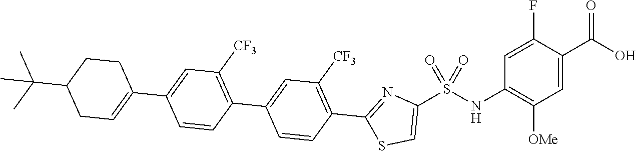 [SMILES: COc1cc(C(=O)O)c(F)cc1NS(=O)(=O)c1csc(-c2ccc(-c3ccc(C4=CCC(C(C)(C)C)CC4)cc3C(F)(F)F)cc2C(F)(F)F)n1] |
| 778 | 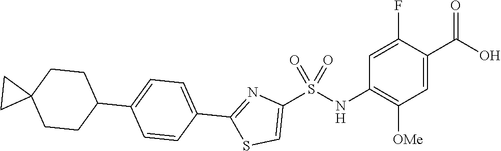 [SMILES: COc1cc(C(=O)O)c(F)cc1NS(=O)(=O)c1csc(-c2ccc(C3CCC4(CC3)CC4)cc2)n1] |
| 779 | 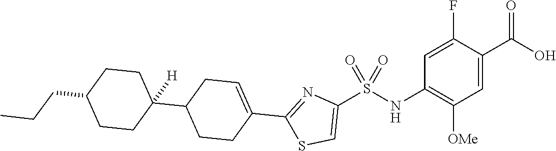 [SMILES: CCC[C@H]1CC[C@H](C2CC=C(c3nc(S(=O)(=O)Nc4cc(F)c(C(=O)O)cc4OC)cs3)CC2)CC1] |
| 780 | 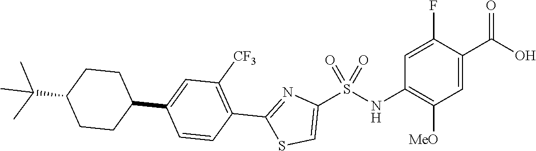 [SMILES: COc1cc(C(=O)O)c(F)cc1NS(=O)(=O)c1csc(-c2ccc([C@H]3CC[C@H](C(C)(C)C)CC3)cc2C(F)(F)F)n1] |
| 781 | 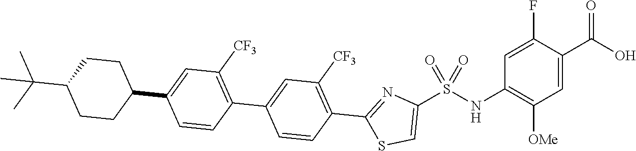 [SMILES: COc1cc(C(=O)O)c(F)cc1NS(=O)(=O)c1csc(-c2ccc(-c3ccc([C@H]4CC[C@H](C(C)(C)C)CC4)cc3C(F)(F)F)cc2C(F)(F)F)n1] |
| 782 | 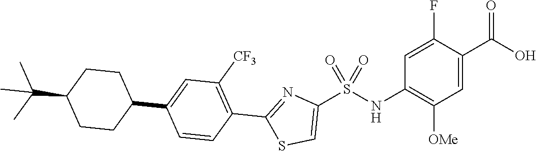 [SMILES: COc1cc(C(=O)O)c(F)cc1NS(=O)(=O)c1csc(-c2ccc([C@H]3CC[C@@H](C(C)(C)C)CC3)cc2C(F)(F)F)n1] |
| 783 | 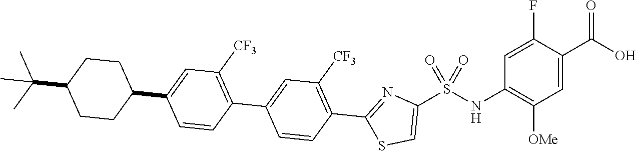 [SMILES: COc1cc(C(=O)O)c(F)cc1NS(=O)(=O)c1csc(-c2ccc(-c3ccc([C@H]4CC[C@@H](C(C)(C)C)CC4)cc3C(F)(F)F)cc2C(F)(F)F)n1] |
| 784 | 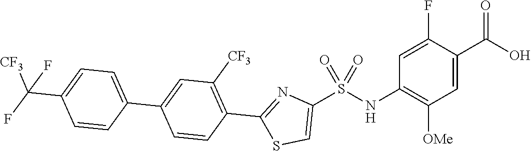 [SMILES: COc1cc(C(=O)O)c(F)cc1NS(=O)(=O)c1csc(-c2ccc(-c3ccc(C(F)(F)C(F)(F)F)cc3)cc2C(F)(F)F)n1] |
| 785 | 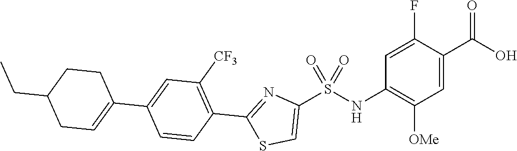 [SMILES: CCC1CC=C(c2ccc(-c3nc(S(=O)(=O)Nc4cc(F)c(C(=O)O)cc4OC)cs3)c(C(F)(F)F)c2)CC1] |
| 786 | 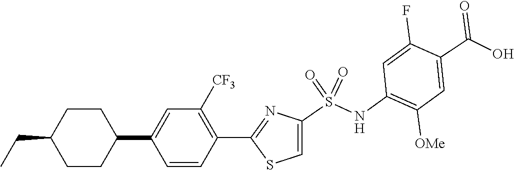 |
| 787 | 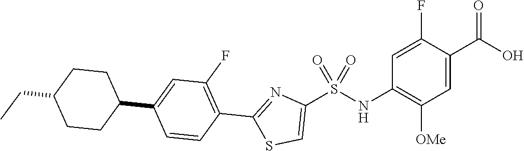 [SMILES: CC[C@H]1CC[C@H](c2ccc(-c3nc(S(=O)(=O)Nc4cc(F)c(C(=O)O)cc4OC)cs3)c(F)c2)CC1] |
| 788 | 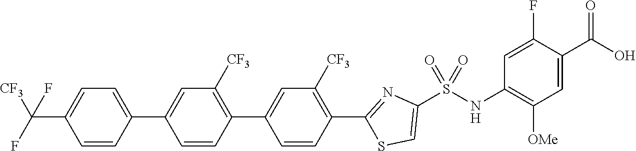 [SMILES: COc1cc(C(=O)O)c(F)cc1NS(=O)(=O)c1csc(-c2ccc(-c3ccc(-c4ccc(C(F)(F)C(F)(F)F)cc4)cc3C(F)(F)F)cc2C(F)(F)F)n1] |
| 789 | 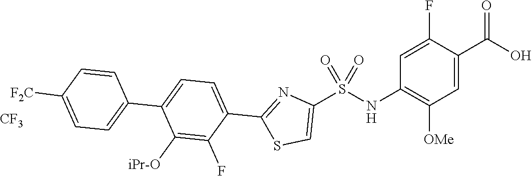 |
| 790 | 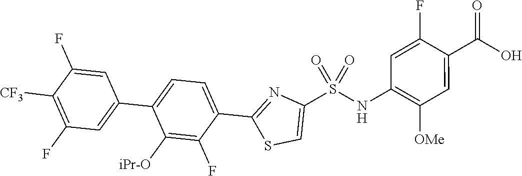 |
| 791 | 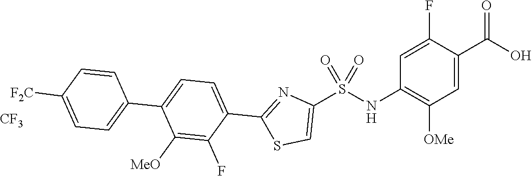 |
| 792 | 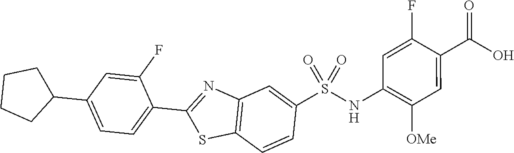 [SMILES: COc1cc(C(=O)O)c(F)cc1NS(=O)(=O)c1ccc2sc(-c3ccc(C4CCCC4)cc3F)nc2c1] |
| 793 | 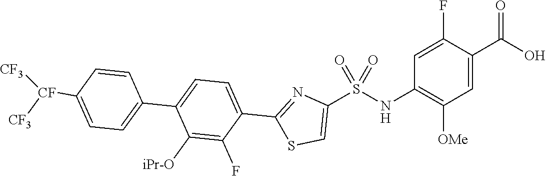 |
| 794 | 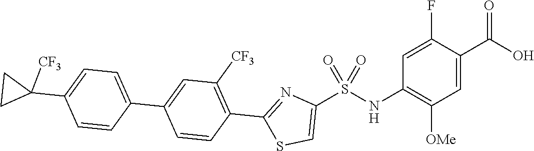 [SMILES: COc1cc(C(=O)O)c(F)cc1NS(=O)(=O)c1csc(-c2ccc(-c3ccc(C4(C(F)(F)F)CC4)cc3)cc2C(F)(F)F)n1] |
| 795 | 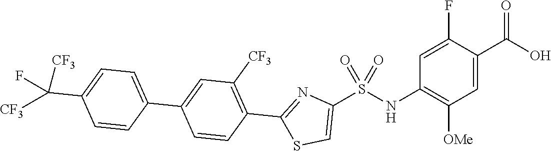 [SMILES: COc1cc(C(=O)O)c(F)cc1NS(=O)(=O)c1csc(-c2ccc(-c3ccc(C(F)(C(F)(F)F)C(F)(F)F)cc3)cc2C(F)(F)F)n1] |
| 796 | 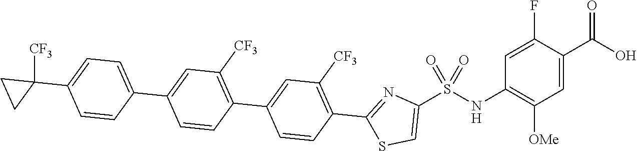 [SMILES: COc1cc(C(=O)O)c(F)cc1NS(=O)(=O)c1csc(-c2ccc(-c3ccc(-c4ccc(C5(C(F)(F)F)CC5)cc4)cc3C(F)(F)F)cc2C(F)(F)F)n1] |
| 797 | 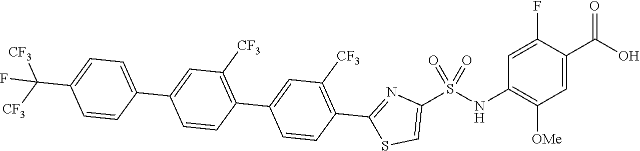 [SMILES: COc1cc(C(=O)O)c(F)cc1NS(=O)(=O)c1csc(-c2ccc(-c3ccc(-c4ccc(C(F)(C(F)(F)F)C(F)(F)F)cc4)cc3C(F)(F)F)cc2C(F)(F)F)n1] |
| 798 | 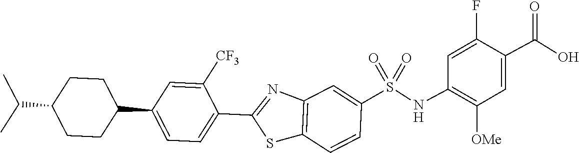 |
| 799 | 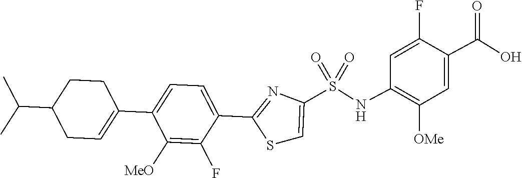 [SMILES: COc1cc(C(=O)O)c(F)cc1NS(=O)(=O)c1csc(-c2ccc(C3=CCC(C(C)C)CC3)c(OC)c2F)n1] |
| 800 | 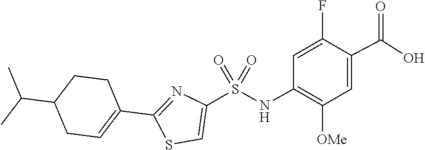 [SMILES: COc1cc(C(=O)O)c(F)cc1NS(=O)(=O)c1csc(C2=CCC(C(C)C)CC2)n1] |
| 801 | 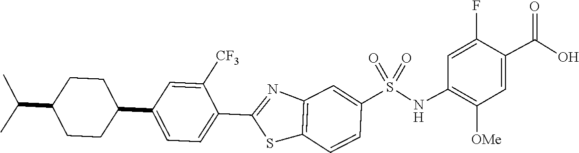 [SMILES: COc1cc(C(=O)O)c(F)cc1NS(=O)(=O)c1ccc2sc(-c3ccc([C@H]4CC[C@@H](C(C)C)CC4)cc3C(F)(F)F)nc2c1] |
| 802 | 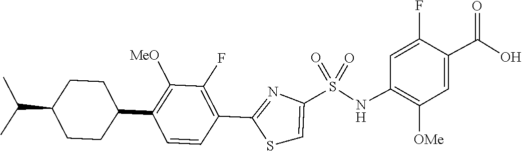 [SMILES: COc1cc(C(=O)O)c(F)cc1NS(=O)(=O)c1csc(-c2ccc([C@H]3CC[C@@H](C(C)C)CC3)c(OC)c2F)n1] |
| 803 | 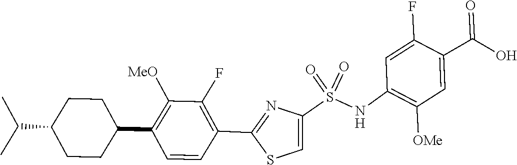 |
| 804 | 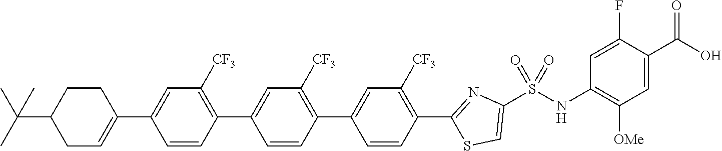 [SMILES: COc1cc(C(=O)O)c(F)cc1NS(=O)(=O)c1csc(-c2ccc(-c3ccc(-c4ccc(C5=CCC(C(C)(C)C)CC5)cc4C(F)(F)F)cc3C(F)(F)F)cc2C(F)(F)F)n1] |
| 805 | 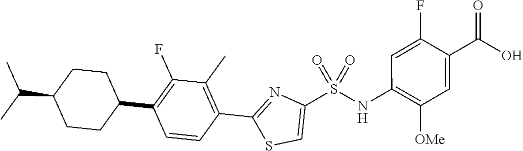 [SMILES: COc1cc(C(=O)O)c(F)cc1NS(=O)(=O)c1csc(-c2ccc([C@H]3CC[C@@H](C(C)C)CC3)c(F)c2C)n1] |
| 806 | 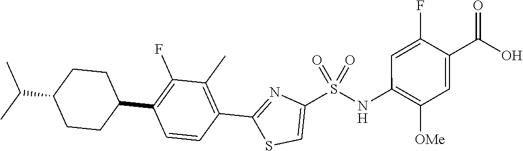 [SMILES: COc1cc(C(=O)O)c(F)cc1NS(=O)(=O)c1csc(-c2ccc([C@H]3CC[C@H](C(C)C)CC3)c(F)c2C)n1] |
| 807 | 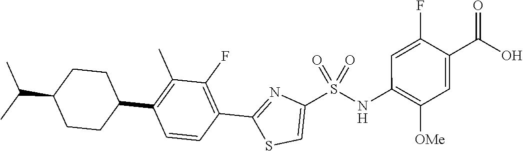 [SMILES: COc1cc(C(=O)O)c(F)cc1NS(=O)(=O)c1csc(-c2ccc([C@H]3CC[C@@H](C(C)C)CC3)c(C)c2F)n1] |
| 808 | 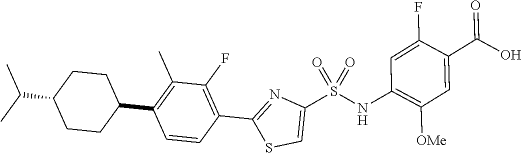 [SMILES: COc1cc(C(=O)O)c(F)cc1NS(=O)(=O)c1csc(-c2ccc([C@H]3CC[C@H](C(C)C)CC3)c(C)c2F)n1] |
| 809 | 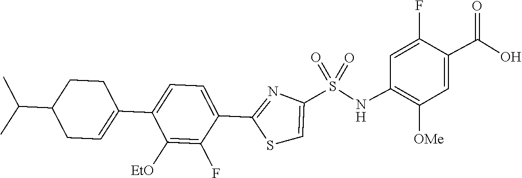 [SMILES: CCOc1c(C2=CCC(C(C)C)CC2)ccc(-c2nc(S(=O)(=O)Nc3cc(F)c(C(=O)O)cc3OC)cs2)c1F] |
| 810 | 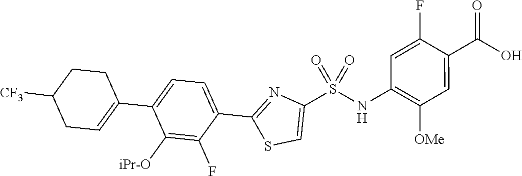 |
| 811 | 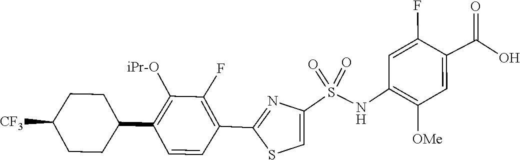 |
| 812 | 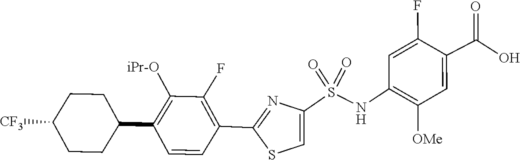 |
| 813 | 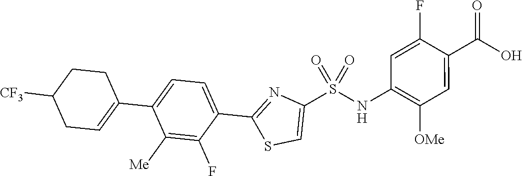 [SMILES: COc1cc(C(=O)O)c(F)cc1NS(=O)(=O)c1csc(-c2ccc(C3=CCC(C(F)(F)F)CC3)c(C)c2F)n1] |
| 814 | 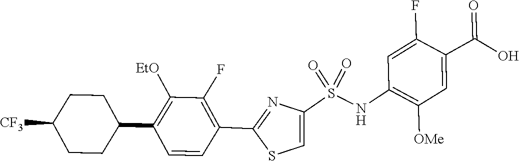 |
| 815 | 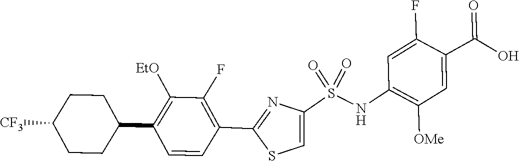 |
| 816 | 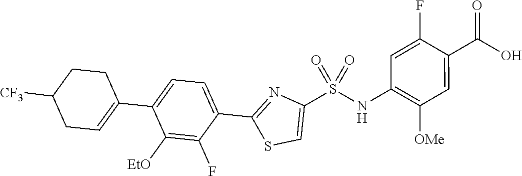 [SMILES: CCOc1c(C2=CCC(C(F)(F)F)CC2)ccc(-c2nc(S(=O)(=O)Nc3cc(F)c(C(=O)O)cc3OC)cs2)c1F] |
| 817 | 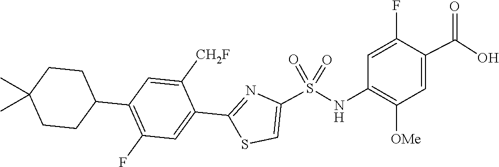 |
| 818 | 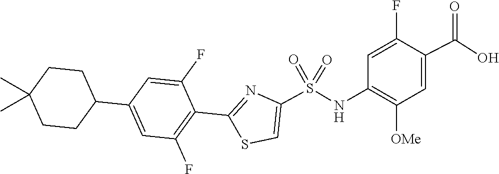 [SMILES: COc1cc(C(=O)O)c(F)cc1NS(=O)(=O)c1csc(-c2c(F)cc(C3CCC(C)(C)CC3)cc2F)n1] |
| 819 | 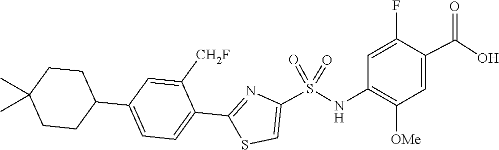 |
| 820 | 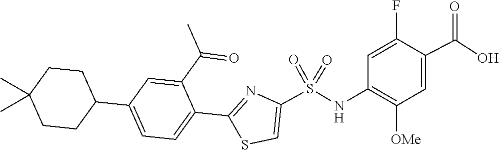 [SMILES: COc1cc(C(=O)O)c(F)cc1NS(=O)(=O)c1csc(-c2ccc(C3CCC(C)(C)CC3)cc2C(C)=O)n1] |
| 821 | 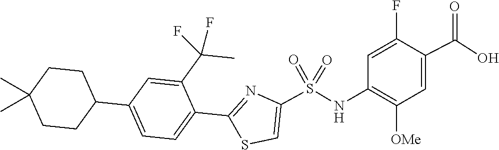 [SMILES: COc1cc(C(=O)O)c(F)cc1NS(=O)(=O)c1csc(-c2ccc(C3CCC(C)(C)CC3)cc2C(C)(F)F)n1] |
| 822 | 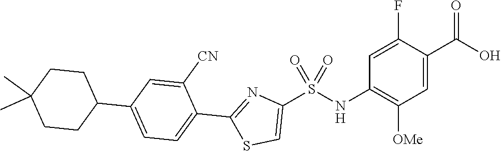 [SMILES: COc1cc(C(=O)O)c(F)cc1NS(=O)(=O)c1csc(-c2ccc(C3CCC(C)(C)CC3)cc2C#N)n1] |
| 823 | 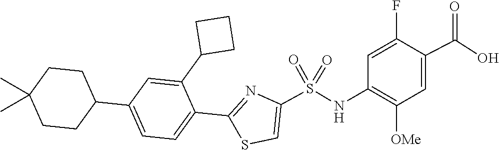 [SMILES: COc1cc(C(=O)O)c(F)cc1NS(=O)(=O)c1csc(-c2ccc(C3CCC(C)(C)CC3)cc2C2CCC2)n1] |
| 824 | 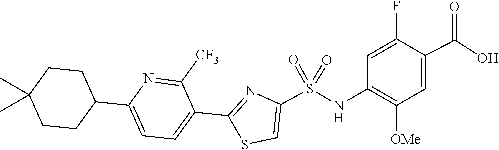 [SMILES: COc1cc(C(=O)O)c(F)cc1NS(=O)(=O)c1csc(-c2ccc(C3CCC(C)(C)CC3)nc2C(F)(F)F)n1] |
| 825 | 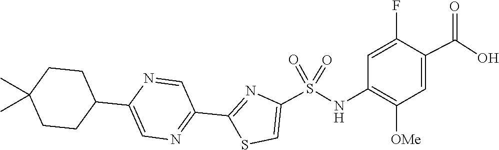 [SMILES: COc1cc(C(=O)O)c(F)cc1NS(=O)(=O)c1csc(-c2cnc(C3CCC(C)(C)CC3)cn2)n1] |
| 826 | 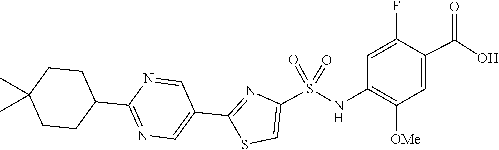 [SMILES: COc1cc(C(=O)O)c(F)cc1NS(=O)(=O)c1csc(-c2cnc(C3CCC(C)(C)CC3)nc2)n1] |
| 827 | 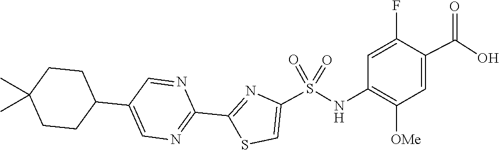 [SMILES: COc1cc(C(=O)O)c(F)cc1NS(=O)(=O)c1csc(-c2ncc(C3CCC(C)(C)CC3)cn2)n1] |
| 828 | 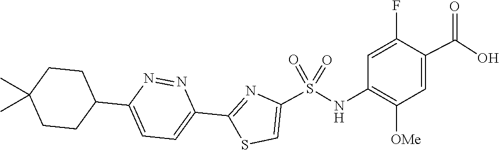 [SMILES: COc1cc(C(=O)O)c(F)cc1NS(=O)(=O)c1csc(-c2ccc(C3CCC(C)(C)CC3)nn2)n1] |
| 829 | 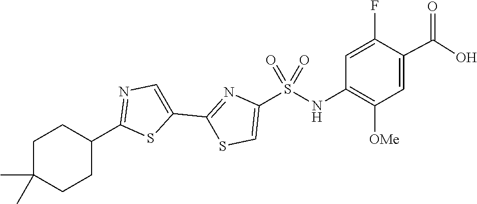 [SMILES: COc1cc(C(=O)O)c(F)cc1NS(=O)(=O)c1csc(-c2cnc(C3CCC(C)(C)CC3)s2)n1] |
| 830 | 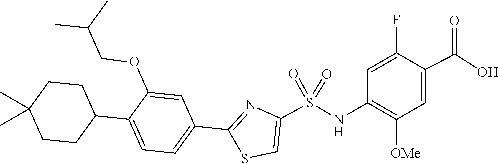 [SMILES: COc1cc(C(=O)O)c(F)cc1NS(=O)(=O)c1csc(-c2ccc(C3CCC(C)(C)CC3)c(OCC(C)C)c2)n1] |
| 831 | 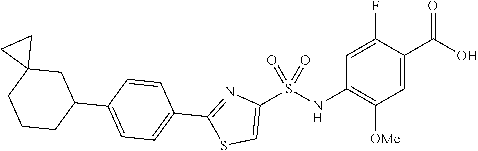 [SMILES: COc1cc(C(=O)O)c(F)cc1NS(=O)(=O)c1csc(-c2ccc(C3CCCC4(CC4)C3)cc2)n1] |
| 832 | 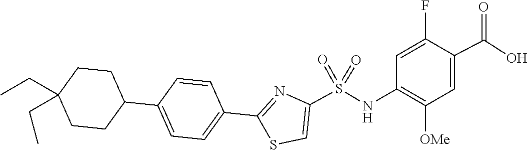 [SMILES: CCC1(CC)CCC(c2ccc(-c3nc(S(=O)(=O)Nc4cc(F)c(C(=O)O)cc4OC)cs3)cc2)CC1] |
| 833 | 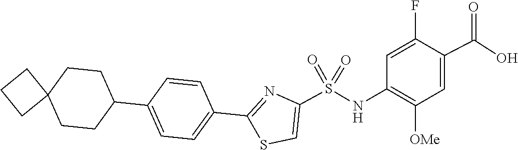 [SMILES: COc1cc(C(=O)O)c(F)cc1NS(=O)(=O)c1csc(-c2ccc(C3CCC4(CCC4)CC3)cc2)n1] |
| 834 | 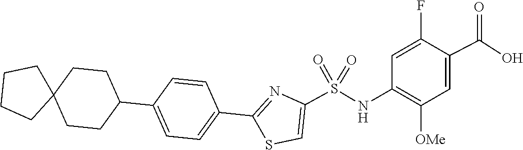 [SMILES: COc1cc(C(=O)O)c(F)cc1NS(=O)(=O)c1csc(-c2ccc(C3CCC4(CCCC4)CC3)cc2)n1] |
| 835 | 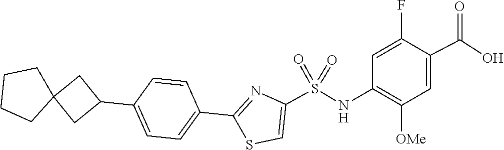 [SMILES: COc1cc(C(=O)O)c(F)cc1NS(=O)(=O)c1csc(-c2ccc(C3CC4(CCCC4)C3)cc2)n1] |
| 836 | 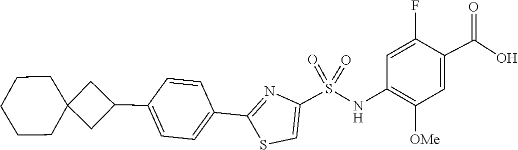 [SMILES: COc1cc(C(=O)O)c(F)cc1NS(=O)(=O)c1csc(-c2ccc(C3CC4(CCCCC4)C3)cc2)n1] |
| 837 | 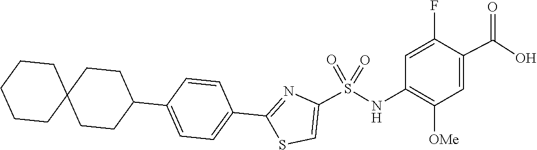 [SMILES: COc1cc(C(=O)O)c(F)cc1NS(=O)(=O)c1csc(-c2ccc(C3CCC4(CCCCC4)CC3)cc2)n1] |
| 838 | 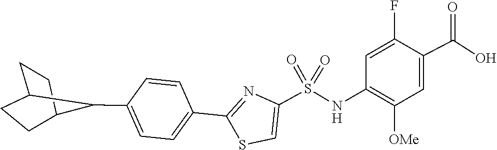 [SMILES: COc1cc(C(=O)O)c(F)cc1NS(=O)(=O)c1csc(-c2ccc(C3C4CCC3CC4)cc2)n1] |
| 839 | 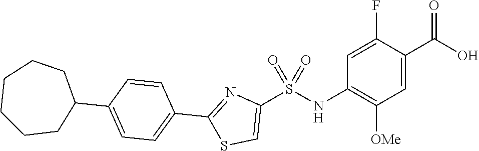 [SMILES: COc1cc(C(=O)O)c(F)cc1NS(=O)(=O)c1csc(-c2ccc(C3CCCCCC3)cc2)n1] |
| 840 | 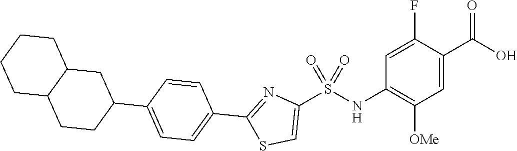 [SMILES: COc1cc(C(=O)O)c(F)cc1NS(=O)(=O)c1csc(-c2ccc(C3CCC4CCCCC4C3)cc2)n1] |
| 841 | 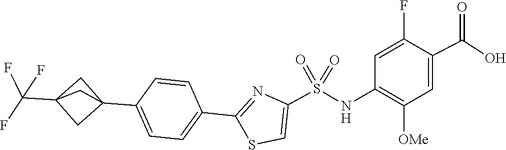 [SMILES: COc1cc(C(=O)O)c(F)cc1NS(=O)(=O)c1csc(-c2ccc(C34CC(C(F)(F)F)(C3)C4)cc2)n1] |
| 842 | 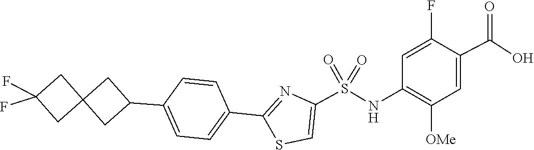 [SMILES: COc1cc(C(=O)O)c(F)cc1NS(=O)(=O)c1csc(-c2ccc(C3CC4(C3)CC(F)(F)C4)cc2)n1] |
| 843 | 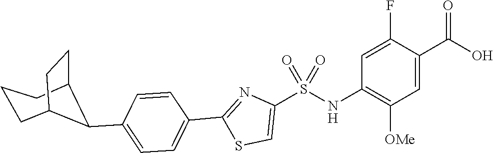 [SMILES: COc1cc(C(=O)O)c(F)cc1NS(=O)(=O)c1csc(-c2ccc(C3C4CCCC3CC4)cc2)n1] |
| 844 | 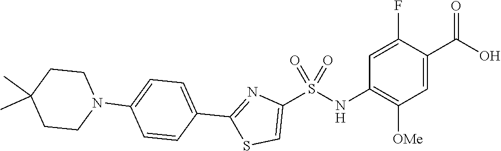 [SMILES: COc1cc(C(=O)O)c(F)cc1NS(=O)(=O)c1csc(-c2ccc(N3CCC(C)(C)CC3)cc2)n1] |
| 845 | 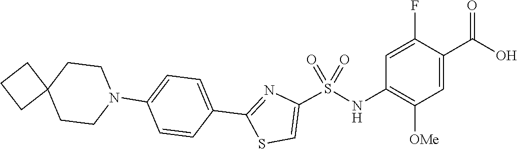 [SMILES: COc1cc(C(=O)O)c(F)cc1NS(=O)(=O)c1csc(-c2ccc(N3CCC4(CCC4)CC3)cc2)n1] |
| 846 | 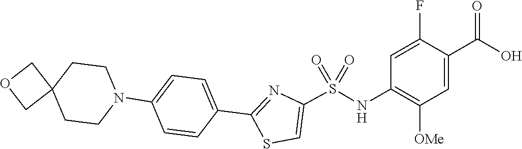 [SMILES: COc1cc(C(=O)O)c(F)cc1NS(=O)(=O)c1csc(-c2ccc(N3CCC4(CC3)COC4)cc2)n1] |
| 847 | 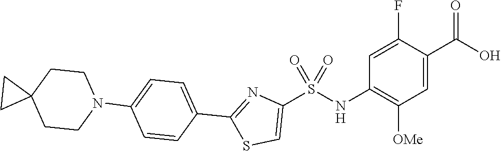 [SMILES: COc1cc(C(=O)O)c(F)cc1NS(=O)(=O)c1csc(-c2ccc(N3CCC4(CC3)CC4)cc2)n1] |
| 848 | 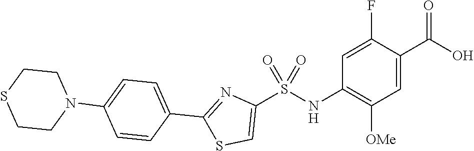 [SMILES: COc1cc(C(=O)O)c(F)cc1NS(=O)(=O)c1csc(-c2ccc(N3CCSCC3)cc2)n1] |
| 849 | 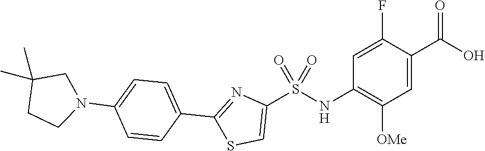 [SMILES: COc1cc(C(=O)O)c(F)cc1NS(=O)(=O)c1csc(-c2ccc(N3CCC(C)(C)C3)cc2)n1] |
| 850 | 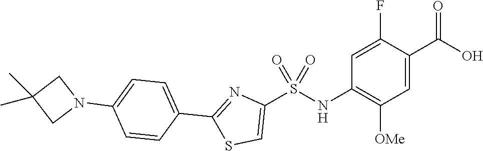 [SMILES: COc1cc(C(=O)O)c(F)cc1NS(=O)(=O)c1csc(-c2ccc(N3CC(C)(C)C3)cc2)n1] |
| 851 | 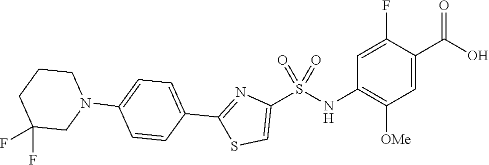 [SMILES: COc1cc(C(=O)O)c(F)cc1NS(=O)(=O)c1csc(-c2ccc(N3CCCC(F)(F)C3)cc2)n1] |
| 852 | 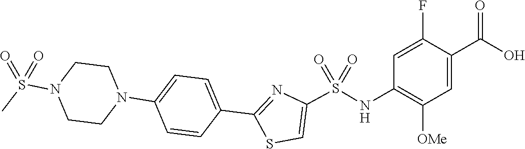 [SMILES: COc1cc(C(=O)O)c(F)cc1NS(=O)(=O)c1csc(-c2ccc(N3CCN(S(C)(=O)=O)CC3)cc2)n1] |
| 853 | 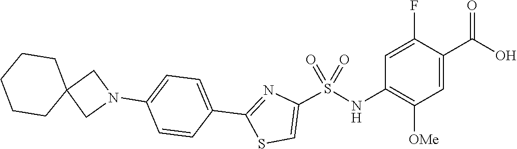 [SMILES: COc1cc(C(=O)O)c(F)cc1NS(=O)(=O)c1csc(-c2ccc(N3CC4(CCCCC4)C3)cc2)n1] |
| 854 | 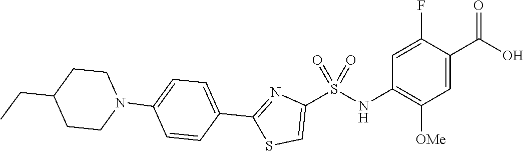 [SMILES: CCC1CCN(c2ccc(-c3nc(S(=O)(=O)Nc4cc(F)c(C(=O)O)cc4OC)cs3)cc2)CC1] |
| 855 | 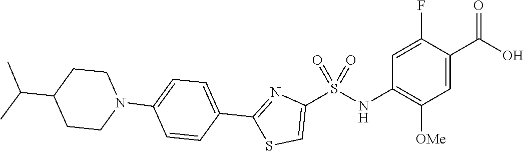 [SMILES: COc1cc(C(=O)O)c(F)cc1NS(=O)(=O)c1csc(-c2ccc(N3CCC(C(C)C)CC3)cc2)n1] |
| 856 | 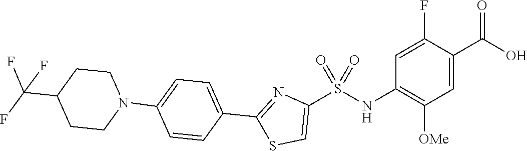 [SMILES: COc1cc(C(=O)O)c(F)cc1NS(=O)(=O)c1csc(-c2ccc(N3CCC(C(F)(F)F)CC3)cc2)n1] |
| 857 | 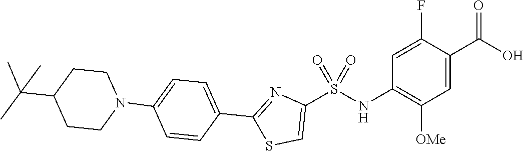 [SMILES: COc1cc(C(=O)O)c(F)cc1NS(=O)(=O)c1csc(-c2ccc(N3CCC(C(C)(C)C)CC3)cc2)n1] |
| 858 | 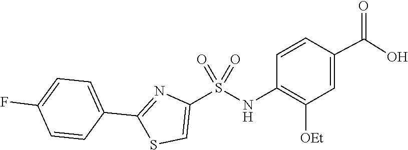 [SMILES: CCOc1cc(C(=O)O)ccc1NS(=O)(=O)c1csc(-c2ccc(F)cc2)n1] |
| 859 | 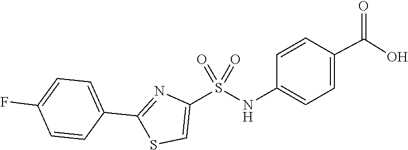 [SMILES: O=C(O)c1ccc(NS(=O)(=O)c2csc(-c3ccc(F)cc3)n2)cc1] |
| 860 | 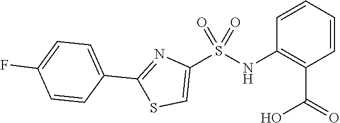 [SMILES: O=C(O)c1ccccc1NS(=O)(=O)c1csc(-c2ccc(F)cc2)n1] |
| 861 | 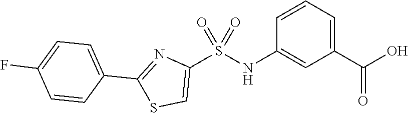 [SMILES: O=C(O)c1cccc(NS(=O)(=O)c2csc(-c3ccc(F)cc3)n2)c1] |
| 862 | 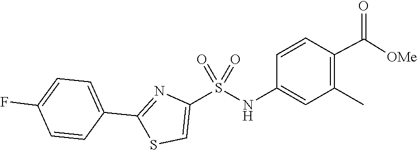 [SMILES: COC(=O)c1ccc(NS(=O)(=O)c2csc(-c3ccc(F)cc3)n2)cc1C] |
| 863 | 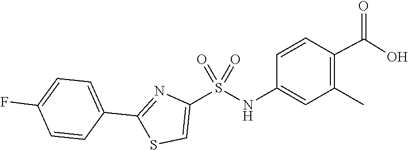 [SMILES: Cc1cc(NS(=O)(=O)c2csc(-c3ccc(F)cc3)n2)ccc1C(=O)O] |
| 864 | 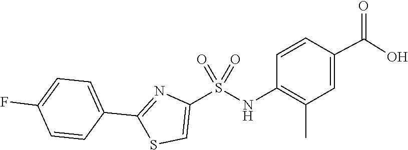 [SMILES: Cc1cc(C(=O)O)ccc1NS(=O)(=O)c1csc(-c2ccc(F)cc2)n1] |
| 865 | 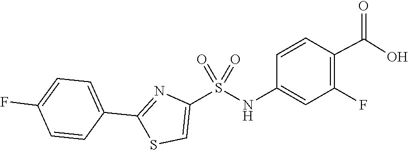 [SMILES: O=C(O)c1ccc(NS(=O)(=O)c2csc(-c3ccc(F)cc3)n2)cc1F] |
| 866 | 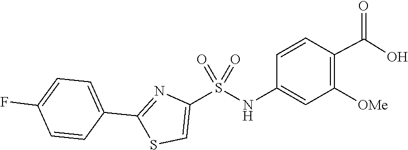 [SMILES: COc1cc(NS(=O)(=O)c2csc(-c3ccc(F)cc3)n2)ccc1C(=O)O] |
| 867 | 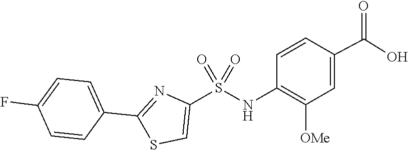 [SMILES: COc1cc(C(=O)O)ccc1NS(=O)(=O)c1csc(-c2ccc(F)cc2)n1] |
| 868 | 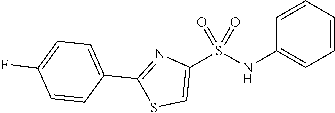 [SMILES: O=S(=O)(Nc1ccccc1)c1csc(-c2ccc(F)cc2)n1] |
| 869 | 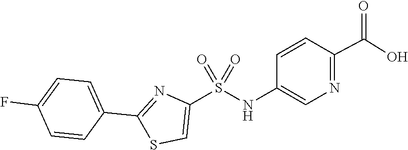 [SMILES: O=C(O)c1ccc(NS(=O)(=O)c2csc(-c3ccc(F)cc3)n2)cn1] |
| 870 | 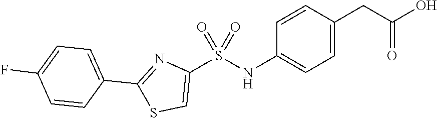 [SMILES: O=C(O)Cc1ccc(NS(=O)(=O)c2csc(-c3ccc(F)cc3)n2)cc1] |
| 871 | 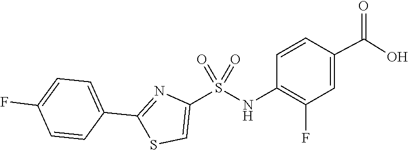 [SMILES: O=C(O)c1ccc(NS(=O)(=O)c2csc(-c3ccc(F)cc3)n2)c(F)c1] |
| 872 | 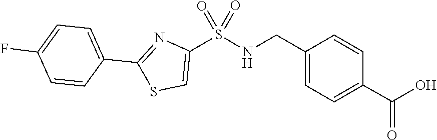 [SMILES: O=C(O)c1ccc(CNS(=O)(=O)c2csc(-c3ccc(F)cc3)n2)cc1] |
| 873 | 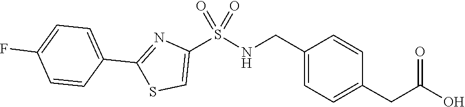 [SMILES: O=C(O)Cc1ccc(CNS(=O)(=O)c2csc(-c3ccc(F)cc3)n2)cc1] |
| 874 | 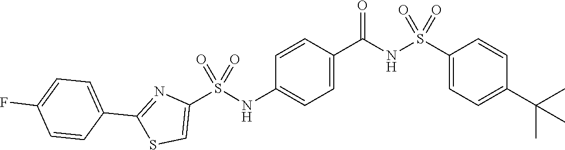 [SMILES: CC(C)(C)c1ccc(S(=O)(=O)NC(=O)c2ccc(NS(=O)(=O)c3csc(-c4ccc(F)cc4)n3)cc2)cc1] |
| 875 | 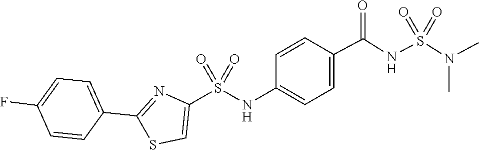 [SMILES: CN(C)S(=O)(=O)NC(=O)c1ccc(NS(=O)(=O)c2csc(-c3ccc(F)cc3)n2)cc1] |
| 876 | 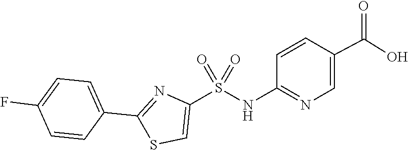 [SMILES: O=C(O)c1ccc(NS(=O)(=O)c2csc(-c3ccc(F)cc3)n2)nc1] |
| 877 | 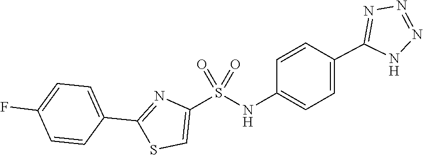 [SMILES: O=S(=O)(Nc1ccc(-c2nnn[nH]2)cc1)c1csc(-c2ccc(F)cc2)n1] |
| 878 | 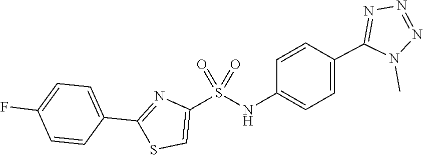 [SMILES: Cn1nnnc1-c1ccc(NS(=O)(=O)c2csc(-c3ccc(F)cc3)n2)cc1] |
| 879 | 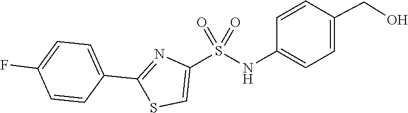 [SMILES: O=S(=O)(Nc1ccc(CO)cc1)c1csc(-c2ccc(F)cc2)n1] |
| 880 | 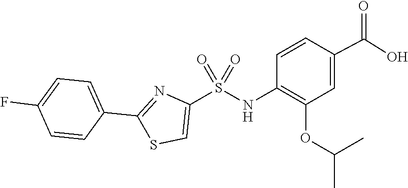 [SMILES: CC(C)Oc1cc(C(=O)O)ccc1NS(=O)(=O)c1csc(-c2ccc(F)cc2)n1] |
| 881 | 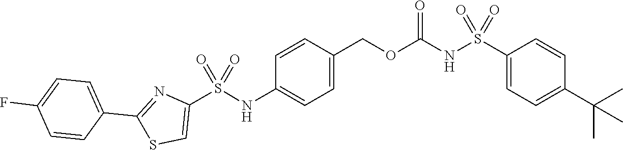 [SMILES: CC(C)(C)c1ccc(S(=O)(=O)NC(=O)OCc2ccc(NS(=O)(=O)c3csc(-c4ccc(F)cc4)n3)cc2)cc1] |
| 882 | 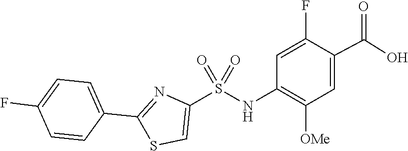 [SMILES: COc1cc(C(=O)O)c(F)cc1NS(=O)(=O)c1csc(-c2ccc(F)cc2)n1] |
| 883 | 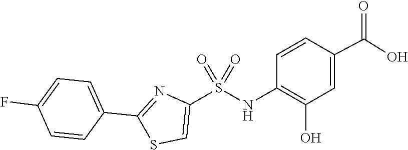 [SMILES: O=C(O)c1ccc(NS(=O)(=O)c2csc(-c3ccc(F)cc3)n2)c(O)c1] |
| 884 | 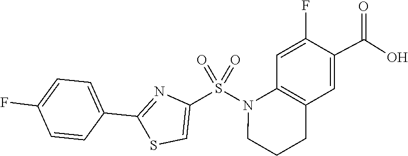 [SMILES: O=C(O)c1cc2c(cc1F)N(S(=O)(=O)c1csc(-c3ccc(F)cc3)n1)CCC2] |
| 885 | 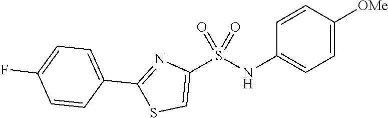 [SMILES: COc1ccc(NS(=O)(=O)c2csc(-c3ccc(F)cc3)n2)cc1] |
| 886 | 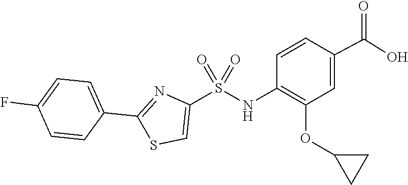 [SMILES: O=C(O)c1ccc(NS(=O)(=O)c2csc(-c3ccc(F)cc3)n2)c(OC2CC2)c1] |
| 887 | 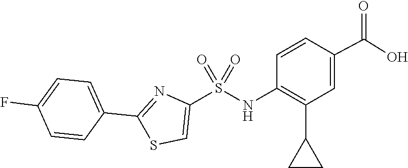 [SMILES: O=C(O)c1ccc(NS(=O)(=O)c2csc(-c3ccc(F)cc3)n2)c(C2CC2)c1] |
| 888 | 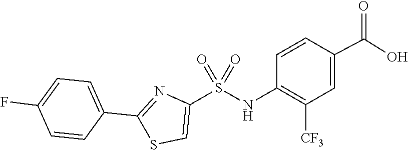 [SMILES: O=C(O)c1ccc(NS(=O)(=O)c2csc(-c3ccc(F)cc3)n2)c(C(F)(F)F)c1] |
| 889 | 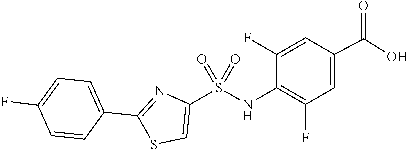 [SMILES: O=C(O)c1cc(F)c(NS(=O)(=O)c2csc(-c3ccc(F)cc3)n2)c(F)c1] |
| 890 | 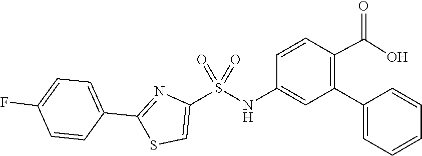 [SMILES: O=C(O)c1ccc(NS(=O)(=O)c2csc(-c3ccc(F)cc3)n2)cc1-c1ccccc1] |
| 891 | 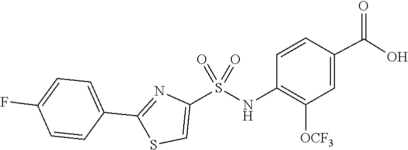 [SMILES: O=C(O)c1ccc(NS(=O)(=O)c2csc(-c3ccc(F)cc3)n2)c(OC(F)(F)F)c1] |
| 892 | 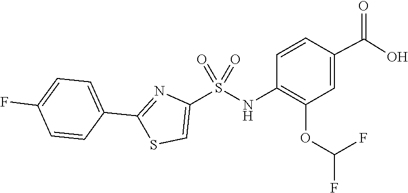 [SMILES: O=C(O)c1ccc(NS(=O)(=O)c2csc(-c3ccc(F)cc3)n2)c(OC(F)F)c1] |
| 893 | 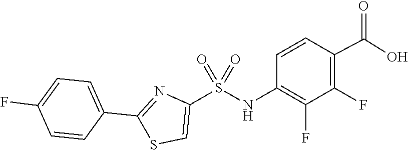 [SMILES: O=C(O)c1ccc(NS(=O)(=O)c2csc(-c3ccc(F)cc3)n2)c(F)c1F] |
| 894 | 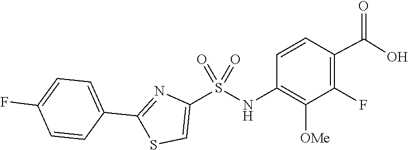 [SMILES: COc1c(NS(=O)(=O)c2csc(-c3ccc(F)cc3)n2)ccc(C(=O)O)c1F] |
| 895 | 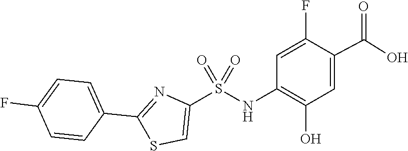 [SMILES: O=C(O)c1cc(O)c(NS(=O)(=O)c2csc(-c3ccc(F)cc3)n2)cc1F] |
| 896 | 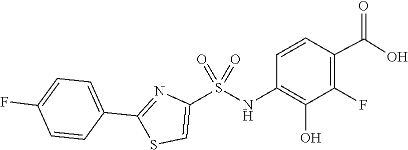 [SMILES: O=C(O)c1ccc(NS(=O)(=O)c2csc(-c3ccc(F)cc3)n2)c(O)c1F] |
| 897 | 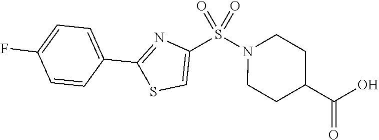 [SMILES: O=C(O)C1CCN(S(=O)(=O)c2csc(-c3ccc(F)cc3)n2)CC1] |
| 898 | 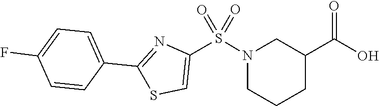 [SMILES: O=C(O)C1CCCN(S(=O)(=O)c2csc(-c3ccc(F)cc3)n2)C1] |
| 899 | 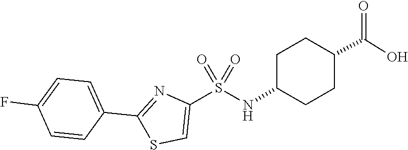 [SMILES: O=C(O)[C@H]1CC[C@@H](NS(=O)(=O)c2csc(-c3ccc(F)cc3)n2)CC1] |
| 900 | 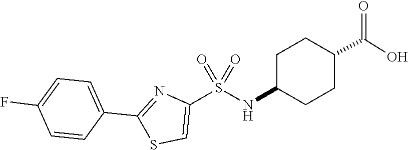 [SMILES: O=C(O)[C@H]1CC[C@H](NS(=O)(=O)c2csc(-c3ccc(F)cc3)n2)CC1] |
| 901 | 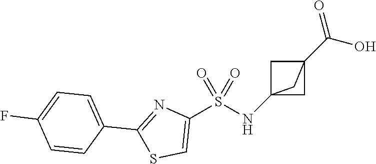 [SMILES: O=C(O)C12CC(NS(=O)(=O)c3csc(-c4ccc(F)cc4)n3)(C1)C2] |
| 902 | 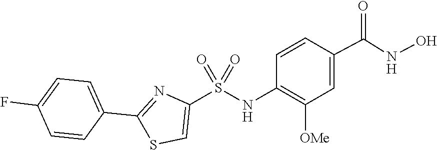 [SMILES: COc1cc(C(=O)NO)ccc1NS(=O)(=O)c1csc(-c2ccc(F)cc2)n1] |
| 903 | 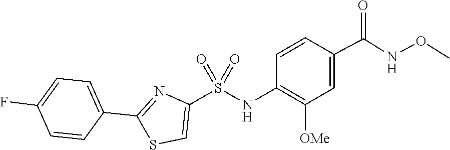 [SMILES: CONC(=O)c1ccc(NS(=O)(=O)c2csc(-c3ccc(F)cc3)n2)c(OC)c1] |
| 904 | 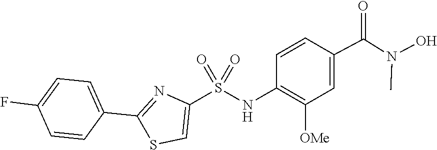 [SMILES: COc1cc(C(=O)N(C)O)ccc1NS(=O)(=O)c1csc(-c2ccc(F)cc2)n1] |
| 905 | 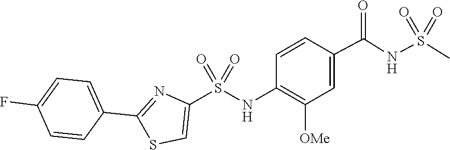 [SMILES: COc1cc(C(=O)NS(C)(=O)=O)ccc1NS(=O)(=O)c1csc(-c2ccc(F)cc2)n1] |
| 906 | 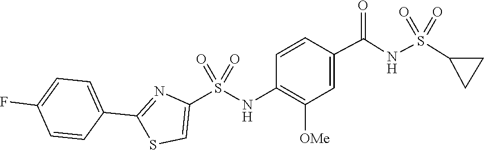 [SMILES: COc1cc(C(=O)NS(=O)(=O)C2CC2)ccc1NS(=O)(=O)c1csc(-c2ccc(F)cc2)n1] |
| 907 | 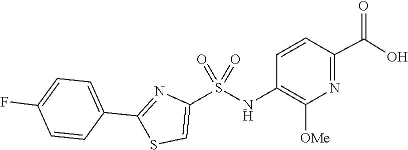 [SMILES: COc1nc(C(=O)O)ccc1NS(=O)(=O)c1csc(-c2ccc(F)cc2)n1] |
| 908 | 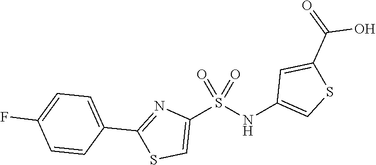 [SMILES: O=C(O)c1cc(NS(=O)(=O)c2csc(-c3ccc(F)cc3)n2)cs1] |
| 909 | 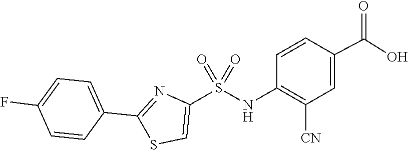 [SMILES: N#Cc1cc(C(=O)O)ccc1NS(=O)(=O)c1csc(-c2ccc(F)cc2)n1] |
| 910 | 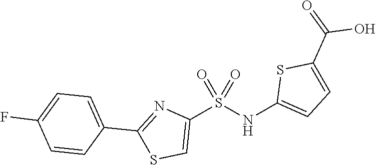 [SMILES: O=C(O)c1ccc(NS(=O)(=O)c2csc(-c3ccc(F)cc3)n2)s1] |
| 911 | 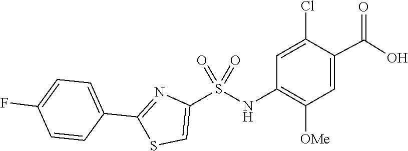 [SMILES: COc1cc(C(=O)O)c(Cl)cc1NS(=O)(=O)c1csc(-c2ccc(F)cc2)n1] |
| 912 | 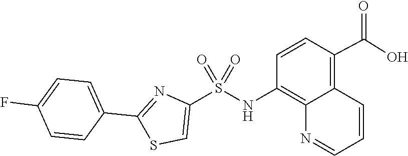 [SMILES: O=C(O)c1ccc(NS(=O)(=O)c2csc(-c3ccc(F)cc3)n2)c2ncccc12] |
| 913 | 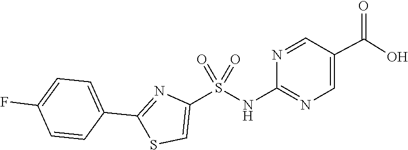 [SMILES: O=C(O)c1cnc(NS(=O)(=O)c2csc(-c3ccc(F)cc3)n2)nc1] |
| 914 | 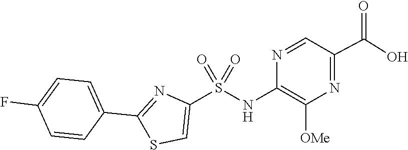 [SMILES: COc1nc(C(=O)O)cnc1NS(=O)(=O)c1csc(-c2ccc(F)cc2)n1] |
| 915 | 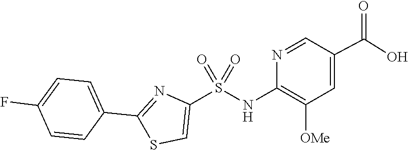 [SMILES: COc1cc(C(=O)O)cnc1NS(=O)(=O)c1csc(-c2ccc(F)cc2)n1] |
| 916 | 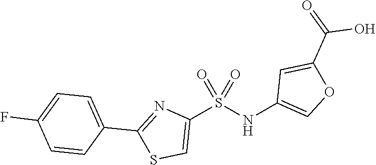 [SMILES: O=C(O)c1cc(NS(=O)(=O)c2csc(-c3ccc(F)cc3)n2)co1] |
| 917 | 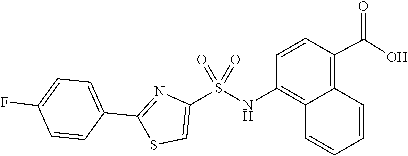 [SMILES: O=C(O)c1ccc(NS(=O)(=O)c2csc(-c3ccc(F)cc3)n2)c2ccccc12] |
| 918 | 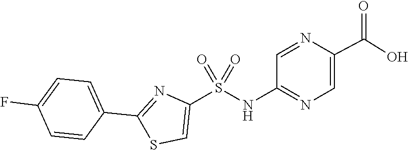 [SMILES: O=C(O)c1cnc(NS(=O)(=O)c2csc(-c3ccc(F)cc3)n2)cn1] |
| 919 | 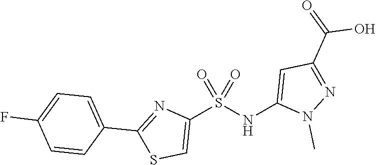 [SMILES: Cn1nc(C(=O)O)cc1NS(=O)(=O)c1csc(-c2ccc(F)cc2)n1] |
| 920 | 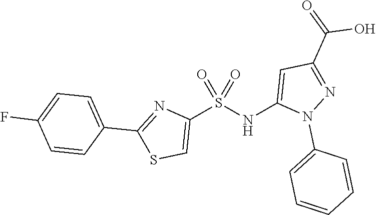 [SMILES: O=C(O)c1cc(NS(=O)(=O)c2csc(-c3ccc(F)cc3)n2)n(-c2ccccc2)n1] |
| 921 | 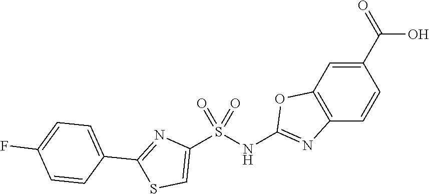 [SMILES: O=C(O)c1ccc2nc(NS(=O)(=O)c3csc(-c4ccc(F)cc4)n3)oc2c1] |
| 922 | 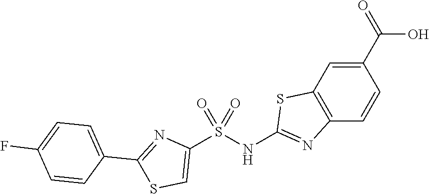 [SMILES: O=C(O)c1ccc2nc(NS(=O)(=O)c3csc(-c4ccc(F)cc4)n3)sc2c1] |
| 923 | 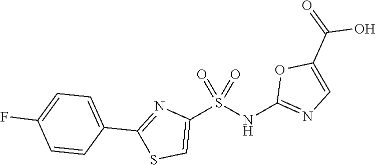 [SMILES: O=C(O)c1cnc(NS(=O)(=O)c2csc(-c3ccc(F)cc3)n2)o1] |
| 924 | 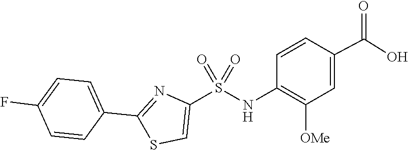 [SMILES: COc1cc(C(=O)O)ccc1NS(=O)(=O)c1csc(-c2ccc(F)cc2)n1] |
| 925 | 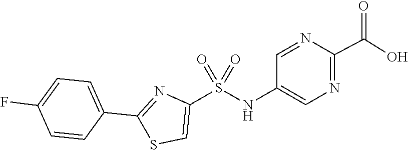 [SMILES: O=C(O)c1ncc(NS(=O)(=O)c2csc(-c3ccc(F)cc3)n2)cn1] |
| 926 | 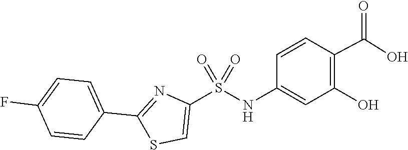 [SMILES: O=C(O)c1ccc(NS(=O)(=O)c2csc(-c3ccc(F)cc3)n2)cc1O] |
| 927 | 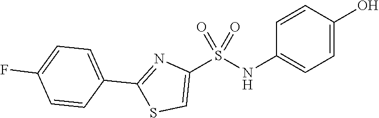 [SMILES: O=S(=O)(Nc1ccc(O)cc1)c1csc(-c2ccc(F)cc2)n1] |
| 928 | 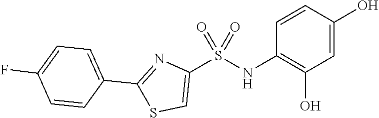 [SMILES: O=S(=O)(Nc1ccc(O)cc1O)c1csc(-c2ccc(F)cc2)n1] |
| 929 | 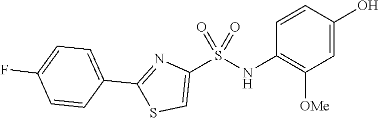 [SMILES: COc1cc(O)ccc1NS(=O)(=O)c1csc(-c2ccc(F)cc2)n1] |
| 930 | 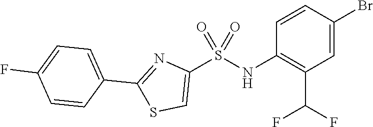 [SMILES: O=S(=O)(Nc1ccc(Br)cc1C(F)F)c1csc(-c2ccc(F)cc2)n1] |
| 931 | 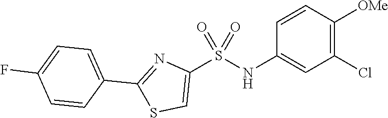 [SMILES: COc1ccc(NS(=O)(=O)c2csc(-c3ccc(F)cc3)n2)cc1Cl] |
| 932 | 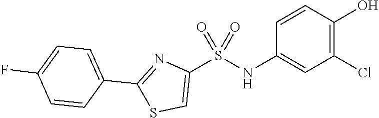 [SMILES: O=S(=O)(Nc1ccc(O)c(Cl)c1)c1csc(-c2ccc(F)cc2)n1] |
| 933 | 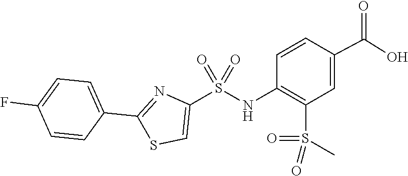 [SMILES: CS(=O)(=O)c1cc(C(=O)O)ccc1NS(=O)(=O)c1csc(-c2ccc(F)cc2)n1] |
| 934 | 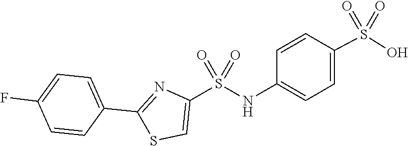 [SMILES: O=S(=O)(O)c1ccc(NS(=O)(=O)c2csc(-c3ccc(F)cc3)n2)cc1] |
| 935 | 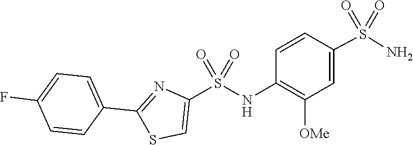 [SMILES: COc1cc(S(N)(=O)=O)ccc1NS(=O)(=O)c1csc(-c2ccc(F)cc2)n1] |
| 936 | 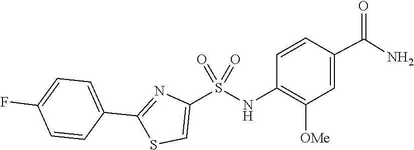 [SMILES: COc1cc(C(N)=O)ccc1NS(=O)(=O)c1csc(-c2ccc(F)cc2)n1] |
| 937 | 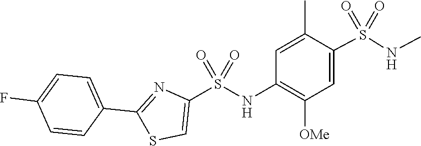 [SMILES: CNS(=O)(=O)c1cc(OC)c(NS(=O)(=O)c2csc(-c3ccc(F)cc3)n2)cc1C] |
| 938 | 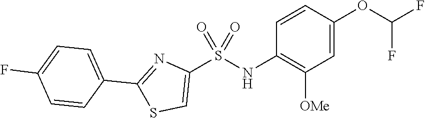 [SMILES: COc1cc(OC(F)F)ccc1NS(=O)(=O)c1csc(-c2ccc(F)cc2)n1] |
| 939 | 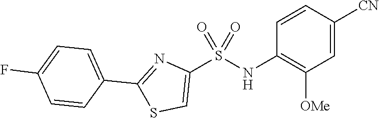 [SMILES: COc1cc(C#N)ccc1NS(=O)(=O)c1csc(-c2ccc(F)cc2)n1] |
| 940 | 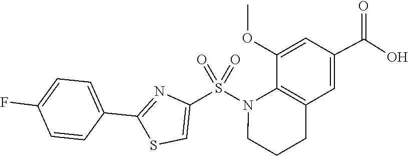 [SMILES: COc1cc(C(=O)O)cc2c1N(S(=O)(=O)c1csc(-c3ccc(F)cc3)n1)CCC2] |
| 941 | 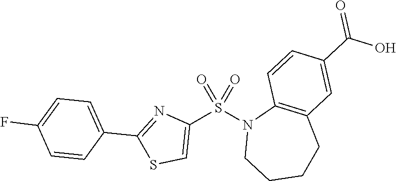 [SMILES: O=C(O)c1ccc2c(c1)CCCCN2S(=O)(=O)c1csc(-c2ccc(F)cc2)n1] |
| 942 | 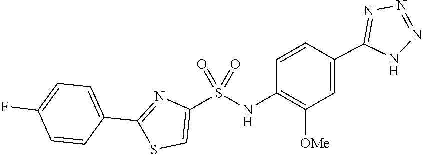 [SMILES: COc1cc(-c2nnn[nH]2)ccc1NS(=O)(=O)c1csc(-c2ccc(F)cc2)n1] |
| 943 | 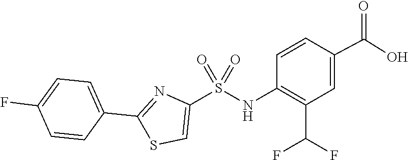 [SMILES: O=C(O)c1ccc(NS(=O)(=O)c2csc(-c3ccc(F)cc3)n2)c(C(F)F)c1] |
| 944 | 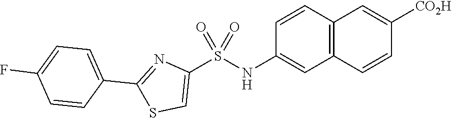 [SMILES: O=C(O)c1ccc2cc(NS(=O)(=O)c3csc(-c4ccc(F)cc4)n3)ccc2c1] |
| 945 | 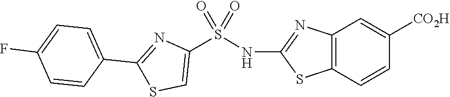 [SMILES: O=C(O)c1ccc2sc(NS(=O)(=O)c3csc(-c4ccc(F)cc4)n3)nc2c1] |
| 946 | 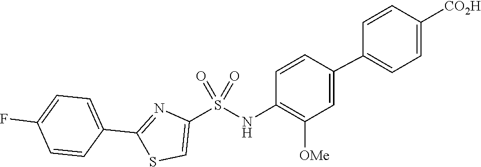 [SMILES: COc1cc(-c2ccc(C(=O)O)cc2)ccc1NS(=O)(=O)c1csc(-c2ccc(F)cc2)n1] |
| 947 | 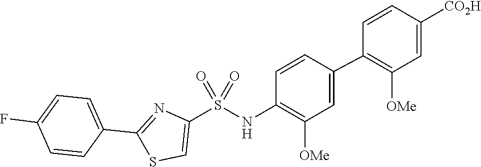 [SMILES: COc1cc(-c2ccc(C(=O)O)cc2OC)ccc1NS(=O)(=O)c1csc(-c2ccc(F)cc2)n1] |

Compounds of Formula (I) as disclosed herein can be prepared as described in WO2023/023310, the contents of which are incorporated herein by reference in their entirety. WO2023/023310 also discloses methods for determining the HSD17B13 inhibitory activity of such compounds.

It will be appreciated that the description of the present invention herein should be construed in congruity with the laws and principles of chemical bonding. In some instances, it may be necessary to remove a hydrogen atom in order to accommodate a substituent at any given location.

It will be yet appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

In another embodiment, the present invention provides a pharmaceutical composition comprising a therapeutically effective amount of a compound or combination of compounds of the present invention, or a pharmaceutically acceptable salt form, stereoisomer, solvate, hydrate or combination thereof, in combination with a pharmaceutically acceptable carrier or excipient.

In another embodiment, described herein is a method of treatment or prevention of a metabolic or liver condition in a mammal comprising administering a therapeutically effective amount of a compound described herein, or a pharmaceutically acceptable salt, or solvate thereof, to the mammal in need thereof.

In another embodiment, the present invention provides a method of modulating a HSD17B13 protein for treatment of metabolic disease or lung condition. The method comprises administering a therapeutically effective amount of a compound of Formula (I).

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of C₁-C₈ alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of C₃-C₁₂ cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of C₃-C₁₂ cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” is a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy are C₁-C₃ alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic groups may be further substituted. Heteroaryl or Heterocyclic groups can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —C₁, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH— heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, CO₂—C₃-C₁₂-cycloalkyl, —CO₂— aryl, CO₂-heteroaryl, CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocyclo-alkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocyclo-alkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Methods of Treating a Fibrotic Disease

The present invention provides methods of treating a fibrotic disease. A “fibrotic disease” is a disease characterized by fibrosis, the replacement of normal tissue with fibrotic tissue in one or more organs. Fibrosis can occur in a variety of organs, including lungs, liver, kidney, brain and heart. Interstitial lung fibrotic diseases (ILD) broadly include Idiopathic Pulmonary Fibrosis (IPF), Acute Interstitial Pneumonia, Non-specific Interstitial Pneumonia, Cryptogenic Organizing Pneumonia, Systemic Lupus Erythematosus-related ILD, Scleroderma-related ILD, Rheumatoid Arthritis-related ILD, Drug-induced (ie chemotherapy) ILD, Environmentally-induced (ie Radiation) ILD, in addition to Asthma.

In certain embodiments, the fibrotic disease to be treated is not a fibrotic disease of the liver. In certain embodiments, the fibrotic disease to be treated is an interstitial lung fibrotic disease, preferably idiopathic pulmonary fibrosis.

The subject to be treated is a human or non-human animal, for example, a companion animal, such as a dog or cat, or livestock animal, such as an equine, bovine, ovine or porcine animal. Preferably the subject is a human.

According to the methods of the invention, the compound of Formula (I) or pharmaceutically acceptable salt thereof is administered to the subject in need thereof in a therapeutically effective amount. A “therapeutically effective amount” of a compound of the invention is an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). For example, in the treatment or prevention of a fibrotic disease, a therapeutically effective amount is an amount which results in a reduction in the amount of fibrotic tissue or inhibition or slowing of further development of fibrotic tissue.

The compounds of the present invention can be dosed, for example, in an amount from about 0.01 mg/Kg to about 500 mg/Kg, alternatively from about 1 to about 50 mg/Kg. Effective amounts or doses will also vary depending on route of administration, as well as the possibility of co-usage with other agents. The total daily dose of the compound of Formula (I) or salt thereof administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a subject in need of such treatment from about 10 mg to about 1000 mg of the compound(s) of this invention per day in single or multiple doses.

The total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The compound of Formula (I) or salt thereof can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage, for example, ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

### EXAMPLES

### Example 1 HSD17B13 Presence in Mouse Lung

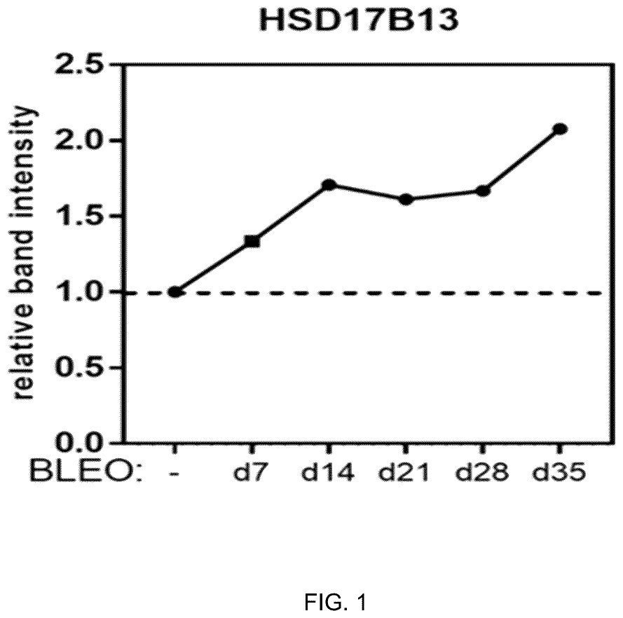

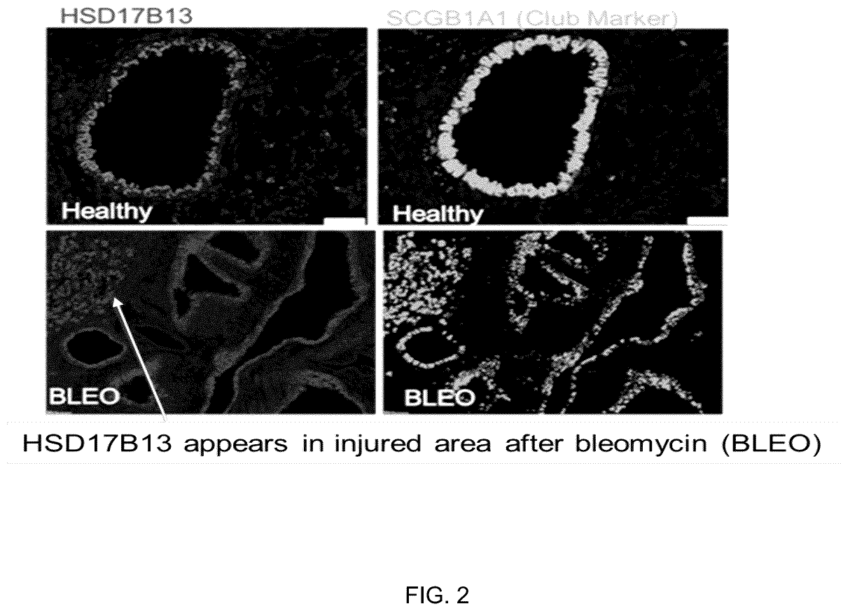

This study was to analyze immunohistochemical detection of HSD17B13 and a club cell marker (SCGB1A1) in a bleomycin-induced (BLEO) mouse model of lung fibrosis (BLEO-IPF). Male C57BL/6JRj mice received a single dose of bleomycin (1.5 mg/kg, n=7) or control (saline) (n=8) by intratracheal instillation (II) at day 0. Animals were sacrificed at various time points after bleomycin; lung samples were collected and embedded in paraffin or homogenized for protein lysates. A time course of HSD17B13 protein abundance after bleomycin induced lung fibrosis was evaluated (FIG. 1). HSD17B13 increases over time in line with fibrosis development. For HSD17B13 localization, immunostaining was performed. The tissue sections were boiled in TEG-buffer (pH 9) for 15 minutes. Lung samples were stained for HSD17B13 (Thermo PA5-109834) and SCGB1A1 (Sigma AbS1673) by immunofluorescence (IF), followed by a qualitative assessment. Both HSD17B13 and SCGB1A1 could be identified in lung using IF and co-localization of the two markers was observed (FIG. 2). HSD17B13 and SCGB1A1 were found in the air-conducting system of lungs from control and BLEO-IPF mice. In BLEO-IPF lung tissue both HSD17B13 and SCGB1A1 were found in areas of ectopic bronchiolar tissue. HSD17B13 and SCGB1A1 were present and co-localize in lung tissue from BLEO-IPF mice.

### Example 2 HSD17B13 Presence in Human Lung

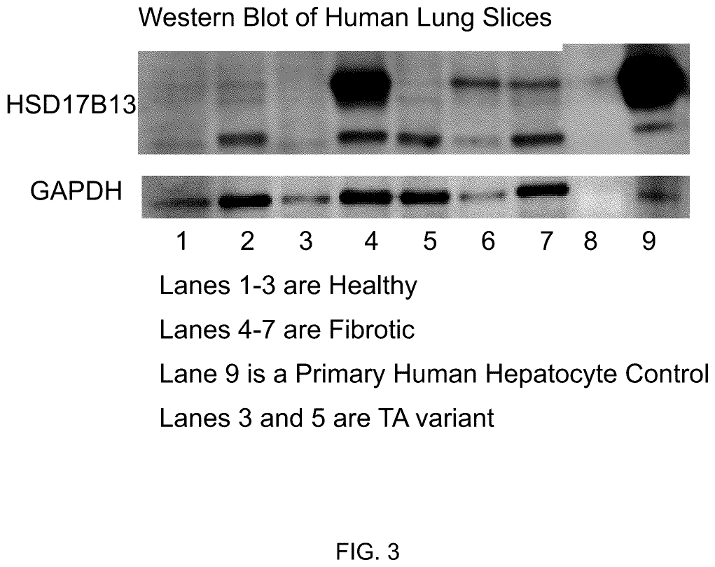

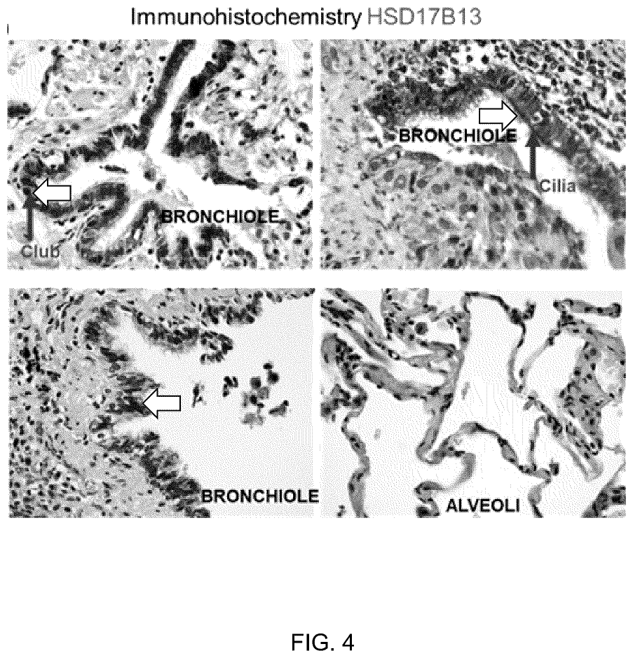

This study was to analyze HSD17B13 protein presence in human lung via western blot (FIG. 3) or immunohistochemical detection of HSD17B13 and a club cell marker (SCGB1A1) (FIG. 4).

Lung protein lysates (Origene) were evaluated by western blot (Thermo PA5-109834). HSD17B13 protein was present in normal and fibrotic lung (FIG. 3). Tubulin was used as loading control. Protein lysate from a primary human hepatocyte control (lane 9) as used as control for protein size. Origene lung lysates tested: CP565443, CP565446, CP565578, CP565542, CP565715, CP565759, CP565580, CP565677, CP565565, CP565663, CP565539.

Lung sections (PA0F334840, PA000040IC, PA0000002A), showed positive staining for SCGB1A1 (SC-365992) and HSD17B13 (Thermo PA5-109834) in non-ciliated bronchiolar epithelial cells that were characterized by intense dark brown cytoplasmic staining (FIG. 4). In three of the sections (PA00004A4A, PA154772F6, PA154768CD), bronchioles were not readily apparent and as a result, club cells were not available for evaluation; staining was negative for both SCGB1A1 and HSD17B13. This suggests localized presence of HSD17B13 in bronchiolar epithelia and colocalization with club cells.

### Example 3 HSD17B13 Expression in Air-Liquid Interface (ALI) Cultures of Human Small Airway Epithelial Cell (HSAEC)

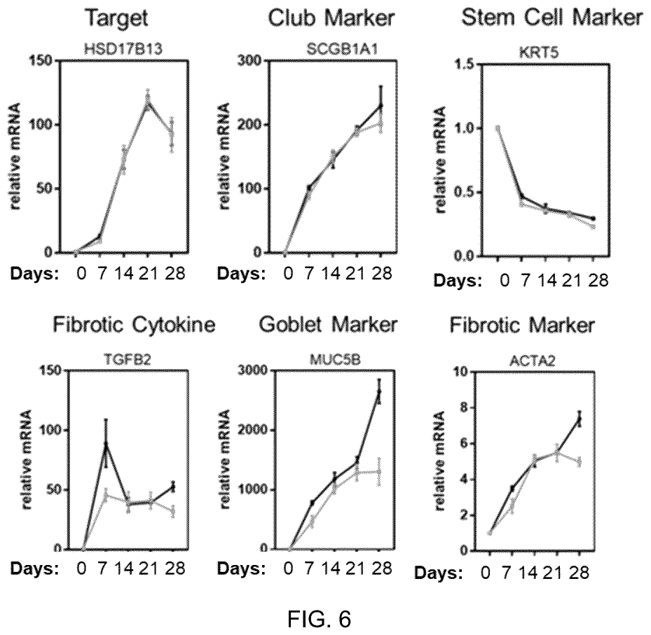

Primary human small airway epithelial cells (HSAECs) were obtained from ATCC (PCS-301-010). Cells were cultured in PneumaCult-Ex Plus media (StemCell Technologies, Cat No. 05040) and grown to confluence on the 6.5 mm Transwell® inserts with 0.4 m pores of a 24-well plate (StemCell Technologies, Cat No. 38024) under 37° C., 5% CO₂ and 95% humidity. Cells were airlifted after 2-4 days by removing the media from the apical chamber and providing the PneumaCult-ALI-S media (StemCell Technologies, Cat No. 05050) in the basal chamber only for air-liquid interface culture. Cells achieved full differentiation 28 days after airlifting and were prepared for the treatment. HSD17B13 gene expression increased robustly in parallel with club cell markers during 28-day differentiation. Addition of Compound 812 as HSD17B13 inhibitors during differentiation did not interfere with club cell marker SCGB1A1 expression but did decrease expression of IPF-related gene markers by day 28 (MUC5B, TGFB2, ACTA2) (FIG. 6).

### Example 4 HSD17B13 Inhibitors in Precision Cut Lung Slice (PCLS) Culture

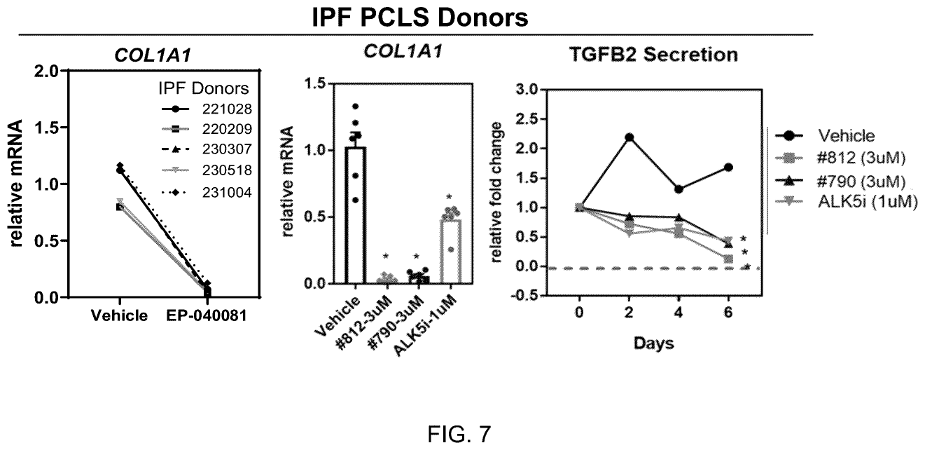

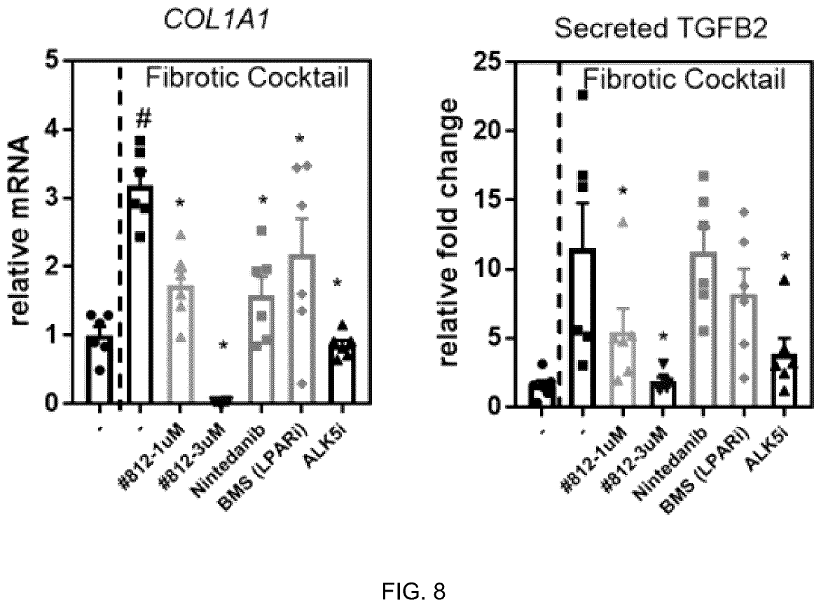

Human PCLS were generated from lung explants of patients with pulmonary fibrosis (donor ID #221028) or normal donor (donor ID #220730). Cryopreserved hPCLSs were obtained from Institute for In Vitro Sciences (IIVS) and maintained with air-liquid interface culture approach under 37° C., 5% CO₂ and 95% humidity. hPCLS were cultured in DMEM-F12 (Invitrogen, Cat No. 11330032) containing 0.2% primocin (Invivogen, Cat No. ant-pm-1) and 1% insulin-transferrin-selenium (ITS-G; Invitrogen, Cat No. 41400045), after initial acclimation in acclimation media, including culture media supplemented with 1% antibiotic antimycotic solution (Invitrogen, Cat No. 15240062), 2 mM Hydrocortisone (Millipore Sigma,) and 5 mg/ml 2-phospho-L-abscorbic acid trisodium salt (Millipore Sigma, Cat No. 49752-10G) for 3 days. Slices of tissue were flatly grown in the tissue culture inserts with 0.4 m pores of a 12-well plate (StemCell Technologies, Cat No. 1001027), supplemented with 100 μl culture media in the apical side containing PCLSs, and 900 μL culture media in the basal compartment. Media were changed every 48 hours. hPCLS were treated and randomly assigned in replicates of 6-8 per condition 7 days after recovery from cryopreservation. Supernatants were collected at day 0, 2, 4, 6 after treatment for secreted protein analysis. IPF donor PCLS were collected at day 7 for subsequent RNA isolation and gene expression quantification. Normal donor PCLS were treated for 4 days with a control cocktail (CC) including all vehicles or a pro-fibrotic cocktail (FC) consisting of transforming growth factor-β (TGF-β) (5 ng/ml, Bio-Techne), platelet-derived growth factor-AB (PDGF-AB) (10 ng/ml, Thermo Fisher), tumor necrosis factor-α (TNF-α) (10 ng/ml, Bio-Techne), and lysophosphatidic acid (LPA) (5 μM, Cayman chemical) as described before. Compounds 790 and 812 were used as HSD17B13 inhibitors. HSD17B13 inhibitors decreased gene and secreted protein markers of fibrosis in IPF donor PCLS (FIG. 7) and FC-treated normal donor PCLS (FIG. 8). IPF PCLS donors were tested and efficacy by compounds was observed in all cases.

### Example 5 HSD17B13 Inhibitors in Human Lung Cancer Cell Lines (11441)

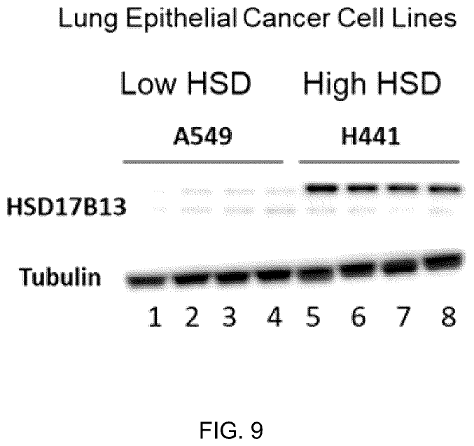

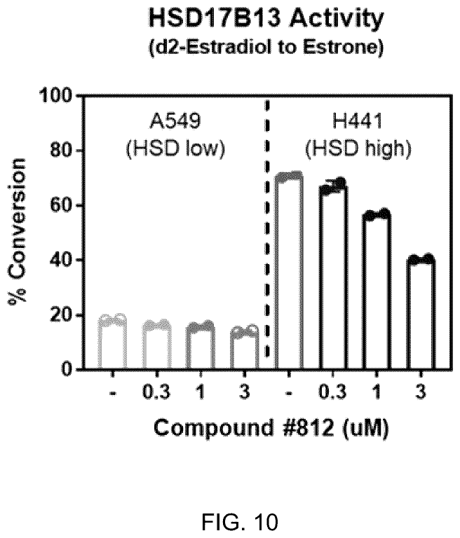

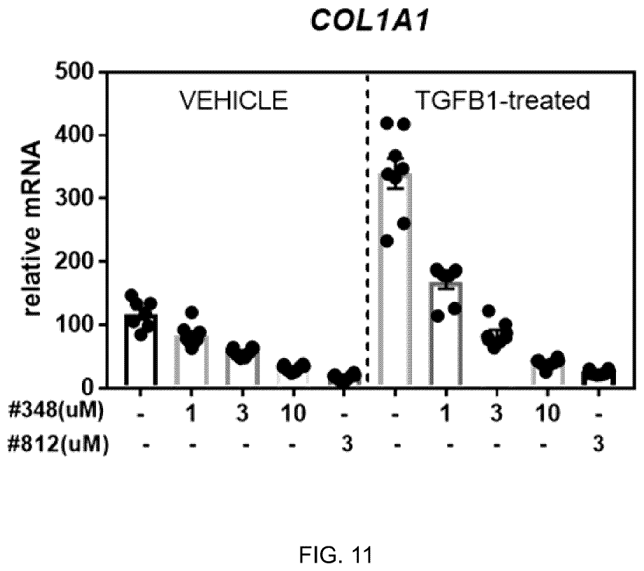

Human lung cancer cell lines with high (H441) or low (A549) expression of HSD17B13, as confirmed by western blot, were acquired from ATCC and maintained according to manufacturer's instructions. Cells were seeded at a confluency of 2e5 cells/well (A549) or 4e5 cells/well (H441) in a 6-well format. Once cells were 70% confluent, media was removed and replenished with starvation media for 24 hours. Afterward starvation media was removed and replenished with treatment media for 24 hours. Compound 348 and Compound 812 were used as HSD17B13 inhibitors. For gene expression analysis, treatment media consisted of +/−TGFB1 (10 ng/mL) and +/−HSD17B13 inhibitor (3 uM). For HSD17B13 enzymatic activity, treatment media consisted of +/−d2-Estradiol (Cayman Chemicals) and +/−HSD17B13 inhibitor (0.3 uM, 1 uM, 3 uM); supernatants were collected to measure estradiol conversion to estrone by rapid fire mass spectrometry. As shown in FIGS. 9-11, compounds 348 and 812 both decreased COL1A1 (Collagen) and enzymatic activity only in cells with high HSD17B13 (H441) suggesting an on-target mechanism by the HSD17B13 inhibitor.

While this invention has been particularly shown and described with references to preferred embodiments thereof, it will be understood by those skilled in the art that various changes in form and details may be made therein without departing from the scope of the invention encompassed by the appended claims.

1. A method of treating a fibrotic disease in a subject in need thereof, comprising administering to the subject a therapeutically effective amount of a compound of Formula I, 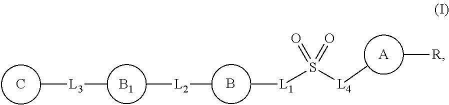
or a pharmaceutically acceptable salt thereof, wherein:
L₁ and L₄ are each independently absent or NR₁;
R₁ is hydrogen, optionally substituted methyl, or optionally substituted acetyl;
\textcircled{B} is absent or selected from the group consisting of optionally substituted aryl, optionally substituted heteroaryl, optionally substituted arylalkyl, optionally substituted heteroarylalkyl, optionally substituted —C₃-C₁₂ cycloalkyl, optionally substituted 3- to 12-membered heterocycloalkyl, and optionally substituted —C₃-C₁₂ cycloalkenyl;
alternatively, L₁ is NR₁, R₁ is connected to \textcircled{B}, and R₁ and the nitrogen atom to which it is attached form a heterocyclic ring;
L₂ is absent or selected from the groups consisting of —NR₂—, —O—, optionally substituted —C₁-C₆ alkyl, optionally substituted —C₂-C₆ alkenyl and optionally substituted —C₂-C₆ alkynyl;
R₂ is hydrogen, optionally substituted —C₁-C₆ alkyl, optionally substituted —C₃-C₁₂ cycloalkyl, and optionally substituted 3- to 12-membered heterocycloalkyl;
\textcircled{B₁} is absent or selected from the groups consisting of optionally substituted aryl, optionally substituted heteroaryl, optionally substituted —C₃-C₁₂ cycloalkyl, optionally substituted 3- to 12-membered heterocycloalkyl, and optionally substituted —C₃-C₁₂ cycloalkenyl;
L₃ is absent or selected from the group consisting of optionally substituted —C₁-C₆ alkyl, optionally substituted —C₂-C₆ alkenyl, optionally substituted —C₂-C₆ alkynyl, optional substituted-aliphatic which contains 1-6 carbon atoms and at least one other atom selected from oxygen, nitrogen and sulfur atom, optionally substituted —C₃-C₁₂ cycloalkyl, optionally substituted 3- to 12-membered heterocycloalkyl, —O—, —NR₂—, —C(O)—, —OC(O)—, —C(O)O—, —NR₂C(O)—, —C(O)NR₂—, —S(O)₂—, —S(O)₂NR₂—, and —NR₂S(O)₂—, wherein R₂ is as previously defined;
\textcircled{C} is absent or selected from the groups consisting of optionally substituted aryl, optionally substituted heteroaryl, optionally substituted arylalkyl, optionally substituted heteroarylalkyl optionally substituted —C₃-C₁₂ cycloalkyl, optionally substituted 3- to 12-membered heterocycloalkyl, and optionally substituted —C₃-C₁₂ cycloalkenyl;
\textcircled{A} is optionally substituted aryl, optionally substituted heteroaryl, optionally substituted arylalkyl, optionally substituted heteroarylalkyl, optionally substituted —C₃-C₁₂ cycloalkyl, optionally substituted 3- to 12-membered heterocycloalkyl; or optionally substituted —C₃-C₁₂ cycloalkenyl;
alternatively, L₄ is NR₁, R₁ is connected to \textcircled{A}, and R₁ and the nitrogen atom to which it is attached form a heterocyclic ring;
R is absent or selected from the group consisting of hydroxy, protected hydroxy, —O-(hydroxy prodrug group), tetrazolyl, 1-methyltetrazolyl, —CH₂CO₂R₃, —CO₂R₃, —C(O)N(R₃)O(R₃), —C(O)NR₄R₅, —P(O)(OR₆)₂, —C(O)NR₇S(O)₂R₈, —S(O)₂R₈, —CH₂OH, —CH₂OC(O)NR₄R₅, and —CH₂OC(O)NR₇S(O)₂R₈;
each R₃ is independently selected from the groups consisting of hydrogen, optionally substituted —C₁-C₈ alkyl, and optionally substituted —C₃-C₈ cycloalkyl;
R₄ and R₅ are each independently selected from the group consisting of hydrogen, optionally substituted —C₁-C₈ alkyl, and optionally substituted —C₃-C₈ cycloalkyl; alternatively, R₄ and R₅ are taken together with the nitrogen atom to which they are attached to form a heterocyclic ring;
R₆ is selected from the group consisting of hydrogen, optionally substituted —C₁-C₈ alkyl, optionally substituted —C₃-C₈ cycloalkyl, optionally substituted 3- to 8-membered heterocycloalkyl, optionally substituted aryl, and optionally substituted heteroaryl;
R₇ is selected from the group consisting of hydrogen, optionally substituted —C₁-C₆ alkyl, and optionally substituted —C₃-C₆ cycloalkyl;
R₈ is selected from the group consisting of NR₄R₅ optionally substituted —C₁-C₈ alkyl, optionally substituted —C₃-C₈ cycloalkyl, optionally substituted 3- to 8-membered heterocycloalkyl, optionally substituted aryl, and optionally substituted heteroaryl;
provided that at least one of \textcircled{B}, \textcircled{B₁}, and \textcircled{C} is not absent;
wherein the fibrotic disease is not liver fibrosis.

2. The method of claim 1, wherein the compound is represented by one of Formulae (V-1) to (V-10) or a pharmaceutically acceptable salt thereof: 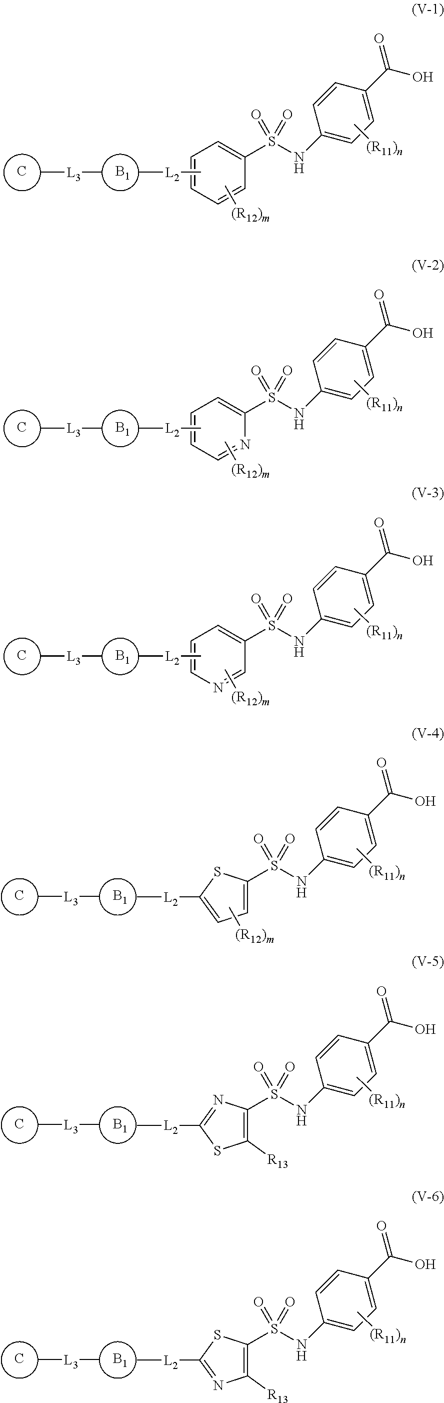 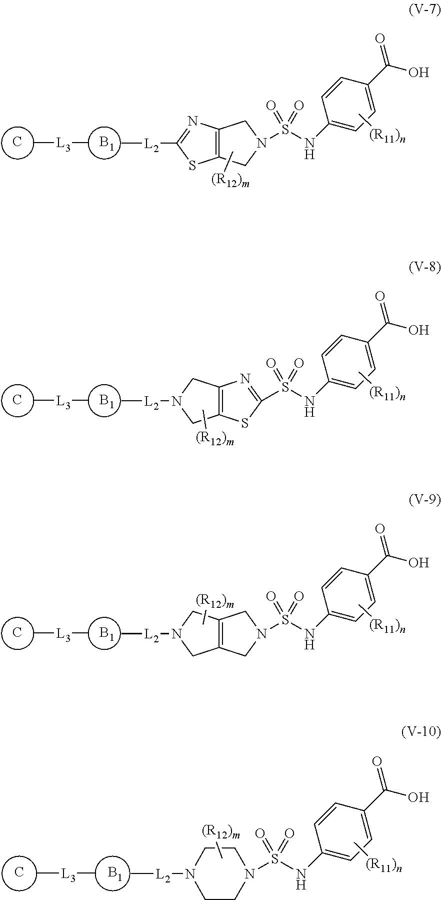
wherein each R₁₁ is independently selected from the groups consisting of halogen, hydroxy, —NH₂, —NHMe, —NMe₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; n is 0, 1, 2, or 3; each R₁₂ is independently selected from the groups consisting of halogen, hydroxy, —NH₂, —NHMe, —NMe₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; m is 0, 1 or 2; each R₁₃ is independently selected from the groups consisting of hydrogen, halogen, hydroxy, —NH₂, —NHMe, —NMe₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; \textcircled{B₁}, \textcircled{C}, L₂, and L₃ are as defined in claim 1.

3. The method of claim 1, wherein the compound is represented by one of Formulae (X-1) to (X-12), or a pharmaceutically acceptable salt thereof: 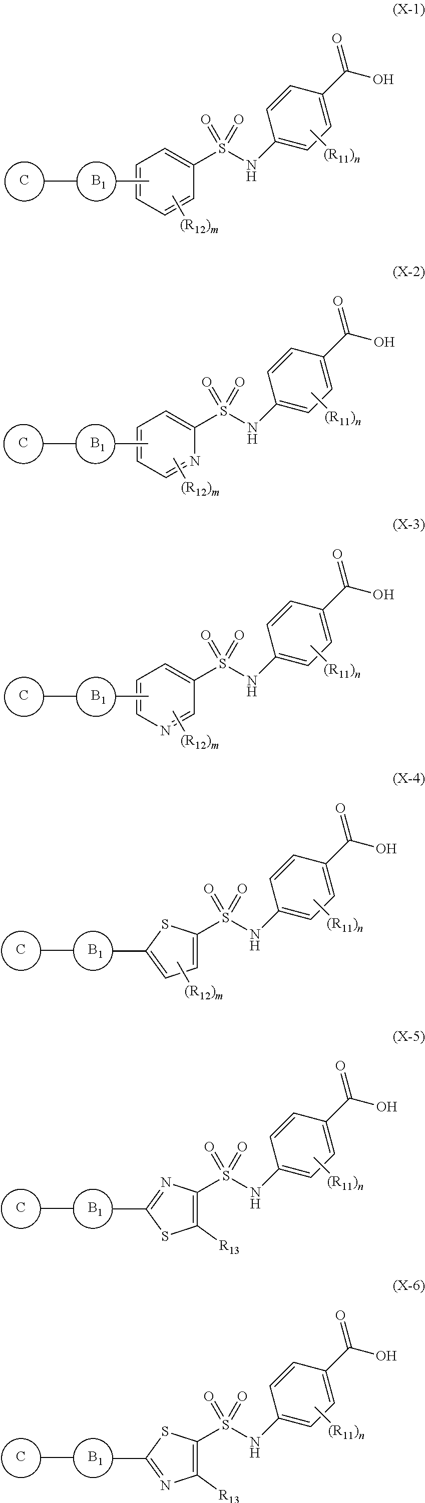 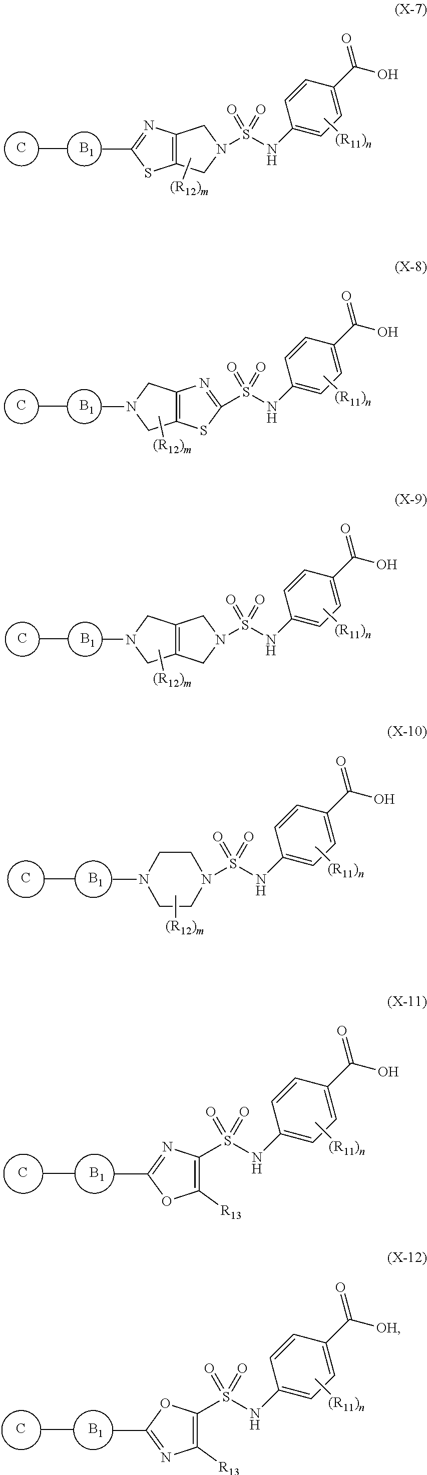
wherein each R₁₁ is independently selected from the groups consisting of halogen, hydroxy, —NH₂, —NHMe, —NMe₂, —CN, —NO₂, optionally substituted —C₁-C₆ alkyl, optionally substituted —C₁-C₆ alkoxyl, optionally substituted —C₃-C₆ cycloalkyl, optionally substituted —C₃-C₆ cycloalkenyl, optionally substituted aryl, and optionally substituted heteroaryl; n is 0, 1, 2, or 3; each R₁₂ is independently selected from the groups consisting of halogen, hydroxy, —NH₂, —NHMe, -Nme₂, —CN, —NO₂, optionally substituted —C₁-C₆ alkyl, optionally substituted —C₁-C₆ alkoxyl, optionally substituted —C₃-C₆ cycloalkyl, optionally substituted —C₃-C₆ cycloalkenyl, optionally substituted aryl, and optionally substituted heteroaryl; m is 0, 1 or 2; each R₁₃ is independently selected from the group consisting of hydrogen, halogen, hydroxy, —NH₂, —NHMe, -Nme₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; \textcircled{B₁}, and \textcircled{C} are as defined in claim 1.

4. The method of claim 1, wherein the compound is represented by one of Formulae (XI-1) to (XI-22), or a pharmaceutically acceptable salt thereof: 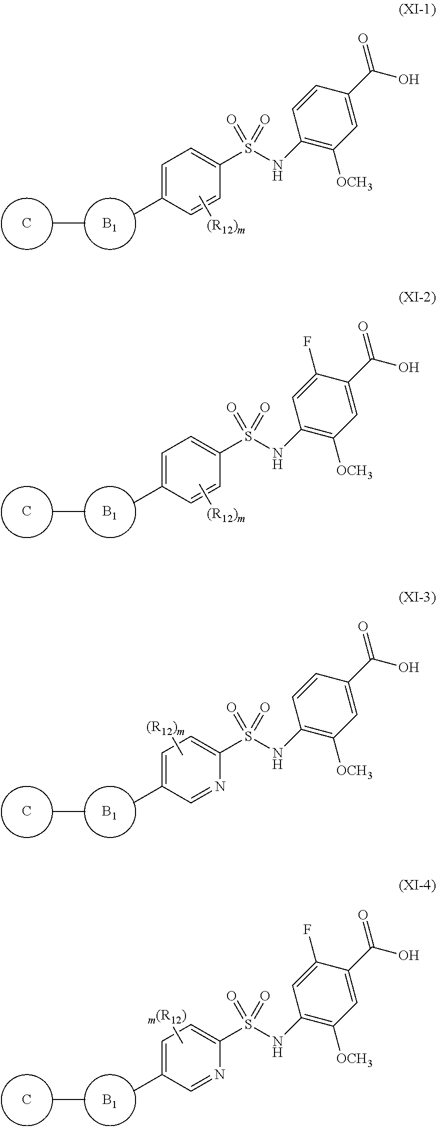 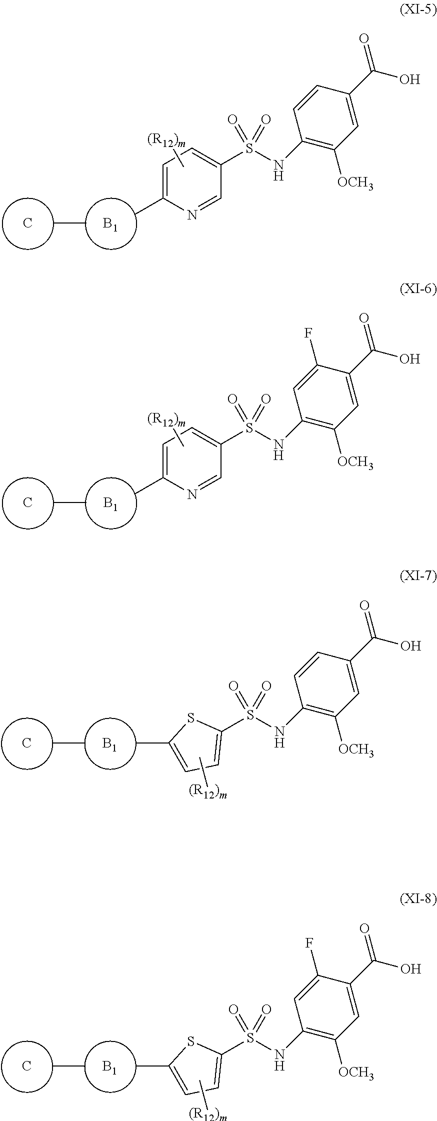 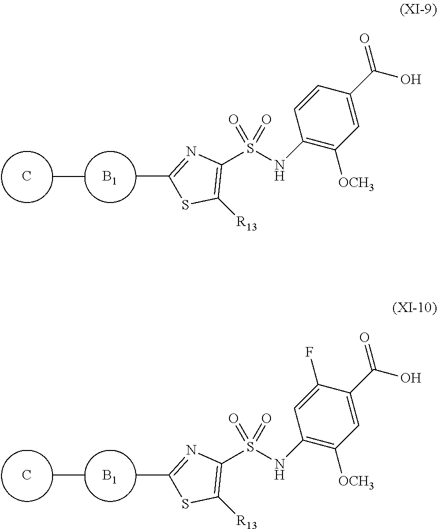 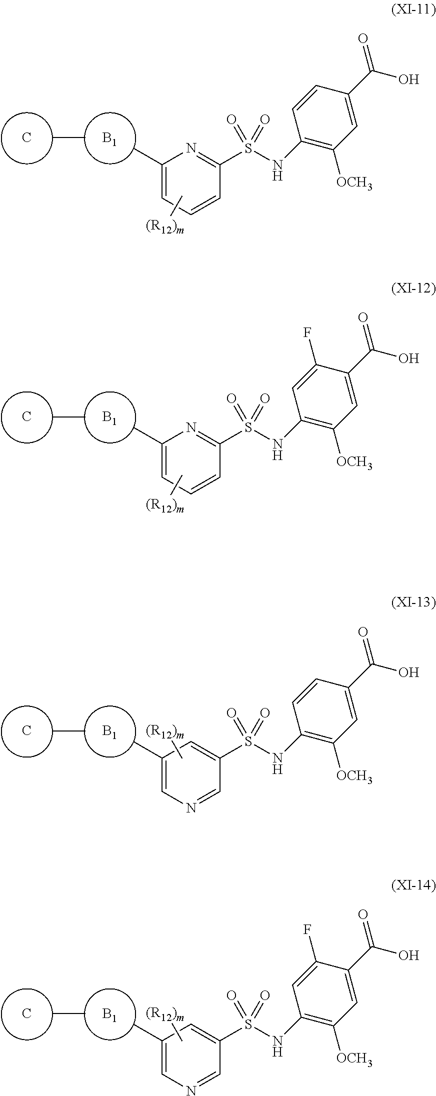 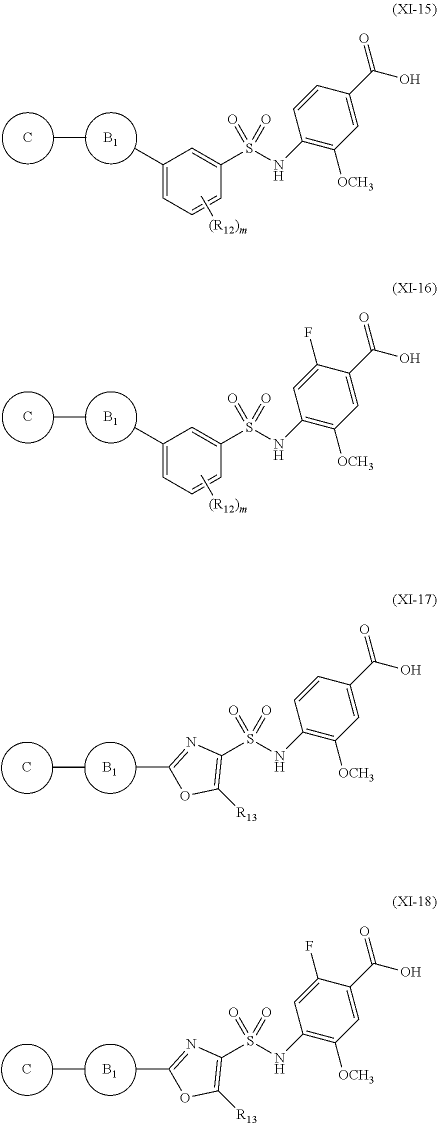 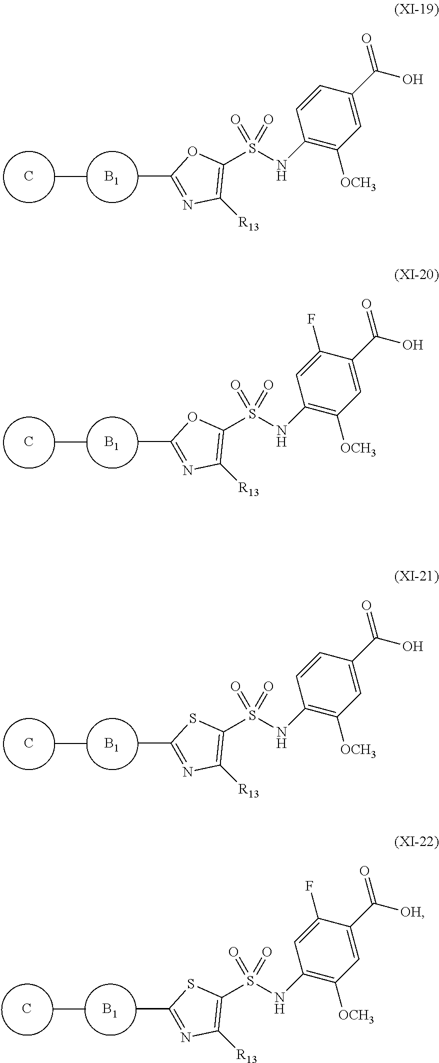
wherein each R₁₂ is independently selected from the groups consisting of halogen, hydroxy, —NH₂, —NHMe, -Nme₂, —CN, —NO₂, optionally substituted —C₁-C₆ alkyl, optionally substituted —C₁-C₆ alkoxyl, optionally substituted —C₃-C₆ cycloalkyl, optionally substituted —C₃-C₆ cycloalkenyl, optionally substituted aryl, and optionally substituted heteroaryl; m is 0, 1 or 2; each R₁₃ is independently selected from the group consisting of hydrogen, halogen, hydroxy, —NH₂, —NHMe, -Nme₂, —CN, —NO₂, optionally substituted —C₁-C₆ alkyl, optionally substituted —C₁-C₆ alkoxyl, optionally substituted C₃-C₆ cycloalkyl, optionally substituted C₃-C₆ cycloalkenyl, optionally substituted aryl, and optionally substituted heteroaryl; \textcircled{B₁}, and \textcircled{C} are as defined in claim 1.

5. The method of claim 1, wherein the fibrotic disease is lung fibrosis, kidney fibrosis, brain fibrosis or heart fibrosis.

6. The method of claim 5, wherein the fibrotic disease is an interstitial lung fibrotic disease.

7. The method of claim 6, wherein the interstitial lung fibrotic disease (ILD) is idiopathic pulmonary fibrosis, acute interstitial pneumonia, non-specific interstitial pneumonia, cryptogenic organizing pneumonia, systemic lupus erythematosus-related ILD, scleroderma-related ILD, rheumatoid arthritis-related ILD, drug-induced ILD, environmentally-induced ILD, or asthma.

8. The method of claim 7, wherein interstitial lung fibrotic disease is idiopathic pulmonary fibrosis.
